SEQUENCE LISTING (171 sequences)

SEQ ID NO: 1 - DNA, 1395 bp, Myceliophthora thermophila
atggggcagaagactctccaggggctggtggcggcggcggcactggcagcctcggtggcgaacgcgcagcaaccgggcaccttcacgcccgaggtgcatccgacgctgccgacgtggaagtgcacgacgagcggcgggtgcgtccagcaggacacgtcggtggtgctcgactggaactaccgctggttccacaccgaggacggtagcaagtcgtgcatcacctctagcggcgtcgaccggaccctgtgcccggacgaggcgacgtgcgccaagaactgcttcgtcgagggcgtcaactacacgagcagcggggtcgagacgtccggcagctccctcaccctccgccagttcttcaagggctccgacggcgccatcaacagcgtctccccgcgcgtctacctgctcgggggagacggcaactatgtcgtgctcaagctcctcggccaggagctgagcttcgacgtggacgtatcgtcgctcccgtgcggcgagaacgcggccctgtacctgtccgagatggacgcgacgggaggacggaacgagtacaacacgggcggggccgagtacgggtcgggctactgtgacgcccagtgccccgtgcagaactggaacaacgggacgctcaacacgggccgggtgggctcgtgctgcaacgagatggacatcctcgaggccaactccaaggccgaggccttcacgccgcacccctgcatcggcaactcgtgcgacaagagcgggtgcggcttcaacgcgtacgcgcgcggttaccacaactactgggcccccggcggcacgctcgacacgtcccggcctttcaccatgatcacccgcttcgtcaccgacgacggcaccacctcgggcaagctcgcccgcatcgagcgcgtctacgtccaggacggcaagaaggtgcccagcgcggcgcccgggggggacgtcatcacggccgacgggtgcacctccgcgcagccctacggcggcctttccggcatgggcgacgccctcggccgcggcatggtcctggccctgagcatctggaacgacgcgtccgggtacatgaactggctcgacgccggcagcaacggcccctgcagcgacaccgagggtaacccgtccaacatcctggccaaccacccggacgcccacgtcgtgctctccaacatccgctggggcgacatcggctccaccgtcgacaccggcgatggcgacaacaacggcggcggccccaacccgtcatccaccaccaccgctaccgctaccaccacctcctccggcccggccgagcctacccagacccactacggccagtgtggagggaaaggatggacgggccctacccgctgcgagacgccctacacctgcaagtaccagaacgactggtactcgcagtgcctgtag

SEQ ID NO: 2 - protein, 464 aa, Myceliophthora thermophila
MGQKTLQGLVAAAALAASVANAQQPGTFTPEVHPTLPTWKCTTSGGCVQQDTSVVLDWNYRWFHTEDGSKSCITSSGVDRTLCPDEATCAKNCFVEGVNYTSSGVETSGSSLTLRQFFKGSDGAINSVSPRVYLLGGDGNYVVLKLLGQELSFDVDVSSLPCGENAALYLSEMDATGGRNEYNTGGAEYGSGYCDAQCPVQNWNNGTLNTGRVGSCCNEMDILEANSKAEAFTPHPCIGNSCDKSGCGFNAYARGYHNYWAPGGTLDTSRPFTMITRFVTDDGTTSGKLARIERVYVQDGKKVPSAAPGGDVITADGCTSAQPYGGLSGMGDALGRGMVLALSIWNDASGYMNWLDAGSNGPCSDTEGNPSNILANHPDAHVVLSNIRWGDIGSTVDTGDGDNNGGGPNPSSTTTATATTTSSGPAEPTQTHYGQCGGKGWTGPTRCETPYTCKYQNDWYSQCL

SEQ ID NO: 3 - protein, 442 aa, Myceliophthora thermophila
QQPGTFTPEVHPTLPTWKCTTSGGCVQQDTSVVLDWNYRWFHTEDGSKSCITSSGVDRTLCPDEATCAKNCFVEGVNYTSSGVETSGSSLTLRQFFKGSDGAINSVSPRVYLLGGDGNYVVLKLLGQELSFDVDVSSLPCGENAALYLSEMDATGGRNEYNTGGAEYGSGYCDAQCPVQNWNNGTLNTGRVGSCCNEMDILEANSKAEAFTPHPCIGNSCDKSGCGFNAYARGYHNYWAPGGTLDTSRPFTMITRFVTDDGTTSGKLARIERVYVQDGKKVPSAAPGGDVITADGCTSAQPYGGLSGMGDALGRGMVLALSIWNDASGYMNWLDAGSNGPCSDTEGNPSNILANHPDAHVVLSNIRWGDIGSTVDTGDGDNNGGGPNPSSTTTATATTTSSGPAEPTQTHYGQCGGKGWTGPTRCETPYTCKYQNDWYSQCL

SEQ ID NO: 4 - DNA, 1029 bp, Myceliophthora thermophila
atgtccaaggcctctgctctcctcgctggcctgacgggcgcggccctcgtcgctgcacatggccacgtcagccacatcgtcgtcaacggcgtctactacaggaactacgaccccacgacagactggtaccagcccaacccgccaacagtcatcggctggacggcagccgatcaggataatggcttcgttgaacccaacagctttggcacgccagatatcatctgccacaagagcgccacccccggcggcggccacgctaccgttgctgccggagacaagatcaacatcgtctggacccccgagtggcccgaatcccacatcggccccgtcattgactacctagccgcctgcaacggtgactgcgagaccgtcgacaagtcgtcgctgcgctggttcaagattgacggcgccggctacgacaaggccgccggccgctgggccgccgacgctctgcgcgccaacggcaacagctggctcgtccagatcccgtcggatctcaaggccggcaactacgtcctccgccacgagatcatcgccctccacggtgctcagagccccaacggcgcccaggcctacccgcagtgcatcaacctccgcgtcaccggcggcggcagcaacctgcccagcggcgtcgccggcacctcgctgtacaaggcgaccgacccgggcatcctcttcaacccctacgtctcctccccggattacaccgtccccggcccggccctcattgccggcgccgccagctcgatcgcccagagcacgtcggtcgccactgccaccggcacggccaccgttcccggcggcggcggcgccaaccctaccgccaccaccaccgccgccacctccgccgccccgagcaccaccctgaggacgaccactacctcggccgcgcagactaccgccccgccctccggcgatgtgcagaccaagtacggccagtgtggtggcaacggatggacgggcccgacggtgtgcgcccccggctcgagctgctccgtcctcaacgagtggtactcccagtgtttgtaa

SEQ ID NO: 5 - protein, 342 aa, Myceliophthora thermophila
MSKASALLAGLTGAALVAAHGHVSHIVVNGVYYRNYDPTTDWYQPNPPTVIGWTAADQDNGFVEPNSFGTPDIICHKSATPGGGHATVAAGDKINIVWTPEWPESHIGPVIDYLAACNGDCETVDKSSLRWFKIDGAGYDKAAGRWAADALRANGNSWLVQIPSDLKAGNYVLRHEIIALHGAQSPNGAQAYPQCINLRVTGGGSNLPSGVAGTSLYKATDPGILFNPYVSSPDYTVPGPALIAGAASSIAQSTSVATATGTATVPGGGGANPTATTTAATSAAPSTTLRTTTTSAAQTTAPPSGDVQTKYGQCGGNGWTGPTVCAPGSSCSVLNEWYSQCL

SEQ ID NO: 6 - protein, 323 aa, Myceliophthora thermophila
HGHVSHIVVNGVYYRNYDPTTDWYQPNPPTVIGWTAADQDNGFVEPNSFGTPDIICHKSATPGGGHATVAAGDKINIVWTPEWPESHIGPVIDYLAACNGDCETVDKSSLRWFKIDGAGYDKAAGRWAADALRANGNSWLVQIPSDLKAGNYVLRHEIIALHGAQSPNGAQAYPQCINLRVTGGGSNLPSGVAGTSLYKATDPGILFNPYVSSPDYTVPGPALIAGAASSIAQSTSVATATGTATVPGGGGANPTATTTAATSAAPSTTLRTTTTSAAQTTAPPSGDVQTKYGQCGGNGWTGPTVCAPGSSCSVLNEWYSQCL

SEQ ID NO: 7 - DNA, 1029 bp, Artificial Sequence
  Synthetic polynucleotide for GH61a Variant 1
atgtccaaggcctctgctctcctcgctggcctgacgggcgcggccctcgtcgctgcacacggccacgtcagccacatcgtcgtcaacggcgtctactacaggggctacgaccccacgacagactggtaccagcccaacccgccaacagtcatcggctggacggcagccgatcaggataatggcttcgttgaacccaacagctttggcacgccagatatcatctgccacaagagcgccacccccggcggcggccacgctaccgttgctgccggagacaagatcaacatcgtctggacccccgagtggccccactcccacatcggccccgtcattgactacctagccgcctgcaacggtgactgcgagaccgtcgacaagtcgtcgctgcgctggttcaagattgacggcgccggctacgacaaggccgccggccgctgggccgccgacgctctgcgcgccaacggcaacagctggctcgtccagatcccgtcggatctcaagcccggcaactacgtcctccgccacgagatcatcgccctccacggtgctcagagccccaacggcgcccaggcgtacccgcagtgcatcaacctccgcgtcaccggcggcggcagcaacctgcccagcggcgtcgccggcacctcgctgtacaaggcgaccgacccgggcatcctcttcaacccctacgtctcctccccggattacaccgtccccggcccggccctcattgccggcgccgccagctcgatcgcccagagcacgtcggtcgccactgccaccggcacggccaccgttcccggcggcggcggcgccaaccctaccgccaccaccaccgccgccacctccgccgccccgagcaccaccctgaggacgaccactacctcggccgcgcagactaccgccccgccctccggcgatgtgcagaccaagtacggccagtgtggtggcaacggatggacgggcccgacggtgtgcgcccccggctcgagctgctccgtcctcaacgagtggtactcccagtgtttgtaa

SEQ ID NO: 8 - protein, 342 aa, Artificial Sequence
  Synthetic polypeptide for GH61a Variant 1
MSKASALLAGLTGAALVAAHGHVSHIVVNGVYYRGYDPTTDWYQPNPPTVIGWTAADQDNGFVEPNSFGTPDIICHKSATPGGGHATVAAGDKINIVWTPEWPHSHIGPVIDYLAACNGDCETVDKSSLRWFKIDGAGYDKAAGRWAADALRANGNSWLVQIPSDLKPGNYVLRHEIIALHGAQSPNGAQAYPQCINLRVTGGGSNLPSGVAGTSLYKATDPGILFNPYVSSPDYTVPGPALIAGAASSIAQSTSVATATGTATVPGGGGANPTATTTAATSAAPSTTLRTTTTSAAQTTAPPSGDVQTKYGQCGGNGWTGPTVCAPGSSCSVLNEWYSQCL

SEQ ID NO: 9 - protein, 323 aa, Artificial Sequence
  Synthetic polypeptide for GH61a Variant 1
HGHVSHIVVNGVYYRGYDPTTDWYQPNPPTVIGWTAADQDNGFVEPNSFGTPDIICHKSATPGGGHATVAAGDKINIVWTPEWPHSHIGPVIDYLAACNGDCETVDKSSLRWFKIDGAGYDKAAGRWAADALRANGNSWLVQIPSDLKPGNYVLRHEIIALHGAQSPNGAQAYPQCINLRVTGGGSNLPSGVAGTSLYKATDPGILFNPYVSSPDYTVPGPALIAGAASSIAQSTSVATATGTATVPGGGGANPTATTTAATSAAPSTTLRTTTTSAAQTTAPPSGDVQTKYGQCGGNGWTGPTVCAPGSSCSVLNEWYSQCL

SEQ ID NO: 10 - DNA, 1035 bp, Artificial Sequence
  Synthetic polynucleotide for GH61a Variant 5
acacaaatgtccaaggcctctgctctcctcgctggcctgacgggcgcggccctcgtcgctgcacacggccacgtcagccacatcgtcgtcaacggcgtctactacaggaactacgaccccacgacagactggtaccagcccaacccgccaacagtcatcggctggacggcagccgatcaggataatggcttcgttgaacccaacagctttggcacgccagatatcatctgccacaagagcgccacccccggcggcggccacgctaccgttgctgccggagacaagatcaacatcgtatggacccccgagtggccccactcccacatcggccccgtcattgactacctagccgcctgcaacggtgactgcgagaccgtcgacaagtcgtcgctgcgctggttcaagattgacggcgccggctacgacaaggccgccggccgctgggccgccgacgctctgcgcgccaacggcaacagctggctcgtccagatcccgtcggatctcgcggccggcaactacgtcctccgccacgagatcatcgccctccacggtgctcagagccccaacggcgcccaggcgtacccgcagtgcatcaacctccgcgtcaccggcggcggcagcaacctgcccagcggcgtcgccggcacctcgctgtacaaggcgaccgacccgggcatcctcttcaacccctacgtctcctccccggattacaccgtccccggcccggccctcattgccggcgccgccagctcgatcgcccagagcacgtcggtcgccactgccaccggcacggccaccgttcccggcggcggcggcgccaaccctaccgccaccaccaccgccgccacctccgccgccccgagcaccaccctgaggacgaccactacctcggccgcgcagactaccgccccgccctccggcgatgtgcagaccaagtacggccagtgtggtggcaacggatggacgggcccgacggtgtgcgcccccggctcgagctgctccgtcctcaacgagtggtactcccagtgtttgtaa

SEQ ID NO: 11 - protein, 342 aa, Artificial Sequence
  Synthetic polypeptide for GH61a Variant 5
MSKASALLAGLTGAALVAAHGHVSHIVVNGVYYRNYDPTTDWYQPNPPTVIGWTAADQDNGFVEPNSFGTPDIICHKSATPGGGHATVAAGDKINIVWTPEWPHSHIGPVIDYLAACNGDCETVDKSSLRWFKIDGAGYDKAAGRWAADALRANGNSWLVQIPSDLAAGNYVLRHEIIALHGAQSPNGAQAYPQCINLRVTGGGSNLPSGVAGTSLYKATDPGILFNPYVSSPDYTVPGPALIAGAASSIAQSTSVATATGTATVPGGGGANPTATTTAATSAAPSTTLRTTTTSAAQTTAPPSGDVQTKYGQCGGNGWTGPTVCAPGSSCSVLNEWYSQCL

SEQ ID NO: 12 - protein, 323 aa, Artificial Sequence
  Synthetic polypeptide for GH61a Variant 5
HGHVSHIVVNGVYYRNYDPTTDWYQPNPPTVIGWTAADQDNGFVEPNSFGTPDIICHKSATPGGGHATVAAGDKINIVWTPEWPHSHIGPVIDYLAACNGDCETVDKSSLRWFKIDGAGYDKAAGRWAADALRANGNSWLVQIPSDLAAGNYVLRHEIIALHGAQSPNGAQAYPQCINLRVTGGGSNLPSGVAGTSLYKATDPGILFNPYVSSPDYTVPGPALIAGAASSIAQSTSVATATGTATVPGGGGANPTATTTAATSAAPSTTLRTTTTSAAQTTAPPSGDVQTKYGQCGGNGWTGPTVCAPGSSCSVLNEWYSQCL

SEQ ID NO: 13 - DNA, 1035 bp, Artificial Sequence
  Synthetic polynucleotide for GH61a Variant 9
acaaacatgtccaaggcctctgctctcctcgctggcctgacgggcgcggccctcgtcgctgcacatggccacgtcagccacatcgtcgtcaacggcgtctactacaggaactacgaccccacgacagactggtaccagcccaacccgccaacagtcatcggctggacggcagccgatcaggataatggcttcgttgaacccaacagctttggcacgccagatatcatctgccacaagagcgccacccccggcggcggccacgctaccgttgctgccggagacaagatcaacatccagtggacccccgagtggcccgaatcccacatcggccccgtcattgactacctagccgcctgcaacggtgactgcgagaccgtcgacaagtcgtcgctgcgctggttcaagattgacggcgccggctacgacaaggccgccggccgctgggccgccgacgctctgcgcgccaacggcaacagctggctcgtccagatcccgtcggatctcaaggccggcaactacgtcctccgccacgagatcatcgccctccacggtgctcagagccccaacggcgcccagaactacccgcagtgcatcaacctccgcgtcaccggcggcggcagcaacctgcccagcggcgtcgccggcacctcgctgtacaaggcgaccgacccgggcatcctcttcaacccctacgtctcctccccggattacaccgtccccggcccggccctcattgccggcgccgccagctcgatcgcccagagcacgtcggtcgccactgccaccggcacggccaccgttcccggcggcggcggcgccaaccctaccgccaccaccaccgccgccacctccgccgccccgagcaccaccctgaggacgaccactacctcggccgcgcagactaccgccccgccctccggcgatgtgcagaccaagtacggccagtgtggtggcaacggatggacgggcccgacggtgtgcgcccccggctcgagctgctccgtcctcaacgagtggtactcccagtgtttgtaa

SEQ ID NO: 14 - protein, 342 aa, Artificial Sequence
  Synthetic polypeptide for GH61a Variant 9
MSKASALLAGLTGAALVAAHGHVSHIVVNGVYYRNYDPTTDWYQPNPPTVIGWTAADQDNGFVEPNSFGTPDIICHKSATPGGGHATVAAGDKINIQWTPEWPESHIGPVIDYLAACNGDCETVDKSSLRWFKIDGAGYDKAAGRWAADALRANGNSWLVQIPSDLKAGNYVLRHEIIALHGAQSPNGAQNYPQCINLRVTGGGSNLPSGVAGTSLYKATDPGILFNPYVSSPDYTVPGPALIAGAASSIAQSTSVATATGTATVPGGGGANPTATTTAATSAAPSTTLRTTTTSAAQTTAPPSGDVQTKYGQCGGNGWTGPTVCAPGSSCSVLNEWYSQCL

SEQ ID NO: 15 - protein, 323 aa, Artificial Sequence
  Synthetic polypeptide for GH61a Variant 9
HGHVSHIVVNGVYYRNYDPTTDWYQPNPPTVIGWTAADQDNGFVEPNSFGTPDIICHKSATPGGGHATVAAGDKINIQWTPEWPESHIGPVIDYLAACNGDCETVDKSSLRWFKIDGAGYDKAAGRWAADALRANGNSWLVQIPSDLKAGNYVLRHEIIALHGAQSPNGAQNYPQCINLRVTGGGSNLPSGVAGTSLYKATDPGILFNPYVSSPDYTVPGPALIAGAASSIAQSTSVATATGTATVPGGGGANPTATTTAATSAAPSTTLRTTTTSAAQTTAPPSGDVQTKYGQCGGNGWTGPTVCAPGSSCSVLNEWYSQCL

SEQ ID NO: 16 - DNA, 738 bp, Myceliophthora thermophila
atgaagctctccctcttttccgtcctggccactgccctcaccgtcgaggggcatgccatcttccagaaggtctccgtcaacggagcggaccagggctccctcaccggcctccgcgctcccaacaacaacaaccccgtgcagaatgtcaacagccaggacatgatctgcggccagtcgggatcgacgtcgaacactatcatcgaggtcaaggccggcgataggatcggtgcctggtatcagcatgtcatcggcggtgcccagttccccaacgacccagacaacccgattgccaagtcgcacaagggccccgtcatggcctacctcgccaaggttgacaatgccgcaaccgccagcaagacgggcctgaagtggttcaagatttgggaggatacctttaatcccagcaccaagacctggggtgtcgacaacctcatcaacaacaacggctgggtgtacttcaacctcccgcagtgcatcgccgacggcaactacctcctccgcgtcgaggtcctcgctctgcactcggcctactcccagggccaggctcagttctaccagtcctgcgcccagatcaacgtatccggcggcggctccttcacgccggcgtcgactgtcagcttcccgggtgcctacagcgccagcgaccccggtatcctgatcaacatctacggcgccaccggccagcccgacaacaacggccagccgtacactgcccctgggcccgcgcccatctcctgc

SEQ ID NO: 17 - protein, 246 aa, Myceliophthora thermophila
MKLSLFSVLATALTVEGHAIFQKVSVNGADQGSLTGLRAPNNNNPVQNVNSQDMICGQSGSTSNTIIEVKAGDRIGAWYQHVIGGAQFPNDPDNPIAKSHKGPVMAYLAKVDNAATASKTGLKWFKIWEDTFNPSTKTWGVDNLINNNGWVYFNLPQCIADGNYLLRVEVLALHSAYSQGQAQFYQSCAQINVSGGGSFTPASTVSFPGAYSASDPGILINIYGATGQPDNNGQPYTAPGPAPISC

SEQ ID NO: 18 - protein, 227 aa, Myceliophthora thermophila
IFQKVSVNGADQGSLTGLRAPNNNNPVQNVNSQDMICGQSGSTSNTIIEVKAGDRIGAWYQHVIGGAQFPNDPDNPIAKSHKGPVMAYLAKVDNAATASKTGLKWFKIWEDTFNPSTKTWGVDNLINNNGWVYFNLPQCIADGNYLLRVEVLALHSAYSQGQAQFYQSCAQINVSGGGSFTPASTVSFPGAYSASDPGILINIYGATGQPDNNGQPYTAPGPAPISC

SEQ ID NO: 19 - DNA, 762 bp, Myceliophthora thermophila
atggccctccagctcttggcgagcttggccctcctctcagtgccggcccttgcccacggtggcttggccaactacaccgtcggtgatacttggtacagaggctacgacccaaacctgccgccggagacgcagctcaaccagacctggatgatccagcggcaatgggccaccatcgaccccgtcttcaccgtgtcggagccgtacctggcctgcaacaacccgggcgcgccgccgccctcgtacatccccatccgcgccggtgacaagatcacggccgtgtactggtactggctgcacgccatcgggcccatgagcgtctggctcgcgcggtgcggcgacacgcccgcggccgactgccgcgacgtcgacgtcaaccgggtcggctggttcaagatctgggagggcggcctgctggagggtcccaacctggccgaggggctctggtaccaaaaggacttccagcgctgggacggctccccgtccctctggcccgtcacgatccccaaggggctcaagagcgggacctacatcatccggcacgagatcctgtcgcttcacgtcgccctcaagccccagttttacccggagtgtgcgcatctgaatattactgggggcggagacttgctgccacccgaagagactctggtgcggtttccgggggtttacaaagaggacgatccctctatcttcatcgatgtctactcggaggagaacgcgaaccggacagattatacggttccgggagggccaatctgggaaggg

SEQ ID NO: 20 - protein, 254 aa, Myceliophthora thermophila
MALQLLASLALLSVPALAHGGLANYTVGDTWYRGYDPNLPPETQLNQTWMIQRQWATIDPVFTVSEPYLACNNPGAPPPSYIPIRAGDKITAVYWYWLHAIGPMSVWLARCGDTPAADCRDVDVNRVGWFKIWEGGLLEGPNLAEGLWYQKDFQRWDGSPSLWPVTIPKGLKSGTYIIRHEILSLHVALKPQFYPECAHLNITGGGDLLPPEETLVRFPGVYKEDDPSIFIDVYSEENANRTDYTVPGGPIWEG

SEQ ID NO: 21 - protein, 231 aa, Myceliophthora thermophila
NYTVGDTWYRGYDPNLPPETQLNQTWMIQRQWATIDPVFTVSEPYLACNNPGAPPPSYIPIRAGDKITAVYWYWLHAIGPMSVWLARCGDTPAADCRDVDVNRVGWFKIWEGGLLEGPNLAEGLWYQKDFQRWDGSPSLWPVTIPKGLKSGTYIIRHEILSLHVALKPQFYPECAHLNITGGGDLLPPEETLVRFPGVYKEDDPSIFIDVYSEENANRTDYTVPGGPIWEG

SEQ ID NO: 22 - DNA, 705 bp, Myceliophthora thermophila
atgaaggccctctctctccttgcggctgccggggcagtctctgcgcataccatcttcgtccagctcgaagcagacggcacgaggtacccggtttcgtacgggatccgggacccaacctacgacggccccatcaccgacgtcacatccaacgacgttgcttgcaacggcggtccgaacccgacgaccccctccagcgacgtcatcaccgtcaccgcgggcaccaccgtcaaggccatctggaggcacaccctccaatccggcccggacgatgtcatggacgccagccacaagggcccgaccctggcctacatcaagaaggtcggcgatgccaccaaggactcgggcgtcggcggtggctggttcaagatccaggaggacggttacaacaacggccagtggggcaccagcaccgttatctccaacggcggcgagcactacattgacatcccggcctgcatccccgagggtcagtacctcctccgcgccgagatgatcgccctccacgcggccgggtcccccggcggcgctcagctctacatggaatgtgcccagatcaacatcgtcggcggctccggctcggtgcccagctcgacggtcagcttccccggcgcgtatagccccaacgacccgggtctcctcatcaacatctattccatgtcgccctcgagctcgtacaccatcccgggcccgcccgttttcaagtgc

SEQ ID NO: 23 - protein, 235 aa, Myceliophthora thermophila
MKALSLLAAAGAVSAHTIFVQLEADGTRYPVSYGIRDPTYDGPITDVTSNDVACNGGPNPTTPSSDVITVTAGTTVKAIWRHTLQSGPDDVMDASHKGPTLAYIKKVGDATKDSGVGGGWFKIQEDGYNNGQWGTSTVISNGGEHYIDIPACIPEGQYLLRAEMIALHAAGSPGGAQLYMECAQINIVGGSGSVPSSTVSFPGAYSPNDPGLLINIYSMSPSSSYTIPGPPVFKC

SEQ ID NO: 24 - protein, 220 aa, Myceliophthora thermophila
HTIFVQLEADGTRYPVSYGIRDPTYDGPITDVTSNDVACNGGPNPTTPSSDVITVTAGTTVKAIWRHTLQSGPDDVMDASHKGPTLAYIKKVGDATKDSGVGGGWFKIQEDGYNNGQWGTSTVISNGGEHYIDIPACIPEGQYLLRAEMIALHAAGSPGGAQLYMECAQINIVGGSGSVPSSTVSFPGAYSPNDPGLLINIYSMSPSSSYTIPGPPVFKC

SEQ ID NO: 25 - DNA, 915 bp, Myceliophthora thermophila
atgaagtcgtctaccccggccttgttcgccgctgggctccttgctcagcatgctgcggcccactccatcttccagcaggcgagcagcggctcgaccgactttgatacgctgtgcacccggatgccgcccaacaatagccccgtcactagtgtgaccagcggcgacatgacctgcaaagtcggcggcaccaagggggtgtccggcttctgcgaggtgaacgccggcgacgagttcacggttgagatgcacgcgcagcccggcgaccgctcgtgcgccaacgaggccatcggcgggaaccacttcggcccggtcctcatctacatgagcaaggtcgacgacgcctccaccgccgacgggtccggcgactggttcaaggtggacgagttcggctacgacgcaagcaccaagacctggggcaccgacaagctcaacgagaactgcggcaagcgcaccttcaacatccccagccacatccccgcgggcgactatctcgtccgggccgaggctatcgcgctacacactgccaaccagccaggcggcgcgcagttctacatgagctgctatcaagtcaggatttccggcggcgaagggggccagctgcctgccggagtcaagatcccgggcgcgtacagtgccaacgaccccggcatccttgtcgacatctggggtaacgatttcaacgaccctccaggacactcggcccgtcacgccatcatcatcatcagcagcagcagcaacaacagcggcgccaagatgaccaagaagatccaggagcccaccatcacatcggtcacggacctccccaccgacgaggccaagtggatcgcgctccaaaagatctcgtacgtggaccagacgggcacggcgcggacatacgagccggcgtcgcgcaagacgcggtcgccaagagtctag

SEQ ID NO: 26 - protein, 304 aa, Myceliophthora thermophila
MKSSTPALFAAGLLAQHAAAHSIFQQASSGSTDFDTLCTRMPPNNSPVTSVTSGDMTCKVGGTKGVSGFCEVNAGDEFTVEMHAQPGDRSCANEAIGGNHFGPVLIYMSKVDDASTADGSGDWFKVDEFGYDASTKTWGTDKLNENCGKRTFNIPSHIPAGDYLVRAEAIALHTANQPGGAQFYMSCYQVRISGGEGGQLPAGVKIPGAYSANDPGILVDIWGNDFNDPPGHSARHAIIIISSSSNNSGAKMTKKIQEPTITSVTDLPTDEAKWIALQKISYVDQTGTARTYEPASRKTRSPRV

SEQ ID NO: 27 - protein, 284 aa, Myceliophthora thermophila
HSIFQQASSGSTDFDTLCTRMPPNNSPVTSVTSGDMTCKVGGTKGVSGFCEVNAGDEFTVEMHAQPGDRSCANEAIGGNHFGPVLIYMSKVDDASTADGSGDWFKVDEFGYDASTKTWGTDKLNENCGKRTFNIPSHIPAGDYLVRAEAIALHTANQPGGAQFYMSCYQVRISGGEGGQLPAGVKIPGAYSANDPGILVDIWGNDFNDPPGHSARHAIIIISSSSNNSGAKMTKKIQEPTITSVTDLPTDEAKWIALQKISYVDQTGTARTYEPASRKTRSPRV

SEQ ID NO: 28 - DNA, 726 bp, Myceliophthora thermophila
atgaagtcgtctaccccggccttgttcgccgctgggctccttgctcagcatgctgcggcccactccatcttccagcaggcgagcagcggctcgaccgactttgatacgctgtgcacccggatgccgcccaacaatagccccgtcactagtgtgaccagcggcgacatgacctgcaacgtcggcggcaccaagggggtgtcgggcttctgcgaggtgaacgccggcgacgagttcacggttgagatgcacgcgcagcccggcgaccgctcgtgcgccaacgaggccatcggcgggaaccacttcggcccggtcctcatctacatgagcaaggtcgacgacgcctccactgccgacgggtccggcgactggttcaaggtggacgagttcggctacgacgcaagcaccaagacctggggcaccgacaagctcaacgagaactgcggcaagcgcaccttcaacatccccagccacatccccgcgggcgactatctcgtccgggccgaggctatcgcgctacacactgccaaccagccaggcggcgcgcagttctacatgagctgctatcaagtcaggatttccggcggcgaagggggccagctgcctgccggagtcaagatcccgggcgcgtacagtgccaacgaccccggcatccttgtcgacatctggggtaacgatttcaacgagtacgttattccgggccccccggtcatcgacagcagctacttc

SEQ ID NO: 29 - protein, 242 aa, Myceliophthora thermophila
MKSSTPALFAAGLLAQHAAAHSIFQQASSGSTDFDTLCTRMPPNNSPVTSVTSGDMTCNVGGTKGVSGFCEVNAGDEFTVEMHAQPGDRSCANEAIGGNHFGPVLIYMSKVDDASTADGSGDWFKVDEFGYDASTKTWGTDKLNENCGKRTFNIPSHIPAGDYLVRAEAIALHTANQPGGAQFYMSCYQVRISGGEGGQLPAGVKIPGAYSANDPGILVDIWGNDFNEYVIPGPPVIDSSYF

SEQ ID NO: 30 - protein, 222 aa, Myceliophthora thermophila
HSIFQQASSGSTDFDTLCTRMPPNNSPVTSVTSGDMTCNVGGTKGVSGFCEVNAGDEFTVEMHAQPGDRSCANEAIGGNHFGPVLIYMSKVDDASTADGSGDWFKVDEFGYDASTKTWGTDKLNENCGKRTFNIPSHIPAGDYLVRAEAIALHTANQPGGAQFYMSCYQVRISGGEGGQLPAGVKIPGAYSANDPGILVDIWGNDFNEYVIPGPPVIDSSYF

SEQ ID NO: 31 - DNA, 969 bp, Myceliophthora thermophila
atgaagtccttcaccctcaccactctggccgccctggctggcaacgccgccgctcacgcgaccttccaggccctctgggtcgacggcgtcgactacggcgcgcagtgtgcccgtctgcccgcgtccaactcgccggtcaccgacgtgacctccaacgcgatccgctgcaacgccaacccctcgcccgctcggggcaagtgcccggtcaaggccggctcgaccgttacggtcgagatgcatcagcaacccggtgaccgctcgtgcagcagcgaggcgatcggcggggcgcactacggccccgtgatggtgtacatgtccaaggtgtcggacgcggcgtcggcggacgggtcgtcgggctggttcaaggtgttcgaggacggctgggccaagaacccgtccggcgggtcgggcgacgacgactactggggcaccaaggacctgaactcgtgctgcgggaagatgaacgtcaagatccccgccgacctgccctcgggcgactacctgctccgggccgaggccctcgcgctgcacacggccggcagcgcgggcggcgcccagttctacatgacctgctaccagctcaccgtgaccggctccggcagcgccagcccgcccaccgtctccttcccgggcgcctacaaggccaccgacccgggcatcctcgtcaacatccacgccccgctgtccggctacaccgtgcccggcccggccgtctactcgggcggctccaccaagaaggccggcagcgcctgcaccggctgcgagtccacttgcgccgtcggctccggccccaccgccaccgtctcccagtcgcccggttccaccgccacctcggcccccggcggcggcggcggctgcaccgtccagaagtaccagcagtgcggcggccagggctacaccggctgcaccaactgcgcgtccggctccacctgcagcgcggtctcgccgccctactactcgcagtgcgtc

SEQ ID NO: 32 - protein, 323 aa, Myceliophthora thermophila
MKSFTLTTLAALAGNAAAHATFQALWVDGVDYGAQCARLPASNSPVTDVTSNAIRCNANPSPARGKCPVKAGSTVTVEMHQQPGDRSCSSEAIGGAHYGPVMVYMSKVSDAASADGSSGWFKVFEDGWAKNPSGGSGDDDYWGTKDLNSCCGKMNVKIPADLPSGDYLLRAEALALHTAGSAGGAQFYMTCYQLTVTGSGSASPPTVSFPGAYKATDPGILVNIHAPLSGYTVPGPAVYSGGSTKKAGSACTGCESTCAVGSGPTATVSQSPGSTATSAPGGGGGCTVQKYQQCGGQGYTGCTNCASGSTCSAVSPPYYSQCV

SEQ ID NO: 33 - protein, 305 aa, Myceliophthora thermophila
HATFQALWVDGVDYGAQCARLPASNSPVTDVTSNAIRCNANPSPARGKCPVKAGSTVTVEMHQQPGDRSCSSEAIGGAHYGPVMVYMSKVSDAASADGSSGWFKVFEDGWAKNPSGGSGDDDYWGTKDLNSCCGKMNVKIPADLPSGDYLLRAEALALHTAGSAGGAQFYMTCYQLTVTGSGSASPPTVSFPGAYKATDPGILVNIHAPLSGYTVPGPAVYSGGSTKKAGSACTGCESTCAVGSGPTATVSQSPGSTATSAPGGGGGCTVQKYQQCGGQGYTGCTNCASGSTCSAVSPPYYSQCV

SEQ ID NO: 34 - DNA, 870 bp, Myceliophthora thermophila
atgaagggactcctcggcgccgccgccctctcgctggccgtcagcgatgtctcggcccactacatctttcagcagctgacgacgggcggcgtcaagcacgctgtgtaccagtacatccgcaagaacaccaactataactcgcccgtgaccgatctgacgtccaacgacctccgctgcaatgtgggtgctaccggtgcgggcaccgataccgtcacggtgcgcgccggcgattcgttcaccttcacgaccgatacgcccgtttaccaccagggcccgacctcgatctacatgtccaaggcccccggcagcgcgtccgactacgacggcagcggcggctggttcaagatcaaggactgggctgactacaccgccacgattccggaatgtattccccccggcgactacctgcttcgcatccagcaactcggcatccacaacccttggcccgcgggcatcccccagttctacatctcttgtgcccagatcaccgtgactggtggcggcagtgccaaccccggcccgaccgtctccatcccaggcgccttcaaggagaccgacccgggctacactgtcaacatctacaacaacttccacaactacaccgtccctggcccagccgtcttcacctgcaacggtagcggcggcaacaacggcggcggctccaacccagtcaccaccaccaccaccaccaccaccaggccgtccaccagcaccgcccagtcccagccgtcgtcgagcccgaccagcccctccagctgcaccgtcgcgaagtggggccagtgcggaggacagggttacagcggctgcaccgtgtgcgcggccgggtcgacctgccagaagaccaacgactactacagccagtgcttgtag

SEQ ID NO: 35 - protein, 289 aa, Myceliophthora thermophila
MKGLLGAAALSLAVSDVSAHYIFQQLTTGGVKHAVYQYIRKNTNYNSPVTDLTSNDLRCNVGATGAGTDTVTVRAGDSFTFTTDTPVYHQGPTSIYMSKAPGSASDYDGSGGWFKIKDWADYTATIPECIPPGDYLLRIQQLGIHNPWPAGIPQFYISCAQITVTGGGSANPGPTVSIPGAFKETDPGYTVNIYNNFHNYTVPGPAVFTCNGSGGNNGGGSNPVTTTTTTTTRPSTSTAQSQPSSSPTSPSSCTVAKWGQCGGQGYSGCTVCAAGSTCQKTNDYYSQCL

SEQ ID NO: 36 - protein, 270 aa, Myceliophthora thermophila
HYIFQQLTTGGVKHAVYQYIRKNTNYNSPVTDLTSNDLRCNVGATGAGTDTVTVRAGDSFTFTTDTPVYHQGPTSIYMSKAPGSASDYDGSGGWFKIKDWADYTATIPECIPPGDYLLRIQQLGIHNPWPAGIPQFYISCAQITVTGGGSANPGPTVSIPGAFKETDPGYTVNIYNNFHNYTVPGPAVFTCNGSGGNNGGGSNPVTTTTTTTTRPSTSTAQSQPSSSPTSPSSCTVAKWGQCGGQGYSGCTVCAAGSTCQKTNDYYSQCL

SEQ ID NO: 37 - DNA, 834 bp, Myceliophthora thermophila
ctgacgacgggcggcgtcaagcacgctgtgtaccagtacatccgcaagaacaccaactataactcgcccgtgaccgatctgacgtccaacgacctccgctgcaatgtgggtgctaccggtgcgggcaccgataccgtcacggtgcgcgccggcgattcgttcaccttcacgaccgatacgcccgtttaccaccagggcccgacctcgatctacatgtccaaggcccccggcagcgcgtccgactacgacggcagcggcggctggttcaagatcaaggactggggtgccgactttagcagcggccaggccacctggaccttggcgtctgactacaccgccacgattccggaatgtattccccccggcgactacctgcttcgcatccagcaactcggcatccacaacccttggcccgcgggcatcccccagttctacatctcttgtgcccagatcaccgtgactggtggcggcagtgccaaccccggcccgaccgtctccatcccaggcgccttcaaggagaccgacccgggctacactgtcaacatctacaacaacttccacaactacaccgtccctggcccagccgtcttcacctgcaacggtagcggcggcaacaacggcggcggctccaacccagtcaccaccaccaccaccaccaccaccaggccgtccaccagcaccgcccagtcccagccgtcgtcgagcccgaccagcccctccagctgcaccgtcgcgaagtggggccagtgcggaggacagggttacagcggctgcaccgtgtgcgcggccgggtcgacctgccagaagaccaacgactactacagccagtgcttg

SEQ ID NO: 38 - protein, 303 aa, Myceliophthora thermophila
MKGLLGAAALSLAVSDVSAHYIFQQLTTGGVKHAVYQYIRKNTNYNSPVTDLTSNDLRCNVGATGAGTDTVTVRAGDSFTFTTDTPVYHQGPTSIYMSKAPGSASDYDGSGGWFKIKDWGADFSSGQATWTLASDYTATIPECIPPGDYLLRIQQLGIHNPWPAGIPQFYISCAQITVTGGGSANPGPTVSIPGAFKETDPGYTVNIYNNFHNYTVPGPAVFTCNGSGGNNGGGSNPVTTTTTTTTRPSTSTAQSQPSSSPTSPSSCTVAKWGQCGGQGYSGCTVCAAGSTCQKTNDYYSQCL

SEQ ID NO: 39 - protein, 284 aa, Myceliophthora thermophila
HYIFQQLTTGGVKHAVYQYIRKNTNYNSPVTDLTSNDLRCNVGATGAGTDTVTVRAGDSFTFTTDTPVYHQGPTSIYMSKAPGSASDYDGSGGWFKIKDWGADFSSGQATWTLASDYTATIPECIPPGDYLLRIQQLGIHNPWPAGIPQFYISCAQITVTGGGSANPGPTVSIPGAFKETDPGYTVNIYNNFHNYTVPGPAVFTCNGSGGNNGGGSNPVTTTTTTTTRPSTSTAQSQPSSSPTSPSSCTVAKWGQCGGQGYSGCTVCAAGSTCQKTNDYYSQCL

SEQ ID NO: 40 - DNA, 1038 bp, Myceliophthora thermophila
atgtcttccttcacctccaagggtctcctttccgccctcatgggcgcggcaacggttgccgcccacggtcacgtcaccaacatcgtcatcaacggcgtctcataccagaacttcgacccattcacgcacccttatatgcagaaccctccgacggttgtcggctggaccgcgagcaacacggacaacggcttcgtcggccccgagtccttctctagcccggacatcatctgccacaagtccgccaccaacgctggcggccatgccgtcgtcgcggccggcgataaggtcttcatccagtgggacacctggcccgagtcgcaccacggtccggtcatcgactatctcgccgactgcggcgacgcgggctgcgagaaggtcgacaagaccacgctcaagttcttcaagatcagcgagtccggcctgctcgacggcactaacgcccccggcaagtgggcgtccgacacgctgatcgccaacaacaactcgtggctggtccagatcccgcccaacatcgccccgggcaactacgtcctgcgccacgagatcatcgccctgcacagcgccggccagcagaacggcgcccagaactaccctcagtgcttcaacctgcaggtcaccggctccggcactcagaagccctccggcgtcctcggcaccgagctctacaaggccaccgacgccggcatcctggccaacatctacacctcgcccgtcacctaccagatccccggcccggccatcatctcgggcgcctccgccgtccagcagaccacctcggccatcaccgcctctgctagcgccatcaccggctccgctaccgccgcgcccacggctgccaccaccaccgccgccgccgccgccaccactaccaccaccgctggctccggtgctaccgccacgccctcgaccggcggctctccttcttccgcccagcctgctcctaccaccgctgccgctacctccagccctgctcgcccgacccgctgcgctggtctgaagaagcgccgtcgccacgcccgtgacgtcaaggttgccctc

SEQ ID NO: 41 - protein, 346 aa, Myceliophthora thermophila
MSSFTSKGLLSALMGAATVAAHGHVTNIVINGVSYQNFDPFTHPYMQNPPTVVGWTASNTDNGFVGPESFSSPDIICHKSATNAGGHAVVAAGDKVFIQWDTWPESHHGPVIDYLADCGDAGCEKVDKTTLKFFKISESGLLDGTNAPGKWASDTLIANNNSWLVQIPPNIAPGNYVLRHEIIALHSAGQQNGAQNYPQCFNLQVTGSGTQKPSGVLGTELYKATDAGILANIYTSPVTYQIPGPAIISGASAVQQTTSAITASASAITGSATAAPTAATTTAAAAATTTTTAGSGATATPSTGGSPSSAQPAPTTAAATSSPARPTRCAGLKKRRRHARDVKVAL

SEQ ID NO: 42 - protein, 326 aa, Myceliophthora thermophila
AHGHVTNIVINGVSYQNFDPFTHPYMQNPPTVVGWTASNTDNGFVGPESFSSPDIICHKSATNAGGHAVVAAGDKVFIQWDTWPESHHGPVIDYLADCGDAGCEKVDKTTLKFFKISESGLLDGTNAPGKWASDTLIANNNSWLVQIPPNIAPGNYVLRHEIIALHSAGQQNGAQNYPQCFNLQVTGSGTQKPSGVLGTELYKATDAGILANIYTSPVTYQIPGPAIISGASAVQQTTSAITASASAITGSATAAPTAATTTAAAAATTTTTAGSGATATPSTGGSPSSAQPAPTTAAATSSPARPTRCAGLKKRRRHARDVKVAL

SEQ ID NO: 43 - DNA, 714 bp, Myceliophthora thermophila
atgaagacgctcgccgccctcgtggtctcggccgccctcgtggccgcgcacggctatgttgaccacgccacgatcggtggcaaggattatcagttctaccagccgtaccaggacccttacatgggcgacaacaagcccgatagggtttcccgctccatcccgggcaacggccccgtggaggacgtcaactccatcgacctccagtgccacgccggtgccgaaccggccaagctccacgcccccgccgccgccggctcgaccgtgacgctctactggaccctctggcccgactcccacgtcggccccgtcatcacctacatggctcgctgccccgacaccggctgccaggactggtccccgggaactaagcccgtttggttcaagatcaaggaaggcggccgtgagggcacctccaataccccgctcatgacggccccctccgcctacacctacacgatcccgtcctgcctcaagagcggctactacctcgtccgccacgagatcatcgccctgcactcggcctggcagtaccccggcgcccagttctacccgggctgccaccagctccaggtcaccggcggcggctccaccgtgccctctaccaacctggtctccttccccggcgcctacaaggggagcgaccccggcatcacctacgacgcttacaaggcgcaaccttacaccatccctggcccggccgtgtttacctgctga

SEQ ID NO: 44 - protein, 237 aa, Myceliophthora thermophila
MKTLAALVVSAALVAAHGYVDHATIGGKDYQFYQPYQDPYMGDNKPDRVSRSIPGNGPVEDVNSIDLQCHAGAEPAKLHAPAAAGSTVTLYWTLWPDSHVGPVITYMARCPDTGCQDWSPGTKPVWFKIKEGGREGTSNTPLMTAPSAYTYTIPSCLKSGYYLVRHEIIALHSAWQYPGAQFYPGCHQLQVTGGGSTVPSTNLVSFPGAYKGSDPGITYDAYKAQPYTIPGPAVFTC

SEQ ID NO: 45 - protein, 219 aa, Myceliophthora thermophila
YVDHATIGGKDYQFYQPYQDPYMGDNKPDRVSRSIPGNGPVEDVNSIDLQCHAGAEPAKLHAPAAAGSTVTLYWTLWPDSHVGPVITYMARCPDTGCQDWSPGTKPVWFKIKEGGREGTSNTPLMTAPSAYTYTIPSCLKSGYYLVRHEIIALHSAWQYPGAQFYPGCHQLQVTGGGSTVPSTNLVSFPGAYKGSDPGITYDAYKAQPYTIPGPAVFTC

SEQ ID NO: 46 - DNA, 723 bp, Myceliophthora thermophila
atgaagacgctcgccgccctcgtggtctcggccgccctcgtggccgcgcacggctatgttgaccacgccacgatcggtggcaaggattatcagttctaccagccgtaccaggacccttacatgggcgacaacaagcccgatagggtttcccgctccatcccgggcaacggccccgtggaggacgtcaactccatcgacctccagtgccacgccggtgccgaaccggccaagctccacgcccccgccgccgccggctcgaccgtgacgctctactggaccctctggcccgactcccacgtcggccccgtcatcacctacatggctcgctgccccgacaccggctgccaggactggtccccgggaactaagcccgtttggttcaagatcaaggaaggcggccgtgagggcacctccaatgtctgggctgctaccccgctcatgacggccccctccgcctacacctacacgatcccgtcctgcctcaagagcggctactacctcgtccgccacgagatcatcgccctgcactcggcctggcagtaccccggcgcccagttctacccgggctgccaccagctccaggtcaccggcggcggctccaccgtgccctctaccaacctggtctccttccccggcgcctacaaggggagcgaccccggcatcacctacgacgcttacaaggcgcaaccttacaccatccctggcccggccgtgtttacctgc

SEQ ID NO: 47 - protein, 241 aa, Myceliophthora thermophila
MKTLAALVVSAALVAAHGYVDHATIGGKDYQFYQPYQDPYMGDNKPDRVSRSIPGNGPVEDVNSIDLQCHAGAEPAKLHAPAAAGSTVTLYWTLWPDSHVGPVITYMARCPDTGCQDWSPGTKPVWFKIKEGGREGTSNVWAATPLMTAPSAYTYTIPSCLKSGYYLVRHEIIALHSAWQYPGAQFYPGCHQLQVTGGGSTVPSTNLVSFPGAYKGSDPGITYDAYKAQPYTIPGPAVFTC

SEQ ID NO: 48 - protein, 223 aa, Myceliophthora thermophila
YVDHATIGGKDYQFYQPYQDPYMGDNKPDRVSRSIPGNGPVEDVNSIDLQCHAGAEPAKLHAPAAAGSTVTLYWTLWPDSHVGPVITYMARCPDTGCQDWSPGTKPVWFKIKEGGREGTSNVWAATPLMTAPSAYTYTIPSCLKSGYYLVRHEIIALHSAWQYPGAQFYPGCHQLQVTGGGSTVPSTNLVSFPGAYKGSDPGITYDAYKAQPYTIPGPAVFTC

SEQ ID NO: 49 - DNA, 675 bp, Myceliophthora thermophila
atgagatacttcctccagctcgctgcggccgcggcctttgccgtgaacagcgcggcgggtcactacatcttccagcagttcgcgacgggcgggtccaagtacccgccctggaagtacatccggcgcaacaccaacccggactggctgcagaacgggccggtgacggacctgtcgtcgaccgacctgcgctgcaacgtgggcgggcaggtcagcaacgggaccgagaccatcaccttgaacgccggcgacgagttcagcttcatcctcgacacgcccgtctaccatgccggccccacctcgctctacatgtccaaggcgcccggagctgtggccgactacgacggcggcggggcctggttcaagatctacgactggggtccgtcggggacgagctggacgttgagtggcacgtacactcagagaattcccaagtgcatccctgacggcgagtacctcctccgcatccagcagatcgggctccacaaccccggcgccgcgccacagttctacatcagctgcgctcaagtcaaggtcgtcgatggcggcagcaccaatccgaccccgaccgcccagattccgggagccttccacagcaacgaccctggcttgactgtcaatatctacaacgaccctctcaccaactacgtcgtcccgggacctagagtttcgcactgg

SEQ ID NO: 50 - protein, 225 aa, Myceliophthora thermophila
MRYFLQLAAAAAFAVNSAAGHYIFQQFATGGSKYPPWKYIRRNTNPDWLQNGPVTDLSSTDLRCNVGGQVSNGTETITLNAGDEFSFILDTPVYHAGPTSLYMSKAPGAVADYDGGGAWFKIYDWGPSGTSWTLSGTYTQRIPKCIPDGEYLLRIQQIGLHNPGAAPQFYISCAQVKVVDGGSTNPTPTAQIPGAFHSNDPGLTVNIYNDPLTNYVVPGPRVSHW

SEQ ID NO: 51 - protein, 205 aa, Myceliophthora thermophila
HYIFQQFATGGSKYPPWKYIRRNTNPDWLQNGPVTDLSSTDLRCNVGGQVSNGTETITLNAGDEFSFILDTPVYHAGPTSLYMSKAPGAVADYDGGGAWFKIYDWGPSGTSWTLSGTYTQRIPKCIPDGEYLLRIQQIGLHNPGAAPQFYISCAQVKVVDGGSTNPTPTAQIPGAFHSNDPGLTVNIYNDPLTNYVVPGPRVSHW

SEQ ID NO: 52 - DNA, 1332 bp, Myceliophthora thermophila
atgcacccctcccttcttttcacgcttgggctggcgagcgtgcttgtccccctctcgtctgcacacactaccttcacgaccctcttcgtcaacgatgtcaaccaaggtgatggtacctgcattcgcatggcgaagaagggcaatgtcgccacccatcctctcgcaggcggtctcgactccgaagacatggcctgtggtcgggatggtcaagaacccgtggcatttacgtgtccggccccagctggtgccaagttgactctcgagtttcgcatgtgggccgatgcttcgcagtccggatcgatcgatccatcccaccttggcgtcatggccatctacctcaagaaggtttccgacatgaaatctgacgcggccgctggcccgggctggttcaagatttgggaccaaggctacgacttggcggccaagaagtgggccaccgagaagctcatcgacaacaacggcctcctgagcgtcaaccttccaaccggcttaccaaccggctactacctcgcccgccaggagatcatcacgctccaaaacgttaccaatgacaggccagagccccagttctacgtcggctgcgcacagctctacgtcgagggcacctcggactcacccatcccctcggacaagacggtctccattcccggccacatcagcgacccggccgacccgggcctgaccttcaacgtctacacgggcgacgcatccacctacaagccgcccggccccgaggtttacttccccaccaccaccaccaccacctcctcctcctcctccggaagcagcgacaacaagggagccaggcgccagcaaacccccgacgacaagcaggccgacggcctcgttccagccgactgcctcgtcaagaacgcgaactggtgcgccgctgccctgccgccgtacaccgacgaggccggctgctgggccgccgccgaggactgcaacaagcagctggacgcgtgctacaccagcgcacccccctcgggcagcaaggggtgcaaggtctgggaggagcaggtgtgcaccgtcgtctcgcagaagtgcgaggccggggatttcaaggggcccccgcagctcgggaaggagctcggcgaggggatcgatgagcctattccggggggaaagctgcccccggcggtcaacgcgggagagaacgggaatcatggcggaggtggtggtgatgatggtgatgatgataatgatgaggccggggctggggcagcgtcgactccgacttttgctgctcctggtgcggccaagactccccaaccaaactccgagagggcccggcgccgtgaggcgcattggcggcgactggaatctgctgag

SEQ ID NO: 53 - protein, 444 aa, Myceliophthora thermophila
MHPSLLFTLGLASVLVPLSSAHTTFTTLFVNDVNQGDGTCIRMAKKGNVATHPLAGGLDSEDMACGRDGQEPVAFTCPAPAGAKLTLEFRMWADASQSGSIDPSHLGVMAIYLKKVSDMKSDAAAGPGWFKIWDQGYDLAAKKWATEKLIDNNGLLSVNLPTGLPTGYYLARQEIITLQNVTNDRPEPQFYVGCAQLYVEGTSDSPIPSDKTVSIPGHISDPADPGLTFNVYTGDASTYKPPGPEVYFPTTTTTTSSSSSGSSDNKGARRQQTPDDKQADGLVPADCLVKNANWCAAALPPYTDEAGCWAAAEDCNKQLDACYTSAPPSGSKGCKVWEEQVCTVVSQKCEAGDFKGPPQLGKELGEGIDEPIPGGKLPPAVNAGENGNHGGGGGDDGDDDNDEAGAGAASTPTFAAPGAAKTPQPNSERARRREAHWRRLESAE

SEQ ID NO: 54 - protein, 423 aa, Myceliophthora thermophila
HTTFTTLFVNDVNQGDGTCIRMAKKGNVATHPLAGGLDSEDMACGRDGQEPVAFTCPAPAGAKLTLEFRMWADASQSGSIDPSHLGVMAIYLKKVSDMKSDAAAGPGWFKIWDQGYDLAAKKWATEKLIDNNGLLSVNLPTGLPTGYYLARQEIITLQNVTNDRPEPQFYVGCAQLYVEGTSDSPIPSDKTVSIPGHISDPADPGLTFNVYTGDASTYKPPGPEVYFPTTTTTTSSSSSGSSDNKGARRQQTPDDKQADGLVPADCLVKNANWCAAALPPYTDEAGCWAAAEDCNKQLDACYTSAPPSGSKGCKVWEEQVCTVVSQKCEAGDFKGPPQLGKELGEGIDEPIPGGKLPPAVNAGENGNHGGGGGDDGDDDNDEAGAGAASTPTFAAPGAAKTPQPNSERARRREAHWRRLESAE

SEQ ID NO: 55 - DNA, 834 bp, Myceliophthora thermophila
atgttttctctcaagttctttatcttggccggtgggcttgctgtcctcaccgaggctcacataagactagtgtcgcccgccccttttaccaaccctgaccagggccccagcccactcctagaggctggcagcgactatccctgccacaacggcaatgggggcggttatcagggaacgccaacccagatggcaaagggttctaagcagcagctagccttccaggggtctgccgttcatgggggtggctcctgccaagtgtccatcacctacgacgaaaacccgaccgctcagagctccttcaaggtcattcactcgattcaaggtggctgccccgccagggccgagacgatcccggattgcagcgcacaaaatatcaacgcctgcaatataaagcccgataatgcccagatggacaccccggataagtatgagttcacgatcccggaggatctccccagtggcaaggccaccctcgcctggacatggatcaacactatcggcaaccgcgagttttatatggcatgcgccccggttgagatcaccggcgacggcggtagcgagtcggctctggctgcgctgcccgacatggtcattgccaacatcccgtccatcggaggaacctgcgcgaccgaggaggggaagtactacgaatatcccaaccccggtaagtcggtcgaaaccatcccgggctggaccgatttggttcccctgcaaggcgaatgcggtgctgcctccggtgtctcgggctccggcggaaacgccagcagtgctacccctgccgcaggggccgccccgactcctgctgtccgcggccgccgtcccacctggaacgcc

SEQ ID NO: 56 - protein, 278 aa, Myceliophthora thermophila
MFSLKFFILAGGLAVLTEAHIRLVSPAPFTNPDQGPSPLLEAGSDYPCHNGNGGGYQGTPTQMAKGSKQQLAFQGSAVHGGGSCQVSITYDENPTAQSSFKVIHSIQGGCPARAETIPDCSAQNINACNIKPDNAQMDTPDKYEFTIPEDLPSGKATLAWTWINTIGNREFYMACAPVEITGDGGSESALAALPDMVIANIPSIGGTCATEEGKYYEYPNPGKSVETIPGWTDLVPLQGECGAASGVSGSGGNASSATPAAGAAPTPAVRGRRPTWNA

SEQ ID NO: 57 - protein, 259 aa, Myceliophthora thermophila
HIRLVSPAPFTNPDQGPSPLLEAGSDYPCHNGNGGGYQGTPTQMAKGSKQQLAFQGSAVHGGGSCQVSITYDENPTAQSSFKVIHSIQGGCPARAETIPDCSAQNINACNIKPDNAQMDTPDKYEFTIPEDLPSGKATLAWTWINTIGNREFYMACAPVEITGDGGSESALAALPDMVIANIPSIGGTCATEEGKYYEYPNPGKSVETIPGWTDLVPLQGECGAASGVSGSGGNASSATPAAGAAPTPAVRGRRPTWNA

SEQ ID NO: 58 - DNA, 672 bp, Myceliophthora thermophila
atgaagctcgccacgctcctcgccgccctcaccctcggggtggccgaccagctcagcgtcgggtccagaaagtttggcgtgtacgagcacattcgcaagaacacgaactacaactcgcccgttaccgacctgtcggacaccaacctgcgctgcaacgtcggcgggggctcgggcaccagcaccaccgtgctcgacgtcaaggccggagactcgttcaccttcttcagcgacgttgccgtctaccaccaggggcccatctcgctgtgcgtggaccggaccagtgcagagagcatggatggacgggaaccggacatgcgctgccgaactggctcacaagctggctacctggcggtgactgactacgacgggtccggtgactgtttcaagatctatgactggggaccgacgttcaacgggggccaggcgtcgtggccgacgaggaattcgtacgagtacagcatcctcaagtgcatcagggacggcgaatacctactgcggattcagtccctggccatccataacccaggtgcccttccgcagttctacatcagctgcgcccaggtgaatgtgacgggcggaggcaccgtcaccccgagatcaaggcgaccgatcctgatctatttcaacttccactcgtatatcgtccctgggccggcagtgttcaagtgctag

SEQ ID NO: 59 - protein, 223 aa, Myceliophthora thermophila
MKLATLLAALTLGVADQLSVGSRKFGVYEHIRKNTNYNSPVTDLSDTNLRCNVGGGSGTSTTVLDVKAGDSFTFFSDVAVYHQGPISLCVDRTSAESMDGREPDMRCRTGSQAGYLAVTDYDGSGDCFKIYDWGPTFNGGQASWPTRNSYEYSILKCIRDGEYLLRIQSLAIHNPGALPQFYISCAQVNVTGGGTVTPRSRRPILIYFNFHSYIVPGPAVFKC

SEQ ID NO: 60 - protein, 208 aa, Myceliophthora thermophila
DQLSVGSRKFGVYEHIRKNTNYNSPVTDLSDTNLRCNVGGGSGTSTTVLDVKAGDSFTFFSDVAVYHQGPISLCVDRTSAESMDGREPDMRCRTGSQAGYLAVTDYDGSGDCFKIYDWGPTFNGGQASWPTRNSYEYSILKCIRDGEYLLRIQSLAIHNPGALPQFYISCAQVNVTGGGTVTPRSRRPILIYFNFHSYIVPGPAVFKC

SEQ ID NO: 61 - DNA, 642 bp, Myceliophthora thermophila
atgaagctcgccacgctcctcgccgccctcaccctcgggctcagcgtcgggtccagaaagtttggcgtgtacgagcacattcgcaagaacacgaactacaactcgcccgttaccgacctgtcggacaccaacctgcgctgcaacgtcggcgggggctcgggcaccagcaccaccgtgctcgacgtcaaggccggagactcgttcaccttcttcagcgacgttgccgtctaccaccaggggcccatctcgctgtgcgtggaccggaccagtgcagagagcatggatggacgggaaccggacatgcgctgccgaactggctcacaagctggctacctggcggtgactgtgatgactgtgactgactacgacgggtccggtgactgtttcaagatctatgactggggaccgacgttcaacgggggccaggcgtcgtggccgacgaggaattcgtacgagtacagcatcctcaagtgcatcagggacggcgaatacctactgcggattcagtccctggccatccataacccaggtgcccttccgcagttctacatcagctgcgcccaggtgaatgtgacgggcggaggcaccatctatttcaacttccactcgtatatcgtccctgggccggcagtgttcaagtgc

SEQ ID NO: 62 - protein, 214 aa, Myceliophthora thermophila
MKLATLLAALTLGLSVGSRKFGVYEHIRKNTNYNSPVTDLSDTNLRCNVGGGSGTSTTVLDVKAGDSFTFFSDVAVYHQGPISLCVDRTSAESMDGREPDMRCRTGSQAGYLAVTVMTVTDYDGSGDCFKIYDWGPTFNGGQASWPTRNSYEYSILKCIRDGEYLLRIQSLAIHNPGALPQFYISCAQVNVTGGGTIYFNFHSYIVPGPAVFKC

SEQ ID NO: 63 - protein, 196 aa, Myceliophthora thermophila
RKFGVYEHIRKNTNYNSPVTDLSDTNLRCNVGGGSGTSTTVLDVKAGDSFTFFSDVAVYHQGPISLCVDRTSAESMDGREPDMRCRTGSQAGYLAVTVMTVTDYDGSGDCFKIYDWGPTFNGGQASWPTRNSYEYSILKCIRDGEYLLRIQSLAIHNPGALPQFYISCAQVNVTGGGTIYFNFHSYIVPGPAVFKC

SEQ ID NO: 64 - DNA, 579 bp, Myceliophthora thermophila
atgaccaagaatgcgcagagcaagcagggcgttgagaacccaacaagcggcgacatccgctgctacacctcgcagacggcggccaacgtcgtgaccgtgccggccggctcgaccattcactacatctcgacccagcagatcaaccaccccggcccgactcagtactacctggccaaggtaccccccggctcgtcggccaagacctttgacgggtccggcgccgtctggttcaagatctcgaccacgatgcctaccgtggacagcaacaagcagatgttctggccagggcagaacacttatgagacctcaaacaccaccattcccgccaacaccccggacggcgagtacctccttcgcgtcaagcagatcgccctccacatggcgtctcagcccaacaaggtccagttctacctcgcctgcacccagatcaagatcaccggtggtcgcaacggcacccccagcccgctggtcgcgctgcccggagcctacaagagcaccgaccccggcatcctggtcgacatctactccatgaagcccgaatcgtaccagcctcccgggccgcccgtctggcgcggctaa

SEQ ID NO: 65 - protein, 192 aa, Myceliophthora thermophila
MTKNAQSKQGVENPTSGDIRCYTSQTAANVVTVPAGSTIHYISTQQINHPGPTQYYLAKVPPGSSAKTFDGSGAVWFKISTTMPTVDSNKQMFWPGQNTYETSNTTIPANTPDGEYLLRVKQIALHMASQPNKVQFYLACTQIKITGGRNGTPSPLVALPGAYKSTDPGILVDIYSMKPESYQPPGPPVWRG

SEQ ID NO: 66 - DNA, 672 bp, Myceliophthora thermophila
atgaggcttctcgcaagcttgttgctcgcagctacggctgttcaagctcactttgttaacggacagcccgaagagagtgactggtcagccacgcgcatgaccaagaatgcgcagagcaagcagggcgttgagaacccaacaagcggcgacatccgctgctacacctcgcagacggcggccaacgtcgtgaccgtgccggccggctcgaccattcactacatctcgacccagcagatcaaccaccccggcccgactcagtactacctggccaaggtaccccccggctcgtcggccaagacctttgacgggtccggcgccgtctggttcaagatctcgaccacgatgcctaccgtggacagcaacaagcagatgttctggccagggcagaacacttatgagacctcaaacaccaccattcccgccaacaccccggacggcgagtacctccttcgcgtcaagcagatcgccctccacatggcgtctcagcccaacaaggtccagttctacctcgcctgcacccagatcaagatcaccggtggtcgcaacggcacccccagcccgctggtcgcgctgcccggagcctacaagagcaccgaccccggcatcctggtcgacatctactccatgaagcccgaatcgtaccagcctcccgggccgcccgtctggcgcggc

SEQ ID NO: 67 - protein, 224 aa, Myceliophthora thermophila
MRLLASLLLAATAVQAHFVNGQPEESDWSATRMTKNAQSKQGVENPTSGDIRCYTSQTAANVVTVPAGSTIHYISTQQINHPGPTQYYLAKVPPGSSAKTFDGSGAVWFKISTTMPTVDSNKQMFWPGQNTYETSNTTIPANTPDGEYLLRVKQIALHMASQPNKVQFYLACTQIKITGGRNGTPSPLVALPGAYKSTDPGILVDIYSMKPESYQPPGPPVWRG

SEQ ID NO: 68 - protein, 208 aa, Myceliophthora thermophila
HFVNGQPEESDWSATRMTKNAQSKQGVENPTSGDIRCYTSQTAANVVTVPAGSTIHYISTQQINHPGPTQYYLAKVPPGSSAKTFDGSGAVWFKISTTMPTVDSNKQMFWPGQNTYETSNTTIPANTPDGEYLLRVKQIALHMASQPNKVQFYLACTQIKITGGRNGTPSPLVALPGAYKSTDPGILVDIYSMKPESYQPPGPPVWRG

SEQ ID NO: 69 - DNA, 849 bp, Myceliophthora thermophila
atgaagccctttagcctcgtcgccctggcgactgccgtgagcggccatgccatcttccagcgggtgtcggtcaacgggcaggaccagggccagctcaagggggtgcgggcgccgtcgagcaactccccgatccagaacgtcaacgatgccaacatggcctgcaacgccaacattgtgtaccacgacaacaccatcatcaaggtgcccgcgggagcccgcgtcggcgcgtggtggcagcacgtcatcggcgggccgcagggcgccaacgacccggacaacccgatcgccgcctcccacaagggccccatccaggtctacctggccaaggtggacaacgcggcgacggcgtcgccgtcgggcctcaagtggttcaaggtggccgagcgcggcctgaacaacggcgtgtgggcctacctgatgcgcgtcgagctgctcgccctgcacagcgcctcgagccccggcggcgcccagttctacatgggctgtgcacagatcgaagtcactggctccggcaccaactcgggctccgactttgtctcgttccccggcgcctactcggccaacgacccgggcatcttgctgagcatctacgacagctcgggcaagcccaacaatggcgggcgctcgtacccgatccccggcccgcgccccatctcctgctccggcagcggcggcggcggcaacaacggcggcgacggcggcgacgacaacaacggtggtggcaacaacaacggcggcggcagcgtccccctgtacgggcagtgcggcggcatcggctacacgggcccgaccacctgtgcccagggaacttgcaaggtgtcgaacgaatactacagccagtgcctcccc

SEQ ID NO: 70 - protein, 283 aa, Myceliophthora thermophila
MKPFSLVALATAVSGHAIFQRVSVNGQDQGQLKGVRAPSSNSPIQNVNDANMACNANIVYHDNTIIKVPAGARVGAWWQHVIGGPQGANDPDNPIAASHKGPIQVYLAKVDNAATASPSGLKWFKVAERGLNNGVWAYLMRVELLALHSASSPGGAQFYMGCAQIEVTGSGTNSGSDFVSFPGAYSANDPGILLSIYDSSGKPNNGGRSYPIPGPRPISCSGSGGGGNNGGDGGDDNNGGGNNNGGGSVPLYGQCGGIGYTGPTTCAQGTCKVSNEYYSQCLP

SEQ ID NO: 71 - protein, 268 aa, Myceliophthora thermophila
HAIFQRVSVNGQDQGQLKGVRAPSSNSPIQNVNDANMACNANIVYHDNTIIKVPAGARVGAWWQHVIGGPQGANDPDNPIAASHKGPIQVYLAKVDNAATASPSGLKWFKVAERGLNNGVWAYLMRVELLALHSASSPGGAQFYMGCAQIEVTGSGTNSGSDFVSFPGAYSANDPGILLSIYDSSGKPNNGGRSYPIPGPRPISCSGSGGGGNNGGDGGDDNNGGGNNNGGGSVPLYGQCGGIGYTGPTTCAQGTCKVSNEYYSQCLP

SEQ ID NO: 72 - DNA, 639 bp, Myceliophthora thermophila
atgaagctcacctcgtccctcgctgtcctggccgctgccggcgcccaggctcactataccttccctagggccggcactggtggttcgctctctggcgagtgggaggtggtccgcatgaccgagaaccattactcgcacggcccggtcaccgatgtcaccagccccgagatgacctgctatcagtccggcgtgcagggtgcgccccagaccgtccaggtcaaggcgggctcccaattcaccttcagcgtggatccctccatcggccaccccggccctctccagttctacatggctaaggtgccgtcgggccagacggccgccacctttgacggcacgggagccgtgtggttcaagatctaccaagacggcccgaacggcctcggcaccgacagcattacctggcccagcgccggcaaaaccgaggtctcggtcaccatccccagctgcatcgaggatggcgagtacctgctccgggtcgagcacacccccctccctacagcgccagcagcgcaaaaccgagctcgctcgtcaccatccccagctgcatacaaggccaccgacccgggcatcctcttccagctctactggcccatcccgaccgagtacatcaaccccggcccggcccccgtctcttgctaa

SEQ ID NO: 73 - protein, 212 aa, Myceliophthora thermophila
MKLTSSLAVLAAAGAQAHYTFPRAGTGGSLSGEWEVVRMTENHYSHGPVTDVTSPEMTCYQSGVQGAPQTVQVKAGSQFTFSVDPSIGHPGPLQFYMAKVPSGQTAATFDGTGAVWFKIYQDGPNGLGTDSITWPSAGKTEVSVTIPSCIEDGEYLLRVEHTPLPTAPAAQNRARSSPSPAAYKATDPGILFQLYWPIPTEYINPGPAPVSC

SEQ ID NO: 74 - protein, 195 aa, Myceliophthora thermophila
HYTFPRAGTGGSLSGEWEVVRMTENHYSHGPVTDVTSPEMTCYQSGVQGAPQTVQVKAGSQFTFSVDPSIGHPGPLQFYMAKVPSGQTAATFDGTGAVWFKIYQDGPNGLGTDSITWPSAGKTEVSVTIPSCIEDGEYLLRVEHTPLPTAPAAQNRARSSPSPAAYKATDPGILFQLYWPIPTEYINPGPAPVSC

SEQ ID NO: 75 - DNA, 695 bp, Myceliophthora thermophila
atgaagctcacctcgtccctcgctgtcctggccgctgccggcgcccaggctcactataccttccctagggccggcactggtggttcgctctctggcgagtgggaggtggtccgcatgaccgagaccattactcgcacggcccggtcaccgatgtcaccagccccgagatgacctgctatcagtccggcgtgcagggtgcgccccagaccgtccaggtcaaggcgggctcccaattcaccttcagcgtggatccctccatcggccaccccggccctctccagttctacatggctaaggtgccgtcgggccagacggccgccacctttgacggcacgggagccgtgtggttcaagatctaccaagacggcccgaacggcctcggcaccgacagcattacctggcccagcgccggcaaaaccgaggtctcggtcaccatccccagctgcatcgaggatggcgagtacctgctccgggtcgagcacatcgcgctccacagcgccagcagcgtgggcggcgcccagttctacatcgcctgcgcccagctctccgtcaccggcggctccggcaccctcaacacgggctcgctcgtctccctgcccggcgcctacaaggccaccgacccgggcatcctcttccagctctactggcccatcccgaccgagtacatcaaccccggcccggcccccgtctcttgc

SEQ ID NO: 76 - protein, 232 aa, Myceliophthora thermophila
MKLTSSLAVLAAAGAQAHYTFPRAGTGGSLSGEWEVVRMTENHYSHGPVTDVTSPEMTCYQSGVQGAPQTVQVKAGSQFTFSVDPSIGHPGPLQFYMAKVPSGQTAATFDGTGAVWFKIYQDGPNGLGTDSITWPSAGKTEVSVTIPSCIEDGEYLLRVEHIALHSASSVGGAQFYIACAQLSVTGGSGTLNTGSLVSLPGAYKATDPGILFQLYWPIPTEYINPGPAPVSC

SEQ ID NO: 77 - protein, 215 aa, Myceliophthora thermophila
HYTFPRAGTGGSLSGEWEVVRMTENHYSHGPVTDVTSPEMTCYQSGVQGAPQTVQVKAGSQFTFSVDPSIGHPGPLQFYMAKVPSGQTAATFDGTGAVWFKIYQDGPNGLGTDSITWPSAGKTEVSVTIPSCIEDGEYLLRVEHIALHSASSVGGAQFYIACAQLSVTGGSGTLNTGSLVSLPGAYKATDPGILFQLYWPIPTEYINPGPAPVSC

SEQ ID NO: 78 - DNA, 447 bp, Myceliophthora thermophila
atgccgccaccacgactgagcaccctccttcccctcctagccttaatagcccccaccgccctggggcactcccacctcgggtacatcatcatcaacggcgaggtataccaaggattcgacccgcggccggagcaggcgaactcgccgttgcgcgtgggctggtcgacgggggcaatcgacgacgggttcgtggcgccggccaactactcgtcgcccgacatcatctgccacatcgagggggccagcccgccggcgcacgcgcccgtccgggcgggcgaccgggtgcacgtgcaatggaacggctggccgctcggacacgtggggccggtgctgtcgtacctggcgccctgcggcgggctggaggggtccgagagcgggtgcgccggggtggacaagcggcagctgcggtggaccaaggtggacgactcgctgccggcgatggagctg

SEQ ID NO: 79 - protein, 149 aa, Myceliophthora thermophila
MPPPRLSTLLPLLALIAPTALGHSHLGYIIINGEVYQGFDPRPEQANSPLRVGWSTGAIDDGFVAPANYSSPDIICHIEGASPPAHAPVRAGDRVHVQWNGWPLGHVGPVLSYLAPCGGLEGSESGCAGVDKRQLRWTKVDDSLPAMEL

SEQ ID NO: 80 - protein, 127 aa, Myceliophthora thermophila
HSHLGYIIINGEVYQGFDPRPEQANSPLRVGWSTGAIDDGFVAPANYSSPDIICHIEGASPPAHAPVRAGDRVHVQWNGWPLGHVGPVLSYLAPCGGLEGSESGCAGVDKRQLRWTKVDDSLPAMEL

SEQ ID NO: 81 - DNA, 1176 bp, Myceliophthora thermophila
atgccgccaccacgactgagcaccctccttcccctcctagccttaatagcccccaccgccctggggcactcccacctcgggtacatcatcatcaacggcgaggtataccaaggattcgacccgcggccggagcaggcgaactcgccgttgcgcgtgggctggtcgacgggggcaatcgacgacgggttcgtggcgccggccaactactcgtcgcccgacatcatctgccacatcgagggggccagcccgccggcgcacgcgcccgtccgggcgggcgaccgggtgcacgtgcaatggaaacggctggccgctcggacacgtggggccggtgctgtcgtacctggcgccctgcggcgggctggaggggtccgagagcgggtggacgactcgctgccggcgatggagctggtcggggccgcggggggcgcggggggcgaggacgacggcagcggcagcgacggcagcggcagcggcggcagcggacgcgtcggcgtgcccgggcagcgctgggccaccgacgtgttgatcgcggccaacaacagctggcaggtcgagatcccgcgcgggctgcgggacgggccgtacgtgctgcgccacgagatcgtcgcgctgcactacgcggccgagcccggcggcgcgcagaactacccgctctgcgtcaacctgtgggtcgagggcggcgacggcagcatggagctggaccacttcgacgccacccagttctaccggcccgacgacccgggcatcctgctcaacgtgacggccggcctgcgctcatacgccgtgccgggcccgacgctggccgcgggggcgacgccggtgccgtacgcgcagcagaacatcagctcggcgagggcggatggaacccccgtgattgtcaccaggagcacggagacggtgcccttcaccgcggcacccacgccagccgagacggcagaagccaaaggggggaggtatgatgaccaaacccgaactaaagacctaaatgaacgcttcttttatagtagccggccagaacagaagaggctgacagcgacctcaagaagggaactagttgatcatcgtacccggtacctctccgtagctgtctgcgcagatttcggcgctcataaggcagcagaaaccaaccacgaagctttgagaggcggcaataagcaccatggcggtgtttcagag

SEQ ID NO: 82 - protein, 392 aa, Myceliophthora thermophila
MPPPRLSTLLPLLALIAPTALGHSHLGYIIINGEVYQGFDPRPEQANSPLRVGWSTGAIDDGFVAPANYSSPDIICHIEGASPPAHAPVRAGDRVHVQWKRLAARTRGAGAVVPGALRRAGGVRERVDDSLPAMELVGAAGGAGGEDDGSGSDGSGSGGSGRVGVPGQRWATDVLIAANNSWQVEIPRGLRDGPYVLRHEIVALHYAAEPGGAQNYPLCVNLWVEGGDGSMELDHFDATQFYRPDDPGILLNVTAGLRSYAVPGPTLAAGATPVPYAQQNISSARADGTPVIVTRSTETVPFTAAPTPAETAEAKGGRYDDQTRTKDLNERFFYSSRPEQKRLTATSRRELVDHRTRYLSVAVCADFGAHKAAETNHEALRGGNKHHGGVSE

SEQ ID NO: 83 - protein, 370 aa, Myceliophthora thermophila
HSHLGYIIINGEVYQGFDPRPEQANSPLRVGWSTGAIDDGFVAPANYSSPDIICHIEGASPPAHAPVRAGDRVHVQWKRLAARTRGAGAVVPGALRRAGGVRERVDDSLPAMELVGAAGGAGGEDDGSGSDGSGSGGSGRVGVPGQRWATDVLIAANNSWQVEIPRGLRDGPYVLRHEIVALHYAAEPGGAQNYPLCVNLWVEGGDGSMELDHFDATQFYRPDDPGILLNVTAGLRSYAVPGPTLAAGATPVPYAQQNISSARADGTPVIVTRSTETVPFTAAPTPAETAEAKGGRYDDQTRTKDLNERFFYSSRPEQKRLTATSRRELVDHRTRYLSVAVCADFGAHKAAETNHEALRGGNKHHGGVSE

SEQ ID NO: 84 - DNA, 453 bp, Myceliophthora thermophila
atgaggtcgacattggccggtgccctggcagccatcgctgctcagaaagtagccggccacgccacgtttcagcagctctggcacggctcctcctgtgtccgccttccggctagcaactcacccgtcaccaatgtgggaagcagagacttcgtctgcaacgctggcacccgccccgtcagtggcaagtgccccgtgaaggctggcggcaccgtcaccatcgagatgcaccagcaacccggcgaccgcagctgcaacaacgaagccatcggaggggcgcattggggccccgtccaggtgtacctgaccaaggttcaggacgccgcgacggccgacggctcgacgggctggttcaagatcttctccgactcgtggtccaagaagcccgggggcaacttgggcgacgacgacaactggggcacgcgcgacctgaacgcctgctgcgggaagatggac

SEQ ID NO: 85 - protein, 151 aa, Myceliophthora thermophila
MRSTLAGALAAIAAQKVAGHATFQQLWHGSSCVRLPASNSPVTNVGSRDFVCNAGTRPVSGKCPVKAGGTVTIEMHQQPGDRSCNNEAIGGAHWGPVQVYLTKVQDAATADGSTGWFKIFSDSWSKKPGGNLGDDDNWGTRDLNACCGKMD

SEQ ID NO: 86 - protein, 132 aa, Myceliophthora thermophila
HATFQQLWHGSSCVRLPASNSPVTNVGSRDFVCNAGTRPVSGKCPVKAGGTVTIEMHQQPGDRSCNNEAIGGAHWGPVQVYLTKVQDAATADGSTGWFKIFSDSWSKKPGGNLGDDDNWGTRDLNACCGKMD

SEQ ID NO: 87 - DNA, 837 bp, Myceliophthora thermophila
atgaggtcgacattggccggtgccctggcagccatcgctgctcagaaagtagccggccacgccacgtttcagcagctctggcacggctcctcctgtgtccgccttccggctagcaactcacccgtcaccaatgtgggaagcagagacttcgtctgcaacgctggcacccgccccgtcagtggcaagtgccccgtgaaggctggcggcaccgtcaccatcgagatgcaccagcaacccggcgaccgcagctgcaacaacgaagccatcggaggggcgcattggggccccgtccaggtgtacctgaccaaggttcaggacgccgcgacggccgacggctcgacgggctggttcaagatcttctccgactcgtggtccaagaagcccgggggcaactcgggcgacgacgacaactggggcacgcgcgacctgaacgcctgctgcgggaagatggacgtggccatcccggccgacatcgcgtcgggcgactacctgctgcgggccgaggcgctggccctgcacacggccggacaggccggcggcgcccagttctacatgagctgctaccagatgacggtcgagggcggctccgggaccgccaacccgcccaccgtcaagttcccgggcgcctacagcgccaacgacccgggcatcctcgtcaacatccacgcccccctttccagctacaccgcgcccggcccggccgtctacgcgggcggcaccatccgcgaggccggctccgcctgcaccggctgcgcgcagacctgcaaggtcgggtcgtccccgagcgccgttgcccccggcagcggcgcgggcaacggcggcgggttccaaccccga

SEQ ID NO: 88 - protein, 279 aa, Myceliophthora thermophila
MRSTLAGALAAIAAQKVAGHATFQQLWHGSSCVRLPASNSPVTNVGSRDFVCNAGTRPVSGKCPVKAGGTVTIEMHQQPGDRSCNNEAIGGAHWGPVQVYLTKVQDAATADGSTGWFKIFSDSWSKKPGGNSGDDDNWGTRDLNACCGKMDVAIPADIASGDYLLRAEALALHTAGQAGGAQFYMSCYQMTVEGGSGTANPPTVKFPGAYSANDPGILVNIHAPLSSYTAPGPAVYAGGTIREAGSACTGCAQTCKVGSSPSAVAPGSGAGNGGGFQPR

SEQ ID NO: 89 - protein, 260 aa, Myceliophthora thermophila
HATFQQLWHGSSCVRLPASNSPVTNVGSRDFVCNAGTRPVSGKCPVKAGGTVTIEMHQQPGDRSCNNEAIGGAHWGPVQVYLTKVQDAATADGSTGWFKIFSDSWSKKPGGNSGDDDNWGTRDLNACCGKMDVAIPADIASGDYLLRAEALALHTAGQAGGAQFYMSCYQMTVEGGSGTANPPTVKFPGAYSANDPGILVNIHAPLSSYTAPGPAVYAGGTIREAGSACTGCAQTCKVGSSPSAVAPGSGAGNGGGFQPR

SEQ ID NO: 90 - DNA, 735 bp, Myceliophthora thermophila
atgctcctcctcaccctagccacactcgtcaccctcctggcgcgccacgtctcggctcacgcccggctgttccgcgtctctgtcgacgggaaagaccagggcgacgggctgaacaagtacatccgctcgccggcgaccaacgaccccgtgcgcgacctctcgagcgccgccatcgtgtgcaacacccaggggtccaaggccgccccggacttcgtcagggccgcggccggcgacaagctgaccttcctctgggcgcacgacaacccggacgacccggtcgactacgtcctcgacccgtcccacaagggcgccatcctgacctacgtcgccgcctacccctccggggacccgaccggccccatctggagcaagcttgccgaggaaggattcaccggcgggcagtgggcgaccatcaagatgatcgacaacggcggcaaggtcgacgtgacgctgcccgaggcccttgcgccgggaaagtacctgatccgccaggagctgctggccctgcaccgggccgactttgcctgcgacgacccggcccaccccaaccgcggcgccgagtcgtaccccaactgcgtccaggtggaggtgtcgggcagcggcgacaagaagccggaccagaactttgacttcaacaagggctatacctgcgataacaaaggactccactttaagatctacatcggtcaggacagccagtatgtggccccggggccgcggccttggaatgggagc

SEQ ID NO: 91 - protein, 245 aa, Myceliophthora thermophila
MLLLTLATLVTLLARHVSAHARLFRVSVDGKDQGDGLNKYIRSPATNDPVRDLSSAAIVCNTQGSKAAPDFVRAAAGDKLTFLWAHDNPDDPVDYVLDPSHKGAILTYVAAYPSGDPTGPIWSKLAEEGFTGGQWATIKMIDNGGKVDVTLPEALAPGKYLIRQELLALHRADFACDDPAHPNRGAESYPNCVQVEVSGSGDKKPDQNFDFNKGYTCDNKGLHFKIYIGQDSQYVAPGPRPWNGS

SEQ ID NO: 92 - protein, 226 aa, Myceliophthora thermophila
HARLFRVSVDGKDQGDGLNKYIRSPATNDPVRDLSSAAIVCNTQGSKAAPDFVRAAAGDKLTFLWAHDNPDDPVDYVLDPSHKGAILTYVAAYPSGDPTGPIWSKLAEEGFTGGQWATIKMIDNGGKVDVTLPEALAPGKYLIRQELLALHRADFACDDPAHPNRGAESYPNCVQVEVSGSGDKKPDQNFDFNKGYTCDNKGLHFKIYIGQDSQYVAPGPRPWNGS

SEQ ID NO: 93 - DNA, 600 bp, Myceliophthora thermophila
atgttcacttcgctttgcatcacagatcattggaggactcttagcagccactctgggccagtcatgaactatctcgcccattgcaccaatgacgactgcaagtctttcaagggcgacagcggcaacgtctgggtcaagatcgagcagctcgcgtacaacccgtcagccaaccccccctgggcgtctgacctcctccgtgagcacggtgccaagtggaaggtgacgatcccgcccagtcttgtccccggcgaatatctgctgcggcacgagatcctggggttgcacgtcgcaggaaccgtgatgggcgcccagttctaccccggctgcacccagatcagggtcaccgaaggcgggagcacgcagctgccctcgggtattgcgctcccaggcgcttacggcccacaagacgagggtatcttggtcgacttgtggagggttaaccagggccaggtcaactacacggcgcctggaggacccgtttggagcgaagcgtgggacaccgagtttggcgggtccaacacgaccgagtgcgccaccatgctcgacgacctgctcgactacatggcggccaacgacgagtggatcggctggacggcctag

SEQ ID NO: 94 - protein, 199 aa, Myceliophthora thermophila
MFTSLCITDHWRTLSSHSGPVMNYLAHCTNDDCKSFKGDSGNVWVKIEQLAYNPSANPPWASDLLREHGAKWKVTIPPSLVPGEYLLRHEILGLHVAGTVMGAQFYPGCTQIRVTEGGSTQLPSGIALPGAYGPQDEGILVDLWRVNQGQVNYTAPGGPVWSEAWDTEFGGSNTTECATMLDDLLDYMAANDEWIGWTA

SEQ ID NO: 95 - DNA, 693 bp, Myceliophthora thermophila
atgaactatctcgcccattgcaccaatgacgactgcaagtctttcaagggcgacagcggcaacgtctgggtcaagatcgagcagctcgcgtacaacccgtcagccaaccccccctgggcgtctgacctcctccgtgagcacggtgccaagtggaaggtgacgatcccgcccagtcttgtccccggcgaatatctgctgcggcacgagatcctggggttgcacgtcgcaggaaccgtgatgggcgcccagttctaccccggctgcacccagatcagggtcaccgaaggcgggagcacgcagctgccctcgggtattgcgctcccaggcgcttacggcccacaagacgagggtatcttggtcgacttgtggagggttaaccagggccaggtcaactacacggcgcctggaggacccgtttggagcgaagcgtgggacaccgagtttggcgggtccaacacgaccgagtgcgccaccatgctcgacgacctgctcgactacatggcggccaacgacgacccatgctgcaccgaccagaaccagttcgggagtctcgagccggggagcaaggcggccggcggctcgccgagcctgtacgataccgtcttggtccccgttctccagaagaaagtgccgacaaagctgcagtggagcggaccggcgagcgtcaacggggatgagttgacagagaggccc

SEQ ID NO: 96 - protein, 231 aa, Myceliophthora thermophila
MNYLAHCTNDDCKSFKGDSGNVWVKIEQLAYNPSANPPWASDLLREHGAKWKVTIPPSLVPGEYLLRHEILGLHVAGTVMGAQFYPGCTQIRVTEGGSTQLPSGIALPGAYGPQDEGILVDLWRVNQGQVNYTAPGGPVWSEAWDTEFGGSNTTECATMLDDLLDYMAANDDPCCTDQNQFGSLEPGSKAAGGSPSLYDTVLVPVLQKKVPTKLQWSGPASVNGDELTERP

SEQ ID NO: 97 - DNA, 681 bp, Myceliophthora thermophila
atgaagctgagcgctgccatcgccgtgctcgcggccgcccttgccgaggggcactataccttccccagcatcgccaacacggccgactggcaatatgtgcgcatcacgaccaacttccagagcaacggccccgtgacggacgtcaactcggaccagatccggtgctacgagcgcaacccgggcaccggcgcccccggcatctacaacgtcacggccggcacaaccatcaactacaacgccaagtcgtccatctcccacccgggacccatggccttctacattgccaaggttcccgccggccagtcggccgccacctgggacggtaagggcgccgtctggtccaagatccaccaggagatgccgcactttggcaccagcctcacctgggactccaacggccgcacctccatgcccgtcaccatcccccgctgtctgcaggacggcgagtatctgctgcgtgcagagcacattgccctccacagcgccggcagccccggcggcgcccagttctacatttcttgtgcccagctctcagtcaccggcggcagcgggacctggaaccccaggaacaaggtgtcgttccccggcgcctacaaggccactgacccgggcatcctgatcaacatctactaccccgtcccgactagctacactcccgctggtccccccgtcgacacctgc

SEQ ID NO: 98 - protein, 227 aa, Myceliophthora thermophila
MKLSAAIAVLAAALAEGHYTFPSIANTADWQYVRITTNFQSNGPVTDVNSDQIRCYERNPGTGAPGIYNVTAGTTINYNAKSSISHPGPMAFYIAKVPAGQSAATWDGKGAVWSKIHQEMPHFGTSLTWDSNGRTSMPVTIPRCLQDGEYLLRAEHIALHSAGSPGGAQFYISCAQLSVTGGSGTWNPRNKVSFPGAYKATDPGILINIYYPVPTSYTPAGPPVDTC

SEQ ID NO: 99 - protein, 210 aa, Myceliophthora thermophila
HYTFPSIANTADWQYVRITTNFQSNGPVTDVNSDQIRCYERNPGTGAPGIYNVTAGTTINYNAKSSISHPGPMAFYIAKVPAGQSAATWDGKGAVWSKIHQEMPHFGTSLTWDSNGRTSMPVTIPRCLQDGEYLLRAEHIALHSAGSPGGAQFYISCAQLSVTGGSGTWNPRNKVSFPGAYKATDPGILINIYYPVPTSYTPAGPPVDTC

SEQ ID NO: 100 - DNA, 765 bp, Myceliophthora thermophila
atgtaccgcacgctcggttccattgccctgctcgcggggggcgctgccgcccacggcgccgtgaccagctacaacattgcgggcaaggactaccctggatactcgggcttcgcccctaccggccaggatgtcatccagtggcaatggcccgactataaccccgtgctgtccgccagcgaccccaagctccgctgcaacggcggcaccggggcggcgctgtatgccgaggcggcccccggcgacaccatcacggccacctgggcccagtggacgcactcccagggcccgatcctggtgtggatgtacaagtgccccggcgacttcagctcctgcgacggctccggcgcgggttggttcaagatcgacgaggccggcttccacggcgacggcacgaccgtcttcctcgacaccgagaccccctcgggctgggacattgccaagctggtcggcggcaacaagtcgtggagcagcaagatccctgacggcctcgccccgggcaattacctggtccgccacgagctcatcgccctgcaccaggccaacaacccgcaattctaccccgagtgcgcccagatcaaggtcaccggctctggcaccgccgagcccgccgcctcctacaaggccgccatccccggctactgccagcagagcgaccccaacatttcgttcaacatcaacgaccactccctcccgcaggagtacaagatccccggtcccccggtcttcaagggcaccgcctccgccaaggctcgcgctttccaggcc

SEQ ID NO: 101 - protein, 255 aa, Myceliophthora thermophila
MYRTLGSIALLAGGAAAHGAVTSYNIAGKDYPGYSGFAPTGQDVIQWQWPDYNPVLSASDPKLRCNGGTGAALYAEAAPGDTITATWAQWTHSQGPILVWMYKCPGDFSSCDGSGAGWFKIDEAGFHGDGTTVFLDTETPSGWDIAKLVGGNKSWSSKIPDGLAPGNYLVRHELIALHQANNPQFYPECAQIKVTGSGTAEPAASYKAAIPGYCQQSDPNISFNINDHSLPQEYKIPGPPVFKGTASAKARAFQA

SEQ ID NO: 102 - protein, 236 aa, Myceliophthora thermophila
AVTSYNIAGKDYPGYSGFAPTGQDVIQWQWPDYNPVLSASDPKLRCNGGTGAALYAEAAPGDTITATWAQWTHSQGPILVWMYKCPGDFSSCDGSGAGWFKIDEAGFHGDGTTVFLDTETPSGWDIAKLVGGNKSWSSKIPDGLAPGNYLVRHELIALHQANNPQFYPECAQIKVTGSGTAEPAASYKAAIPGYCQQSDPNISFNINDHSLPQEYKIPGPPVFKGTASAKARAFQA

SEQ ID NO: 103 - DNA, 675 bp, Myceliophthora thermophila
atgctgacaacaaccttcgccctcctgacggccgctctcggcgtcagcgcccattataccctccccagggtcgggaccggttccgactggcagcacgtgcggcgggctgacaactggcaaaacaacggcttcgtcggcgacgtcaactcggagcagatcaggtgcttccaggcgacccctgccggcgcccaagacgtctacactgttcaggcgggatcgaccgtgacctaccacgccaaccccagtatctaccaccccggccccatgcagttctacctggcccgcgttccggacggacaggacgtcaagtcgtggaccggcgagggtgccgtgtggttcaaggtgtacgaggagcagcctcaatttggcgcccagctgacctggcctagcaacggcaagagctcgttcgaggttcctatccccagctgcattcgggcgggcaactacctcctccgcgctgagcacatcgccctgcacgttgcccaaagccagggcggcgcccagttctacatctcgtgcgcccagctccaggtcactggtggcggcagcaccgagccttctcagaaggtttccttcccgggtgcctacaagtccaccgaccccggcattcttatcaacatcaactaccccgtccctacctcgtaccagaatccgggtccggctgtcttccgttgc

SEQ ID NO: 104 - protein, 225 aa, Myceliophthora thermophila
MLTTTFALLTAALGVSAHYTLPRVGTGSDWQHVRRADNWQNNGFVGDVNSEQIRCFQATPAGAQDVYTVQAGSTVTYHANPSIYHPGPMQFYLARVPDGQDVKSWTGEGAVWFKVYEEQPQFGAQLTWPSNGKSSFEVPIPSCIRAGNYLLRAEHIALHVAQSQGGAQFYISCAQLQVTGGGSTEPSQKVSFPGAYKSTDPGILININYPVPTSYQNPGPAVFRC

SEQ ID NO: 105 - protein, 208 aa, Myceliophthora thermophila
HYTLPRVGTGSDWQHVRRADNWQNNGFVGDVNSEQIRCFQATPAGAQDVYTVQAGSTVTYHANPSIYHPGPMQFYLARVPDGQDVKSWTGEGAVWFKVYEEQPQFGAQLTWPSNGKSSFEVPIPSCIRAGNYLLRAEHIALHVAQSQGGAQFYISCAQLQVTGGGSTEPSQKVSFPGAYKSTDPGILININYPVPTSYQNPGPAVFRC

SEQ ID NO: 106 - DNA, 711 bp, Myceliophthora thermophila
atgaaggttctcgcgcccctgattctggccggtgccgccagcgcccacaccatcttctcatccctcgaggtgggcggcgtcaaccagggcatcgggcagggtgtccgcgtgccgtcgtacaacggtccgatcgaggacgtgacgtccaactcgatcgcctgcaacgggccccccaacccgacgacgccgaccaacaaggtcatcacggtccgggccggcgagacggtgacggccgtctggcggtacatgctgagcaccaccggctcggcccccaacgacatcatggacagcagccacaagggcccgaccatggcctacctcaagaaggtcgacaacgccaccaccgactcgggcgtcggcggcggctggttcaagatccaggaggacggccttaccaacggcgtctggggcaccgagcgcgtcatcaacggccagggccgccacaacatcaagatccccgagtgcatcgcccccggccagtacctcctccgcgccgagatgcttgccctgcacggagcttccaactaccccggcgctcagttctacatggagtgcgcccagctcaatatcgtcggcggcaccggcagcaagacgccgtccaccgtcagcttcccgggcgcttacaagggtaccgaccccggagtcaagatcaacatctactggccccccgtcaccagctaccagattcccggccccggcgtgttcacctgc

SEQ ID NO: 107 - protein, 237 aa, Myceliophthora thermophila
MKVLAPLILAGAASAHTIFSSLEVGGVNQGIGQGVRVPSYNGPIEDVTSNSIACNGPPNPTTPTNKVITVRAGETVTAVWRYMLSTTGSAPNDIMDSSHKGPTMAYLKKVDNATTDSGVGGGWFKIQEDGLTNGVWGTERVINGQGRHNIKIPECIAPGQYLLRAEMLALHGASNYPGAQFYMECAQLNIVGGTGSKTPSTVSFPGAYKGTDPGVKINIYWPPVTSYQIPGPGVFTC

SEQ ID NO: 108 - protein, 222 aa, Myceliophthora thermophila
HTIFSSLEVGGVNQGIGQGVRVPSYNGPIEDVTSNSIACNGPPNPTTPTNKVITVRAGETVTAVWRYMLSTTGSAPNDIMDSSHKGPTMAYLKKVDNATTDSGVGGGWFKIQEDGLTNGVWGTERVINGQGRHNIKIPECIAPGQYLLRAEMLALHGASNYPGAQFYMECAQLNIVGGTGSKTPSTVSFPGAYKGTDPGVKINIYWPPVTSYQIPGPGVFTC

SEQ ID NO: 109 - DNA, 225 bp, Myceliophthora thermophila
atgatcgacaacctccctgatgactccctacaacccgcctgcctccgcccgggccactacctcgtccgccacgagatcatcgcgctgcactcggcctgggccgagggcgaggcccagttctaccccttccccctttttcctttttttccctcccttcttttgtccggtaactacacgattcccggtcccgcgatctggaagtgcccagaggcacagcagaacgag

SEQ ID NO: 110 - protein, 75 aa, Myceliophthora thermophila
MIDNLPDDSLQPACLRPGHYLVRHEIIALHSAWAEGEAQFYPFPLFPFFPSLLLSGNYTIPGPAIWKCPEAQQNE

SEQ ID NO: 111 - protein, 57 aa, Myceliophthora thermophila
HYLVRHEIIALHSAWAEGEAQFYPFPLFPFFPSLLLSGNYTIPGPAIWKCPEAQQNE

SEQ ID NO: 112 - DNA, 1170 bp, Myceliophthora thermophila
atgaagtcctccatcctcgccagcgtcttcgccacgggcgccgtggctcaaagtggtccgtggcagcaatgtggtggcatcggatggcaaggatcgaccgactgtgtgtcgggttaccactgcgtctaccagaacgattggtacagccagtgcgtgcctggcgcggcgtcgacaacgctccagacatctaccacgtccaggcccaccgccaccagcaccgcccctccgtcgtccaccacctcgcctagcaagggcaagctcaagtggctcggcagcaacgagtcgggcgccgagttcggggagggcaactaccccggcctctggggcaagcacttcatcttcccgtcgacttcggcgattcagacgctcatcaatgatggatacaacatcttccggatcgacttctcgatggagcgtctggtgcccaaccagttgacgtcgtccttcgacgagggctacctccgcaacctgaccgaggtggtcaacttcgtgacgaacgcgggcaagtacgccgtcctggacccgcacaactacggccggtactacggcaacgtcatcacggacacgaacgcgttccggaccttctggaccaacctggccaagcagttcgcctccaactcgctcgtcatcttcgacaccaacaacgagtacaacacgatggaccagaccctggtgctcaacctcaaccaggccgccatcgacggcatccgggccgccggcgcgacctcgcagtacatcttcgtcgagggcaacgcgtggagcggggcctggagctggaacacgaccaacaccaacatggccgccctgacggacccgcagaacaagatcgtgtacgagatgcaccagtacctcgactcggacagctcgggcacccacgccgagtgcgtcagcagcaacatcggcgcccagcgcgtcgtcggagccacccagtggctccgcgccaacggcaagctcggcgtcctcggcgagttcgccggcggcgccaacgccgtctgccagcaggccgtcaccggcctcctcgaccacctccaggacaacagcgacgtctggctgggtgccctctggtgggccgccggtccctggtggggcgactacatgtactcgttcgagcctccttcgggcaccggctatgtcaactacaactcgatcctaaagaagtacttgccgtaa

SEQ ID NO: 113 - protein, 389 aa, Myceliophthora thermophila
MKSSILASVFATGAVAQSGPWQQCGGIGWQGSTDCVSGYHCVYQNDWYSQCVPGAASTTLQTSTTSRPTATSTAPPSSTTSPSKGKLKWLGSNESGAEFGEGNYPGLWGKHFIFPSTSAIQTLINDGYNIFRIDFSMERLVPNQLTSSFDEGYLRNLTEVVNFVTNAGKYAVLDPHNYGRYYGNVITDTNAFRTFWTNLAKQFASNSLVIFDTNNEYNTMDQTLVLNLNQAAIDGIRAAGATSQYIFVEGNAWSGAWSWNTTNTNMAALTDPQNKIVYEMHQYLDSDSSGTHAECVSSNIGAQRVVGATQWLRANGKLGVLGEFAGGANAVCQQAVTGLLDHLQDNSEVWLGALWWAAGPWWGDYMYSFEPPSGTGYVNYNSILKKYLP

SEQ ID NO: 114 - protein, 373 aa, Myceliophthora thermophila
QSGPWQQCGGIGWQGSTDCVSGYHCVYQNDWYSQCVPGAASTTLQTSTTSRPTATSTAPPSSTTSPSKGKLKWLGSNESGAEFGEGNYPGLWGKHFIFPSTSAIQTLINDGYNIFRIDFSMERLVPNQLTSSFDEGYLRNLTEVVNFVTNAGKYAVLDPHNYGRYYGNVITDTNAFRTFWTNLAKQFASNSLVIFDTNNEYNTMDQTLVLNLNQAAIDGIRAAGATSQYIFVEGNAWSGAWSWNTTNTNMAALTDPQNKIVYEMHQYLDSDSSGTHAECVSSNIGAQRVVGATQWLRANGKLGVLGEFAGGANAVCQQAVTGLLDHLQDNSEVWLGALWWAAGPWWGDYMYSFEPPSGTGYVNYNSILKKYLP

SEQ ID NO: 115 - DNA, 2613 bp, Myceliophthora thermophila
atgaaggctgctgcgctttcctgcctcttcggcagtacccttgccgttgcaggcgccattgaatcgagaaaggttcaccagaagcccctcgcgagatctgaacctttttacccgtcgccatggatgaatcccaacgccgacggctgggcggaggcctatgcccaggccaagtcctttgtctcccaaatgactctgctagagaaggtcaacttgaccacgggagtcggctggggggctgagcagtgcgtcggccaagtgggcgcgatccctcgccttggacttcgcagtctgtgcatgcatgactcccctctcggcatccgaggagccgactacaactcagcgttcccctctggccagaccgttgctgctacctgggatcgcggtctgatgtaccgtcgcggctacgcaatgggccaggaggccaaaggcaagggcatcaatgtccttctcggaccagtcgccggcccccttggccgcatgcccgagggcggtcgtaactgggaaggcttcgctccggatcccgtccttaccggcatcggcatgtccgagacgatcaagggcattcaggatgctggcgtcatcgcttgtgcgaagcactttattggaaacgagcaggagcacttcagacaggtgccagaagcccagggatacggttacaacatcagcgaaaccctctcctccaacattgacgacaagaccatgcacgagctctacctttggccgtttgccgatgccgtccgggccggcgtcggctctgtcatgtgctcgtaccagcaggtcaacaactcgtacgcctgccagaactcgaagctgctgaacgacctcctcaagaacgagcttgggtttcagggcttcgtcatgagcgactggcaggcacagcacactggcgcagcaagcgccgtggctggtctcgatatgtccatgccgggcgacacccagttcaacactggcgtcagtttctggggcgccaatctcaccctcgccgtcctcaacggcacagtccctgcctaccgtctcgacgacatggccatgcgcatcatggccgccctcttcaaggtcaccaagaccaccgacctggaaccgatcaacttctccttctggaccgacgacacttatggcccgatccactgggccgccaagcagggctaccaggagattaattcccacgttgacgtccgcgccgaccacggcaacctcatccgggagattgccgccaagggtacggtgctgctgaagaataccggctctctacccctgaacaagccaaagttcgtggccgtcatcggcgaggatgctgggtcgagccccaacgggcccaacggctgcagcgaccgcggctgtaacgaaggcacgctcgccatgggctggggatccggcacagccaactatccgtacctcgtttcccccgacgccgcgctccaggcccgggccatccaggacggcacgaggtacgagagcgtcctgtccaactacgccgaggaaaagacaaaggctctggtctcgcaggccaatgcaaccgccatcgtcttcgtcaatgccgactcaggcgagggctacatcaacgtggacggtaacgagggcgaccgtaagaacctgactctctggaacaacggtgatactctggtcaagaacgtctcgagctggtgcagcaacaccatcgtcgtcatccactcggtcggcccggtcctcctgaccgattggtacgacaaccccaacatcacggccattctctgggctggtcttccgggccaggagtcgggcaactccatcaccgacgtgctttacggcaaggtcaaccccgccgcccgctcgcccttcacttggggcaagacccgcgaaagctatggcgcggacgtcctgtacaagccgaataatggcaatggtgcgccccaacaggacttcaccgagggcgtcttcatcgactaccgctacttcgacaaggttgacgatgactcggtcatctacgagttcggccacggcctgagctacaccaccttcgagtacagcaacatccgcgtcgtcaagtccaacgtcagcgagtaccggcccacgacgggcaccacggcccaggccccgacgtttggcaacttctccaccgacctcgaggactatctcttccccaaggacgagttcccctacatctaccagtacatctacccgtacctcaacacgaccgacccccggagggcctcggccgatccccactacggccagaccgccgaggagttcctcccgccccacgccaccgatgacgacccccagccgctcctccggtcctcgggcggaaactcccccggcggcaaccgccagctgtacgacattgtctacacaatcacggccgacatcacgaatacgggctccgttgtaggcgaggaggtaccgcagctctacgtctcgctgggcggtcccgaggatcccaaggtgcagctgcgcgactttgacaggatgcggatcgaacccggcgagacgaggcagttcaccggccgcctgacgcgcagagatctgagcaactgggacgtcacggtgcaggactgggtcatcagcaggtatcccaagacggcatatgttgggaggagcagccggaagttggatctcaagattgagcttccttga

SEQ ID NO: 116 - protein, 870 aa, Myceliophthora thermophila
MKAAALSCLFGSTLAVAGAIESRKVHQKPLARSEPFYPSPWMNPNADGWAEAYAQAKSFVSQMTLLEKVNLTTGVGWGAEQCVGQVGAIPRLGLRSLCMHDSPLGIRGADYNSAFPSGQTVAATWDRGLMYRRGYAMGQEAKGKGINVLLGPVAGPLGRMPEGGRNWEGFAPDPVLTGIGMSETIKGIQDAGVIACAKHFIGNEQEHFRQVPEAQGYGYNISETLSSNIDDKTMHELYLWPFADAVRAGVGSVMCSYQQVNNSYACQNSKLLNDLLKNELGFQGFVMSDWQAQHTGAASAVAGLDMSMPGDTQFNTGVSFWGANLTLAVLNGTVPAYRLDDMAMRIMAALFKVTKTTDLEPINFSFWTDDTYGPIHWAAKQGYQEINSHVDVRADHGNLIREIAAKGTVLLKNTGSLPLNKPKFVAVIGEDAGSSPNGPNGCSDRGCNEGTLAMGWGSGTANYPYLVSPDAALQARAIQDGTRYESVLSNYAEEKTKALVSQANATAIVFVNADSGEGYINVDGNEGDRKNLTLWNNGDTLVKNVSSWCSNTIVVIHSVGPVLLTDWYDNPNITAILWAGLPGQESGNSITDVLYGKVNPAARSPFTWGKTRESYGADVLYKPNNGNGAPQQDFTEGVFIDYRYFDKVDDDSVIYEFGHGLSYTTFEYSNIRVVKSNVSEYRPTTGTTAQAPTFGNFSTDLEDYLFPKDEFPYIYQYIYPYLNTTDPRRASADPHYGQTAEEFLPPHATDDDPQPLLRSSGGNSPGGNRQLYDIVYTITADITNTGSVVGEEVPQLYVSLGGPEDPKVQLRDFDRMRIEPGETRQFTGRLTRRDLSNWDVTVQDWVISRYPKTAYVGRSSRKLDLKIELP

SEQ ID NO: 117 - protein, 851 aa, Myceliophthora thermophila
IESRKVHQKPLARSEPFYPSPWMNPNADGWAEAYAQAKSFVSQMTLLEKVNLTTGVGWGAEQCVGQVGAIPRLGLRSLCMHDSPLGIRGADYNSAFPSGQTVAATWDRGLMYRRGYAMGQEAKGKGINVLLGPVAGPLGRMPEGGRNWEGFAPDPVLTGIGMSETIKGIQDAGVIACAKHFIGNEQEHFRQVPEAQGYGYNISETLSSNIDDKTMHELYLWPFADAVRAGVGSVMCSYQQVNNSYACQNSKLLNDLLKNELGFQGFVMSDWQAQHTGAASAVAGLDMSMPGDTQFNTGVSFWGANLTLAVLNGTVPAYRLDDMAMRIMAALFKVTKTTDLEPINFSFWTDDTYGPIHWAAKQGYQEINSHVDVRADHGNLIREIAAKGTVLLKNTGSLPLNKPKFVAVIGEDAGSSPNGPNGCSDRGCNEGTLAMGWGSGTANYPYLVSPDAALQARAIQDGTRYESVLSNYAEEKTKALVSQANATAIVFVNADSGEGYINVDGNEGDRKNLTLWNNGDTLVKNVSSWCSNTIVVIHSVGPVLLTDWYDNPNITAILWAGLPGQESGNSITDVLYGKVNPAARSPFTWGKTRESYGADVLYKPNNGNGAPQQDFTEGVFIDYRYFDKVDDDSVIYEFGHGLSYTTFEYSNIRVVKSNVSEYRPTTGTTAQAPTFGNFSTDLEDYLFPKDEFPYIYQYIYPYLNTTDPRRASADPHYGQTAEEFLPPHATDDDPQPLLRSSGGNSPGGNRQLYDIVYTITADITNTGSVVGEEVPQLYVSLGGPEDPKVQLRDFDRMRIEPGETRQFTGRLTRRDLSNWDVTVQDWVISRYPKTAYVGRSSRKLDLKIELP

SEQ ID NO: 118 - DNA, 2613 bp, Artificial Sequence
  Synthetic polynucleotide for BGL Variant 883
atgaaggctgctgcgctttcctgcctcttcggcagtacccttgccgttgcaggcgccattgaatcgagaaaggttcaccagaagcccctcgcgagatctgaacctttttacccgtcgccatggatgaatcccaacgccgacggctgggcggaggcctatgcccaggccaagtcctttgtctcccaaatgactctgctagagaaggtcaacttgaccacgggagtcggctggggggctgagcagtgcgtcggccaagtgggcgcgatccctcgccttggacttcgcagtctgtgcatgcatgactcccctctcggcatccgaggagccgactacaactcagcgttcccctctggccagaccgttgctgctacctgggatcgcggtctgatgtaccgtcgcggctacgcaatgggccaggaggccaaaggcaagggcatcaatgtccttctcggaccagtcgccggcccccttggccgcatgcccgagggcggtcgtaactgggaaggcttcgctccggatcccgtccttaccggcatcggcatgtccgagacgatcaagggcattcaggatgctggcgtcatcgcttgtgcgaagcactttattggaaacgagcaggagcacttcagacaggtgccagaagcccagggatacggttacaacatcagcgaaaccctctcctccaacattgacgacaagaccatgcacgagctctacctttggccgtttgccgatgccgtccgggccggcgtcggctctgtcatgtgctcgtacaaccaggtcaacaactcgtacgcctgccagaactcgaagctgctgaacgacctcctcaagaacgagcttgggtttcagggcttcgtcatgagcgactggtgggcacagcacactggcgcagcaagcgccgtggctggtctcgatatgtccatgccgggcgacaccatgttcaacactggcgtcagtttctggggcgccaatctcaccctcgccgtcctcaacggcacagtccctgcctaccgtctcgacgacatggccatgcgcatcatggccgccctcttcaaggtcaccaagaccaccgacctggaaccgatcaacttctccttctggacccgcgacacttatggcccgatccactgggccgccaagcagggctaccaggagattaattcccacgttgacgtccgcgccgaccacggcaacctcatccggaacattgccgccaagggtacggtgctgctgaagaataccggctctctacccctgaacaagccaaagttcgtggccgtcatcggcgaggatgctgggccgagccccaacgggcccaacggctgcagcgaccgcggctgtaacgaaggcacgctcgccatgggctggggatccggcacagccaactatccgtacctcgtttcccccgacgccgcgctccagttgcgggccatccaggacggcacgaggtacgagagcgtcctgtccaactacgccgaggaaaatacaaaggctctggtctcgcaggccaatgcaaccgccatcgtcttcgtcaatgccgactcaggcgagggctacatcaacgtggacggtaacgagggcgaccgtaagaacctgactctctggaacaacggtgatactctggtcaagaacgtctcgagctggtgcagcaacaccatcgtcgtcatccactcggtcggcccggtcctcctgaccgattggtacgacaaccccaacatcacggccattctctgggctggtcttccgggccaggagtcgggcaactccatcaccgacgtgctttacggcaaggtcaaccccgccgcccgctcgcccttcacttggggcaagacccgcgaaagctatggcgcggacgtcctgtacaagccgaataatggcaattgggcgccccaacaggacttcaccgagggcgtcttcatcgactaccgctacttcgacaaggttgacgatgactcggtcatctacgagttcggccacggcctgagctacaccaccttcgagtacagcaacatccgcgtcgtcaagtccaacgtcagcgagtaccggcccacgacgggcaccacgattcaggccccgacgtttggcaacttctccaccgacctcgaggactatctcttccccaaggacgagttcccctacatcccgcagtacatctacccgtacctcaacacgaccgacccccggagggcctcggccgatccccactacggccagaccgccgaggagttcctcccgccccacgccaccgatgacgacccccagccgctcctccggtcctcgggcggaaactcccccggcggcaaccgccagctgtacgacattgtctacacaatcacggccgacatcacgaatacgggctccgttgtaggcgaggaggtaccgcagctctacgtctcgctgggcggtcccgaggatcccaaggtgcagctgcgcgactttgacaggatgcggatcgaacccggcgagacgaggcagttcaccggccgcctgacgcgcagagatctgagcaactgggacgtcacggtgcaggactgggtcatcagcaggtatcccaagacggcatatgttgggaggagcagccggaagttggatctcaagattgagcttccttga

SEQ ID NO: 119 - protein, 870 aa, Artificial Sequence
  Synthetic polypeptide for BGL Variant 883
MKAAALSCLFGSTLAVAGAIESRKVHQKPLARSEPFYPSPWMNPNADGWAEAYAQAKSFVSQMTLLEKVNLTTGVGWGAEQCVGQVGAIPRLGLRSLCMHDSPLGIRGADYNSAFPSGQTVAATWDRGLMYRRGYAMGQEAKGKGINVLLGPVAGPLGRMPEGGRNWEGFAPDPVLTGIGMSETIKGIQDAGVIACAKHFIGNEQEHFRQVPEAQGYGYNISETLSSNIDDKTMHELYLWPFADAVRAGVGSVMCSYNQVNNSYACQNSKLLNDLLKNELGFQGFVMSDWWAQHTGAASAVAGLDMSMPGDTMFNTGVSFWGANLTLAVLNGTVPAYRLDDMAMRIMAALFKVTKTTDLEPINFSFWTRDTYGPIHWAAKQGYQEINSHVDVRADHGNLIRNIAAKGTVLLKNTGSLPLNKPKFVAVIGEDAGPSPNGPNGCSDRGCNEGTLAMGWGSGTANYPYLVSPDAALQLRAIQDGTRYESVLSNYAEENTKALVSQANATAIVFVNADSGEGYINVDGNEGDRKNLTLWNNGDTLVKNVSSWCSNTIVVIHSVGPVLLTDWYDNPNITAILWAGLPGQESGNSITDVLYGKVNPAARSPFTWGKTRESYGADVLYKPNNGNWAPQQDFTEGVFIDYRYFDKVDDDSVIYEFGHGLSYTTFEYSNIRVVKSNVSEYRPTTGTTIQAPTFGNFSTDLEDYLFPKDEFPYIPQYIYPYLNTTDPRRASADPHYGQTAEEFLPPHATDDDPQPLLRSSGGNSPGGNRQLYDIVYTITADITNTGSVVGEEVPQLYVSLGGPEDPKVQLRDFDRMRIEPGETRQFTGRLTRRDLSNWDVTVQDWVISRYPKTAYVGRSSRKLDLKIELP

SEQ ID NO: 120 - protein, 851 aa, Artificial Sequence
  Synthetic polypeptide for BGL Variant 883
IESRKVHQKPLARSEPFYPSPWMNPNADGWAEAYAQAKSFVSQMTLLEKVNLTTGVGWGAEQCVGQVGAIPRLGLRSLCMHDSPLGIRGADYNSAFPSGQTVAATWDRGLMYRRGYAMGQEAKGKGINVLLGPVAGPLGRMPEGGRNWEGFAPDPVLTGIGMSETIKGIQDAGVIACAKHFIGNEQEHFRQVPEAQGYGYNISETLSSNIDDKTMHELYLWPFADAVRAGVGSVMCSYNQVNNSYACQNSKLLNDLLKNELGFQGFVMSDWWAQHTGAASAVAGLDMSMPGDTMFNTGVSFWGANLTLAVLNGTVPAYRLDDMAMRIMAALFKVTKTTDLEPINFSFWTRDTYGPIHWAAKQGYQEINSHVDVRADHGNLIRNIAAKGTVLLKNTGSLPLNKPKFVAVIGEDAGPSPNGPNGCSDRGCNEGTLAMGWGSGTANYPYLVSPDAALQLRAIQDGTRYESVLSNYAEENTKALVSQANATAIVFVNADSGEGYINVDGNEGDRKNLTLWNNGDTLVKNVSSWCSNTIVVIHSVGPVLLTDWYDNPNITAILWAGLPGQESGNSITDVLYGKVNPAARSPFTWGKTRESYGADVLYKPNNGNWAPQQDFTEGVFIDYRYFDKVDDDSVIYEFGHGLSYTTFEYSNIRVVKSNVSEYRPTTGTTIQAPTFGNFSTDLEDYLFPKDEFPYIPQYIYPYLNTTDPRRASADPHYGQTAEEFLPPHATDDDPQPLLRSSGGNSPGGNRQLYDIVYTITADITNTGSVVGEEVPQLYVSLGGPEDPKVQLRDFDRMRIEPGETRQFTGRLTRRDLSNWDVTVQDWVISRYPKTAYVGRSSRKLDLKIELP

SEQ ID NO: 121 - DNA, 2613 bp, Artificial Sequence
  Synthetic polynucleotide for BGL Variant 900
atgaaggctgctgcgctttcctgcctcttcggcagtacccttgccgttgcaggcgccattgaatcgagaaaggttcaccagaagcccctcgcgagatctgaacctttttacccgtcgccatggatgaatcccaacgccatcggctgggcggaggcctatgcccaggccaagtcctttgtctcccaaatgactctgctagagaaggtcaacttgaccacgggagtcggctggggggaggagcagtgcgtcggcaacgtgggcgcgatccctcgccttggacttcgcagtctgtgcatgcatgactcccctctcggcgtgcgaggaaccgactacaactcagcgttcccctctggccagaccgttgctgctacctgggatcgcggtctgatgtaccgtcgcggctacgcaatgggccaggaggccaaaggcaagggcatcaatgtccttctcggaccagtcgccggcccccttggccgcatgcccgagggcggtcgtaactgggaaggcttcgctccggatcccgtccttaccggcatcggcatgtccgagacgatcaagggcattcaggatgctggcgtcatcgcttgtgcgaagcactttattggaaacgagcaggagcacttcagacaggtgccagaagcccagggatacggttacaacatcagcgaaaccctctcctccaacattgacgacaagaccatgcacgagctctacctttggccgtttgccgatgccgtccgggccggcgtcggctctgtcatgtgctcgtacaaccagggcaacaactcgtacgcctgccagaactcgaagctgctgaacgacctcctcaagaacgagcttgggtttcagggcttcgtcatgagcgactggtgggcacagcacactggcgcagcaagcgccgtggctggtctcgatatgtccatgccgggcgacaccatggtcaacactggcgtcagtttctggggcgccaatctcaccctcgccgtcctcaacggcacagtccctgcctaccgtctcgacgacatgtgcatgcgcatcatggccgccctcttcaaggtcaccaagaccaccgacctggaaccgatcaacttctccttctggacccgcgacacttatggcccgatccactgggccgccaagcagggctaccaggagattaattcccacgttgacgtccgcgccgaccacggcaacctcatccggaacattgccgccaagggtacggtgctgctgaagaataccggctctctacccctgaacaagccaaagttcgtggccgtcatcggcgaggatgctgggccgagccccaacgggcccaacggctgcagcgaccgcggctgtaacgaaggcacgctcgccatgggctggggatccggcacagccaactatccgtacctcgtttcccccgacgccgcgctccaggcgcgggccatccaggacggcacgaggtacgagagcgtcctgtccaactacgccgaggaaaatacaaaggctctggtctcgcaggccaatgcaaccgccatcgtcttcgtcaatgccgactcaggcgagggctacatcaacgtggacggtaacgagggcgaccgtaagaacctgactctctggaacaacggtgatactctggtcaagaacgtctcgagctggtgcagcaacaccatcgtcgtcatccactcggtcggcccggtcctcctgaccgattggtacgacaaccccaacatcacggccattctctgggctggtcttccgggccaggagtcgggcaactccatcaccgacgtgctttacggcaaggtcaaccccgccgcccgctcgcccttcacttggggcaagacccgcgaaagctatggcgcggacgtcctgtacaagccgaataatggcaattgggcgccccaacaggacttcaccgagggcgtcttcatcgactaccgctacttcgacaaggttgacgatgactcggtcatctacgagttcggccacggcctgagctacaccaccttcgagtacagcaacatccgcgtcgtcaagtccaacgtcagcgagtaccggcccacgacgggcaccacgattcaggccccgacgtttggcaacttctccaccgacctcgaggactatctcttccccaaggacgagttcccctacatcccgcagtacatctacccgtacctcaacacgaccgacccccggagggcctcgggcgatccccactacggccagaccgccgaggagttcctcccgccccacgccaccgatgacgacccccagccgctcctccggtcctcgggcggaaactcccccggcggcaaccgccagctgtacgacattgtctacacaatcacggccgacatcacgaatacgggctccgttgtaggcgaggaggtaccgcagctctacgtctcgctgggcggtcccgaggatcccaaggtgcagctgcgcgactttgacaggatgcggatcgaacccggcgagacgaggcagttcaccggccgcctgacgcgcagagatctgagcaactgggacgtcacggtgcaggactgggtcatcagcaggtatcccaagacggcatatgttgggaggagcagccggaagttggatctcaagattgagcttccttga

SEQ ID NO: 122 - protein, 870 aa, Artificial Sequence
  Synthetic polypeptide for BGL Variant 900
MKAAALSCLFGSTLAVAGAIESRKVHQKPLARSEPFYPSPWMNPNAIGWAEAYAQAKSFVSQMTLLEKVNLTTGVGWGEEQCVGNVGAIPRLGLRSLCMHDSPLGVRGTDYNSAFPSGQTVAATWDRGLMYRRGYAMGQEAKGKGINVLLGPVAGPLGRMPEGGRNWEGFAPDPVLTGIGMSETIKGIQDAGVIACAKHFIGNEQEHFRQVPEAQGYGYNISETLSSNIDDKTMHELYLWPFADAVRAGVGSVMCSYNQGNNSYACQNSKLLNDLLKNELGFQGFVMSDWWAQHTGAASAVAGLDMSMPGDTMVNTGVSFWGANLTLAVLNGTVPAYRLDDMCMRIMAALFKVTKTTDLEPINFSFWTRDTYGPIHWAAKQGYQEINSHVDVRADHGNLIRNIAAKGTVLLKNTGSLPLNKPKFVAVIGEDAGPSPNGPNGCSDRGCNEGTLAMGWGSGTANYPYLVSPDAALQARAIQDGTRYESVLSNYAEENTKALVSQANATAIVFVNADSGEGYINVDGNEGDRKNLTLWNNGDTLVKNVSSWCSNTIVVIHSVGPVLLTDWYDNPNITAILWAGLPGQESGNSITDVLYGKVNPAARSPFTWGKTRESYGADVLYKPNNGNWAPQQDFTEGVFIDYRYFDKVDDDSVIYEFGHGLSYTTFEYSNIRVVKSNVSEYRPTTGTTIQAPTFGNFSTDLEDYLFPKDEFPYIPQYIYPYLNTTDPRRASGDPHYGQTAEEFLPPHATDDDPQPLLRSSGGNSPGGNRQLYDIVYTITADITNTGSVVGEEVPQLYVSLGGPEDPKVQLRDFDRMRIEPGETRQFTGRLTRRDLSNWDVTVQDWVISRYPKTAYVGRSSRKLDLKIELP

SEQ ID NO: 123 - protein, 851 aa, Artificial Sequence
  Synthetic polypeptide for BGL Variant 900
IESRKVHQKPLARSEPFYPSPWMNPNAIGWAEAYAQAKSFVSQMTLLEKVNLTTGVGWGEEQCVGNVGAIPRLGLRSLCMHDSPLGVRGTDYNSAFPSGQTVAATWDRGLMYRRGYAMGQEAKGKGINVLLGPVAGPLGRMPEGGRNWEGFAPDPVLTGIGMSETIKGIQDAGVIACAKHFIGNEQEHFRQVPEAQGYGYNISETLSSNIDDKTMHELYLWPFADAVRAGVGSVMCSYNQGNNSYACQNSKLLNDLLKNELGFQGFVMSDWWAQHTGAASAVAGLDMSMPGDTMVNTGVSFWGANLTLAVLNGTVPAYRLDDMCMRIMAALFKVTKTTDLEPINFSFWTRDTYGPIHWAAKQGYQEINSHVDVRADHGNLIRNIAAKGTVLLKNTGSLPLNKPKFVAVIGEDAGPSPNGPNGCSDRGCNEGTLAMGWGSGTANYPYLVSPDAALQARAIQDGTRYESVLSNYAEENTKALVSQANATAIVFVNADSGEGYINVDGNEGDRKNLTLWNNGDTLVKNVSSWCSNTIVVIHSVGPVLLTDWYDNPNITAILWAGLPGQESGNSITDVLYGKVNPAARSPFTWGKTRESYGADVLYKPNNGNWAPQQDFTEGVFIDYRYFDKVDDDSVIYEFGHGLSYTTFEYSNIRVVKSNVSEYRPTTGTTIQAPTFGNFSTDLEDYLFPKDEFPYIPQYIYPYLNTTDPRRASGDPHYGQTAEEFLPPHATDDDPQPLLRSSGGNSPGGNRQLYDIVYTITADITNTGSVVGEEVPQLYVSLGGPEDPKVQLRDFDRMRIEPGETRQFTGRLTRRDLSNWDVTVQDWVISRYPKTAYVGRSSRKLDLKIELP

SEQ ID NO: 124 - DNA, 1368 bp, Talaromyces emersonii
atgcttcgacgggctcttcttctatcctcttccgccatccttgctgtcaaggcacagcaggccggcacggcgacggcagagaaccacccgcccctgacatggcaggaatgcaccgcccctgggagctgcaccacccagaacggggcggtcgttcttgatgcgaactggcgttgggtgcacgatgtgaacggatacaccaactgctacacgggcaatacctgggaccccacgtactgccctgacgacgaaacctgcgcccagaactgtgcgctggacggcgcggattacgagggcacctacggcgtgacttcgtcgggcagctccttgaaactcaatttcgtcaccgggtcgaacgtcggatcccgtctctacctgctgcaggacgactcgacctatcagatcttcaagcttctgaaccgcgagttcagctttgacgtcgatgtctccaatcttccgtgcggattgaacggcgctctgtactttgtcgccatggacgccgacggcggcgtgtccaagtacccgaacaacaaggctggtgccaagtacggaaccgggtattgcgactcccaatgcccacgggacctcaagttcatcgacggcgaggccaacgtcgagggctggcagccgtcttcgaacaacgccaacaccggaattggcgaccacggctcctgctgtgcggagatggatgtctgggaagcaaacagcatctccaatgcggtcactccgcacccgtgcgacacgccaggccagacgatgtgctctggagatgactgcggtggcacatactctaacgatcgctacgcgggaacctgcgatcctgacggctgtgacttcaacccttaccgcatgggcaacacttctttctacgggcctggcaagatcatcgataccaccaagcccttcactgtcgtgacgcagttcctcactgatgatggtacggatactggaactctcagcgagatcaagcgcttctacatccagaacagcaacgtcattccgcagcccaactcggacatcagtggcgtgaccggcaactcgatcacgacggagttctgcactgctcagaagcaggcctttggcgacacggacgacttctctcagcacggtggcctggccaagatgggagcggccatgcagcagggtatggtcctggtgatgagtttgtgggacgactacgccgcgcagatgctgtggttggattccgactacccgacggatgcggaccccacgacccctggtattgcccgtggaacgtgtccgacggactcgggcgtcccatcggatgtcgagtcgcagagccccaactcctacgtgacctactcgaacattaagtttggtccgatcaactcgaccttcaccgcttcgtga

SEQ ID NO: 125 - protein, 455 aa, Talaromyces emersonii
MLRRALLLSSSAILAVKAQQAGTATAENHPPLTWQECTAPGSCTTQNGAVVLDANWRWVHDVNGYTNCYTGNTWDPTYCPDDETCAQNCALDGADYEGTYGVTSSGSSLKLNFVTGSNVGSRLYLLQDDSTYQIFKLLNREFSFDVDVSNLPCGLNGALYFVAMDADGGVSKYPNNKAGAKYGTGYCDSQCPRDLKFIDGEANVEGWQPSSNNANTGIGDHGSCCAEMDVWEANSISNAVTPHPCDTPGQTMCSGDDCGGTYSNDRYAGTCDPDGCDFNPYRMGNTSFYGPGKIIDTTKPFTVVTQFLTDDGTDTGTLSEIKRFYIQNSNVIPQPNSDISGVTGNSITTEFCTAQKQAFGDTDDFSQHGGLAKMGAAMQQGMVLVMSLWDDYAAQMLWLDSDYPTDADPTTPGIARGTCPTDSGVPSDVESQSPNSYVTYSNIKFGPINSTFTAS

SEQ ID NO: 126 - protein, 437 aa, Talaromyces emersonii
QQAGTATAENHPPLTWQECTAPGSCTTQNGAVVLDANWRWVHDVNGYTNCYTGNTWDPTYCPDDETCAQNCALDGADYEGTYGVTSSGSSLKLNFVTGSNVGSRLYLLQDDSTYQIFKLLNREFSFDVDVSNLPCGLNGALYFVAMDADGGVSKYPNNKAGAKYGTGYCDSQCPRDLKFIDGEANVEGWQPSSNNANTGIGDHGSCCAEMDVWEANSISNAVTPHPCDTPGQTMCSGDDCGGTYSNDRYAGTCDPDGCDFNPYRMGNTSFYGPGKIIDTTKPFTVVTQFLTDDGTDTGTLSEIKRFYIQNSNVIPQPNSDISGVTGNSITTEFCTAQKQAFGDTDDFSQHGGLAKMGAAMQQGMVLVMSLWDDYAAQMLWLDSDYPTDADPTTPGIARGTCPTDSGVPSDVESQSPNSYVTYSNIKFGPINSTFTAS

SEQ ID NO: 127 - DNA, 1581 bp, Myceliophthora thermophila
atgtacgccaagttcgcgaccctcgccgcccttgtggctggcgccgctgctcagaacgcctgcactctgaccgctgagaaccacccctcgctgacgtggtccaagtgcacgtctggcggcagctgcaccagcgtccagggttccatcaccatcgacgccaactggcggtggactcaccggaccgatagcgccaccaactgctacgagggcaacaagtgggatacttcgtactgcagcgatggtccttcttgcgcctccaagtgctgcatcgacggcgctgactactcgagcacctatggcatcaccacgagcggtaactccctgaacctcaagttcgtcaccaagggccagtactcgaccaacatcggctcgcgtacctacctgatggagagcgacaccaagtaccagatgttccagctcctcggcaacgagttcaccttcgatgtcgacgtctccaacctcggctgcggcctcaatggcgccctctacttcgtgtccatggatgccgatggtggcatgtccaagtactcgggcaacaaggcaggtgccaagtacggtaccggctactgtgattctcagtgcccccgcgacctcaagttcatcaacggcgaggccaacgtagagaactggcagagctcgaccaacgatgccaacgccggcacgggcaagtacggcagctgctgctccgagatggacgtctgggaggccaacaacatggccgccgccttcactccccacccttgcaccgtgatcggccagtcgcgctgcgagggcgactcgtgcggcggtacctacagcaccgaccgctatgccggcatctgcgaccccgacggatgcgacttcaactcgtaccgccagggcaacaagaccttctacggcaagggcatgacggtcgacacgaccaagaagatcacggtcgtcacccagttcctcaagaactcggccggcgagctctccgagatcaagcggttctacgtccagaacggcaaggtcatccccaactccgagtccaccatcccgggcgtcgagggcaactccatcacccaggactggtgcgaccgccagaaggccgccttcggcgacgtgaccgacttccaggacaagggcggcatggtccagatgggcaaggccctcgcggggcccatggtcctcgtcatgtccatctgggacgaccacgccgtcaacatgctctggctcgactccacctggcccatcgacggcgccggcaagccgggcgccgagcgcggtgcctgccccaccacctcgggcgtccccgctgaggtcgaggccgaggcccccaactccaacgtcatcttctccaacatccgcttcggccccatcggctccaccgtctccggcctgcccgacggcggcagcggcaaccccaacccgcccgtcagctcgtccaccccggtcccctcctcgtccaccacatcctccggttcctccggcccgactggcggcacgggtgtcgctaagcactatgagcaatgcggaggaatcgggttcactggccctacccagtgcgagagcccctacacttgcaccaagctgaatgactggtactcgcagtgcctgtaa

SEQ ID NO: 128 - protein, 526 aa, Myceliophthora thermophila
MYAKFATLAALVAGAAAQNACTLTAENHPSLTYSKCTSGGSCTSVQGSITIDANWRWTHRTDSATNCYEGNKWDTSWCSDGPSCASKCCIDGADYSSTYGITTSGNSLNLKFVTKGQYSTNIGSRTYLMESDTKYQMFQLLGNEFTFDVDVSNLGCGLNGALYFVSMDADGGMSKYSGNKAGAKYGTGYCDSQCPRDLKFINGEANVENWQSSTNDANAGTGKYGSCCSEMDVWEANNMAAAFTPHPCTVIGQSRCEGDSCGGTYSTDRYAGICDPDGCDFNSYRQGNKTFYGKGMTVDTTKKITVVTQFLKNSAGELSEIKRFYVQNGKVIPNSESTIPGVEGNSITQDWCDRQKAAFGDVTDFQDKGGMVQMGKALAGPMVLVMSIWDDHAVNMLWLDSTWPIDGAGKPGAERGACPTTSGVPAEVEAEAPNSNVIFSNIRFGPIGSTVSGLPDGGSGNPNPPVSSSTPVPSSSTTSSGSSGPTGGTGVAKHYEQCGGIGFTGPTQCESPYTCTKLNDWYSQCL

SEQ ID NO: 129 - protein, 509 aa, Myceliophthora thermophila
QNACTLTAENHPSLTYSKCTSGGSCTSVQGSITIDANWRWTHRTDSATNCYEGNKWDTSWCSDGPSCASKCCIDGADYSSTYGITTSGNSLNLKFVTKGQYSTNIGSRTYLMESDTKYQMFQLLGNEFTFDVDVSNLGCGLNGALYFVSMDADGGMSKYSGNKAGAKYGTGYCDSQCPRDLKFINGEANVENWQSSTNDANAGTGKYGSCCSEMDVWEANNMAAAFTPHPCTVIGQSRCEGDSCGGTYSTDRYAGICDPDGCDFNSYRQGNKTFYGKGMTVDTTKKITVVTQFLKNSAGELSEIKRFYVQNGKVIPNSESTIPGVEGNSITQDWCDRQKAAFGDVTDFQDKGGMVQMGKALAGPMVLVMSIWDDHAVNMLWLDSTWPIDGAGKPGAERGACPTTSGVPAEVEAEAPNSNVIFSNIRFGPIGSTVSGLPDGGSGNPNPPVSSSTPVPSSSTTSSGSSGPTGGTGVAKHYEQCGGIGFTGPTQCESPYTCTKLNDWYSQCL

SEQ ID NO: 130 - DNA, 1581 bp, Artificial Sequence
  Synthetic polynucleotide for CBH1a Variant 145
atgtacgccaagttcgcgaccctcgccgcccttgtggctggcgccgctgctcagaacgcctgcactctgaccgctgagaaccacccctcgctgacgtggtccaagtgcacgtctggcggcagctgcaccagcgtccagggttccatcaccatcgacgccaactggcggtggactcaccggaccgatagcgccaccaactgctacgagggcaacaagtgggatacttcgtggtgcagcgatggtccttcttgcgcctccaagtgctgcatcgacggcgctgactactcgagcacctatggcatcaccacgagcggtaactccctgaacctcaagttcgtcaccaagggccagtactcgaccaacatcggctcgcgtacctacctgatggagagcgacaccaagtaccagatgttccagctcctcggcaacgagttcaccttcgatgtcgacgtctccaacctcggctgcggcctcaatggcgccctctacttcgtgtccatggatgccgatggtggcatgtccaagtactcgggcaacaaggcaggtgccaagtacggtaccggctactgtgattctcagtgcccccgcgacctcaagttcatcaacggcgaggccaacgtagagaactggcagagctcgaccaacgatgccaacgccggcacgggcaagtacggcagctgctgctccgagatggacgtctgggaggccaacaacatggccgccgccttcactccccacccttgcaccgtgatcggccagtcgcgctgcgagggcgactcgtgcggcggtacctacagcaccgaccgctatgccggcatctgcgaccccgacggatgcgacttcaactcgtaccgccagggcaacaagaccttctacggcaagggcatgacggtcgacacgaccaagaagatcacggtcgtcacccagttcctcaagaactcggccggcgagctctccgagatcaagcggttctacgtccagaacggcaaggtcatccccaactccgagtccaccatcccgggcgtcgagggcaactccatcacccaggactggtgcgaccgccagaaggccgccttcggcgacgtgaccgacttccaggacaagggcggcatggtccagatgggcaaggccctcgcggggcccatggtcctcgtcatgtccatctgggacgaccacgccgtcaacatgctctggctcgactccacctggcccatcgacggcgccggcaagccgggcgccgagcgcggtgcctgccccaccacctcgggcgtccccgctgaggtcgaggccgaggcccccaactccaacgtcatcttctccaacatccgcttcggccccatcggctccaccgtctccggcctgcccgacggcggcagcggcaaccccaacccgcccgtcagctcgtccaccccggtcccctcctcgtccaccacatcctccggttcctccggcccgactggcggcacgggtgtcgctaagcactatgagcaatgcggaggaatcgggttcactggccctacccagtgcgagagcccctacacttgcaccaagctgaatgactggtactcgcagtgcctgtaa

SEQ ID NO: 131 - protein, 526 aa, Artificial Sequence
  Synthetic polypeptide for CBH1a Variant 145
MYAKFATLAALVAGAAAQNACTLTAENHPSLTWSKCTSGGSCTSVQGSITIDANWRWTHRTDSATNCYEGNKWDTSWCSDGPSCASKCCIDGADYSSTYGITTSGNSLNLKFVTKGQYSTNIGSRTYLMESDTKYQMFQLLGNEFTFDVDVSNLGCGLNGALYFVSMDADGGMSKYSGNKAGAKYGTGYCDSQCPRDLKFINGEANVENWQSSTNDANAGTGKYGSCCSEMDVWEANNMAAAFTPHPCTVIGQSRCEGDSCGGTYSTDRYAGICDPDGCDFNSYRQGNKTFYGKGMTVDTTKKITVVTQFLKNSAGELSEIKRFYVQNGKVIPNSESTIPGVEGNSITQDWCDRQKAAFGDVTDFQDKGGMVQMGKALAGPMVLVMSIWDDHAVNMLWLDSTWPIDGAGKPGAERGACPTTSGVPAEVEAEAPNSNVIFSNIRFGPIGSTVSGLPDGGSGNPNPPVSSSTPVPSSSTTSSGSSGPTGGTGVAKHYEQCGGIGFTGPTQCESPYTCTKLNDWYSQCL

SEQ ID NO: 132 - protein, 509 aa, Artificial Sequence
  Synthetic polypeptide for CBH1a Variant 145
QNACTLTAENHPSLTWSKCTSGGSCTSVQGSITIDANWRWTHRTDSATNCYEGNKWDTSWCSDGPSCASKCCIDGADYSSTYGITTSGNSLNLKFVTKGQYSTNIGSRTYLMESDTKYQMFQLLGNEFTFDVDVSNLGCGLNGALYFVSMDADGGMSKYSGNKAGAKYGTGYCDSQCPRDLKFINGEANVENWQSSTNDANAGTGKYGSCCSEMDVWEANNMAAAFTPHPCTVIGQSRCEGDSCGGTYSTDRYAGICDPDGCDFNSYRQGNKTFYGKGMTVDTTKKITVVTQFLKNSAGELSEIKRFYVQNGKVIPNSESTIPGVEGNSITQDWCDRQKAAFGDVTDFQDKGGMVQMGKALAGPMVLVMSIWDDHAVNMLWLDSTWPIDGAGKPGAERGACPTTSGVPAEVEAEAPNSNVIFSNIRFGPIGSTVSGLPDGGSGNPNPPVSSSTPVPSSSTTSSGSSGPTGGTGVAKHYEQCGGIGFTGPTQCESPYTCTKLNDWYSQCL

SEQ ID NO: 133 - DNA, 1581 bp, Artificial Sequence
  Synthetic polynucleotide for CBH1a Variant 983
atgtacgccaagttcgcgaccctcgccgcccttgtggctggcgccgctgctcagaacgcctgcactctgaacgctgagaaccacccctcgctgacgtggtccaagtgcacgtctggcggcagctgcaccagcgtccagggttccatcaccatcgacgccaactggcggtggactcaccggaccgatagcgccaccaactgctacgagggcaacaagtgggatacttcgtactgcagcgatggtccttcttgcgcctccaagtgctgcatcgacggcgctgactactcgagcacctatggcatcaccacgagcggtaactccctgaacctcaagttcgtcaccaagggccagtactcgaccaacatcggctcgcgtacctacctgatggagagcgacaccaagtaccagatgttccagctcctcggcaacgagttcaccttcgatgtcgacgtctccaacctcggctgcggcctcaatggcgccctctacttcgtgtccatggatgccgatggtggcatgtccaagtactcgggcaacaaggcaggtgccaagtacggtaccggctactgtgattctcagtgcccccgcgacctcaagttcatcaacggcgaggccaacgtagagaactggcagagctcgaccaacgatgccaacgccggcacgggcaagtacggcagctgctgctccgagatggacgtctgggaggccaacaacatggccgccgccttcactccccacccttgcaccgtgatcggccagtcgcgctgcgagggcgactcgtgcggcggtacctacagcaccgaccgctatgccggcatctgcgaccccgacggatgcgacttcaactcgtaccgccagggcaacaagaccttctacggcaagggcatgacggtcgacacgaccaagaagatcacggtcgtcacccagttcctcaagaactcggccggcgagctctccgagatcaagcggttctacgtccagaacggcaaggtcatccccaactccgagtccaccatcccgggcgtcgagggcaactccatcacccaggagtactgcgaccgccagaaggccgccttcggcgacgtgaccgacttccaggacaagggcggcatggtccagatgggcaaggccctcgcggggcccatggtcctcgtcatgtccatctgggacgaccacgccgacaacatgctctggctcgactccacctggcccatcgacggcgccggcaagccgggcgccgagcgcggtgcctgccccaccacctcgggcgtccccgctgaggtcgaggccgaggcccccaactccaacgtcatcttctccaacatccgcttcggccccatcggctccaccgtctccggcctgcccgacggcggcagcggcaaccccaacccgcccgtcagctcgtccaccccggtcccctcctcgtccaccacatcctccggttcctccggcccgactggcggcacgggtgtcgctaagcactatgagcaatgcggaggaatcgggttcactggccctacccagtgcgagagcccctacacttgcaccaagctgaatgactggtactcgcagtgcctgtaa

SEQ ID NO: 134 - protein, 526 aa, Artificial Sequence
  Synthetic polypeptide for CBH1a Variant 983
MYAKFATLAALVAGAAAQNACTLNAENHPSLTWSKCTSGGSCTSVQGSITIDANWRWTHRTDSATNCYEGNKWDTSYCSDGPSCASKCCIDGADYSSTYGITTSGNSLNLKFVTKGQYSTNIGSRTYLMESDTKYQMFQLLGNEFTFDVDVSNLGCGLNGALYFVSMDADGGMSKYSGNKAGAKYGTGYCDSQCPRDLKFINGEANVENWQSSTNDANAGTGKYGSCCSEMDVWEANNMAAAFTPHPCTVIGQSRCEGDSCGGTYSTDRYAGICDPDGCDFNSYRQGNKTFYGKGMTVDTTKKITVVTQFLKNSAGELSEIKRFYVQNGKVIPNSESTIPGVEGNSITQEYCDRQKAAFGDVTDFQDKGGMVQMGKALAGPMVLVMSIWDDHADNMLWLDSTWPIDGAGKPGAERGACPTTSGVPAEVEAEAPNSNVIFSNIRFGPIGSTVSGLPDGGSGNPNPPVSSSTPVPSSSTTSSGSSGPTGGTGVAKHYEQCGGIGFTGPTQCESPYTCTKLNDWYSQCL

SEQ ID NO: 135 - protein, 509 aa, Artificial Sequence
  Synthetic polypeptide for CBH1a Variant 983
QNACTLNAENHPSLTWSKCTSGGSCTSVQGSITIDANWRWTHRTDSATNCYEGNKWDTSYCSDGPSCASKCCIDGADYSSTYGITTSGNSLNLKFVTKGQYSTNIGSRTYLMESDTKYQMFQLLGNEFTFDVDVSNLGCGLNGALYFVSMDADGGMSKYSGNKAGAKYGTGYCDSQCPRDLKFINGEANVENWQSSTNDANAGTGKYGSCCSEMDVWEANNMAAAFTPHPCTVIGQSRCEGDSCGGTYSTDRYAGICDPDGCDFNSYRQGNKTFYGKGMTVDTTKKITVVTQFLKNSAGELSEIKRFYVQNGKVIPNSESTIPGVEGNSITQEYCDRQKAAFGDVTDFQDKGGMVQMGKALAGPMVLVMSIWDDHADNMLWLDSTWPIDGAGKPGAERGACPTTSGVPAEVEAEAPNSNVIFSNIRFGPIGSTVSGLPDGGSGNPNPPVSSSTPVPSSSTTSSGSSGPTGGTGVAKHYEQCGGIGFTGPTQCESPYTCTKLNDWYSQCL

SEQ ID NO: 136 - DNA, 1449 bp, Myceliophthora thermophila
atggccaagaagcttttcatcaccgccgcgcttgcggctgccgtgttggcggcccccgtcattgaggagcgccagaactgcggcgctgtgtggactcaatgcggcggtaacgggtggcaaggtcccacatgctgcgcctcgggctcgacctgcgttgcgcagaacgagtggtactctcagtgcctgcccaacagccaggtgacgagttccaccactccgtcgtcgacttccacctcgcagcgcagcaccagcacctccagcagcaccaccaggagcggcagctcctcctcctcctccaccacgcccccgcccgtctccagccccgtgaccagcattcccggcggtgcgacctccacggcgagctactctggcaaccccttctcgggcgtccggctcttcgccaacgactactacaggtccgaggtccacaatctcgccattcctagcatgactggtactctggcggccaaggcttccgccgtcgccgaagtccctagcttccagtggctcgaccggaacgtcaccatcgacaccctgatggtccagactctgtcccaggtccgggctctcaataaggccggtgccaatcctccctatgctgcccaactcgtcgtctacgacctccccgaccgtgactgtgccgccgctgcgtccaacggcgagttttcgattgcaaacggcggcgccgccaactacaggagctacatcgacgctatccgcaagcacatcattgagtactcggacatccggatcatcctggttatcgagcccgactcgatggccaacatggtgaccaacatgaacgtggccaagtgcagcaacgccgcgtcgacgtaccacgagttgaccgtgtacgcgctcaagcagctgaacctgcccaacgtcgccatgtatctcgacgccggccacgccggctggctcggctggcccgccaacatccagcccgccgccgagctgtttgccggcatctacaatgatgccggcaagccggctgccgtccgcggcctggccactaacgtcgccaactacaacgcctggagcatcgcttcggccccgtcgtacacgtcgcctaaccctaactacgacgagaagcactacatcgaggccttcagcccgctcttgaactcggccggcttccccgcacgcttcattgtcgacactggccgcaacggcaaacaacctaccggccaacaacagtggggtgactggtgcaatgtcaagggcaccggctttggcgtgcgcccgacggccaacacgggccacgagctggtcgatgcctttgtctgggtcaagcccggcggcgagtccgacggcacaagcgacaccagcgccgcccgctacgactaccactgcggcctgtccgatgccctgcagcctgcccccgaggctggacagtggttccaggcctacttcgagcagctgctcaccaacgccaacccgcccttctaa

SEQ ID NO: 137 - protein, 482 aa, Myceliophthora thermophila
MAKKLFITAALAAAVLAAPVIEERQNCGAVWTQCGGNGWQGPTCCASGSTCVAQNEWYSQCLPNSQVTSSTTPSSTSTSQRSTSTSSSTTRSGSSSSSSTTPPPVSSPVTSIPGGATSTASYSGNPFSGVRLFANDYYRSEVHNLAIPSMTGTLAAKASAVAEVPSFQWLDRNVTIDTLMVQTLSQVRALNKAGANPPYAAQLVVYDLPDRDCAAAASNGEFSIANGGAANYRSYIDAIRKHIIEYSDIRIILVIEPDSMANMVTNMNVAKCSNAASTYHELTVYALKQLNLPNVAMYLDAGHAGWLGWPANIQPAAELFAGIYNDAGKPAAVRGLATNVANYNAWSIASAPSYTSPNPNYDEKHYIEAFSPLLNSAGFPARFIVDTGRNGKQPTGQQQWGDWCNVKGTGFGVRPTANTGHELVDAFVWVKPGGESDGTSDTSAARYDYHCGLSDALQPAPEAGQWFQAYFEQLLTNANPPF

SEQ ID NO: 138 - protein, 465 aa, Myceliophthora thermophila
APVIEERQNCGAVWTQCGGNGWQGPTCCASGSTCVAQNEWYSQCLPNSQVTSSTTPSSTSTSQRSTSTSSSTTRSGSSSSSSTTPPPVSSPVTSIPGGATSTASYSGNPFSGVRLFANDYYRSEVHNLAIPSMTGTLAAKASAVAEVPSFQWLDRNVTIDTLMVQTLSQVRALNKAGANPPYAAQLVVYDLPDRDCAAAASNGEFSIANGGAANYRSYIDAIRKHIIEYSDIRIILVIEPDSMANMVTNMNVAKCSNAASTYHELTVYALKQLNLPNVAMYLDAGHAGWLGWPANIQPAAELFAGIYNDAGKPAAVRGLATNVANYNAWSIASAPSYTSPNPNYDEKHYIEAFSPLLNSAGFPARFIVDTGRNGKQPTGQQQWGDWCNVKGTGFGVRPTANTGHELVDAFVWVKPGGESDGTSDTSAARYDYHCGLSDALQPAPEAGQWFQAYFEQLLTNANPPF

SEQ ID NO: 139 - DNA, 1449 bp, Artificial Sequence
  Synthetic polynucleotide for CBH2b Variant 196
atggccaagaagcttttcatcaccgccgcgcttgcggctgccgtgttggcggcccccgtcattgaggagcgccagaactgcggcgctgtgtggactcaatgcggcggtaacgggtggcaaggtcccacatgctgcgcctcgggctcgacctgcgttgcgcagaacgagtggtactctcagtgcctgcccaacagccaggtgacgagttccaccactccgtcgtcgacttccacctcgcagcgcagcaccagcacctccagcagcaccaccaggagcggcagctcctcctcctcctccaccacgcccacccccgtctccagccccgtgaccagcattcccggcggtgcgacctccacggcgagctactctggcaaccccttctcgggcgtccggctcttcgccaacgactactacaggtccgaggtccacaatctcgccattcctagcatgactggtactctggcggccaaggcttccgccgtcgccgaagtccctagcttccagtggctcgaccggaacgtcaccatcgacaccctgatggtcccgactctgtcccgcgtccgggctctcaataaggccggtgccaatcctccctatgctgcccaactcgtcgtctacgacctccccgaccgtgactgtgccgccgctgcgtccaacggcgagttttcgattgcaaacggcggcgccgccaactacaggagctacatcgacgctatccgcaagcacatcattgagtactcggacatccggatcatcctggttatcgagcccgactcgatggccaacatggtgaccaacatgaacgtggccaagtgcagcaacgccgcgtcgacgtaccacgagttgaccgtgtacgcgctcaagcagctgaacctgcccaacgtcgccatgtatctcgacgccggccacgccggctggctcggctggcccgccaacatccagcccgccgccgagctgtttgccggcatctacaatgatgccggcaagccggctgccgtccgcggcctggccactaacgtcgccaactacaacgcctggagcatcgcttcggccccgtcgtacacgtcgcctaaccctaactacgacgagaagcactacatcgaggccttcagcccgctcttgaactcggccggcttccccgcacgcttcattgtcgacactggccgcaacggcaaacaacctaccggccaacaacagtggggtgactggtgcaatgtcaagggcaccggctttggcgtgcgcccgacggccaacacgggccacgagctggtcgatgcctttgtctgggtcaagcccggcggcgagtccgacggcacaagcgacaccagcgccgcccgctacgactaccactgcggcctgtccgatgccctgcagcctgcccccgaggctggacagtggttccaggcctacttcgagcagctgctcaccaacgccaacccgcccttctaa

SEQ ID NO: 140 - protein, 482 aa, Artificial Sequence
  Synthetic polypeptide for CBH2b Variant 196
MAKKLFITAALAAAVLAAPVIEERQNCGAVWTQCGGNGWQGPTCCASGSTCVAQNEWYSQCLPNSQVTSSTTPSSTSTSQRSTSTSSSTTRSGSSSSSSTTPTPVSSPVTSIPGGATSTASYSGNPFSGVRLFANDYYRSEVHNLAIPSMTGTLAAKASAVAEVPSFQWLDRNVTIDTLMVPTLSRVRALNKAGANPPYAAQLVVYDLPDRDCAAAASNGEFSIANGGAANYRSYIDAIRKHIIEYSDIRIILVIEPDSMANMVTNMNVAKCSNAASTYHELTVYALKQLNLPNVAMYLDAGHAGWLGWPANIQPAAELFAGIYNDAGKPAAVRGLATNVANYNAWSIASAPSYTSPNPNYDEKHYIEAFSPLLNSAGFPARFIVDTGRNGKQPTGQQQWGDWCNVKGTGFGVRPTANTGHELVDAFVWVKPGGESDGTSDTSAARYDYHCGLSDALQPAPEAGQWFQAYFEQLLTNANPPF

SEQ ID NO: 141 - protein, 465 aa, Artificial Sequence
  Synthetic polypeptide for CBH2b Variant 196
APVIEERQNCGAVWTQCGGNGWQGPTCCASGSTCVAQNEWYSQCLPNSQVTSSTTPSSTSTSQRSTSTSSSTTRSGSSSSSSTTPTPVSSPVTSIPGGATSTASYSGNPFSGVRLFANDYYRSEVHNLAIPSMTGTLAAKASAVAEVPSFQWLDRNVTIDTLMVPTLSRVRALNKAGANPPYAAQLVVYDLPDRDCAAAASNGEFSIANGGAANYRSYIDAIRKHIIEYSDIRIILVIEPDSMANMVTNMNVAKCSNAASTYHELTVYALKQLNLPNVAMYLDAGHAGWLGWPANIQPAAELFAGIYNDAGKPAAVRGLATNVANYNAWSIASAPSYTSPNPNYDEKHYIEAFSPLLNSAGFPARFIVDTGRNGKQPTGQQQWGDWCNVKGTGFGVRPTANTGHELVDAFVWVKPGGESDGTSDTSAARYDYHCGLSDALQPAPEAGQWFQAYFEQLLTNANPPF

SEQ ID NO: 142 - DNA, 1449 bp, Artificial Sequence
  Synthetic polynucleotide for CBH2b Variant 287
atggccaagaagcttttcatcaccgccgcgcttgcggctgccgtgttggcggcccccgtcattgaggagcgccagaactgcggcgctgtgtggactcaatgcggcggtaacgggtggcaaggtcccacatgctgcgcctcgggctcgacctgcgttgcgcagaacgagtggtactctcagtgcctgcccaacagccaggtgacgagttccaccactccgtcgtcgacttccacctcgcagcgcagcaccagcacctccagcagcaccaccaggagcggcagctcctcctcctcctccaccacgcccccgcccgtctccagccccgtgaccagcattcccggcggtgcgacctccacggcgagctactctggcaaccccttctcgggcgtccggctcttcgccaacgactactacaggtccgaggtccacaatctcgccattcctagcatgactggtactctggcggccaaggcttccgccgtcgccgaagtccctagcttccagtggctcgaccggaacgtcaccatcgacaccctgatggtcccgactctgtcccgcgtccgggctctcaataaggccggtgccaatcctccctatgctgcccaactcgtcgtctacgacctccccgaccgtgactgtgccgccgctgcgtccaacggcgagttttcgattgcaaacggcggcgccgccaactacaggagctacatcgacgctatccgcaagcacatcaaggagtactcggacatccggatcatcctggttatcgagcccgactcgatggccaacatggtgaccaacatgaacgtggccaagtgcagcaacgccgcgtcgacgtaccacgagttgaccgtgtacgcgctcaagcagctgaacctgcccaacgtcgccatgtatctcgacgccggccacgccggctggctcggctggcccgccaacatccagcccgccgccgagctgtttgccggcatctacaatgatgccggcaagccggctgccgtccgcggcctggccactaacgtcgccaactacaacgcctggagcatcgcttcggccccgtcgtacacgtcgcctaaccctaactacgacgagaagcactacatcgaggccttcagcccgctcttgaacgacgccggcttccccgcacgcttcattgtcgacactggccgcaacggcaaacaacctaccggccaacaacagtggggtgactggtgcaatgtcaagggcaccggctttggcgtgcgcccgacggccaacacgggccacgagctggtcgatgcctttgtctgggtcaagcccggcggcgagtccgacggcacaagcgacaccagcgccgcccgctacgactaccactgcggcctgtccgatgccctgcagcctgcccccgaggctggacagtggttccaggcctacttcgagcagctgctcaccaacgccaacccgcccttctaa

SEQ ID NO: 143 - protein, 482 aa, Artificial Sequence
  Synthetic polypeptide for CBH2b Variant 287
MAKKLFITAALAAAVLAAPVIEERQNCGAVWTQCGGNGWQGPTCCASGSTCVAQNEWYSQCLPNSQVTSSTTPSSTSTSQRSTSTSSSTTRSGSSSSSSTTPPPVSSPVTSIPGGATSTASYSGNPFSGVRLFANDYYRSEVHNLAIPSMTGTLAAKASAVAEVPSFQWLDRNVTIDTLMVPTLSRVRALNKAGANPPYAAQLVVYDLPDRDCAAAASNGEFSIANGGAANYRSYIDAIRKHIKEYSDIRIILVIEPDSMANMVTNMNVAKCSNAASTYHELTVYALKQLNLPNVAMYLDAGHAGWLGWPANIQPAAELFAGIYNDAGKPAAVRGLATNVANYNAWSIASAPSYTSPNPNYDEKHYIEAFSPLLNDAGFPARFIVDTGRNGKQPTGQQQWGDWCNVKGTGFGVRPTANTGHELVDAFVWVKPGGESDGTSDTSAARYDYHCGLSDALQPAPEAGQWFQAYFEQLLTNANPPF

SEQ ID NO: 144 - protein, 465 aa, Artificial Sequence
  Synthetic polypeptide for CBH2b Variant 287
APVIEERQNCGAVWTQCGGNGWQGPTCCASGSTCVAQNEWYSQCLPNSQVTSSTTPSSTSTSQRSTSTSSSTTRSGSSSSSSTTPPPVSSPVTSIPGGATSTASYSGNPFSGVRLFANDYYRSEVHNLAIPSMTGTLAAKASAVAEVPSFQWLDRNVTIDTLMVPTLSRVRALNKAGANPPYAAQLVVYDLPDRDCAAAASNGEFSIANGGAANYRSYIDAIRKHIKEYSDIRIILVIEPDSMANMVTNMNVAKCSNAASTYHELTVYALKQLNLPNVAMYLDAGHAGWLGWPANIQPAAELFAGIYNDAGKPAAVRGLATNVANYNAWSIASAPSYTSPNPNYDEKHYIEAFSPLLNDAGFPARFIVDTGRNGKQPTGQQQWGDWCNVKGTGFGVRPTANTGHELVDAFVWVKPGGESDGTSDTSAARYDYHCGLSDALQPAPEAGQWFQAYFEQLLTNANPPF

SEQ ID NO: 145 - DNA, 1449 bp, Artificial Sequence
  Synthetic polynucleotide for CBH2b Variant 962
atggccaagaagcttttcatcaccgccgcgcttgcggctgccgtgttggcggcccccgtcattgaggagcgccagaactgcggcgctgtgtggactcaatgcggcggtaacgggtggcaaggtcccacatgctgcgcctcgggctcgacctgcgttgcgcagaacgagtggtactctcagtgcctgcccaacagccaggtgacgagttccaccactccgtcgtcgacttccacctcgcagcgcagcaccagcacctccagcagcaccaccaggagcggcagctcctcctcctcctccaccacgcccacccccgtctccagccccgtgaccagcattcccggcggtgcgacctccacggcgagctactctggcaaccccttctcgggcgtccggctcttcgccaacgactactacaggtccgaggtcatgaatctcgccattcctagcatgactggtactctggcggccaaggcttccgccgtcgccgaagtccctagcttccagtggctcgaccggaacgtcaccatcgacaccctgatggtcaccactctgtcccaggtccgggctctcaataaggccggtgccaatcctccctatgctgcccaactcgtcgtctacgacctccccgaccgtgactgtgccgccgctgcgtccaacggcgagttttcgattgcaaacggcggcagcgccaactacaggagctacatcgacgctatccgcaagcacatcattgagtactcggacatccggatcatcctggttatcgagcccgactcgatggccaacatggtgaccaacatgaacgtggccaagtgcagcaacgccgcgtcgacgtaccacgagttgaccgtgtacgcgctcaagcagctgaacctgcccaacgtcgccatgtatctcgacgccggccacgccggctggctcggctggcccgccaacatccagcccgccgccgagctgtttgccggcatctacaatgatgccggcaagccggctgccgtccgcggcctggccactaacgtcgccaactacaacgcctggagcatcgcttcggccccgtcgtacacgcagcctaaccctaactacgacgagaagcactacatcgaggccttcagcccgctcttgaactcggccggcttccccgcacgcttcattgtcgacactggccgcaacggcaaacaacctaccggccaacaacagtggggtgactggtgcaatgtcaagggcaccggctttggcgtgcgcccgacggccaacacgggccacgagctggtcgatgcctttgtctgggtcaagcccggcggcgagtccgacggcacaagcgacaccagcgccgcccgctacgactaccactgcggcctgtccgatgccctgcagcctgcccccgaggctggacagtggttccaggcctacttcgagcagctgctcaccaacgccaacccgcccttctaa

SEQ ID NO: 146 - protein, 482 aa, Artificial Sequence
  Synthetic polypeptide for CBH2b Variant 962
MAKKLFITAALAAAVLAAPVIEERQNCGAVWTQCGGNGWQGPTCCASGSTCVAQNEWYSQCLPNSQVTSSTTPSSTSTSQRSTSTSSSTTRSGSSSSSSTTPTPVSSPVTSIPGGATSTASYSGNPFSGVRLFANDYYRSEVMNLAIPSMTGTLAAKASAVAEVPSFQWLDRNVTIDTLMVTTLSQVRALNKAGANPPYAAQLVVYDLPDRDCAAAASNGEFSIANGGSANYRSYIDAIRKHIIEYSDIRIILVIEPDSMANMVTNMNVAKCSNAASTYHELTVYALKQLNLPNVAMYLDAGHAGWLGWPANIQPAAELFAGIYNDAGKPAAVRGLATNVANYNAWSIASAPSYTQPNPNYDEKHYIEAFSPLLNSAGFPARFIVDTGRNGKQPTGQQQWGDWCNVKGTGFGVRPTANTGHELVDAFVWVKPGGESDGTSDTSAARYDYHCGLSDALQPAPEAGQWFQAYFEQLLTNANPPF

SEQ ID NO: 147 - protein, 464 aa, Artificial Sequence
  Synthetic polypeptide for CBH2b Variant 962
APVIEERQNCGAVWTQCGGNGWQGPTCCASGSTCVAQNEWYSQCLPNSQVTSSTTPSSTSTSQRSTSTSSSTTRSGSSSSSSTTPTPVSSPVTSIPGGATSTASYSGNPFSGVRLFANDYYRSEVMNLAIPSMTGTLAAKASAVAEVPSFQWLDRNVTIDTLMVTTLSQVRALNKAGANPPYAAQLVVYDLPDRDCAAAASNGEFSIANGGSANYRSYIDAIRKHIIEYSDIRIILVIEPDSMANMVTNMNVAKCSNAASTYHELTVYALKQLNLPNVAMYLDAGHAGWLGWPANIQPAAELFAGIYNDGKPAAVRGLATNVANYNAWSIASAPSYTQPNPNYDEKHYIEAFSPLLNSAGFPARFIVDTGRNGKQPTGQQQWGDWCNVKGTGFGVRPTANTGHELVDAFVWVKPGGESDGTSDTSAARYDYHCGLSDALQPAPEAGQWFQAYFEQLLTNANPPF

SEQ ID NO: 148 - DNA, 1239 bp, Myceliophthora thermophila
atgcactccaaagctttcttggcagcgcttcttgcgcctgccgtctcagggcaactgaacgacctcgccgtcagggctggactcaagtactttggtactgctcttagcgagagcgtcatcaacagtgatactcggtatgctgccatcctcagcgacaagagcatgttcggccagctcgtccccgagaatggcatgaagtgggatgctactgagccgtcccgtggccagttcaactacgcctcgggcgacatcacggccaacacggccaagaagaatggccagggcatgcgttgccacaccatggtctggtacagccagctcccgagctgggtctcctcgggctcgtggaccagggactcgctcacctcggtcatcgagacgcacatgaacaacgtcatgggccactacaagggccaatgctacgcctgggatgtcatcaacgaggccatcaatgacgacggcaactcctggcgcgacaacgtctttctccggacctttgggaccgactacttcgccctgtccttcaacctagccaagaaggccgatcccgataccaagctgtactacaacgactacaacctcgagtacaaccaggccaagacggaccgcgctgttgagctcgtcaagatggtccaggccgccggcgcgcccatcgacggtgtcggcttccagggccacctcattgtcggctcgaccccgacgcgctcgcagctggccaccgccctccagcgcttcaccgcgctcggcctcgaggtcgcctacaccgagctcgacatccgccactcgagcctgccggcctcttcgtcggcgctcgcgacccagggcaacgacttcgccaacgtggtcggctcttgcctcgacaccgccggctgcgtcggcgtcaccgtctggggcttcaccgatgcgcactcgtggatcccgaacacgttccccggccagggcgacgccctgatctacgacagcaactacaacaagaagcccgcgtggacctcgatctcgtccgtcctggccgccaaggccaccggcgccccgcccgcctcgtcctccaccaccctcgtcaccatcaccacccctccgccggcatccaccaccgcctcctcctcctccagtgccacgcccacgagcgtcccgacgcagacgaggtggggacagtgcggcggcatcggatggacggggccgacccagtgcgagagcccatggacctgccagaagctgaacgactggtactggcagtgcctg

SEQ ID NO: 149 - protein, 413 aa, Myceliophthora thermophila
MHSKAFLAALLAPAVSGQLNDLAVRAGLKYFGTALSESVINSDTRYAAILSDKSMFGQLVPENGMKWDATEPSRGQFNYASGDITANTAKKNGQGMRCHTMVWYSQLPSWVSSGSWTRDSLTSVIETHMNNVMGHYKGQCYAWDVINEAINDDGNSWRDNVFLRTFGTDYFALSFNLAKKADPDTKLYYNDYNLEYNQAKTDRAVELVKMVQAAGAPIDGVGFQGHLIVGSTPTRSQLATALQRFTALGLEVAYTELDIRHSSLPASSSALATQGNDFANVVGSCLDTAGCVGVTVWGFTDAHSWIPNTFPGQGDALIYDSNYNKKPAWTSISSVLAAKATGAPPASSSTTLVTITTPPPASTTASSSSSATPTSVPTQTRWGQCGGIGWTGPTQCESPWTCQKLNDWYWQCL

SEQ ID NO: 150 - protein, 396 aa, Myceliophthora thermophila
QLNDLAVRAGLKYFGTALSESVINSDTRYAAILSDKSMFGQLVPENGMKWDATEPSRGQFNYASGDITANTAKKNGQGMRCHTMVWYSQLPSWVSSGSWTRDSLTSVIETHMNNVMGHYKGQCYAWDVINEAINDDGNSWRDNVFLRTFGTDYFALSFNLAKKADPDTKLYYNDYNLEYNQAKTDRAVELVKMVQAAGAPIDGVGFQGHLIVGSTPTRSQLATALQRFTALGLEVAYTELDIRHSSLPASSSALATQGNDFANVVGSCLDTAGCVGVTVWGFTDAHSWIPNTFPGQGDALIYDSNYNKKPAWTSISSVLAAKATGAPPASSSTTLVTITTPPPASTTASSSSSATPTSVPTQTRWGQCGGIGWTGPTQCESPWTCQKLNDWYWQCL

SEQ ID NO: 151 - DNA, 654 bp, Myceliophthora thermophila
atggtctcgttcactctcctcctcacggtcatcgccgctgcggtgacgacggccagccctctcgaggtggtcaagcgcggcatccagccgggcacgggcacccacgaggggtacttctactcgttctggaccgacggccgtggctcggtcgacttcaaccccgggccccgcggctcgtacagcgtcacctggaacaacgtcaacaactgggttggcggcaagggctggaacccgggcccgccgcgcaagattgcgtacaacggcacctggaacaactacaacgtgaacagctacctcgccctgtacggctggactcgcaacccgctggtcgagtattacatcgtggaggcatacggcacgtacaacccctcgtcgggcacggcgcggctgggcaccatcgaggacgacggcggcgtgtacgacatctacaagacgacgcggtacaaccagccgtccatcgaggggacctccaccttcgaccagtactggtccgtccgccgccagaagcgcgtcggcggcactatcgacacgggcaagcactttgacgagtggaagcgccagggcaacctccagctcggcacctggaactacatgatcatggccaccgagggctaccagagctctggttcggccactatcgaggtccgggaggcc

SEQ ID NO: 152 - protein, 218 aa, Myceliophthora thermophila
MVSFTLLLTVIAAAVTTASPLEVVKRGIQPGTGTHEGYFYSFWTDGRGSVDFNPGPRGSYSVTWNNVNNWVGGKGWNPGPPRKIAYNGTWNNYNVNSYLALYGWTRNPLVEYYIVEAYGTYNPSSGTARLGTIEDDGGVYDIYKTTRYNQPSIEGTSTFDQYWSVRRQKRVGGTIDTGKHFDEWKRQGNLQLGTWNYMIMATEGYQSSGSATIEVREA

SEQ ID NO: 153 - protein, 218 aa, Myceliophthora thermophila; identical to SEQ ID NO: 152

SEQ ID NO: 154 - DNA, 1155 bp, Myceliophthora thermophila
atgcgtactcttacgttcgtgctggcagccgccccggtggctgtgcttgcccaatctcctctgtggggccagtgcggcggtcaaggctggacaggtcccacgacctgcgtttctggcgcagtatgccaattcgtcaatgactggtactcccaatgcgtgcccggatcgagcaaccctcctacgggcaccaccagcagcaccactggaagcaccccggctcctactggcggcggcggcagcggaaccggcctccacgacaaattcaaggccaagggcaagctctacttcggaaccgagatcgatcactaccatctcaacaacaatgccttgaccaacattgtcaagaaagactttggtcaagtcactcacgagaacagcttgaagtgggatgctactgagccgagccgcaatcaattcaactttgccaacgccgacgcggttgtcaactttgcccaggccaacggcaagctcatccgcggccacaccctcctctggcactctcagctgccgcagtgggtgcagaacatcaacgaccgcaacaccttgacccaggtcatcgagaaccacgtcaccacccttgtcactcgctacaagggcaagatcctccactgggacgtcgttaacgagatctttgccgaggacggctcgctccgcgacagcgtcttcagccgcgtcctcggcgaggactttgtcggcatcgccttccgcgccgcccgcgccgccgatcccaacgccaagctctacatcaacgactacaacctcgacattgccaactacgccaaggtgacccggggcatggtcgagaaggtcaacaagtggatcgcccagggcatcccgatcgacggcatcggcacccagtgccacctggccgggcccggcgggtggaacacggccgccggcgtccccgacgccctcaaggccctcgccgcggccaacgtcaaggagatcgccatcaccgagctcgacatcgccggcgcctccgccaacgactacctcaccgtcatgaacgcctgcctccaggtctccaagtgcgtcggcatcaccgtctggggcgtctctgacaaggacagctggaggtcgagcagcaacccgctcctcttcgacagcaactaccagccaaaggcggcatacaatgctctgattaatgccttgtaa

SEQ ID NO: 155 - protein, 384 aa, Myceliophthora thermophila
MRTLTFVLAAAPVAVLAQSPLWGQCGGQGWTGPTTCVSGAVCQFVNDWYSQCVPGSSNPPTGTTSSTTGSTPAPTGGGGSGTGLHDKFKAKGKLYFGTEIDHYHLNNNALTNIVKKDFGQVTHENSLKWDATEPSRNQFNFANADAVVNFAQANGKLIRGHTLLWHSQLPQWVQNINDRNTLTQVIENHVTTLVTRYKGKILHWDVVNEIFAEDGSLRDSVFSRVLGEDFVGIAFRAARAADPNAKLYINDYNLDIANYAKVTRGMVEKVNKWIAQGIPIDGIGTQCHLAGPGGWNTAAGVPDALKALAAANVKEIAITELDIAGASANDYLTVMNACLQVSKCVGITVWGVSDKDSWRSSSNPLLFDSNYQPKAAYNALINAL

SEQ ID NO: 156 - protein, 367 aa, Myceliophthora thermophila
QSPLWGQCGGQGWTGPTTCVSGAVCQFVNDWYSQCVPGSSNPPTGTTSSTTGSTPAPTGGGGSGTGLHDKFKAKGKLYFGTEIDHYHLNNNALTNIVKKDFGQVTHENSLKWDATEPSRNQFNFANADAVVNFAQANGKLIRGHTLLWHSQLPQWVQNINDRNTLTQVIENHVTTLVTRYKGKILHWDVVNEIFAEDGSLRDSVFSRVLGEDFVGIAFRAARAADPNAKLYINDYNLDIANYAKVTRGMVEKVNKWIAQGIPIDGIGTQCHLAGPGGWNTAAGVPDALKALAAANVKEIAITELDIAGASANDYLTVMNACLQVSKCVGITVWGVSDKDSWRSSSNPLLFDSNYQPKAAYNALINAL

SEQ ID NO: 157 - DNA, 687 bp, Myceliophthora thermophila
atggtctcgctcaagtccctcctcctcgccgcggcggcgacgttgacggcggtgacggcgcgcccgttcgactttgacgacggcaactcgaccgaggcgctggccaagcgccaggtcacgcccaacgcgcagggctaccactcgggctacttctactcgtggtggtccgacggcggcggccaggccaccttcaccctgctcgagggcagccactaccaggtcaactggaggaacacgggcaactttgtcggtggcaagggctggaacccgggtaccggccggaccatcaactacggcggctcgttcaacccgagcggcaacggctacctggccgtctacggctggacgcacaacccgctgatcgagtactacgtggtcgagtcgtacgggacctacaacccgggcagccaggcccagtacaagggcagcttccagagcgacggcggcacctacaacatctacgtctcgacccgctacaacgcgccctcgatcgagggcacccgcaccttccagcagtactggtccatccgcacctccaagcgcgtcggcggctccgtcaccatgcagaaccacttcaacgcctgggcccagcacggcatgcccctcggctcccacgactaccagatcgtcgccaccgagggctaccagagcagcggctcctccgacatctacgtccagactcactag

SEQ ID NO: 158 - protein, 228 aa, Myceliophthora thermophila
MVSLKSLLLAAAATLTAVTARPFDFDDGNSTEALAKRQVTPNAQGYHSGYFYSWWSDGGGQATFTLLEGSHYQVNWRNTGNFVGGKGWNPGTGRTINYGGSFNPSGNGYLAVYGWTHNPLIEYYVVESYGTYNPGSQAQYKGSFQSDGGTYNIYVSTRYNAPSIEGTRTFQQYWSIRTSKRVGGSVTMQNHFNAWAQHGMPLGSHDYQIVATEGYQSSGSSDIYVQTH

SEQ ID NO: 159 - protein, 208 aa, Myceliophthora thermophila
RPFDFDDGNSTEALAKRQVTPNAQGYHSGYFYSWWSDGGGQATFTLLEGSHYQVNWRNTGNFVGGKGWNPGTGRTINYGGSFNPSGNGYLAVYGWTHNPLIEYYVVESYGTYNPGSQAQYKGSFQSDGGTYNIYVSTRYNAPSIEGTRTFQQYWSIRTSKRVGGSVTMQNHFNAWAQHGMPLGSHDYQIVATEGYQSSGSSDIYVQTH

SEQ ID NO: 160 - DNA, 681 bp, Myceliophthora thermophila
atggttaccctcactcgcctggcggtcgccgcggcggccatgatctccagcactggcctggctgccccgacgcccgaagctggccccgaccttcccgactttgagctcggggtcaacaacctcgcccgccgcgcgctggactacaaccagaactacaggaccagcggcaacgtcaactactcgcccaccgacaacggctactcggtcagcttctccaacgcgggagattttgtcgtcgggaagggctggaggacgggagccaccagaaacatcaccttctcgggatcgacacagcatacctcgggcaccgtgctcgtctccgtctacggctggacccggaacccgctgatcgagtactacgtgcaggagtacacgtccaacggggccggctccgctcagggcgagaagctgggcacggtcgagagcgacgggggcacgtacgagatctggcggcaccagcaggtcaaccagccgtcgatcgagggcacctcgaccttctggcagtacatctcgaaccgcgtgtccggccagcggcccaacggcggcaccgtcaccctcgccaaccacttcgccgcctggcagaagctcggcctgaacctgggccagcacgactaccaggtcctggccaccgagggctggggcaacgccggcggcagctcccagtacaccgtcagcggctga

SEQ ID NO: 161 - protein, 226 aa, Myceliophthora thermophila
MVTLTRLAVAAAAMISSTGLAAPTPEAGPDLPDFELGVNNLARRALDYNQNYRTSGNVNYSPTDNGYSVSFSNAGDFVVGKGWRTGATRNITFSGSTQHTSGTVLVSVYGWTRNPLIEYYVQEYTSNGAGSAQGEKLGTVESDGGTYEIWRHQQVNQPSIEGTSTFWQYISNRVSGQRPNGGTVTLANHFAAWQKLGLNLGQHDYQVLATEGWGNAGGSSQYTVSG

SEQ ID NO: 162 - protein, 205 aa, Myceliophthora thermophila
APTPEAGPDLPDFELGVNNLARRALDYNQNYRTSGNVNYSPTDNGYSVSFSNAGDFVVGKGWRTGATRNITFSGSTQHTSGTVLVSVYGWTRNPLIEYYVQEYTSNGAGSAQGEKLGTVESDGGTYEIWRHQQVNQPSIEGTSTFWQYISNRVSGQRPNGGTVTLANHFAAWQKLGLNLGQHDYQVLATEGWGNAGGSSQYTVSG

SEQ ID NO: 163 - DNA, 1833 bp, Myceliophthora thermophila
atgttcttcgcttctctgctgctcggtctcctggcgggcgtgtccgcttcaccgggacacgggcggaattccaccttctacaaccccatcttccccggcttctaccccgatccgagctgcatctacgtgcccgagcgtgaccacaccttcttctgtgcctcgtcgagcttcaacgccttcccgggcatcccgattcatgccagcaaggacctgcagaactggaagttgatcggccatgtgctgaatcgcaaggaacagcttccccggctcgctgagaccaaccggtcgaccagcggcatctgggcacccaccctccggttccatgacgacaccttctggttggtcaccacactagtggacgacgaccggccgcaggaggacgcttccagatgggacaatattatcttcaaggcaaagaatccgtatgatccgaggtcctggtccaaggccgtccacttcaacttcactggctacgacacggagcctttctgggacgaagatggaaaggtgtacatcaccggcgcccatgcttggcatgttggcccatacatccagcaggccgaagtcgatctcgacacgggggccgtcggcgagtggcgcatcatctggaacggaacgggcggcatggctcctgaagggccgcacatctaccgcaaagatgggtggtactacttgctggctgctgaaggggggaccggcatcgaccatatggtgaccatggcccggtcgagaaaaatctccagtccttacgagtccaacccaaacaaccccgtgttgaccaacgccaacacgaccagttactttcaaaccgtcgggcattcagacctgttccatgacagacatgggaactggtgggcagtcgccctctccacccgctccggtccagaatatcttcactaccccatgggccgcgagaccgtcatgacagccgtgagctggccgaaggacgagtggccaaccttcacccccatatctggcaagatgagcggctggccgatgcctccttcgcagaaggacattcgcggagtcggcccctacgtcaactcccccgacccggaacacctgaccttcccccgctcggcgcccctgccggcccacctcacctactggcgatacccgaacccgtcctcctacacgccgtccccgcccgggcaccccaacaccctccgcctgaccccgtcccgcctgaacctgaccgccctcaacggcaactacgcgggggccgaccagaccttcgtctcgcgccggcagcagcacaccctcttcacctacagcgtcacgctcgactacgcgccgcggaccgccggggaggaggccggcgtgaccgccttcctgacgcagaaccaccacctcgacctgggcgtcgtcctgctccctcgcggctccgccaccgcgccctcgctgccgggcctgagtagtagtacaactactactagtagtagtagtagtcgtccggacgaggaggaggagcgcgaggcgggcgaagaggaagaagagggcggacaagacttgatgatcccgcatgtgcggttcaggggcgagtcgtacgtgcccgtcccggcgcccgtcgtgtacccgataccccgggcctggagaggcgggaagcttgtgttagagatccgggcttgtaattcgactcacttctcgttccgtgtcgggccggacgggagacggtctgagcggacggtggtcatggaggcttcgaacgaggccgttagctggggctttactggaacgctgctgggcatctatgcgaccagtaatggtggcaacggaaccacgccggcgtatttttcggattggaggtacacaccattggagcagtttagggat

SEQ ID NO: 164 - protein, 611 aa, Myceliophthora thermophila
MFFASLLLGLLAGVSASPGHGRNSTFYNPIFPGFYPDPSCIYVPERDHTFFCASSSFNAFPGIPIHASKDLQNWKLIGHVLNRKEQLPRLAETNRSTSGIWAPTLRFHDDTFWLVTTLVDDDRPQEDASRWDNIIFKAKNPYDPRSWSKAVHFNFTGYDTEPFWDEDGKVYITGAHAWHVGPYIQQAEVDLDTGAVGEWRIIWNGTGGMAPEGPHIYRKDGWYYLLAAEGGTGIDHMVTMARSRKISSPYESNPNNPVLTNANTTSYFQTVGHSDLFHDRHGNWWAVALSTRSGPEYLHYPMGRETVMTAVSWPKDEWPTFTPISGKMSGWPMPPSQKDIRGVGPYVNSPDPEHLTFPRSAPLPAHLTYWRYPNPSSYTPSPPGHPNTLRLTPSRLNLTALNGNYAGADQTFVSRRQQHTLFTYSVTLDYAPRTAGEEAGVTAFLTQNHHLDLGVVLLPRGSATAPSLPGLSSSTTTTSSSSSRPDEEEEREAGEEEEEGGQDLMIPHVRFRGESYVPVPAPVVYPIPRAWRGGKLVLEIRACNSTHFSFRVGPDGRRSERTVVMEASNEAVSWGFTGTLLGIYATSNGGNGTTPAYFSDWRYTPLEQFRD

SEQ ID NO: 165 - protein, 595 aa, Myceliophthora thermophila
SPGHGRNSTFYNPIFPGFYPDPSCIYVPERDHTFFCASSSFNAFPGIPIHASKDLQNWKLIGHVLNRKEQLPRLAETNRSTSGIWAPTLRFHDDTFWLVTTLVDDDRPQEDASRWDNIIFKAKNPYDPRSWSKAVHFNFTGYDTEPFWDEDGKVYITGAHAWHVGPYIQQAEVDLDTGAVGEWRIIWNGTGGMAPEGPHIYRKDGWYYLLAAEGGTGIDHMVTMARSRKISSPYESNPNNPVLTNANTTSYFQTVGHSDLFHDRHGNWWAVALSTRSGPEYLHYPMGRETVMTAVSWPKDEWPTFTPISGKMSGWPMPPSQKDIRGVGPYVNSPDPEHLTFPRSAPLPAHLTYWRYPNPSSYTPSPPGHPNTLRLTPSRLNLTALNGNYAGADQTFVSRRQQHTLFTYSVTLDYAPRTAGEEAGVTAFLTQNHHLDLGVVLLPRGSATAPSLPGLSSSTTTTSSSSSRPDEEEEREAGEEEEEGGQDLMIPHVRFRGESYVPVPAPVVYPIPRAWRGGKLVLEIRACNSTHFSFRVGPDGRRSERTVVMEASNEAVSWGFTGTLLGIYATSNGGNGTTPAYFSDWRYTPLEQFRD

SEQ ID NO: 166 - DNA, 942 bp, Myceliophthora thermophila
atgaagctcctgggcaaactctcggcggcactcgccctcgcgggcagcaggctggctgccgcgcacccggtcttcgacgagctgatgcggccgacggcgccgctggtgcgcccgcgggcggccctgcagcaggtgaccaactttggcagcaacccgtccaacacgaagatgttcatctacgtgcccgacaagctggcccccaacccgcccatcatagtggccatccactactgcaccggcaccgcccaggcctactactcgggctccccttacgcccgcctcgccgaccagaagggcttcatcgtcatctacccggagtccccctacagcggcacctgttgggacgtctcgtcgcgcgccgccctgacccacaacggcggcggcgacagcaactcgatcgccaacatggtcacctacaccctcgaaaagtacaatggcgacgccagcaaggtctttgtcaccggctcctcgtccggcgccatgatgacgaacgtgatggccgccgcgtacccggaactgttcgcggcaggaatcgcctactcgggcgtgcccgccggctgcttctacagccagtccggaggcaccaacgcgtggaacagctcgtgcgccaacgggcagatcaactcgacgccccaggtgtgggccaagatggtcttcgacatgtacccggaatacgacggcccgcgccccaagatgcagatctaccacggctcggccgacggcacgctcagacccagcaactacaacgagaccatcaagcagtggtgcggcgtcttcggcttcgactacacccgccccgacaccacccaggccaactccccgcaggccggctacaccacctacacctggggcgagcagcagctcgtcggcatctacgcccagggcgtcggacacacggtccccatccgcggcagcgacgacatggccttctttggcctgtga

SEQ ID NO: 167 - protein, 313 aa, Myceliophthora thermophila
MKLLGKLSAALALAGSRLAAAHPVFDELMRPTAPLVRPRAALQQVTNFGSNPSNTKMFIYVPDKLAPNPPIIVAIHYCTGTAQAYYSGSPYARLADQKGFIVIYPESPYSGTCWDVSSRAALTHNGGGDSNSIANMVTYTLEKYNGDASKVFVTGSSSGAMMTNVMAAAYPELFAAGIAYSGVPAGCFYSQSGGTNAWNSSCANGQINSTPQVWAKMVFDMYPEYDGPRPKMQIYHGSADGTLRPSNYNETIKQWCGVFGFDYTRPDTTQANSPQAGYTTYTWGEQQLVGIYAQGVGHTVPIRGSDDMAFFGL

SEQ ID NO: 168 - protein, 292 aa, Myceliophthora thermophila
HPVFDELMRPTAPLVRPRAALQQVTNFGSNPSNTKMFIYVPDKLAPNPPIIVAIHYCTGTAQAYYSGSPYARLADQKGFIVIYPESPYSGTCWDVSSRAALTHNGGGDSNSIANMVTYTLEKYNGDASKVFVTGSSSGAMMTNVMAAAYPELFAAGIAYSGVPAGCFYSQSGGTNAWNSSCANGQINSTPQVWAKMVFDMYPEYDGPRPKMQIYHGSADGTLRPSNYNETIKQWCGVFGFDYTRPDTTQANSPQAGYTTYTWGEQQLVGIYAQGVGHTVPIRGSDDMAFFGL

SEQ ID NO: 169 - DNA, 840 bp, Myceliophthora thermophila
atgatctcggttcctgctctcgctctggcccttctggccgccgtccaggtcgtcgagtctgcctcggctggctgtggcaaggcgcccccttcctcgggcaccaagtcgatgacggtcaacggcaagcagcgccagtacattctccagctgcccaacaactacgacgccaacaaggcccacagggtggtgatcgggtaccactggcgcgacggatccatgaacgacgtggccaacggcggcttctacgatctgcggtcccgggcgggcgacagcaccatcttcgttgcccccaacggcctcaatgccggatgggccaacgtgggcggcgaggacatcacctttacggaccagatcgtagacatgctcaagaacgacctctgcgtggacgagacccagttctttgctacgggctggagctatggcggtgccatgagccatagcgtggcttgttctcggccagacgtcttcaaggccgtcgcggtcatcgccggggcccagctgtccggctgcgccggcggcacgacgcccgtggcgtacctaggcatccacggagccgccgacaacgtcctgcccatcgacctcggccgccagctgcgcgacaagtggctgcagaccaacggctgcaactaccagggcgcccaggaccccgcgccgggccagcaggcccacatcaagaccacctacagctgctcccgcgcgcccgtcacctggatcggccacgggggcggccacgtccccgaccccacgggcaacaacggcgtcaagtttgcgccccaggagacctgggacttctttgatgccgccgtcggagcggccggcgcgcagagcccgatgacataa

SEQ ID NO: 170 - protein, 279 aa, Myceliophthora thermophila
MISVPALALALLAAVQVVESASAGCGKAPPSSGTKSMTVNGKQRQYILQLPNNYDANKAHRVVIGYHWRDGSMNDVANGGFYDLRSRAGDSTIFVAPNGLNAGWANVGGEDITFTDQIVDMLKNDLCVDETQFFATGWSYGGAMSHSVACSRPDVFKAVAVIAGAQLSGCAGGTTPVAYLGIHGAADNVLPIDLGRQLRDKWLQTNGCNYQGAQDPAPGQQAHIKTTYSCSRAPVTWIGHGGGHVPDPTGNNGVKFAPQETWDFFDAAVGAAGAQSPMT

SEQ ID NO: 171 - protein, 259 aa, Myceliophthora thermophila
ASAGCGKAPPSSGTKSMTVNGKQRQYILQLPNNYDANKAHRVVIGYHWRDGSMNDVANGGFYDLRSRAGDSTIFVAPNGLNAGWANVGGEDITFTDQIVDMLKNDLCVDETQFFATGWSYGGAMSHSVACSRPDVFKAVAVIAGAQLSGCAGGTTPVAYLGIHGAADNVLPIDLGRQLRDKWLQTNGCNYQGAQDPAPGQQAHIKTTYSCSRAPVTWIGHGGGHVPDPTGNNGVKFAPQETWDFFDAAVGAAGAQSPMT

(19) United States
(12) Patent Application Publication
(10) Pub. No.: US 2014/0356914 A1
(43) Pub. Date: Dec. 4, 2014

(54) ENDOGLUCANASE 1B

(71) Applicants: Kripa Rao, Union City, CA (US); Xiyun Zhang, Fremont, CA (US); Brian R. Scott, Richmond (CA); John J. Tomashek, Ottawa (CA)

(72) Inventors: Kripa Rao, Union City, CA (US); Xiyun Zhang, Fremont, CA (US); Brian R. Scott, Richmond (CA); John J. Tomashek, Ottawa (CA)

(73) Assignee: Codexis, Inc., Redwood City, CA (US)

(21) Appl. No.: 14/345,442

(22) PCT Filed: Aug. 27, 2012
(86) PCT No.: PCT/US12/52498

§ 371 Date: Mar. 18, 2014

Related U.S. Application Data
(60) Provisional application No. 61/536,856, filed on Sep. 20, 2011.

(51) Int. Cl. C12P 19/14; C12N 9/42

(52) U.S. Cl. CPC C12P 19/14; C12N 9/2437
USPC 435/99; 435/201; 435/254.11; 435/320.1; 536/23.2

(57) ABSTRACT

The present invention provides endoglucanase 1b (EG1b) suitable for use in saccharification reactions.

The present application claims priority to previously filed U.S. Prov. Appin. Ser. No. 61/536,856, filed Sep. 20, 2011, which is hereby incorporated in its entirety for all purposes.

### REFERENCE TO A “SEQUENCE LISTING,” A TABLE, OR A COMPUTER PROGRAM LISTING APPENDIX SUBMITTED AS AN ASCII TEXT FILE

The Sequence Listing written in file CX35-099WO1_ST25.TXT, created on Aug. 27, 2012, 416,957 bytes, machine format IBM-PC, MS-Windows operating system, is hereby incorporated by reference.

### FIELD OF THE INVENTION

The present invention provides endoglucanase 1b (EG1b) suitable for use in saccharification reactions.

### BACKGROUND

Interest has arisen in fermentation of carbohydrate-rich biomass to provide alternatives to petrochemical sources for fuels and organic chemical precursors. “First generation” bioethanol production from carbohydrate sources (e.g., sugar cane, corn, wheat, etc.) have proven to be marginally economically viable on a production scale. “Second generation” bioethanol produced using lignocellulosic feedstocks has faced significant obstacles to commercial viability. Bioethanol is currently produced by the fermentation of hexose sugars that are obtained from carbon feedstocks. There is great interest in using lignocellulosic feedstocks where the plant cellulose is broken down to sugars and subsequently converted to ethanol. Lignocellulosic biomass is primarily composed of cellulose, hemicelluloses, and lignin. Cellulose and hemicellulose can be hydrolyzed in a saccharification process to sugars that can be subsequently converted to ethanol via fermentation. The major fermentable sugars from lignocelluloses are glucose and xylose. For economical ethanol yields, a process that can effectively convert all the major sugars present in cellulosic feedstock would be highly desirable.

### SUMMARY OF THE INVENTION

The present invention provides endoglucanase 1b (EG1b) suitable for use in saccharification reactions.

The present invention provides cells comprising a recombinant nucleic acid sequence encoding (i) an endoglucanase 1b (EG1b) protein comprising SEQ ID NO:2 and (ii) an operably-linked heterologous promoter, wherein the cell produces at least one recombinant cellulase protein selected from beta-glucosidases (BGLs), Type 1 cellobiohydrolases (CBH1s), Type 2 cellobiohydrolases (CBH2s), glycoside hydrolase 61s (GH61s), and/or endoglucanases (EGs). In some embodiments, the recombinant nucleic acid sequence comprises the nucleotide sequence set forth in SEQ ID NO:1. In some embodiments, the cells produce at least one recombinant cellulase protein selected from Myceliophthora thermophila endoglucanases (EGs), beta-glucosidases (BGLs), Type 1 cellobiohydrolases (CBH1s), Type 2 cellobiohydrolases (CBH2s), and/or glycoside hydrolase 61s (GH61s), and/or variants of the cellulase proteins. In some embodiments, the cells produce at least two recombinant cellulases, while in some other embodiments, the cells produce at least three, at least four or at least five recombinant cellulases. In some additional embodiments, the cells are prokaryotic cells, while in some other embodiments, the cells are eukaryotic cells. In some further embodiments, the cells are yeast cells or filamentous fungal cells. In some embodiments, the cells are Saccharomyces or Myceliophthora cells.

The present invention also provides compositions comprising an EG1b protein comprising SEQ ID NO:2, and one or more cellulases selected from endoglucanases (EGs), beta-glucosidases (BGLs), Type 1 cellobiohydrolases (CBH1s), Type 2 cellobiohydrolases (CBH2s), and/or glycoside hydrolase 61s (GH61s), and/or variants of the cellulase proteins. In some embodiments, the EG is EG2, EG3, EG4, EG5, and/or EG6. In some further embodiments, the CBH1 is CBH1a and/or CBH1b. In some still further embodiments, the CBH2 is CBH2b and/or CBH2a. In some additional embodiments, the GH61 is GH61a. In still some additional embodiments, the GH61, CBH1, CBH2, EG, and/or BGL, are contained in a cell culture broth.

The present invention also provides recombinant nucleic acid sequences encoding a protein comprising SEQ ID NO:2. In some embodiments, the protein-encoding sequence is operably linked to a heterologous signal sequence. In some further embodiments, the protein-encoding sequence is operably linked to a heterologous promoter. In some embodiments, the recombinant nucleic acid sequence comprises SEQ ID NO:1. The present invention also provides vectors comprising the recombinant nucleic acid. In some embodiments, the vectors further comprise at least one polynucleotide sequence encoding at least one EG, BGL, CBH1, CHB2, and/or GH61 protein. The present invention also provides host cells comprising at least one vector. In some embodiments, the host cells produce at least one recombinant cellulase protein selected from EGs, BGLs, CBH1s, CBH2s, and GH61s. In some additional embodiments, the host cells produce at least two, three or four recombinant cellulases. In some embodiments, the host cells are prokaryotic cells, while in some alternative embodiments, the host cells are eukaryotic cells. In some embodiments, the host cells are yeast cells or filamentous fungal cells. In some additional embodiments, the host cells are Saccharomyces or Myceliophthora cells. In some embodiments, one, two, three, four, or all five of the CBH1, CBH2, EG, GH61, and/or BGL are variant Myceliophthora cellulase proteins.

The present invention also provides methods for saccharification comprising (a) culturing cells as provided herein, under conditions in which EG1b protein is secreted into a culture broth, and (b) combining the broth and a biomass under conditions in which saccharification occurs, where (a) may take place before or simultaneously with (b).

The present invention also provides methods for saccharification comprising culturing cells as provided herein, under conditions in which EG1b protein is secreted into a culture broth, isolating the EG1b from the broth, and combining the isolated EG1b protein and biomass under conditions in which saccharification occurs. In some embodiments, the biomass is cellulosic biomass.

The present invention also provides methods for reducing viscosity during saccharification reactions comprising providing EG1b in a saccharification reaction mixture under conditions such that the viscosity of the saccharification reaction mixture is less viscous than a saccharification reaction mixture without said EG1b. In some embodiments, the saccharification reaction mixture comprises at least one additional enzyme selected from CBH1, CBH2, BGL, EG2, and GH61. In some additional embodiments, the saccharification reaction mixture does not comprise EG2.

### DESCRIPTION OF THE FIGURES

FIG. 1 provides the map of pYTsec72-EG1b-cDNA.

FIG. 2 provides a graph showing the viscosity reduction effect provided by the inclusion of EG1b in a saccharification reaction.

FIG. 3 provides a graph showing the improvement in glucose yield provided by the inclusion of EG1b in a saccharification reaction.

### DESCRIPTION OF THE INVENTION

The present invention provides endoglucanase 1b (EG1b) suitable for use in saccharification reactions. In some embodiments, the EG1b is obtained from Myceliophthora thermophila.

All patents and publications, including all sequences disclosed within such patents and publications, referred to herein are expressly incorporated by reference. Unless otherwise indicated, the practice of the present invention involves conventional techniques commonly used in molecular biology, fermentation, microbiology, and related fields, which are known to those of skill in the art. Unless defined otherwise herein, all technical and scientific terms used herein have the same meaning as commonly understood by one of ordinary skill in the art to which this invention belongs. Although any methods and materials similar or equivalent to those described herein can be used in the practice or testing of the present invention, some suitable methods and materials are described. Indeed, it is intended that the present invention not be limited to the particular methodology, protocols, and reagents described herein, as these may vary, depending upon the context in which they are used. The headings provided herein are not limitations of the various aspects or embodiments of the present invention.

Nonetheless, in order to facilitate understanding of the present invention, a number of terms are defined below. Numeric ranges are inclusive of the numbers defining the range. Thus, every numerical range disclosed herein is intended to encompass every narrower numerical range that falls within such broader numerical range, as if such narrower numerical ranges were all expressly written herein. It is also intended that every maximum (or minimum) numerical limitation disclosed herein includes every lower (or higher) numerical limitation, as if such lower (or higher) numerical limitations were expressly written herein.

As used herein, the term “comprising” and its cognates are used in their inclusive sense (i.e., equivalent to the term “including” and its corresponding cognates).

As used herein and in the appended claims, the singular “a”, “an” and “the” include the plural reference unless the context clearly dictates otherwise. Thus, for example, reference to a “host cell” includes a plurality of such host cells.

Unless otherwise indicated, nucleic acids are written left to right in 5′ to 3′ orientation; amino acid sequences are written left to right in amino to carboxy orientation, respectively. The headings provided herein are not limitations of the various aspects or embodiments of the invention that can be had by reference to the specification as a whole. Accordingly, the terms defined below are more fully defined by reference to the specification as a whole.

As used herein, the term “cellulase” refers to any enzyme that is capable of degrading cellulose. Thus, the term encompasses enzymes capable of hydrolyzing cellulose (beta-1,4-glucan or beta-D-glucosidic linkages) to shorter cellulose chains, oligosaccharides, cellobiose and/or glucose. “Cellulases” are divided into three sub-categories of enzymes: 1,4-beta-D-glucan glucanohydrolase (“endoglucanase” or “EG”); 1,4-beta-D-glucan cellobiohydrolase (“exoglucanase,” “cellobiohydrolase,” or “CBH”); and beta-D-glucoside-glucohydrolase (“beta-glucosidase,” “cellobiase,” “BG,” or “BGL”). These enzymes act in concert to catalyze the hydrolysis of cellulose-containing substrates. Endoglucanases break internal bonds and disrupt the crystalline structure of cellulose, exposing individual cellulose polysaccharide chains (“glucans”). Cellobiohydrolases incrementally shorten the glucan molecules, releasing mainly cellobiose units (a water-soluble beta-1,4-linked dimer of glucose) as well as glucose, cellotriose, and cellotetrose. beta-glucosidases split the cellobiose into glucose monomers.

A “cellulase-engineered” cell is a cell comprising at least one, at least two, at least three, or at least four recombinant sequences encoding a cellulase or cellulase variant, and in which expression of the cellulase(s) or cellulase variant(s) has been modified relative to the wild-type form. Expression of a cellulase is “modified” when a non-naturally occurring cellulase variant is expressed or when a naturally occurring cellulase is over-expressed. One exemplary means to over-express a cellulase is to operably link a strong (optionally constitutive) promoter to the cellulase encoding sequence. Another exemplary way to over-express a cellulase is to increase the copy number of a heterologous, variant, or endogenous cellulase gene. The cellulase-engineered cell may be any suitable fungal cell, including, but not limited to Myceliophthora, Trichoderma, Aspergillus, cells, etc.

As used herein, the terms “endoglucanase” and “EG” refer to a category of cellulases (EC 3.2.1.4) that catalyze the hydrolysis of internal beta-1,4 glucosidic bonds of cellulose.

As used herein, “EG1” refers to a carbohydrate active enzyme expressed from a nucleic acid sequence coding for a glycohydrolase (GH) Family 7 catalytic domain classified under EC 3.2.1.4 or any protein, polypeptide or catalytically active fragment thereof. In some embodiments, the EG 1 is functionally linked to a carbohydrate binding module (CBM), such as a Family 1 cellulose binding domain.

As used herein, the term “EG1b polypeptide” refers to a polypeptide having EG1b activity. In some embodiments, the EG1b polypeptide comprises the sequence set forth in SEQ ID NO:2.

As used herein, the term “EG1b polynucleotide” refers to a polynucleotide encoding a polypeptide having EG1b activity.

As used herein, the term “EG1b activity” refers to the enzymatic activity of EG1b (i.e., hydrolyzing a cellulose-containing substrate).

As used herein, the terms “wild-type EG1b polynucleotide,” “wild-type EG1b DNA,” and “wild-type EG1b nucleic acid” refer to SEQ ID NO:1. SEQ ID NO:2 is the pre-mature peptide sequence (i.e., containing a signal peptide) of EG1b that is expressed by a naturally occurring Myceliophtora thermophila strain.

As used herein, the term “EG2” refers to a carbohydrate active enzyme expressed from a nucleic acid sequence coding for a glycohydrolase (GH) Family 5 catalytic domain classified under EC 3.2.1.4 or any protein, polypeptide or catalytically active fragment thereof. In some embodiments, the EG2 is functionally linked to a carbohydrate binding module (CBM), such as a Family 1 cellulose binding domain.

As used herein, the term “EG3” refers to a carbohydrate active enzyme expressed from a nucleic acid sequence coding for a glycohydrolase (GH) Family 12 catalytic domain classified under EC 3.2.1.4 or any protein, polypeptide or catalytically active fragment thereof. In some embodiments, the EG3 is functionally linked to a carbohydrate binding module (CBM), such as a Family 1 cellulose binding domain.

As used herein, the term “EG4” refers to a carbohydrate active enzyme expressed from a nucleic acid sequence coding for a glycohydrolase (GH) Family 61 catalytic domain classified under EC 3.2.1.4 or any protein, polypeptide or fragment thereof. In some embodiments, the EG4 is functionally linked to a carbohydrate binding module (CBM), such as a Family 1 cellulose binding domain.

As used herein, the term “EG5” refers to a carbohydrate active enzyme expressed from a nucleic acid sequence coding for a glycohydrolase (GH) Family 45 catalytic domain classified under EC 3.2.1.4 or any protein, polypeptide or fragment thereof. In some embodiments, the EG5 is functionally linked to a carbohydrate binding module (CBM), such as a Family 1 cellulose binding domain.

As used herein, the term “EG6” refers to a carbohydrate active enzyme expressed from a nucleic acid sequence coding for a glycohydrolase (GH) Family 6 catalytic domain classified under EC 3.2.1.4 or any protein, polypeptide or fragment thereof. In some embodiments, the EG6 is functionally linked to a carbohydrate binding module (CBM), such as a Family 1 cellulose binding domain.

As used herein, the terms “cellobiohydrolase” and “CBH” refer to a category of cellulases (EC 3.2.1.91) that hydrolyze glycosidic bonds in cellulose.

As used herein, the terms “CBH1,” “type 1 cellobiohydrolase,” and “cellobiohydrolase 1,” refer to a carbohydrate active enzyme expressed from a nucleic acid sequence coding for a glycohydrolase (GH) Family 7 catalytic domain classified under EC 3.2.1.91 or any protein, polypeptide or catalytically active fragment thereof. In some embodiments, the CBH1 is functionally linked to a carbohydrate binding module (CBM), such as a Family 1 cellulose binding domain.

As used herein, the terms “CBH2,” “type 2 cellobiohydrolase,” and “cellobiohydrolase 2,” refer to a carbohydrate active enzyme expressed from a nucleic sequence coding for a glycohydrolase (GH) Family 6 catalytic domain classified under EC 3.2.1.91 or any protein, polypeptide or catalytically active fragment thereof. Type 2 cellobiohydrolases are also commonly referred to as “the Cel6 family.” The CBH2 may be functionally linked to a carbohydrate binding module (CBM), such as a Family 1 cellulose binding domain.

As used herein, the terms “beta-glucosidase,” “cellobiase,” and “BGL” refers to a category of cellulases (EC 3.2.1.21) that catalyze the hydrolysis of cellobiose to glucose.

As used herein, the term “glycoside hydrolase 61” and “GH61” refers to a category of cellulases that enhance cellulose hydrolysis when used in conjunction with one or more additional cellulases. The GH61 family of cellulases is described, for example, in the Carbohydrate Active Enzymes (CAZY) database (See e.g., Harris et al., Biochem., 49(15):3305-16 [2010]).

A “hemicellulase” as used herein, refers to a polypeptide that can catalyze hydrolysis of hemicellulose into small polysaccharides such as oligosaccharides, or monomeric saccharides. Hemicellulloses include xylan, glucuonoxylan, arabinoxylan, glucomannan and xyloglucan. Hemicellulases include, for example, the following: endoxylanases, b-xylosidases, a-L-arabinofuranosidases, a-D-glucuronidases, feruloyl esterases, coumaroyl esterases, a-galactosidases, b-galactosidases, b-mannanases, and b-mannosidases. In some embodiments, the present invention provides enzyme mixtures that comprise EG1b and one or more hemicellulases.

As used herein, “protease” includes enzymes that hydrolyze peptide bonds (peptidases), as well as enzymes that hydrolyze bonds between peptides and other moieties, such as sugars (glycopeptidases). Many proteases are characterized under EC 3.4, and are suitable for use in the present invention. Some specific types of proteases include but are not limited to, cysteine proteases including pepsin, papain and serine proteases including chymotrypsins, carboxypeptidases and metalloendopeptidases.

As used herein, “lipase” includes enzymes that hydrolyze lipids, fatty acids, and acylglycerides, including phosphoglycerides, lipoproteins, diacylglycerols, and the like. In plants, lipids are used as structural components to limit water loss and pathogen infection. These lipids include waxes derived from fatty acids, as well as cutin and suberin.

As used herein, the terms “isolated” and “purified” are used to refer to a molecule (e.g., an isolated nucleic acid, polypeptide, etc.) or other component that is removed from at least one other component with which it is naturally associated. In some embodiments, the term “isolated” refers to a nucleic acid, polypeptide, or other component that is partially or completely separated from components with which it is normally associated in nature. Thus, the term encompasses a substance in a form or environment that does not occur in nature. Non-limiting examples of isolated substances include, but are not limited to: any non-naturally occurring substance; any substance including, but not limited to, any enzyme, variant, polynucleotide, protein, peptide or cofactor, that is at least partially removed from one or more or all of the naturally occurring constituents with which it is associated in nature; any substance modified by the hand of man relative to that substance found in nature; and/or any substance modified by increasing the amount of the substance relative to other components with which it is naturally associated (e.g., multiple copies of a gene encoding the substance; and/or use of a stronger promoter than the promoter naturally associated with the gene encoding the substance). In some embodiments, a polypeptide of interest is used in industrial applications in the form of a fermentation broth product (i.e., the polypeptide is a component of a fermentation broth) used as a product in industrial applications such as ethanol production. In some embodiments, in addition to the polypeptide of interest (e.g., an EG1b polypeptide), the fermentation broth product further comprises ingredients used in the fermentation process (e.g., cells, including the host cells containing the gene encoding the polypeptide of interest and/or the polypeptide of interest), cell debris, biomass, fermentation media, and/or fermentation products. In some embodiments, the fermentation broth is optionally subjected to one or more purification steps (e.g., filtration) to remove or reduce at least one components of a fermentation process. Accordingly, in some embodiments, an isolated substance is present in such a fermentation broth product.

As used herein, “polynucleotide” refers to a polymer of deoxyribonucleotides or ribonucleotides in either single- or double-stranded form, and complements thereof.

The terms “protein” and “polypeptide” are used interchangeably herein to refer to a polymer of amino acid residues.

The term “EG1b polynucleotide” refers to a polynucleotide that encodes an endoglucanase 1b polypeptide.

In addition, the terms “amino acid” “polypeptide,” and “peptide” encompass naturally-occurring and synthetic amino acids, as well as amino acid analogs. Naturally occurring amino acids are those encoded by the genetic code, as well as those amino acids that are later modified (e.g., hydroxyproline, γ-carboxyglutamate, and O-phosphoserine). As used herein, the term “amino acid analogs” refers to compounds that have the same basic chemical structure as a naturally occurring amino acid (i.e., an alpha-carbon that is bound to a hydrogen, a carboxyl group, an amino group, and an R group, including but not limited to homoserine, norleucine, methionine sulfoxide, and methionine methyl sulfonium). In some embodiments, these analogs have modified R groups (e.g., norleucine) and/or modified peptide backbones, but retain the same basic chemical structure as a naturally occurring amino acid.

Amino acids are referred to herein by either their commonly known three letter symbols or by the one-letter symbols recommended by the IUPAC-IUB Biochemical Nomenclature Commission. Nucleotides, likewise, may be referred to by their commonly accepted single-letter codes.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, the amino acid residue number in a test sequence determined by simply counting from the N-terminus will not necessarily be the same as the number of its corresponding position in the reference sequence. For example, in a case where a test sequence has a deletion relative to an aligned reference sequence, there will be no amino acid in the variant that corresponds to a position in the reference sequence at the site of deletion. Where there is an insertion in an aligned test sequence, that insertion will not correspond to a numbered amino acid position in the reference sequence. In the case of truncations or fusions there can be stretches of amino acids in either the reference or aligned sequence that do not correspond to any amino acid in the corresponding sequence.

As used herein, the terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

As used herein, the term “reference enzyme” refers to an enzyme to which another enzyme of the present invention (e.g., a “test” enzyme) is compared in order to determine the presence of an improved property in the other enzyme being evaluated. In some embodiments, a reference enzyme is a wild-type enzyme (e.g., wild-type EG1b). In some embodiments, the reference enzyme is an enzyme to which a test enzyme of the present invention is compared in order to determine the presence of an improved property in the test enzyme being evaluated, including but not limited to improved thermoactivity, improved thermostability, and/or improved stability. In some embodiments, a reference enzyme is a wild-type enzyme (e.g., wild-type EG1b).

As used herein, the term “biologically active fragment,” refers to a polypeptide that has an amino-terminal and/or carboxy-terminal deletion(s) and/or internal deletion(s), but where the remaining amino acid sequence is identical to the corresponding positions in the sequence to which it is being compared (e.g., a full-length EG1b of the present invention) and that retains substantially all of the activity of the full-length polypeptide. In some embodiments, the biologically active fragment is a biologically active EG1b fragment. A biologically active fragment can comprise about 60%, about 65%, about 70%, about 75%, about 80%, about 85%, at about 90%, about 91%, about 92%, about 93%, about 94%, about 95%, about 96%, about 97%, about 98%, or about 99% of a full-length EG1b polypeptide.

As used herein, the term “overexpress” is intended to encompass increasing the expression of a protein to a level greater than the cell normally produces. It is intended that the term encompass overexpression of endogenous, as well as heterologous proteins.

As used herein, the term “recombinant” refers to a polynucleotide or polypeptide that does not naturally occur in a host cell. In some embodiments, recombinant molecules contain two or more naturally-occurring sequences that are linked together in a way that does not occur naturally. In some embodiments, “recombinant cells” express genes that are not found in identical form within the native (i.e., non-recombinant) form of the cell and/or express native genes that are otherwise abnormally over-expressed, under-expressed, and/or not expressed at all due to deliberate human intervention. Recombinant cells contain at least one recombinant polynucleotide or polypeptide. A nucleic acid construct, nucleic acid (e.g., a polynucleotide), polypeptide, or host cell is referred to herein as “recombinant” when it is non-naturally occurring, artificial or engineered. “Recombination,” “recombining” and generating a “recombined” nucleic acid generally encompass the assembly of at least two nucleic acid fragments.

The present invention also provides a recombinant nucleic acid construct comprising an EG1b polynucleotide sequence that hybridizes under stringent hybridization conditions to the complement of a polynucleotide which encodes a polypeptide having the amino acid sequence of SEQ ID NO:2.

Nucleic acids “hybridize” when they associate, typically in solution. Nucleic acids hybridize due to a variety of well-characterized physico-chemical forces, such as hydrogen bonding, solvent exclusion, base stacking and the like. As used herein, the term “stringent hybridization wash conditions” in the context of nucleic acid hybridization experiments, such as Southern and Northern hybridizations, are sequence dependent, and are different under different environmental parameters. An extensive guide to the hybridization of nucleic acids is found in Tijssen, 1993, “Laboratory Techniques in Biochemistry and Molecular Biology-Hybridization with Nucleic Acid Probes,” Part I, Chapter 2 (Elsevier, New York), which is incorporated herein by reference. For polynucleotides of at least 100 nucleotides in length, low to very high stringency conditions are defined as follows: prehybridization and hybridization at 42° C. in 5×SSPE, 0.3% SDS, 200 μg/ml sheared and denatured salmon sperm DNA, and either 25% formamide for low stringencies, 35% formamide for medium and medium-high stringencies, or 50% formamide for high and very high stringencies, following standard Southern blotting procedures. For polynucleotides of at least 100 nucleotides in length, the carrier material is finally washed three times each for 15 minutes using 2×SSC, 0.2% SDS 50° C. (low stringency), at 55° C. (medium stringency), at 60° C. (medium-high stringency), at 65° C. (high stringency), or at 70° C. (very high stringency).

As used herein, “identity” or “percent identity,” in the context of two or more polypeptide sequences, refers to two or more sequences or subsequences that are the same or have a specified percentage of amino acid residues that are the same (e.g., share at least about 70%, at least about 75%, at least about 80%, at least about 85%, at least about 88% identity, at least about 89%, at least about 90%, at least about 91%, at least about 92%, at least about 93%, at least about 94%, at least about 95%, at least about 96%, at least about 97%, at least about 98%, at least about 99% identity, or at least 100%) over a specified region to a reference sequence, when compared and aligned for maximum correspondence over a comparison window, or designated region as measured using a sequence comparison algorithms or by manual alignment and visual inspection.

In some embodiments, the terms “percent identity,” “% identity”, “percent identical,” and “% identical,” are used interchangeably herein to refer to the percent amino acid or polynucleotide sequence identity that is obtained by ClustalW analysis (version W 1.8 available from European Bioinformatics Institute, Cambridge, UK), counting the number of identical matches in the alignment and dividing such number of identical matches by the length of the reference sequence, and using the following ClustalW parameters to achieve slow/more accurate pairwise optimal alignments—DNA/Protein Gap Open Penalty:15/10; DNA/Protein Gap Extension Penalty:6.66/0.1; Protein weight matrix: Gonnet series; DNA weight matrix: Identity.

As used herein the term “comparison window,” includes reference to a segment of any one of a number of contiguous positions from about 20 to about 464 (e.g., about 50 to about 300 contiguous positions, about 50 to 250 contiguous positions, or also about 100 to about 200 contiguous positions), in which a sequence may be compared to a reference sequence of the same number of contiguous positions after the two sequences are optimally aligned. As noted, in some embodiments the comparison is between the entire length of the two sequences, or, if one sequence is a fragment of the other, the entire length of the shorter of the two sequences. Optimal alignment of sequences for comparison and determination of sequence identity can be determined by a sequence comparison algorithm or by visual inspection, as well-known in the art. When optimally aligning sequences and determining sequence identity by visual inspection, percent sequence identity is calculated as the number of residues of the test sequence that are identical to the reference sequence divided by the number of non-gap positions and multiplied by 100. When using a sequence comparison algorithm, test and reference sequences are entered into a computer, subsequence coordinates and sequence algorithm program parameters are designated. The sequence comparison algorithm then calculates the percent sequence identities for the test sequences relative to the reference sequence, based on the program parameters.

Two sequences are “aligned” when they are aligned for similarity scoring using a defined amino acid substitution matrix (e.g., BLOSUM62), gap existence penalty and gap extension penalty so as to arrive at the highest score possible for that pair of sequences. Amino acid substitution matrices and their use in quantifying the similarity between two sequences are well known in the art (See, e.g., Dayhoff et al., in Dayhoff [ed.], Atlas of Protein Sequence and Structure,” Vol. 5, Suppl. 3, Natl. Biomed. Res. Round., Washington D.C. [1978]; pp. 345-352; and Henikoff et al., Proc. Natl. Acad. Sci. USA, 89:10915-10919 [1992], both of which are incorporated herein by reference). The BLOSUM62 matrix is often used as a default scoring substitution matrix in sequence alignment protocols such as Gapped BLAST 2.0. The gap existence penalty is imposed for the introduction of a single amino acid gap in one of the aligned sequences, and the gap extension penalty is imposed for each additional empty amino acid position inserted into an already opened gap. The alignment is defined by the amino acid position of each sequence at which the alignment begins and ends, and optionally by the insertion of a gap or multiple gaps in one or both sequences so as to arrive at the highest possible score. While optimal alignment and scoring can be accomplished manually, the process is facilitated by the use of a computer-implemented alignment algorithm (e.g., gapped BLAST 2.0; See, Altschul et al., Nucleic Acids Res., 25:3389-3402 [1997], which is incorporated herein by reference), and made available to the public at the National Center for Biotechnology Information Website). Optimal alignments, including multiple alignments can be prepared using readily available programs such as PSI-BLAST (See e.g, Altschul et al., supra).

The present invention also provides a recombinant nucleic acid construct comprising an EG1b polynucleotide sequence that hybridizes under stringent hybridization conditions to the complement of a polynucleotide which encodes a polypeptide having the amino acid sequence of SEQ ID NO:2, wherein the polypeptide is capable of catalyzing the degradation of cellulose. Two nucleic acid or polypeptide sequences that have 100% sequence identity are said to be “identical.” A nucleic acid or polypeptide sequence are said to have “substantial sequence identity” to a reference sequence when the sequences have at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%, or greater sequence identity as determined using the methods described herein, such as BLAST using standard parameters.

As used herein, the term “pre-protein” refers to a protein including an amino-terminal signal peptide (or leader sequence) region attached. The signal peptide is cleaved from the pre-protein by a signal peptidase prior to secretion to result in the “mature” or “secreted” protein.

As used herein, a “vector” is a DNA construct for introducing a DNA sequence into a cell. In some embodiments, the vector is an expression vector that is operably linked to a suitable control sequence capable of effecting the expression in a suitable host of the polypeptide encoded in the DNA sequence. An “expression vector” has a promoter sequence operably linked to the DNA sequence (e.g., transgene) to drive expression in a host cell, and in some embodiments a transcription terminator sequence.

As used herein, the term “expression” includes any step involved in the production of the polypeptide including, but not limited to, transcription, post-transcriptional modification, translation, and post-translational modification. In some embodiments, the term also encompasses secretion of the polypeptide from a cell.

As used herein, the term “produces” refers to the production of proteins and/or other compounds by cells. It is intended that the term encompass any step involved in the production of polypeptides including, but not limited to, transcription, post-transcriptional modification, translation, and post-translational modification. In some embodiments, the term also encompasses secretion of the polypeptide from a cell.

As used herein, the terms “control sequences” and “regulatory sequences” refer to nucleic acid sequences necessary and/or useful for expression of a polynucleotide encoding a polypeptide. In some embodiments, control sequences are native (i.e., from the same gene) or foreign (i.e., from a different gene) to the polynucleotide encoding the polypeptide. Control sequences include, but are not limited to leaders, polyadenylation sequences, propeptide sequences, promoters, signal peptide sequences, and transcription terminators. In some embodiments, at a minimum, control sequences include a promoter, and transcriptional and translational stop signals. In some embodiments, control sequences are provided with linkers for the purpose of introducing specific restriction sites facilitating ligation of the control sequences with the coding region of the polynucleotide encoding the polypeptide.

As used herein, the term “operably linked” refers to a configuration in which a control sequence is appropriately placed at a position relative to the coding sequence of the DNA sequence such that the control sequence influences the expression of a polypeptide.

As used herein, an amino acid or nucleotide sequence (e.g., a promoter sequence, signal peptide, terminator sequence, etc.) is “heterologous” to another sequence with which it is operably linked if the two sequences are not associated in nature.

As used herein, the terms “host cell” and “host strain” refer to suitable hosts for expression vectors comprising DNA provided herein. In some embodiments, the host cells are prokaryotic or eukaryotic cells that have been transformed or transfected with vectors constructed using recombinant DNA techniques as known in the art. Transformed hosts are capable of either replicating vectors encoding at least one protein of interest and/or expressing the desired protein of interest. In addition, reference to a cell of a particular strain refers to a parental cell of the strain as well as progeny and genetically modified derivatives. Genetically modified derivatives of a parental cell include progeny cells that contain a modified genome or episomal plasmids that confer for example, antibiotic resistance, improved fermentation, etc. In some embodiments, host cells are genetically modified to have characteristics that improve protein secretion, protein stability or other properties desirable for expression and/or secretion of a protein. For example, knockout of Alp1 function results in a cell that is protease deficient. Knockout of pyr5 function results in a cell with a pyrimidine deficient phenotype. In some embodiments, host cells are modified to delete endogenous cellulase protein-encoding sequences or otherwise eliminate expression of one or more endogenous cellulases. In some embodiments, expression of one or more endogenous cellulases is inhibited to increase production of cellulases of interest. Genetic modification can be achieved by any suitable genetic engineering techniques and/or classical microbiological techniques (e.g., chemical or UV mutagenesis and subsequent selection). Using recombinant technology, nucleic acid molecules can be introduced, deleted, inhibited or modified, in a manner that results in increased yields of EG1b within the organism or in the culture. For example, knockout of Alp1 function results in a cell that is protease deficient. Knockout of pyr5 function results in a cell with a pyrimidine deficient phenotype. In some genetic engineering approaches, homologous recombination is used to induce targeted gene modifications by specifically targeting a gene in vivo to suppress expression of the encoded protein. In an alternative approach, siRNA, antisense, and/or ribozyme technology finds use in inhibiting gene expression.

As used herein, the term “introduced” used in the context of inserting a nucleic acid sequence into a cell, means transformation, transduction, conjugation, transfection, and/or any other suitable method(s) known in the art for inserting nucleic acid sequences into host cells. Any suitable means for the introduction of nucleic acid into host cells find use in the present invention.

As used herein, the terms “transformed” and “transformation” used in reference to a cell refer to a cell that has a non-native nucleic acid sequence integrated into its genome or has an episomal plasmid that is maintained through multiple generations.

As used herein, the term “C1” refers to Myceliophthora thermophilia, including the fungal strain described by Garg (See, Garg, Mycopathol., 30: 3-4 [1966]). As used herein, “Chrysosporium lucknowense” includes the strains described in U.S. Pat. Nos. 6,015,707, 5,811,381 and 6,573,086; US Pat. Pub. Nos. 2007/0238155, US 2008/0194005, US 2009/0099079; International Pat. Pub. Nos., WO 2008/073914 and WO 98/15633, all of which are incorporated herein by reference, and include, without limitation, Chrysosporium lucknowense Garg 27K, VKM-F 3500 D (Accession No. VKM F-3500-D), C1 strain UV13-6 (Accession No. VKM F-3632 D), C1 strain NG7C-19 (Accession No. VKM F-3633 D), and C1 strain UV18-25 (VKM F-3631 D), all of which have been deposited at the All-Russian Collection of Microorganisms of Russian Academy of Sciences (VKM), Bakhurhina St. 8, Moscow, Russia, 113184, and any derivatives thereof. Although initially described as Chrysosporium lucknowense, C1 may currently be considered a strain of Myceliophthora thermophile. Other C1 strains include cells deposited under accession numbers ATCC 44006, CBS (Centraalbureau voor Schimmelcultures) 122188, CBS 251.72, CBS 143.77, CBS 272.77, CBS122190, CBS122189, and VKM F-3500D. Exemplary C1 derivatives include modified organisms in which one or more endogenous genes or sequences have been deleted or modified and/or one or more heterologous genes or sequences have been introduced. Derivatives include, but are not limited to UV18#100f Δalp1, UV18#100f Δpyr5 Δalp1, UV18#100.f Δalp1 Δpep4 Δalp2, UV18#100.f Δpyr5 Δalp1 Δpep4 Δalp2 and UV18#100.f Δpyr4 Δpyr5 Δalp1 Δpep4 Δalp2, as described in WO2008073914 and WO2010107303, each of which is incorporated herein by reference.

As used herein, the terms “improved thermoactivity” and “increased thermoactivity” refer to an enzyme (e.g., a “test” enzyme of interest) displaying an increase, relative to a reference enzyme, in the amount of enzymatic activity (e.g., substrate hydrolysis) in a specified time under specified reaction conditions, for example, elevated temperature.

As used herein, the terms “improved thermostability” and “increased thermostability” refer to an enzyme (e.g., a “test” enzyme of interest) displaying an increase in “residual activity” relative to a reference enzyme. Residual activity is determined by (1) exposing the test enzyme or reference enzyme to stress conditions of elevated temperature, optionally at lowered pH, for a period of time and then determining EG1b activity; (2) exposing the test enzyme or reference enzyme to unstressed conditions for the same period of time and then determining EG1b activity; and (3) calculating residual activity as the ratio of activity obtained under stress conditions (1) over the activity obtained under unstressed conditions (2). For example, the EG1b activity of the enzyme exposed to stress conditions (“a”) is compared to that of a control in which the enzyme is not exposed to the stress conditions (“b”), and residual activity is equal to the ratio a/b. A test enzyme with increased thermostability will have greater residual activity than the reference enzyme. In some embodiments, the enzymes are exposed to stress conditions of 55° C. at pH 5.0 for 1 hr, but other cultivation conditions can be used.

As used herein, the term “culturing” refers to growing a population of microbial cells under suitable conditions in a liquid, semi-solid, gel, or solid medium.

As used herein, the term “saccharification” refers to the process in which substrates (e.g., cellulosic biomass) are broken down via the action of cellulases to produce fermentable sugars (e.g. monosaccharides such as but not limited to glucose).

As used herein, the term “fermentable sugars” refers to simple sugars (e.g., monosaccharides, disaccharides and short oligosaccharides), including but not limited to glucose, xylose, galactose, arabinose, mannose and sucrose. Indeed, a fermentable sugar is any sugar that a microorganism can utilize or ferment.

As used herein the term “soluble sugars” refers to water-soluble hexose monomers and oligomers of up to about six monomer units.

As used herein, the term “fermentation” is used broadly to refer to the cultivation of a microorganism or a culture of microorganisms that use simple sugars, such as fermentable sugars, as an energy source to obtain a desired product.

The terms “biomass,” and “biomass substrate,” encompass any suitable materials for use in saccharification reactions. The terms encompass, but are not limited to materials that comprise cellulose (i.e., “cellulosic biomass,” “cellulosic feedstock,” and “cellulosic substrate”). Biomass can be derived from plants, animals, or microorganisms, and may include, but is not limited to agricultural, industrial, and forestry residues, industrial and municipal wastes, and terrestrial and aquatic crops grown for energy purposes. Examples of biomass substrates include, but are not limited to, wood, wood pulp, paper pulp, corn fiber, corn grain, corn cobs, crop residues such as corn husks, corn stover, grasses, wheat, wheat straw, barley, barley straw, hay, rice, rice straw, switchgrass, waste paper, paper and pulp processing waste, woody or herbaceous plants, fruit or vegetable pulp, distillers grain, grasses, rice hulls, cotton, hemp, flax, sisal, sugar cane bagasse, sorghum, soy, switchgrass, components obtained from milling of grains, trees, branches, roots, leaves, wood chips, sawdust, shrubs and bushes, vegetables, fruits, and flowers and any suitable mixtures thereof. In some embodiments, the biomass comprises, but is not limited to cultivated crops (e.g., grasses, including C4 grasses, such as switch grass, cord grass, rye grass, miscanthus, reed canary grass, or any combination thereof), sugar processing residues, for example, but not limited to, bagasse (e.g., sugar cane bagasse, beet pulp [e.g., sugar beet], or a combination thereof), agricultural residues (e.g., soybean stover, corn stover, corn fiber, rice straw, sugar cane straw, rice, rice hulls, barley straw, corn cobs, wheat straw, canola straw, oat straw, oat hulls, corn fiber, hemp, flax, sisal, cotton, or any combination thereof), fruit pulp, vegetable pulp, distillers' grains, forestry biomass (e.g., wood, wood pulp, paper pulp, recycled wood pulp fiber, sawdust, hardwood, such as aspen wood, softwood, or a combination thereof). Furthermore, in some embodiments, the biomass comprises cellulosic waste material and/or forestry waste materials, including but not limited to, paper and pulp processing waste, municipal paper waste, newsprint, cardboard and the like. In some embodiments, biomass comprises one species of fiber, while in some alternative embodiments, the biomass comprises a mixture of fibers that originate from different biomasses. In some embodiments, the biomass may also comprise transgenic plants that express ligninase and/or cellulase enzymes (See e.g., US 2008/0104724 A1).

A biomass substrate is said to be “pretreated” when it has been processed by some physical and/or chemical means to facilitate saccharification. As described further herein, in some embodiments, the biomass substrate is “pretreated,” or treated using methods known in the art, such as chemical pretreatment (e.g., ammonia pretreatment, dilute acid pretreatment, dilute alkali pretreatment, or solvent exposure), physical pretreatment (e.g., steam explosion or irradiation), mechanical pretreatment (e.g., grinding or milling) and biological pretreatment (e.g., application of lignin-solubilizing microorganisms) and combinations thereof, to increase the susceptibility of cellulose to hydrolysis. Thus, the term “biomass” encompasses any living or dead biological material that contains a polysaccharide substrate, including but not limited to cellulose, starch, other forms of long-chain carbohydrate polymers, and mixtures of such sources. It may or may not be assembled entirely or primarily from glucose or xylose, and may optionally also contain various other pentose or hexose monomers. Xylose is an aldopentose containing five carbon atoms and an aldehyde group. It is the precursor to hemicellulose, and is often a main constituent of biomass. In some embodiments, the substrate is slurried prior to pretreatment. In some embodiments, the consistency of the slurry is between about 2% and about 30% and more typically between about 4% and about 15%. In some embodiments, the slurry is subjected to a water and/or acid soaking operation prior to pretreatment. In some embodiments, the slurry is dewatered using any suitable method to reduce steam and chemical usage prior to pretreatment. Examples of dewatering devices include, but are not limited to pressurized screw presses (See e.g., WO 2010/022511, incorporated herein by reference) pressurized filters and extruders.

In some embodiments, the pretreatment is carried out to hydrolyze hemicellulose, and/or a portion thereof present in the cellulosic substrate to monomeric pentose and hexose sugars (e.g., xylose, arabinose, mannose, galactose, and/or any combination thereof). In some embodiments, the pretreatment is carried out so that nearly complete hydrolysis of the hemicellulose and a small amount of conversion of cellulose to glucose occurs. In some embodiments, an acid concentration in the aqueous slurry from about 0.02% (w/w) to about 2% (w/w), or any amount therebetween, is typically used for the treatment of the cellulosic substrate. Any suitable acid finds use in these methods, including but not limited to, hydrochloric acid, nitric acid, and/or sulfuric acid. In some embodiments, the acid used during pretreatment is sulfuric acid. Steam explosion is one method of performing acid pretreatment of biomass substrates (See e.g., U.S. Pat. No. 4,461,648). Another method of pretreating the slurry involves continuous pretreatment (i.e., the cellulosic biomass is pumped though a reactor continuously). This methods are well-known to those skilled in the art (See e.g., U.S. Pat. No. 7,754,457).

In some embodiments, alkali is used in the pretreatment. In contrast to acid pretreatment, pretreatment with alkali may not hydrolyze the hemicellulose component of the biomass. Rather, the alkali reacts with acidic groups present on the hemicellulose to open up the surface of the substrate. In some embodiments, the addition of alkali alters the crystal structure of the cellulose so that it is more amenable to hydrolysis. Examples of alkali that find use in the pretreatment include, but are not limited to ammonia, ammonium hydroxide, potassium hydroxide, and sodium hydroxide. One method of alkali pretreatment is Ammonia Freeze Explosion, Ammonia Fiber Explosion or Ammonia Fiber Expansion (“AFEX” process; See e.g., U.S. Pat. Nos. 5,171,592; 5,037,663; 4,600,590; 6,106,888; 4,356,196; 5,939,544; 6,176,176; 5,037,663 and 5,171,592). During this process, the cellulosic substrate is contacted with ammonia or ammonium hydroxide in a pressure vessel for a sufficient time to enable the ammonia or ammonium hydroxide to alter the crystal structure of the cellulose fibers. The pressure is then rapidly reduced, which allows the ammonia to flash or boil and explode the cellulose fiber structure. In some embodiments, the flashed ammonia is then recovered using methods known in the art. In some alternative methods, dilute ammonia pretreatment is utilized. The dilute ammonia pretreatment method utilizes more dilute solutions of ammonia or ammonium hydroxide than AFEX (See e.g., WO2009/045651 and US 2007/0031953). This pretreatment process may or may not produce any monosaccharides.

An additional pretreatment process for use in the present invention includes chemical treatment of the cellulosic substrate with organic solvents, in methods such as those utilizing organic liquids in pretreatment systems (See e.g., U.S. Pat. No. 4,556,430; incorporated herein by reference). These methods have the advantage that the low boiling point liquids easily can be recovered and reused. Other pretreatments, such as the Organosolv™ process, also use organic liquids (See e.g., U.S. Pat. No. 7,465,791, which is also incorporated herein by reference). Subjecting the substrate to pressurized water may also be a suitable pretreatment method (See e.g., Weil et al. (1997) Appl. Biochem. Biotechnol., 68(1-2): 21-40 [1997], which is incorporated herein by reference). In some embodiments, the pretreated cellulosic biomass is processed after pretreatment by any of several steps, such as dilution with water, washing with water, buffering, filtration, or centrifugation, or any combination of these processes, prior to enzymatic hydrolysis, as is familiar to those skilled in the art. The pretreatment produces a pretreated feedstock composition (e.g., a “pretreated feedstock slurry”) that contains a soluble component including the sugars resulting from hydrolysis of the hemicellulose, optionally acetic acid and other inhibitors, and solids including unhydrolyzed feedstock and lignin. In some embodiments, the soluble components of the pretreated feedstock composition are separated from the solids to produce a soluble fraction. In some embodiments, the soluble fraction, including the sugars released during pretreatment and other soluble components (e.g., inhibitors), is then sent to fermentation. However, in some embodiments in which the hemicellulose is not effectively hydrolyzed during the pretreatment one or more additional steps are included (e.g., a further hydrolysis step(s) and/or enzymatic treatment step(s) and/or further alkali and/or acid treatment) to produce fermentable sugars. In some embodiments, the separation is carried out by washing the pretreated feedstock composition with an aqueous solution to produce a wash stream and a solids stream comprising the unhydrolyzed, pretreated feedstock. Alternatively, the soluble component is separated from the solids by subjecting the pretreated feedstock composition to a solids-liquid separation, using any suitable method (e.g., centrifugation, microfiltration, plate and frame filtration, cross-flow filtration, pressure filtration, vacuum filtration, etc.). Optionally, in some embodiments, a washing step is incorporated into the solids-liquids separation. In some embodiments, the separated solids containing cellulose, then undergo enzymatic hydrolysis with cellulase enzymes in order to convert the cellulose to glucose. In some embodiments, the pretreated feedstock composition is fed into the fermentation process without separation of the solids contained therein. In some embodiments, the unhydrolyzed solids are subjected to enzymatic hydrolysis with cellulase enzymes to convert the cellulose to glucose after the fermentation process. In some embodiments, the pretreated cellulosic feedstock is subjected to enzymatic hydrolysis with cellulase enzymes.

As used herein, the term “lignocellulosic biomass” refers to any plant biomass comprising cellulose and hemicellulose, bound to lignin. In some embodiments, the biomass may optionally be pretreated to increase the susceptibility of cellulose to hydrolysis by chemical, physical and biological pretreatments (such as steam explosion, pulping, grinding, acid hydrolysis, solvent exposure, and the like, as well as combinations thereof). Various lignocellulosic feedstocks find use, including those that comprise fresh lignocellulosic feedstock, partially dried lignocellulosic feedstock, fully dried lignocellulosic feedstock, and/or any combination thereof. In some embodiments, lignocellulosic feedstocks comprise cellulose in an amount greater than about 20%, more preferably greater than about 30%, more preferably greater than about 40% (w/w). For example, in some embodiments, the lignocellulosic material comprises from about 20% to about 90% (w/w) cellulose, or any amount therebetween, although in some embodiments, the lignocellulosic material comprises less than about 19%, less than about 18%, less than about 17%, less than about 16%, less than about 15%, less than about 14%, less than about 13%, less than about 12%, less than about 11%, less than about 10%, less than about 9%, less than about 8%, less than about 7%, less than about 6%, or less than about 5% cellulose (w/w). Furthermore, in some embodiments, the lignocellulosic feedstock comprises lignin in an amount greater than about 10%, more typically in an amount greater than about 15% (w/w). In some embodiments, the lignocellulosic feedstock comprises small amounts of sucrose, fructose and/or starch. The lignocellulosic feedstock is generally first subjected to size reduction by methods including, but not limited to, milling, grinding, agitation, shredding, compression/expansion, or other types of mechanical action. Size reduction by mechanical action can be performed by any type of equipment adapted for the purpose, for example, but not limited to, hammer mills, tub-grinders, roll presses, refiners and hydrapulpers. In some embodiments, at least 90% by weight of the particles produced from the size reduction have lengths less than between about 1/16 and about 4 in (the measurement may be a volume or a weight average length). In some embodiments, the equipment used to reduce the particle size reduction is a hammer mill or shredder. Subsequent to size reduction, the feedstock is typically slurried in water, as this facilitates pumping of the feedstock. In some embodiments, lignocellulosic feedstocks of particle size less than about 6 inches do not require size reduction.

As used herein, the term “lignocellulosic feedstock” refers to any type of lignocellulosic biomass that is suitable for use as feedstock in saccharification reactions.

As used herein, the term “pretreated lignocellulosic feedstock,” refers to lignocellulosic feedstocks that have been subjected to physical and/or chemical processes to make the fiber more accessible and/or receptive to the actions of cellulolytic enzymes, as described above.

As used herein, the term “recovered” refers to the harvesting, isolating, collecting, or recovering of protein from a cell and/or culture medium. In the context of saccharification, it is used in reference to the harvesting of fermentable sugars produced during the saccharification reaction from the culture medium and/or cells. In the context of fermentation, it is used in reference to harvesting the fermentation product from the culture medium and/or cells. Thus, a process can be said to comprise “recovering” a product of a reaction (such as a soluble sugar recovered from saccharification) if the process includes separating the product from other components of a reaction mixture subsequent to at least some of the product being generated in the reaction.

As used herein, the term “slurry” refers to an aqueous solution in which are dispersed one or more solid components, such as a cellulosic substrate.

As used herein, “increasing” the yield of a product (such as a fermentable sugar) from a reaction occurs when a particular component of interest is present during the reaction (e.g., EG1b) causes more product to be produced, compared with a reaction conducted under the same conditions with the same substrate and other substituents, but in the absence of the component of interest (e.g., without EG1b).

As used herein, a reaction is said to be “substantially free” of a particular enzyme if the amount of that enzyme compared with other enzymes that participate in catalyzing the reaction is less than about 2%, about 1%, or about 0.1% (wt/wt).

As used herein, “fractionating” a liquid (e.g., a culture broth) means applying a separation process (e.g., salt precipitation, column chromatography, size exclusion, and filtration) or a combination of such processes to provide a solution in which a desired protein (such as an EG1b protein, a cellulase enzyme, and/or a combination thereof) comprises a greater percentage of total protein in the solution than in the initial liquid product.

As used herein, the term “enzymatic hydrolysis”, refers to a process comprising at least one cellulase and at least one glycosidase enzyme and/or a mixture glycosidases that act on polysaccharides, (e.g., cellulose), to convert all or a portion thereof to fermentable sugars. “Hydrolyzing” cellulose or other polysaccharide occurs when at least some of the glycosidic bonds between two monosaccharides present in the substrate are hydrolyzed, thereby detaching from each other the two monomers that were previously bonded.

It is intended that the enzymatic hydrolysis be carried out with any suitable type of cellulase enzymes capable of hydrolyzing the cellulose to glucose, regardless of their source, including those obtained from fungi, such as Trichoderma spp., Aspergillus spp., Hypocrea spp., Humicola spp., Neurospora spp., Orpinomyces spp., Gibberella spp., Emericella spp., Chaetomium spp., Chrysosporium spp., Fusarium spp., Penicillium spp., Magnaporthe spp., Phanerochaete spp., Trametes spp., Lentinula edodes, Gleophyllum trabeiu, Ophiostoma piliferum, Corpinus cinereus, Geomyces pannorum, Cryptococcus laurentii, Aureobasidium pullulans, Amorphotheca resinae, Leucosporidium scotti, Cunninghamella elegans, Thermomyces lanuginosus, Myceliopthora thermophila, and Sporotrichum thermophile, as well as those obtained from bacteria of the genera Bacillus, Thermomyces, Clostridium, Streptomyces and Thermobifida. Cellulase compositions typically comprise one or more cellobiohydrolase, endoglucanase, and beta-glucosidase enzymes. In some cases, the cellulase compositions additionally contain hemicellulases, esterases, swollenins, cips, etc. Many of these enzymes are readily commercially available.

In some embodiments, the enzymatic hydrolysis is carried out at a pH and temperature that is at or near the optimum for the cellulase enzymes being used. For example, the enzymatic hydrolysis may be carried out at about 30° C. to about 75° C., or any suitable temperature therebetween, for example a temperature of about 30° C., about 35° C., about 40° C., about 45° C., about 50° C., about 55° C., about 60° C., about 65° C., about 70° C., about 75° C., or any temperature therebetween, and a pH of about 3.5 to about 7.5, or any pH therebetween (e.g., about 3.5, about 4.0, about 4.5, about 5.0, about 5.5, about 6.0, about 6.5, about 7.0, about 7.5, or any suitable pH therebetween). In some embodiments, the initial concentration of cellulose, prior to the start of enzymatic hydrolysis, is preferably about 0.1% (w/w) to about 20% (w/w), or any suitable amount therebetween (e.g., about 0.1%, about 0.5%, about 1%, about 2%, about 4%, about 6%, about 8%, about 10%, about 12%, about 14%, about 15%, about 18%, about 20%, or any suitable amount therebetween.) In some embodiments, the combined dosage of all cellulase enzymes is about 0.001 to about 100 mg protein per gram cellulose, or any suitable amount therebetween (e.g., about 0.001, about 0.01, about 0.1, about 1, about 5, about 10, about 15, about 20, about 25, about 30, about 40, about 50, about 60, about 70, about 80, about 90, about 100 mg protein per gram cellulose or any amount therebetween. The enzymatic hydrolysis is carried out for any suitable time period. In some embodiments, the enzymatic hydrolysis is carried out for a time period of about 0.5 hours to about 200 hours, or any time therebetween (e.g., about 2 hours to about 100 hours, or any suitable time therebetween). For example, in some embodiments, it is carried out for about 0.5, about 1, about 2, about 5, about 7, about 10, about 12, about 14, about 15, about 20, about 25, about 30, about 35, about 40, about 45, about 50, about 55, about 60, about 65, about 70, about 75, about 80, about 85, about 90, about 95, about 100, about 120, about 140, about 160, about 180, about 200, or any suitable time therebetween.)

In some embodiments, the enzymatic hydrolysis is batch hydrolysis, continuous hydrolysis, and/or a combination thereof. In some embodiments, the hydrolysis is agitated, unmixed, or a combination thereof. The enzymatic hydrolysis is typically carried out in a hydrolysis reactor. The cellulase enzyme composition is added to the pretreated lignocellulosic substrate prior to, during, or after the addition of the substrate to the hydrolysis reactor. Indeed it is not intended that reaction conditions be limited to those provided herein, as modifications are well-within the knowledge of those skilled in the art. In some embodiments, following cellulose hydrolysis, any insoluble solids present in the resulting lignocellulosic hydrolysate, including but not limited to lignin, are removed using conventional solid-liquid separation techniques prior to any further processing. In some embodiments, these solids are burned to provide energy for the entire process.

As used herein, the term “by-product” refers to an organic molecule that is an undesired product of a particular process (e.g., saccharification).

As used herein, the terms “adjunct material,” “adjunct composition,” and “adjunct compound” refer to any composition suitable for use in the compositions and/or saccharification reactions provided herein, including but not limited to cofactors, surfactants, builders, buffers, enzyme stabilizing systems, chelants, dispersants, colorants, preservatives, antioxidants, solublizing agents, carriers, processing aids, pH control agents, etc. In some embodiments, divalent metal cations are used to supplement saccharification reactions and/or the growth of host cells. Any suitable divalent metal cation finds use in the present invention, including but not limited to Cu⁺⁺, Mn⁺⁺, Co⁺⁺, Mg⁺⁺, Ni⁺⁺, Zn⁺⁺, and Ca⁺⁺. In addition, any suitable combination of divalent metal cations finds use in the present invention. Furthermore, divalent metal cations find use from any suitable source.

### DETAILED DESCRIPTION OF THE INVENTION

The present invention provides endoglucanase 1b (EG1b) suitable for use in saccharification reactions. In some embodiments, the present invention provides methods and compositions suitable for use in the degradation of cellulose. In some additional embodiments, the present invention provides EG1b enzymes suitable for use in saccharification reactions to hydrolyze cellulose components in biomass feedstock. In some additional embodiments, the EG1b enzymes are used in combination with additional enzymes, including but not limited to at least one EG (e.g., EG1a, EG2, EG3, EG4, EG5, and/or EG6), cellobiohydrolase, GH61, and/or beta-glucosidases, etc., in saccharification reactions.

Fungi, bacteria, and other organisms produce a variety of cellulases and other enzymes that act in concert to catalyze decrystallization and hydrolysis of cellulose to yield fermentable sugars. One such fungus is M. thermophila, which is described hereinabove. One M. thermophila cellulase of interest is the EG1b enzyme. The EG1b sequences provided herein are particularly useful for the production of fermentable sugars from cellulosic biomass. In another aspect, the present invention relates to methods of generating fermentable sugars from cellulosic biomass, by contacting the biomass with a cellulase composition comprising EG1b as described herein, under conditions suitable for the production of fermentable sugars.

In some embodiments, the polynucleotide that hybridizes to the complement of a polynucleotide which encodes a polypeptide having the amino acid sequence of SEQ ID NO:2, under high or very high stringency conditions to the complement of a reference sequence having the sequence of SEQ ID NO:2 (e.g., over substantially the entire length of the reference sequence).

EG1b activity and thermostability can be determined by any suitable method known in the art. For example, EG1b activity may be determined using an assay that measures the conversion of crystalline cellulose to glucose. For example, EG1b activity can be determined using a cellulose assay, in which the ability of the EG1b to hydrolyze a cellulose substrate to cellobiose (e.g., crystalline cellulose under specific temperature and/or pH conditions is measured, then a beta-glucosidase is added to convert the cellobiose to glucose). In some embodiments, conversion of cellulose substrate (e.g., crystalline cellulose) to fermentable sugar monomers (e.g., glucose) is determined by art-known means, including but not limited to coupled enzymatic assays and colorimetric assays. For example, glucose concentrations can be determined using a coupled enzymatic assay based on glucose oxidase and horseradish peroxidase (e.g., GOPOD assay; See e.g., Trinder, Ann. Clin. Biochem., 6:24-27 [1969], which is incorporated herein by reference in its entirety). GOPOD assay kits are known in the art and are readily commercially available (e.g., from Megazyme (Wicklow, Ireland). In addition, methods for performing GOPOD assays are well-known in the art (See e.g., McCleary et al., J. AOAC Int'l., 85(5):1103-11 [2002], the contents of which are incorporated by reference herein). Additional methods of cellobiose quantification include, but are not limited chromatographic methods (e.g., HPLC; See e.g., U.S. Pat. Nos. 6,090,595 and 7,419,809, both of which are incorporated by reference in their entireties).

In some additional embodiments, EG1b thermostability is determined by exposing the EG1b to stress conditions of elevated temperature and/or low pH for a desired period of time and then determining residual EG1b activity using an assay that measures the conversion of cellulose to glucose, as described herein.

In some embodiments, the EG1b of the present invention further comprises additional sequences which do not alter the encoded activity of the enzyme. For example, in some embodiments, the EG1b is linked to an epitope tag or to another sequence useful in purification.

In some embodiments, the EG1b polypeptides of the present invention are secreted from the host cell in which they are expressed (e.g., a yeast or filamentous fungal host cell) and are expressed as a pre-protein including a signal peptide (i.e., an amino acid sequence linked to the amino terminus of a polypeptide and which directs the encoded polypeptide into the cell secretory pathway). In some embodiments, the signal peptide is an endogenous M. thermophila EG1b signal peptide. In some other embodiments, signal peptides from other M. thermophila secreted proteins are used. In some embodiments, other signal peptides find use, depending on the host cell and other factors. Effective signal peptide coding regions for filamentous fungal host cells include, but are not limited to, the signal peptide coding regions obtained from Aspergillus oryzae TAKA amylase, Aspergillus niger neutral amylase, A. niger glucoamylase, Rhizomucor miehei asparatic proteinase, Humicola insolens cellulase, Humicola lanuginosa lipase, and T. reesei cellobiohydrolase II. Signal peptide coding regions for bacterial host cells include, but are not limited to the signal peptide coding regions obtained from the genes for Bacillus NC1B 11837 maltogenic amylase, Bacillus stearothermophilus alpha-amylase, Bacillus licheniformis subtilisin, Bacillus licheniformis beta-lactamase, Bacillus stearothermophilus neutral proteases (nprT, nprS, nprM), and Bacillus subtilis prsA. In some additional embodiments, other signal peptides find use in the present invention (See e.g., Simonen and Palva, Microbiol Rev., 57: 109-137 [1993], incorporated herein by reference). Additional useful signal peptides for yeast host cells include those from the genes for Saccharomyces cerevisiae alpha-factor, S. cerevisiae SUC2 invertase (See e.g., Taussig and Carlson, Nucleic Acids Res., 11:1943-54 [1983]; SwissProt Accession No. P00724; and Romanos et al., Yeast 8:423-488 [1992]). In some embodiments, variants of these signal peptides and other signal peptides find use.

In some embodiments, the present invention provides polynucleotides encoding EG1b polypeptide, or biologically active fragments thereof, as described herein. In some embodiments, the polynucleotide is operably linked to one or more heterologous regulatory or control sequences that control gene expression to create a recombinant polynucleotide capable of expressing the polypeptide. In some embodiments, expression constructs containing a heterologous polynucleotide encoding EG1b are introduced into appropriate host cells to express the EG1b.

Those of ordinary skill in the art understand that due to the degeneracy of the genetic code, a multitude of nucleotide sequences encoding EG1b polypeptide of the present invention exist. For example, the codons AGA, AGG, CGA, CGC, CGG, and CGU all encode the amino acid arginine. Thus, at every position in the nucleic acids of the invention where an arginine is specified by a codon, the codon can be altered to any of the corresponding codons described above without altering the encoded polypeptide. It is understood that “U” in an RNA sequence corresponds to “T” in a DNA sequence. The invention contemplates and provides each and every possible variation of nucleic acid sequence encoding a polypeptide of the invention that could be made by selecting combinations based on possible codon choices.

A DNA sequence may also be designed for high codon usage bias codons (codons that are used at higher frequency in the protein coding regions than other codons that code for the same amino acid). The preferred codons may be determined in relation to codon usage in a single gene, a set of genes of common function or origin, highly expressed genes, the codon frequency in the aggregate protein coding regions of the whole organism, codon frequency in the aggregate protein coding regions of related organisms, or combinations thereof. A codon whose frequency increases with the level of gene expression is typically an optimal codon for expression. In particular, a DNA sequence can be optimized for expression in a particular host organism. A variety of methods are well-known in the art for determining the codon frequency (e.g., codon usage, relative synonymous codon usage) and codon preference in specific organisms, including multivariate analysis (e.g., using cluster analysis or correspondence analysis,) and the effective number of codons used in a gene. The data source for obtaining codon usage may rely on any available nucleotide sequence capable of coding for a protein. These data sets include nucleic acid sequences actually known to encode expressed proteins (e.g., complete protein coding sequences-CDS), expressed sequence tags (ESTs), or predicted coding regions of genomic sequences, as is well-known in the art. Polynucleotides encoding EG1b can be prepared using any suitable methods known in the art. Typically, oligonucleotides are individually synthesized, then joined (e.g., by enzymatic or chemical ligation methods, or polymerase-mediated methods) to form essentially any desired continuous sequence. In some embodiments, polynucleotides of the present invention are prepared by chemical synthesis using, any suitable methods known in the art, including but not limited to automated synthetic methods. For example, in the phosphoramidite method, oligonucleotides are synthesized (e.g., in an automatic DNA synthesizer), purified, annealed, ligated and cloned in appropriate vectors. In some embodiments, double stranded DNA fragments are then obtained either by synthesizing the complementary strand and annealing the strands together under appropriate conditions, or by adding the complementary strand using DNA polymerase with an appropriate primer sequence. There are numerous general and standard texts that provide methods useful in the present invention are well known to those skilled in the art.

The present invention also provides recombinant constructs comprising a sequence encoding EG1b, as provided herein. In some embodiments, the present invention provides an expression vector comprising an EG1b polynucleotide operably linked to a heterologous promoter. In some embodiments, expression vectors of the present invention are used to transform appropriate host cells to permit the host cells to express the EG1b protein. Methods for recombinant expression of proteins in fungi and other organisms are well known in the art, and a number expression vectors are available or can be constructed using routine methods. In some embodiments, nucleic acid constructs of the present invention comprise a vector, such as, a plasmid, a cosmid, a phage, a virus, a bacterial artificial chromosome (BAC), a yeast artificial chromosome (YAC), and the like, into which a nucleic acid sequence of the invention has been inserted. In some embodiments, polynucleotides of the present invention are incorporated into any one of a variety of expression vectors suitable for expressing EG1b polypeptide. Suitable vectors include, but are not limited to chromosomal, nonchromosomal and synthetic DNA sequences (e.g., derivatives of SV40), as well as bacterial plasmids, phage DNA, baculovirus, yeast plasmids, vectors derived from combinations of plasmids and phage DNA, viral DNA such as vaccinia, adenovirus, fowl pox virus, pseudorabies, adenovirus, adeno-associated virus, retroviruses, and many others. Any suitable vector that transduces genetic material into a cell, and, if replication is desired, which is replicable and viable in the relevant host finds use in the present invention. In some embodiments, the construct further comprises regulatory sequences, including but not limited to a promoter, operably linked to the protein encoding sequence. Large numbers of suitable vectors and promoters are known to those of skill in the art. Indeed, in some embodiments, in order to obtain high levels of expression in a particular host it is often useful to express the EG1b of the present invention under the control of a heterologous promoter. In some embodiments, a promoter sequence is operably linked to the 5′ region of the EG 1 b coding sequence using any suitable method known in the art. Examples of useful promoters for expression of EG1b include, but are not limited to promoters from fungi. In some embodiments, a promoter sequence that drives expression of a gene other than EG1b gene in a fungal strain finds use. As a non-limiting example, a fungal promoter from a gene encoding an endoglucanase may be used. In some embodiments, a promoter sequence that drives the expression of a EG1b gene in a fungal strain other than the fungal strain from which the EG1b was derived finds use. Examples of other suitable promoters useful for directing the transcription of the nucleotide constructs of the present invention in a filamentous fungal host cell are promoters obtained from the genes for A. oryzae TAKA amylase, R. miehei aspartic proteinase, A. niger neutral alpha-amylase, A. niger acid stable alpha-amylase, A. niger or A. awamori glucoamylase (glaA), R. miehei lipase, A. oryzae alkaline protease, A. oryzae triose phosphate isomerase, A. nidulans acetamidase, and F. oxysporum trypsin-like protease (See e.g., WO 96/00787, incorporated herein by reference), as well as the NA2-tpi promoter (a hybrid of the promoters from the genes for A. niger neutral alpha-amylase and A. oryzae triose phosphate isomerase), promoters such as cbh1, cbh2, egl1, egl2, pepA, hfb1, hfb2, xyn1, amy, and glaA (See e.g., Nunberg et al., Mol. Cell Biol., 4:2306-2315 [1984]; Boel et al., EMBO J. 3:1581-85 [1984]; and European Patent Appin. 137280, all of which are incorporated herein by reference), and mutant, truncated, and hybrid promoters thereof. In a yeast host, useful promoters include, but are not limited to those from the genes for S. cerevisiae enolase (eno-1), S. cerevisiae galactokinase (gal1), S. cerevisiae alcohol dehydrogenase/glyceraldehyde-3-phosphate dehydrogenase (ADH2/GAP), and S. cerevisiae 3-phosphoglycerate kinase. Additional useful promoters useful for yeast host cells are known in the art (See e.g., Romanos et al., Yeast 8:423-488 [1992], incorporated herein by reference). In addition, promoters associated with chitinase production in fungi find use in the present invention (See e.g., Blaiseau and Lafay, Gene 120243-248 [1992]; and Limon et al., Curr. Genet, 28:478-83 [1995], both of which are incorporated herein by reference).

In some embodiments, cloned EG1b of the present invention also have a suitable transcription terminator sequence, a sequence recognized by a host cell to terminate transcription. The terminator sequence is operably linked to the 3′ terminus of the nucleic acid sequence encoding the polypeptide. Any terminator that is functional in the host cell of choice finds use in the present invention. Exemplary transcription terminators for filamentous fungal host cells include, but are not limited to those obtained from the genes for A. oryzae TAKA amylase, A. niger glucoamylase, A. nidulans anthranilate synthase, A. niger alpha-glucosidase, and F. oxysporum trypsin-like protease (See also, U.S. Pat. No. 7,399,627, incorporated herein by reference). In some embodiments, exemplary terminators for yeast host cells include those obtained from the genes for S. cerevisiae enolase, S. cerevisiae cytochrome C (CYC1), and S. cerevisiae glyceraldehyde-3-phosphate dehydrogenase. Other useful terminators for yeast host cells are well-known to those skilled in the art (See e.g., Romanos et al., Yeast 8:423-88 [1992]).

In some embodiments, a suitable leader sequence is part of a cloned EG1b sequence, which is a nontranslated region of an mRNA that is important for translation by the host cell. The leader sequence is operably linked to the 5′ terminus of the nucleic acid sequence encoding the polypeptide. Any leader sequence that is functional in the host cell of choice finds use in the present invention. Exemplary leaders for filamentous fungal host cells include, but are not limited to those obtained from the genes for A. oryzae TAKA amylase and A. nidulans triose phosphate isomerase. Suitable leaders for yeast host cells include, but are not limited to those obtained from the genes for S. cerevisiae enolase (ENO-1), S. cerevisiae 3-phosphoglycerate kinase, S. cerevisiae alpha-factor, and S. cerevisiae alcohol dehydrogenase/glyceraldehyde-3-phosphate dehydrogenase (ADH2/GAP).

In some embodiments, the sequences of the present invention also comprise a polyadenylation sequence, which is a sequence operably linked to the 3′ terminus of the nucleic acid sequence and which, when transcribed, is recognized by the host cell as a signal to add polyadenosine residues to transcribed mRNA. Any polyadenylation sequence which is functional in the host cell of choice finds use in the present invention. Exemplary polyadenylation sequences for filamentous fungal host cells include, but are not limited to those obtained from the genes for A. oryzae TAKA amylase, A. niger glucoamylase, A. nidulans anthranilate synthase, F. oxysporum trypsin-like protease, and A. niger alpha-glucosidase. Useful polyadenylation sequences for yeast host cells are known in the art (See e.g., Guo and Sherman, Mol Cell Biol., 15:5983-5990 [1995]).

In some embodiments, the expression vector of the present invention contains one or more selectable markers, which permit easy selection of transformed cells. A “selectable marker” is a gene, the product of which provides for biocide or viral resistance, resistance to antimicrobials or heavy metals, prototrophy to auxotrophs, and the like. Any suitable selectable markers for use in a filamentous fungal host cell find use in the present invention, including, but are not limited to, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Additional markers useful in host cells such as Aspergillus, include but are not limited to the amdS and pyrG genes of A. nidulans or A. oryzae and the bar gene of Streptomyces hygroscopicus. Suitable markers for yeast host cells include, but are not limited to ADE2, HIS3, LEU2, LYS2, MET3, TRP1, and URA3.

In some embodiments, a vector comprising a sequence encoding a EG1b is transformed into a host cell in order to allow propagation of the vector and expression of the EG1b. In some embodiments, the EG1b is post-translationally modified to remove the signal peptide and in some cases may be cleaved after secretion. In some embodiments, the transformed host cell described above is cultured in a suitable nutrient medium under conditions permitting the expression of the EG1b. Any suitable medium useful for culturing the host cells finds use in the present invention, including, but not limited to minimal or complex media containing appropriate supplements. In some embodiments, host cells are grown in HTP media. Suitable media are available from various commercial suppliers or may be prepared according to published recipes (e.g. in catalogues of the American Type Culture Collection).

In some embodiments, the host cell is a eukaryotic cell. Suitable eukaryotic host cells include, but are not limited to, fungal cells, algal cells, insect cells, and plant cells. Suitable fungal host cells include, but are not limited to, Ascomycota, Basidiomycota, Deuteromycota, Zygomycota, Fungi imperfecti. In some embodiments, the fungal host cells are yeast cells and filamentous fungal cells. The filamentous fungal host cells of the present invention include all filamentous forms of the subdivision Eumycotina and Oomycota. Filamentous fungi are characterized by a vegetative mycelium with a cell wall composed of chitin, cellulose and other complex polysaccharides. The filamentous fungal host cells of the present invention are morphologically distinct from yeast.

In some embodiments of the present invention, the filamentous fungal host cells are of any suitable genus and species, including, but not limited to Achlya, Acremonium, Aspergillus, Aureobasidium, Bjerkandera, Ceriporiopsis, Cephalosporium, Chrysosporiurn, Cochliobolus, Corynascus, Cryphonectria, Cryptococcus, Coprinus, Coriolus, Diplodia, Endothis, Fusarium, Gibberella, Gliocladium, Humicola, Hypocrea, Myceliophthora, Mucor, Neurospora, Penicillium, Podospora, Phlebia, Piromyces, Pyricularia, Rhizomucor, Rhizopus, Schizophyllum, Scytalidium, Sporotrichum, Talaromyces, Thermoascus, Thielavia, Trametes, Tolypocladium, Trichoderma, Verticillium, and/or Volvariella, and/or teleomorphs, or anamorphs, and synonyms, basionyms, or taxonomic equivalents thereof.

In some embodiments of the present invention, the filamentous fungal host cell is of the Trichoderma species (e.g., T. longibrachiatum, T. viride [e.g., ATCC 32098 and 32086]), Hypocrea jecorina or T. reesei (NRRL 15709, ATTC 13631, 56764, 56765, 56466, 56767 and RL-P37 and derivatives thereof (See e.g., Sheir-Neiss et al., Appl. Microbiol. Biotechnol., 20:46-53 [1984]), T. koningii, and T. harzianum. In addition, the term “Trichoderma” refers to any fungal strain that was previously and/or currently classified as Trichoderma. In some embodiments of the present invention, the filamentous fungal host cell is of the Aspergillus species (e.g., A. awamori, A. funigatus, A. japonicus, A. nidulans, A. niger, A. aculeatus, A. foetidus, A. oryzae, A. sojae, and A. kawachi; See e.g., Kelly and Hynes, EMBO J., 4:475-479 [1985]; NRRL 3112, ATCC 11490, 22342, 44733, and 14331; Yelton et al., Proc. Natl. Acad. Sci. USA, 81, 1470-1474 [1984]; Tilburn et al., Gene 26:205-221 [1982]; and Johnston, et al., EMBO J., 4:1307-1311 [1985]). In some embodiments of the present invention, the filamentous fungal host cell is a Chrysosporium species (e.g., C. lucknowense, C. keratinophilum, C. tropicum, C. merdarium, C. inops, C. pannicola, and C. zonatum). In some embodiments of the present invention, the filamentous fungal host cell is a Myceliophthora species (e.g., M. thermophila). In some embodiments of the present invention, the filamentous fungal host cell is a Fusarium species (e.g., F. bactridioides, F. cerealis, F. crookwellense, F. culmorum, F. graminearum, F. graminum. F. oxysporum, F. roseum, and F. venenatum). In some embodiments of the present invention, the filamentous fungal host cell is a Neurospora species (e.g., N. crassa; See e.g., Case et al., Proc. Natl. Acad. Sci. USA, 76:5259-5263 [1979]; U.S. Pat. No. 4,486,553; and Kinsey and Rambosek (1984) Mol. Cell. Biol., 4:117-122 [1984], all of which are hereby incorporated by reference). In some embodiments of the present invention, the filamentous fungal host cell is a Humicola species (e.g., H. insolens, H. grisea, and H. lanuginosa). In some embodiments of the present invention, the filamentous fungal host cell is a Mucor species (e.g., M. miehei and M. circinelloides). In some embodiments of the present invention, the filamentous fungal host cell is a Rhizopus species (e.g., R. oryzae and R. niveus.). In some embodiments of the invention, the filamentous fungal host cell is a Penicillum species (e.g., P. purpurogenum, P. chrysogenum, and P. verruculosum). In some embodiments of the invention, the filamentous fungal host cell is a Talaromyces species (e.g., T. emersonii, T. flavus, T. helicus, T. rotundus, and T. stipitatus). In some embodiments of the invention, the filamentous fungal host cell is a Thielavia species (e.g., T. terrestris and T. heterothallica). In some embodiments of the present invention, the filamentous fungal host cell is a Tolypocladium species (e.g., T. inflatum and T. geodes). In some embodiments of the present invention, the filamentous fungal host cell is a Trametes species (e.g., T. villosa and T. versicolor). In some embodiments of the present invention, the filamentous fungal host cell is a Sporotrichium species. In some embodiments of the present invention, the filamentous fungal host cell is a Corynascus species.

In some embodiments of the present invention, the host cell is a yeast cell, including but not limited to cells of Candida, Hansenula, Saccharomyces, Schizosaccharomyces, Pichia, Kluyveromyces, or Yarrowia species. In some embodiments of the present invention, the yeast cell is H. polymorpha, S. cerevisiae, S. carlsbergensis, S. diastaticus, S. norbensis, S. kluyveri, S. pombe, P. pastoris, P. finlandica, P. trehalophila, P. kodamae, P. membranaefaciens, P. opuntiae, P. thermotolerans, P. salictaria, P. quercuum, P. pijperi, P. stipitis, P. methanolica, P. angusta, K. lactic, C. albicans, or Y. lipoiytica.

In some embodiments of the invention, the host cell is an algal cell such as Chlamydomonas (e.g., C. reinhardtii) and Phormidium (P. sp. ATCC29409).

In some other embodiments, the host cell is a prokaryotic cell. Suitable prokaryotic cells include, but are not limited to Gram-positive, Gram-negative and Gram-variable bacterial cells. Any suitable bacterial organism finds use in the present invention, including but not limited to Agrobacterium, Alicyclobacillus, Anabaena, Anacystis, Acinetobacter, Acidothermus, Arthrobacter, Azobacter, Bacillus, Bifidobacterium, Brevibacterium, Butyrivibrio, Buchnera, Campestris, Camplyobacter, Clostridium, Corynebacterium, Chromatium, Coprococcus, Escherichia, Enterococcus, Enterobacter, Erwinia, Fusobacterium, Faecalibacterium, Francisella, Flavobacterium, Geobacillus, Haemophilus, Helicobacter, Klebsiella, Lactobacillus, Lactococcus, Ilyobacter, Micrococcus, Microbacterium, Mesorhizobium, Methylobacterium, Methylobacterium, Mycobacterium, Neisseria, Pantoea, Pseudomonas, Prochlorococcus, Rhodobacter, Rhodopseudomonas, Rhodopseudomonas, Roseburia, Rhodospirillum, Rhodococcus, Scenedesmus, Streptomyces, Streptococcus, Synecoccus, Saccharomonospora, Staphylococcus, Serratia, Salmonella, Shigella, Thermoanaerobacterium, Tropheryma, Tularensis, Temecula, Thermosynechococcus, Thermococcus, Ureaplasma, Xanthomonas, Xylella, Yersinia and Zymomonas. In some embodiments, the host cell is a species of Agrobacterium, Acinetobacter, Azobacter, Bacillus, Bifidobacterium, Buchnera, Geobacillus, Campylobacter, Clostridium, Corynebacterium, Escherichia, Enterococcus, Erwinia, Flavobacterium, Lactobacillus, Lactococcus, Pantoea, Pseudomonas, Staphylococcus, Salmonella, Streptococcus, Streptomyces, or Zymomonas. In some embodiments, the bacterial host strain is non-pathogenic to humans. In some embodiments the bacterial host strain is an industrial strain. Numerous bacterial industrial strains are known and suitable in the present invention. In some embodiments of the present invention, the bacterial host cell is a Agrobacterium species (e.g., A. radiobacter, A. rhizogenes, and A. rubi). In some embodiments of the present invention, the bacterial host cell is a Arthrobacter species (e.g., A. aurescens, A. citreus, A. globformis, A. hydrocarboglutamicus, A. mysorens, A. nicotianae, A. paraffineus, A. protophonniae, A. roseoparqffinus, A. sulfureus, and A. ureafaciens). In some embodiments of the present invention, the bacterial host cell is a Bacillus species (e.g., B. thuringensis, B. anthracis, B. megaterium, B. subtilis, B. lentos, B. circulans, B. pumilus, B. lautus, B. coagulans, B. brevis, B. firmus, B. aikaophius, B. licheniformis, B. clausii, B. stearothermophilus, B. halodurans, and B. amyloliquefaciens). In some embodiments, the host cell is an industrial Bacillus strain including but not limited to B. subtilis, B. pumilus, B. licheniformis, B. megaterium, B. clausii, B. stearothermophilus, or B. amyloliquefaciens. In some embodiments, the Bacillus host cells are B. subtilis, B. licheniformis, B. megaterium, B. stearothermophilus, and/or B. amyloliquefaciens. In some embodiments, the bacterial host cell is a Clostridium species (e.g., C. acetobutylicum, C. tetani E88, C. lituseburense, C. saccharobutylicum, C. perfringens, and C. beijerinckii). In some embodiments, the bacterial host cell is a Corynebacterium species (e.g., C. glutamicum and C. acetoacidophilum). In some embodiments the bacterial host cell is an Escherichia species (e.g., E. coli). In some embodiments, the bacterial host cell is an Erwinia species (e.g., E. uredovora, E. carotovora, E. ananas, E. herbicola, E. punctata, and E. terreus). In some embodiments, the bacterial host cell is a Pantoea species (e.g., P. citrea, and P. agglomerans). In some embodiments the bacterial host cell is a Pseudomonas species (e.g., P. putida, P. aeruginosa, P. mevalonii, and P. sp. D-01 10). In some embodiments, the bacterial host cell is a Streptococcus species (e.g., S. equisiiniles, S. pyogenes, and S. uberis). In some embodiments, the bacterial host cell is a Streptomyces species (e.g., S. ambofaciens, S. achromogenes, S. avermitilis, S. coelicolor, S. aureofaciens, S. aureus, S. fungicidicus, S. griseus, and S. lividans). In some embodiments, the bacterial host cell is a Zymomonas species (e.g., Z. mobilis, and Z. lipolytica).

Many prokaryotic and eukaryotic strains that find use in the present invention are readily available to the public from a number of culture collections such as American Type Culture Collection (ATCC), Deutsche Sammlung von Mikroorganismen and Zellkulturen GmbH (DSM), Centraalbureau Voor Schimmelcultures (CBS), and Agricultural Research Service Patent Culture Collection, Northern Regional Research Center (NRRL).

In some embodiments, host cells are genetically modified to have characteristics that improve protein secretion, protein stability and/or other properties desirable for expression and/or secretion of a protein. For example, knockout of Alp 1 function results in a cell that is protease deficient. Knockout of pyr5 function results in a cell with a pyrimidine deficient phenotype. In some embodiments, the host cells are modified to delete endogenous cellulase protein-encoding sequences or otherwise eliminate expression of one or more endogenous cellulases. In some embodiments, expression of one or more endogenous cellulases is inhibited to increase production of cellulases of interest. Genetic modification can be achieved by genetic engineering techniques and/or classical microbiological techniques (e.g., chemical or UV mutagenesis and subsequent selection). Indeed, in some embodiments, combinations of recombinant modification and classical selection techniques are used to produce the host cells. Using recombinant technology, nucleic acid molecules can be introduced, deleted, inhibited or modified, in a manner that results in increased yields of EG1b within the host cell and/or in the culture medium. For example, knockout of Alp1 function results in a cell that is protease deficient, and knockout of pyr5 function results in a cell with a pyrimidine deficient phenotype. In one genetic engineering approach, homologous recombination is used to induce targeted gene modifications by specifically targeting a gene in vivo to suppress expression of the encoded protein. In alternative approaches, siRNA, antisense and/or ribozyme technology find use in inhibiting gene expression.

In some embodiments, host cells (e.g., Myceliophthora thermophila) used for expression of EG1b have been genetically modified to reduce the amount of endogenous cellobiose dehydrogenase (EC 1.1.3.4) and/or other enzymes activity that is secreted by the cell, including but not limited to the strains described in U.S. Pat. No. 8,236,551 and WO 2012/061382, incorporated by reference herein). A variety of methods are known in the art for reducing expression of protein in cells, including, but not limited to deletion of all or part of the gene encoding the protein and site-specific mutagenesis to disrupt expression or activity of the gene product. (See e.g., Chaveroche et al., Nucl. Acids Res., 28:22 e97 [2000]; Cho et al., MPMI 19: 1:7-15 [2006]; Maruyama and Kitamoto, Biotechnol Left, 30:1811-1817 [2008]; Takahashi et al., Mol. Gen. Genom., 272: 344-352 [2004]; and You et al., Arch Micriobiol., 191:615-622 [2009], all of which are incorporated by reference herein). Random mutagenesis, followed by screening for desired mutations also finds use (See e.g., Combier et al., FEMS Microbiol Lett 220:141-8 [2003]; and Firon et al., Eukary. Cell 2:247-55 [2003], both of which are incorporated by reference). In some embodiments, the host cell is modified to reduce production of endogenous cellobiose dehydrogenases (See e.g., U.S. Pat. No. 8,236,551 and WO 2012/061382, both of which are incorporated by reference). In some embodiments, the cell is modified to reduce production of cellobiose dehydrogenase (e.g., CDH1 or CDH2). In some embodiments, the host cell has less than 75%, sometimes less than 50%, sometimes less than 30%, sometimes less than 25%, sometimes less than 20%, sometimes less than 15%, sometimes less than 10%, sometimes less than 5%, and sometimes less than 1% of the cellobiose dehydrogenase (e.g., CDH1 and/or CDH2) activity of the corresponding cell in which the gene is not disrupted. Exemplary Myceliophthora thermophila cellobiose dehydrogenases include, but are not limited to CDH1 and CDH2. The genomic sequence for the Cdh1 encoding CDH1 has accession number AF074951.1. In one approach, gene disruption is achieved using genomic flanking markers (See e.g., Rothstein, Meth. Enzymol., 101:202-11 [1983]). In some embodiments, site-directed mutagenesis is used to target a particular domain of a protein, in some cases, to reduce enzymatic activity (e.g., glucose-methanol-choline oxido-reductase N and C domains of a cellobiose dehydrogenase or heme binding domain of a cellobiose dehydrogenase; See e.g., Rotsaert et al., Arch. Biochem. Biophys., 390:206-14 [2001], which is incorporated by reference herein in its entirety).

Introduction of a vector or DNA construct into a host cell can be accomplished using any suitable method known in the art, including but not limited to calcium phosphate transfection, DEAE-Dextran mediated transfection, PEG-mediated transformation, electroporation, or other common techniques known in the art.

In some embodiments, the engineered host cells (i.e., “recombinant host cells”) of the present invention are cultured in conventional nutrient media modified as appropriate for activating promoters, selecting transformants, or amplifying the cellobiohydrolase polynucleotide. Culture conditions, such as temperature, pH and the like, are those previously used with the host cell selected for expression, and are well-known to those skilled in the art. As noted, many standard references and texts are available for the culture and production of many cells, including cells of bacterial, plant, animal (especially mammalian) and archebacterial origin.

In some embodiments, cells expressing the EG1b polypeptide of the invention are grown under batch or continuous fermentations conditions. Classical “batch fermentation” is a closed system, wherein the compositions of the medium is set at the beginning of the fermentation and is not subject to artificial alternations during the fermentation. A variation of the batch system is a “fed-batch fermentation” which also finds use in the present invention. In this variation, the substrate is added in increments as the fermentation progresses. Fed-batch systems are useful when catabolite repression is likely to inhibit the metabolism of the cells and where it is desirable to have limited amounts of substrate in the medium. Batch and fed-batch fermentations are common and well known in the art. “Continuous fermentation” is an open system where a defined fermentation medium is added continuously to a bioreactor and an equal amount of conditioned medium is removed simultaneously for processing. Continuous fermentation generally maintains the cultures at a constant high density where cells are primarily in log phase growth. Continuous fermentation systems strive to maintain steady state growth conditions. Methods for modulating nutrients and growth factors for continuous fermentation processes as well as techniques for maximizing the rate of product formation are well known in the art of industrial microbiology.

In some embodiments of the present invention, cell-free transcription/translation systems find use in producing EB1 b. Several systems are commercially available and the methods are well-known to those skilled in the art.

The present invention provides methods of making EG1b polypeptides or biologically active fragments thereof. In some embodiments, the method comprises: providing a host cell transformed with a polynucleotide encoding an amino acid sequence that comprises at least about 70% (or at least about 75%, at least about 80%, at least about 85%, at least about 90%, at least about 95%, at least about 96%, at least about 97%, at least about 98%, or at least about 99%) sequence identity to SEQ ID NO:2; culturing the transformed host cell in a culture medium under conditions in which the host cell expresses the encoded EG1b polypeptide; and optionally recovering or isolating the expressed EG1b polypeptide, and/or recovering or isolating the culture medium containing the expressed EG1b polypeptide. In some embodiments, the methods further provide optionally lysing the transformed host cells after expressing the encoded EG1b polypeptide and optionally recovering and/or isolating the expressed EG1b polypeptide from the cell lysate. The present invention further provides a method of making an EG1b polypeptide, said method comprising cultivating a host cell transformed with an EG1b polypeptide under conditions suitable for the production of the EG1b polypeptide and recovering the EG1b polypeptide. Typically, recovery or isolation of the EG1b polypeptide is from the host cell culture medium, the host cell or both, using protein recovery techniques that are well known in the art, including those described herein. Cells are typically harvested by centrifugation, disrupted by physical or chemical means, and the resulting crude extract may be retained for further purification. Microbial cells employed in expression of proteins can be disrupted by any convenient method, including, but not limited to freeze-thaw cycling, sonication, mechanical disruption, and/or use of cell lysing agents, as well as many other methods, which are well known to those skilled in the art.

In some embodiments, the resulting polypeptide is recovered/isolated and optionally purified by any of a number of methods known in the art. For example, in some embodiments, the polypeptide is isolated from the nutrient medium by conventional procedures including, but not limited to, centrifugation, filtration, extraction, spray-drying, evaporation, chromatography (e.g., ion exchange, affinity, hydrophobic interaction, chromatofocusing, and size exclusion), or precipitation. Protein refolding steps can be used, as desired, in completing the configuration of the mature protein. Finally, high performance liquid chromatography (HPLC) can be employed in the final purification steps. For example, the methods for purifying BGL1 known in the art, find use in the present invention (See e.g., Parry et al., Biochem. J., 353:117 [2001]; and Hong et al., Appl. Microbiol. Biotechnol., 73:1331 [2007], both incorporated herein by reference). Indeed, any suitable purification methods known in the art find use in the present invention.

In some embodiments, immunological methods are used to purify EG1b. In one approach, antibody raised against the EG1b polypeptide (e.g., against a polypeptide comprising SEQ ID NO:2 or an immunogenic fragment thereof) using conventional methods is immobilized on beads, mixed with cell culture media under conditions in which the EG1b is bound, and precipitated. In a related approach, immunochromatography finds use.

In some embodiments, the EG1b is expressed as a fusion protein including a non-enzyme portion. In some embodiments, the EG1b sequence is fused to a purification facilitating domain. As used herein, the term “purification facilitating domain” refers to a domain that mediates purification of the polypeptide to which it is fused. Suitable purification domains include, but are not limited to metal chelating peptides, histidine-tryptophan modules that allow purification on immobilized metals, a sequence which binds glutathione (e.g., GST), a hemagglutinin (HA) tag (corresponding to an epitope derived from the influenza hemagglutinin protein; See e.g., Wilson et al., Cell 37:767 [1984]), maltose binding protein sequences, the FLAG epitope utilized in the FLAGS extension/affinity purification system (e.g., the system available from Immunex Corp, Seattle, Wash.), and the like. One expression vector contemplated for use in the compositions and methods described herein provides for expression of a fusion protein comprising a polypeptide of the invention fused to a polyhistidine region separated by an enterokinase cleavage site. The histidine residues facilitate purification on IMIAC (immobilized metal ion affinity chromatography; See e.g., Porath et al., Prot. Exp. Purif., 3:263-281 [1992]) while the enterokinase cleavage site provides a means for separating the EG1b polypeptide from the fusion protein. pGEX vectors (Promega; Madison, Wis.) may also be used to express foreign polypeptides as fusion proteins with glutathione S-transferase (GST). In general, such fusion proteins are soluble and can easily be purified from lysed cells by adsorption to ligand-agarose beads (e.g., glutathione-agarose in the case of GST-fusions) followed by elution in the presence of free ligand.

The EG1b and biologically active fragments as described herein have multiple industrial applications, including but not limited to, sugar production (e.g., glucose syrups), biofuels production, textile treatment, pulp or paper treatment, and applications in detergents or animal feed. A host cell containing the EG1b of the present invention finds use without recovery and purification of the recombinant EG1b (e.g., for use in a large scale biofermentor). Alternatively, the recombinant EG1b is produced and purified from the host cell.

The EG1b provided herein is particularly useful in methods used to break down cellulose to smaller oligosaccharides, disaccharides and monosaccharides. In some embodiments, the EG1b is used in saccharification methods. In some embodiments, the EG1b is used in combination with other cellulase enzymes including, for example, conventional enzymatic saccharification methods, to produce fermentable sugars. In some embodiments, the present invention provides methods for producing at least one end-product from a cellulosic substrate, the methods comprising contacting the cellulosic substrate with EG1b as described herein (and optionally other cellulases) under conditions in which fermentable sugars are produced. The fermentable sugars are then used in a fermentation reaction comprising a microorganism (e.g., a yeast) to produce the end-product. In some embodiments, the methods further comprise pretreating the cellulosic substrate to increase its susceptibility to hydrolysis prior to contacting the cellulosic substrate with the EG1b (and optionally other cellulases).

In some embodiments, enzyme compositions comprising the EG1b of the present invention are reacted with a biomass substrate in the range of about 25° C. to about 100° C., about 30° C. to about 90° C., about 30° C. to about 80° C., or about 30° C. to about 70° C. Also the biomass may be reacted with the cellobiohydrolase enzyme compositions at about 25° C., at about 30° C., at about 35° C., at about 40° C., at about 45° C., at about 50° C., at about 55° C., at about 60° C., at about 65° C., at about 70° C., at about 75° C., at about 80° C., at about 85° C., at about 90° C., at about 95° C. and at about 100° C. Generally the pH range will be from about pH 3.0 to about 8.5, about pH 3.5 to about 8.5, about pH 4.0 to about 7.5, about pH 4.0 to about 7.0 and about pH 4.0 to about 6.5. In some embodiments, the incubation time varies (e.g., from about 1.0 to about 240 hours, from about 5.0 to about 180 hrs and from about 10.0 to about 150 hrs). In some embodiments, the incubation time is at least about 1 hr, at least about 5 hrs, at least about 10 hrs, at least about 15 hrs, at least about 25 hrs, at least about 50 hr, at least about 100 hrs, at least about 180 hrs, etc. In some embodiments, incubation of the cellulase under these conditions and subsequent contact with the substrate results in the release of substantial amounts of fermentable sugars from the substrate (e.g., glucose when the cellulase is combined with beta-glucosidase). For example, in some embodiments, at least about 20%, at least about 30%, at least about 40%, at least about 50%, at least about 60%, at least about 70%, at least about 80%, at least about 90% or more fermentable sugar is available as compared to the release of sugar by a reference enzyme.

In some embodiments, an “end-product of fermentation” is any product produced by a process including a fermentation step using a fermenting organism. Examples of end-products of a fermentation include, but are not limited to, alcohols (e.g., fuel alcohols such as ethanol and butanol), organic acids (e.g., citric acid, acetic acid, lactic acid, gluconic acid, and succinic acid), glycerol, ketones, diols, amino acids (e.g., glutamic acid), antibiotics (e.g., penicillin and tetracycline), vitamins (e.g., beta-carotene and B12), hormones, and fuel molecules other than alcohols (e.g., hydrocarbons).

In some embodiments, the fermentable sugars produced by the methods of the present invention are used to produce at least one alcohol (e.g., ethanol, butanol, etc.). The EG1b of the present invention finds use in any method suitable for the generation of alcohols or other biofuels from cellulose. It is not intended that the present invention be limited to the specific methods provided herein. Two methods commonly employed are separate saccharification and fermentation (SHF) methods (See e.g., Wilke et al., Biotechnol. Bioengin., 6:155-75 [1976]) and simultaneous saccharification and fermentation (SSF) methods (See e.g., U.S. Pat. Nos. 3,990,944 and 3,990,945). In some embodiments, the SHF saccharification method comprises the steps of contacting a cellulase with a cellulose containing substrate to enzymatically break down cellulose into fermentable sugars (e.g., monosaccharides such as glucose), contacting the fermentable sugars with an alcohol-producing microorganism to produce alcohol (e.g., ethanol or butanol) and recovering the alcohol. In some embodiments, the method of consolidated bioprocessing (CBP) finds use, in which the cellulase production from the host is simultaneous with saccharification and fermentation either from one host or from a mixed cultivation. In addition, SSF methods find use in the present invention. In some embodiments, SSF methods provide a higher efficiency of alcohol production than that provided by SHF methods (See e.g., Drissen et al., Biocat. Biotrans., 27:27-35 [2009]). In some additional embodiments, the methods comprise production of at least one enzyme (e.g., EG1b) simultaneously with hydrolysis and/or fermentation (e.g., “consolidated bioprocessing”; CBP). In some embodiments, the enzyme composition is produced simultaneously with the saccharification and fermentation reactions. In some additional embodiments at least one enzyme of said composition is produced simultaneously with the saccharification and fermentation reactions. In some embodiments, in which at least one enzyme and/or the enzyme composition is produced simultaneously with the saccharification and fermentation reactions, the methods are conducted in a single reaction vessel.

In some embodiments, for cellulosic substances to be effectively used as substrates for the saccharification reaction in the presence of a cellulase of the present invention, it is desirable to pretreat the substrate. Means of pretreating a cellulosic substrate are well-known in the art, including but not limited to chemical pretreatment (e g., ammonia pretreatment, dilute acid pretreatment, dilute alkali pretreatment, or solvent exposure), physical pretreatment (e.g., steam explosion or irradiation), mechanical pretreatment (e.g., grinding or milling) and biological pretreatment (e.g., application of lignin-solubilizing microorganisms), and the present invention is not limited by such methods.

In some embodiments, any suitable alcohol producing microorganism known in the art (e.g., S. cerevisiae), finds use in the present invention for the fermentation of fermentable sugars to alcohols and other end-products. The fermentable sugars produced from the use of the EG1b provided by the present invention find use in the production of other end-products besides alcohols, including, but not limited to biofuels and/or biofuels compounds, acetone, amino acids (e.g., glycine, lysine, etc.), organic acids (e.g., lactic acids, etc.), glycerol, ascorbic acid, diols (e.g., 1,3-propanediol, butanediol, etc.), vitamins, hormones, antibiotics, other chemicals, and animal feeds. In addition, the EG1b provided herein further find use in the pulp and paper industry. Indeed, it is not intended that the present invention be limited to any particular end-products.

In some embodiments, the present invention provides an enzyme mixture that comprises the EG1b polypeptide as provided herein. The enzyme mixture may be cell-free, or in alternative embodiments, may not be separated from host cells that secrete an enzyme mixture component. A cell-free enzyme mixture typically comprises enzymes that have been separated from cells. Cell-free enzyme mixtures can be prepared by any of a variety of methodologies that are known in the art, such as filtration or centrifugation methodologies. In some embodiments, the enzyme mixtures are partially cell-free, substantially cell-free, or entirely cell-free.

In some embodiments, the EG1b and any additional enzymes present in the enzyme mixture are secreted from a single genetically modified fungal cell or by different microbes in combined or separate fermentations. Similarly, in additional embodiments, the EG1b and any additional enzymes present in the enzyme mixture are expressed individually or in sub-groups from different strains of different organisms and the enzymes are combined in vitro to make the enzyme mixture. It is also contemplated that the EG1bs and any additional enzymes in the enzyme mixture will be expressed individually or in sub-groups from different strains of a single organism, and the enzymes combined to make the enzyme mixture. In some embodiments, all of the enzymes are expressed from a single host organism, such as a genetically modified fungal cell.

In some embodiments, the enzyme mixture comprises at least one cellulase, selected from cellobiohydrolase (CBH), endoglucanase (EG), glycoside hydrolase 61 (GH61) and/or beta-glucosidase (BGL) cellulase. In some embodiments, the cellobiohydrolase is T. reesei cellobiohydrolase II. In some embodiments, the endoglucanase comprises a catalytic domain derived from the catalytic domain of a Streptomyces avermitilis endoglucanase. In some embodiments, at least one cellulase is Acidothermus cellulolyticus, Thermobifida fusca, Humicola grisea, and/or a Chrysosporium sp. cellulase. Cellulase enzymes of the cellulase mixture work together in decrystallizing and hydrolyzing the cellulose from a biomass substrate to yield fermentable sugars, such as but not limited to glucose (See e.g., Brigham et al. in Wyman ([ed.], Handbook on Bioethanol, Taylor and Francis, Washington D.C. [1995], pp 119-141, incorporated herein by reference).

Cellulase mixtures for efficient enzymatic hydrolysis of cellulose are known (See e.g., Viikari et al., Adv. Biochem. Eng. Biotechnol., 108:121-45 [2007]; and US Pat. Publns. 2009/0061484; US 2008/0057541; and US 2009/0209009, each of which is incorporated herein by reference). In some embodiments, mixtures of purified naturally occurring or recombinant enzymes are combined with cellulosic feedstock or a product of cellulose hydrolysis. In some embodiments, one or more cell populations, each producing one or more naturally occurring or recombinant cellulases, are combined with cellulosic feedstock or a product of cellulose hydrolysis.

In some embodiments, the EG1b polypeptide of the present invention is present in mixtures comprising enzymes other than cellulases that degrade cellulose, hemicellulose, pectin, and/or lignocellulose.

Cellulase mixtures for efficient enzymatic hydrolysis of cellulose are known (See e.g., Viikari et al., Adv. Biochem. Eng. Biotechnol., 108:121-45 [2007]; and US Pat. Publns. 2009/0061484; US 2008/0057541; and US 2009/0209009, each of which is incorporated herein by reference). In some embodiments, mixtures of purified naturally occurring or recombinant enzymes are combined with cellulosic feedstock or a product of cellulose hydrolysis. In some embodiments, one or more cell populations, each producing one or more naturally occurring or recombinant cellulases, are combined with cellulosic feedstock or a product of cellulose hydrolysis.

In some embodiments, the EG1b polypeptide of the present invention is present in mixtures comprising enzymes other than cellulases that degrade cellulose, hemicellulose, pectin, and/or lignocellulose.

In some additional embodiments, the present invention provides EG 1b and at least one endoxylanase. Endoxylanases (EC 3.2.1.8) catalyze the endohydrolysis of 1,4-beta-D-xylosidic linkages in xylans. This enzyme may also be referred to as endo-1,4-beta-xylanase or 1,4-beta-D-xylan xylanohydrolase. In some embodiments, an alternative is EC 3.2.1.136, a glucuronoarabinoxylan endoxylanase, an enzyme that is able to hydrolyze 1,4 xylosidic linkages in glucuronoarabinoxylans.

In some additional embodiments, the present invention provides EG1b and at least one beta-xylosidase. beta-xylosidases (EC 3.2.1.37) catalyze the hydrolysis of 1,4-beta-D-xylans, to remove successive D-xylose residues from the non-reducing termini. This enzyme may also be referred to as xylan 1,4-beta-xylosidase, 1,4-beta-D-xylan xylohydrolase, exo-1,4-beta-xylosidase or xylobiase.

In some additional embodiments, the present invention provides EG1b and at least one alpha-L-arabinofuranosidase alpha-L-arabinofuranosidases (EC 3.2.1.55) catalyze the hydrolysis of terminal non-reducing alpha-L-arabinofuranoside residues in alpha-L-arabinosides. The enzyme acts on alpha-L-arabinofuranosides, alpha-L-arabinans containing (1,3)- and/or (1,5)-linkages, arabinoxylans, and arabinogalactans. Alpha-L-arabinofuranosidase is also known as arabinosidase, alpha-arabinosidase, alpha-L-arabinosidase, alpha-arabinofuranosidase, arabinofuranosidase, polysaccharide alpha-L-arabinofuranosidase, alpha-L-arabinofuranoside hydrolase, L-arabinosidase and alpha-L-arabinanase.

In some additional embodiments, the present invention provides EG1b and at least one alpha-glucuronidase. Alpha-glucuronidases (EC 3.2.1.139) catalyze the hydrolysis of an alpha-D-glucuronoside to D-glucuronate and an alcohol.

In some additional embodiments, the present invention provides EG1b and at least one acetylxylanesterase. Acetylxylanesterases (EC 3.1.1.72) catalyze the hydrolysis of acetyl groups from polymeric xylan, acetylated xylose, acetylated glucose, alpha-napthyl acetate, and p-nitrophenyl acetate.

In some additional embodiments, the present invention provides EG1b and at least one feruloyl esterase. Feruloyl esterases (EC 3.1.1.73) have 4-hydroxy-3-methoxycinnamoyl-sugar hydrolase activity (EC 3.1.1.73) that catalyzes the hydrolysis of the 4-hydroxy-3-methoxycinnamoyl (feruloyl) group from an esterified sugar, which is usually arabinose in “natural” substrates, to produce ferulate (4-hydroxy-3-methoxycinnamate). Feruloyl esterase is also known as ferulic acid esterase, hydroxycinnamoyl esterase, FAE-III, cinnamoyl ester hydrolase, FAEA, cinnAE, FAE-I, or FAE-II.

In some additional embodiments, the present invention provides EG1b and at least one coumaroyl esterase. Coumaroyl esterases (EC 3.1.1.73) catalyze a reaction of the form: coumaroyl-saccharide+H₂O=coumarate+saccharide. In some embodiments, the saccharide is an oligosaccharide or a polysaccharide. This enzyme may also be referred to as trans-4-coumaroyl esterase, trans-p-coumaroyl esterase, p-coumaroyl esterase or p-coumaric acid esterase. The enzyme also falls within EC 3.1.1.73 so may also be referred to as a feruloyl esterase.

In some additional embodiments, the present invention provides EG1b and at least one alpha-galactosidase. Alpha-galactosidases (EC 3.2.1.22) catalyze the hydrolysis of terminal, non-reducing alpha-D-galactose residues in alpha-D-galactosides, including galactose oligosaccharides, galactomannans, galactans and arabinogalactans. This enzyme may also be referred to as melibiase.

In some additional embodiments, the present invention provides EG1b and at least one beta-galactosidase. Beta-galactosidases (EC 3.2.1.23) catalyze the hydrolysis of terminal non-reducing beta-D-galactose residues in beta-D-galactosides. In some embodiments, the polypeptide is also capable of hydrolyzing alpha-L-arabinosides. This enzyme may also be referred to as exo-(1->4)-beta-D-galactanase or lactase.

In some additional embodiments, the present invention provides EG1b and at least one beta-mannanase. Beta-mannanases (EC 3.2.1.78) catalyze the random hydrolysis of 1,4-beta-D-mannosidic linkages in mannans, galactomannans and glucomannans. This enzyme may also be referred to as mannan endo-1,4-beta-mannosidase or endo-1,4-mannanase.

In some additional embodiments, the present invention provides EG1b and at least one beta-mannosidase. Beta-mannosidases (EC 3.2.1.25) catalyze the hydrolysis of terminal, non-reducing beta-D-mannose residues in beta-D-mannosides. This enzyme may also be referred to as mannanase or mannase.

In some additional embodiments, the present invention provides EG1b and at least one glucoamylase. Glucoamylases (EC 3.2.1.3) catalyzes the release of D-glucose from non-reducing ends of oligo- and poly-saccharide molecules. Glucoamylase is also generally considered a type of amylase known as amylo-glucosidase.

In some additional embodiments, the present invention provides EG1b and at least one amylase. Amylases (EC 3.2.1.1) are starch cleaving enzymes that degrade starch and related compounds by hydrolyzing the alpha-1,4 and/or alpha-1,6 glucosidic linkages in an endo- or an exo-acting fashion. Amylases include alpha-amylases (EC 3.2.1.1); beta-amylases (3.2.1.2), amylo-amylases (EC 3.2.1.3), alpha-glucosidases (EC 3.2.1.20), pullulanases (EC 3.2.1.41), and isoamylases (EC 3.2.1.68). In some embodiments, the amylase is an alpha-amylase. In some embodiments one or more enzymes that degrade pectin are included in enzyme mixtures that comprise EG1B of the present invention. A pectinase catalyzes the hydrolysis of pectin into smaller units such as oligosaccharide or monomeric saccharides. In some embodiments, the enzyme mixtures comprise any pectinase, for example an endo-polygalacturonase, a pectin methyl esterase, an endo-galactanase, a pectin acetyl esterase, an endo-pectin lyase, pectate lyase, alpha rhamnosidase, an exo-galacturonase, an exo-polygalacturonate lyase, a rhamnogalacturonan hydrolase, a rhamnogalacturonan lyase, a rhamnogalacturonan acetyl esterase, a rhamnogalacturonan galacturonohydrolase and/or a xylogalacturonase.

In some additional embodiments, the present invention provides EG1b and at least one endo-polygalacturonase. Endo-polygalacturonases (EC 3.2.1.15) catalyze the random hydrolysis of 1,4-alpha-D-galactosiduronic linkages in pectate and other galacturonans. This enzyme may also be referred to as polygalacturonase pectin depolymerase, pectinase, endopolygalacturonase, pectolase, pectin hydrolase, pectin polygalacturonase, poly-alpha-1,4-galacturonide glycanohydrolase, endogalacturonase; endo-D-galacturonase or poly(1,4-alpha-D-galacturonide) glycanohydrolase.

In some additional embodiments, the present invention provides EG1b and at least one pectin methyl esterase. Pectin methyl esterases (EC 3.1.1.11) catalyze the reaction: pectin+n H₂O=n methanol+pectate. The enzyme may also been known as pectinesterase, pectin demethoxylase, pectin methoxylase, pectin methylesterase, pectase, pectinoesterase or pectin pectylhydrolase.

In some additional embodiments, the present invention provides EG1b and at least one endo-galactanase. Endo-galactanases (EC 3.2.1.89) catalyze the endohydrolysis of 1,4-beta-D-galactosidic linkages in arabinogalactans. The enzyme may also be known as arabinogalactan endo-1,4-beta-galactosidase, endo-1,4-beta-galactanase, galactanase, arabinogalactanase or arabinogalactan 4-beta-D-galactanohydrolase.

In some additional embodiments, the present invention provides EG1b and at least one pectin acetyl esterase. Pectin acetyl esterases catalyze the deacetylation of the acetyl groups at the hydroxyl groups of GaIUA residues of pectin.

In some additional embodiments, the present invention provides EG1b and at least one endo-pectin lyase. Endo-pectin lyases (EC 4.2.2.10) catalyze the eliminative cleavage of (1→4)-alpha-D-galacturonan methyl ester to give oligosaccharides with 4-deoxy-6-O-methyl-alpha-D-galact-4-enuronosyl groups at their non-reducing ends. The enzyme may also be known as pectin lyase, pectin trans-eliminase; endo-pectin lyase, polymethylgalacturonic transeliminase, pectin methyltranseliminase, pectolyase, PL, PNL or PMGL or (1→4)-6-O-methyl-alpha-D-galacturonan lyase.

In some additional embodiments, the present invention provides EG1b and at least one pectate lyase. Pectate lyases (EC 4.2.2.2) catalyze the eliminative cleavage of (1→4)-alpha-D-galacturonan to give oligosaccharides with 4-deoxy-alpha-D-galact-4-enuronosyl groups at their non-reducing ends. The enzyme may also be known polygalacturonic transeliminase, pectic acid transeliminase, polygalacturonate lyase, endopectin methyltranseliminase, pectate transeliminase, endogalacturonate transeliminase, pectic acid lyase, pectic lyase, alpha-1,4-D-endopolygalacturonic acid lyase, PGA lyase, PPase-N, endo-alpha-1,4-polygalacturonic acid lyase, polygalacturonic acid lyase, pectin trans-eliminase, polygalacturonic acid trans-eliminase or (1→4)-alpha-D-galacturonan lyase.

In some additional embodiments, the present invention provides EG1b and at least one alpha-rhamnosidase. Alpha-rhamnosidases (EC 3.2.1.40) catalyze the hydrolysis of terminal non-reducing alpha-L-rhamnose residues in alpha-L-rhamnosides or alternatively in rhanmogalacturonan. This enzyme may also be known as alpha-L-rhamnosidase T, alpha-L-rhamnosidase N or alpha-L-rhamnoside rhamnohydrolase.

In some additional embodiments, the present invention provides EG1b and at least one exo-galacturonase. Exo-galacturonases (EC 3.2.1.82) hydrolyze pectic acid from the non-reducing end, releasing digalacturonate. The enzyme may also be known as exo-poly-alpha-galacturonosidase, exopolygalacturonosidase or exopolygalacturanosidase.

In some additional embodiments, the present invention provides EG1b and at least one -galacturan 1,4-alpha galacturonidase (EC 3.2.1.67). These enzymes catalyze a reaction of the following type: (1,4-alpha-D-galacturonide)n+H2O=(1,4-alpha-D-galacturonide)n-i+D-galacturonate. The enzyme may also be known as poly[1->4) alpha-D-galacturonide]galacturonohydrolase, exopolygalacturonate, poly(galacturonate)hydrolase, exo-D-galacturonase, exo-D-galacturonanase, exopoly-D-galacturonase or poly(1,4-alpha-D-galacturonide) galacturonohydrolase.

In some additional embodiments, the present invention provides EG1b and at least one exopolygalacturonate lyase. Exopolygalacturonate lyases (EC 4.2.2.9) catalyze eliminative cleavage of 4-(4-deoxy-alpha-D-galact-4-enuronosyl)-D-galacturonate from the reducing end of pectate (i.e. de-esterified pectin). This enzyme may be known as pectate disaccharide-lyase, pectate exo-lyase, exopectic acid transeliminase, exopectate lyase, exopolygalacturonic acid-trans-eliminase, PATE, exo-PATE, exo-PGL or (1→4)-alpha-D-galacturonan reducing-end-disaccharide-lyase.

In some additional embodiments, the present invention provides EG1b and at least one rhamnogalacturonanase. Rhamnogalacturonanases hydrolyze the linkage between galactosyluronic acid and rhamnopyranosyl in an endo-fashion in strictly alternating rhamnogalacturonan structures, consisting of the disaccharide [(1,2-alpha-L-rhamnoyl-(1,4)-alpha-galactosyluronic acid].

In some additional embodiments, the present invention provides EG1b and at least one rhamnogalacturonan lyase. Rhamnogalacturonan lyases cleave alpha-L-Rhap-(1→4)-alpha-D-GalpA linkages in an endo-fashion in rhamnogalacturonan by beta-elimination.

In some additional embodiments, the present invention provides EG1b and at least one rhamnogalacturonan acetyl esterase Rhamnogalacturonan acetyl esterases catalyze the deacetylation of the backbone of alternating rhamnose and galacturonic acid residues in rhamnogalacturonan.

In some additional embodiments, the present invention provides EG1b and at least one rhamnogalacturonan galacturonohydrolase. Rhamnogalacturonan galacturonohydrolases hydrolyze galacturonic acid from the non-reducing end of strictly alternating rhamnogalacturonan structures in an exo-fashion. This enzyme may also be known as xylogalacturonan hydrolase.

In some additional embodiments, the present invention provides EG1b and at least one endo-arabinanase. Endo-arabinanases (EC 3.2.1.99) catalyze endohydrolysis of 1,5-alpha-arabinofuranosidic linkages in 1,5-arabinans. The enzyme may also be known as endo-arabinase, arabinan endo-1,5-alpha-L-arabinosidase, endo-1,5-alpha-L-arabinanase, endo-alpha-1,5-arabanase; endo-arabanase or 1,5-alpha-L-arabinan 1,5-alpha-L-arabinanohydrolase.

In some additional embodiments, the present invention provides EG1b and at least one enzyme that participates in lignin degradation in an enzyme mixture. Enzymatic lignin depolymerization can be accomplished by lignin peroxidases, manganese peroxidases, laccases and cellobiose dehydrogenases (CDH), often working in synergy. These extracellular enzymes are often referred to as “lignin-modifying enzymes” or “LMEs.” Three of these enzymes comprise two glycosylated heme-containing peroxidases: lignin peroxidase (LIP); Mn-dependent peroxidase (MNP); and, a copper-containing phenoloxidase laccase (LCC).

In some additional embodiments, the present invention provides EG 1b and at least one laccase. Laccases are copper containing oxidase enzymes that are found in many plants, fungi and microorganisms. Laccases are enzymatically active on phenols and similar molecules and perform a one electron oxidation. Laccases can be polymeric and the enzymatically active form can be a dimer or trimer.

In some additional embodiments, the present invention provides EG1b and at least one Mn-dependent peroxidase. The enzymatic activity of Mn-dependent peroxidase (MnP) in is dependent on Mn²⁺. Without being bound by theory, it has been suggested that the main role of this enzyme is to oxidize Mn²⁺ to Mn³⁺ (See e.g, Glenn et al., Arch. Biochem. Biophys., 251:688-696 [1986]). Subsequently, phenolic substrates are oxidized by the Mn³⁺ generated.

In some additional embodiments, the present invention provides EG1b and at least one lignin peroxidase. Lignin peroxidase is an extracellular heme that catalyses the oxidative depolymerization of dilute solutions of polymeric lignin in vitro. Some of the substrates of LiP, most notably 3,4-dimethoxybenzyl alcohol (veratryl alcohol, VA), are active redox compounds that have been shown to act as redox mediators. VA is a secondary metabolite produced at the same time as LiP by ligninolytic cultures of P. chrysosporium and without being bound by theory, has been proposed to function as a physiological redox mediator in the LiP-catalyzed oxidation of lignin in vivo (See e.g., Harvey, et al., FEBS Lett., 195:242-246 [1986]).

In some additional embodiments, the present invention provides EG1b and at least one protease, amylase, glucoamylase, and/or a lipase that participates in cellulose degradation.

As used herein, the term “protease” includes enzymes that hydrolyze peptide bonds (peptidases), as well as enzymes that hydrolyze bonds between peptides and other moieties, such as sugars (glycopeptidases). Many proteases are characterized under EC 3.4, and are suitable for use in the invention. Some specific types of proteases include, cysteine proteases including pepsin, papain and serine proteases including chymotrypsins, carboxypeptidases and metalloendopeptidases.

As used herein, the term “lipase” includes enzymes that hydrolyze lipids, fatty acids, and acylglycerides, including phosphoglycerides, lipoproteins, diacylglycerols, and the like. In plants, lipids are used as structural components to limit water loss and pathogen infection. These lipids include waxes derived from fatty acids, as well as cutin and suberin.

In some additional embodiments, the present invention provides EG1b and at least one expansin or expansin-like protein, such as a swollenin (See e.g., Salheimo et al., Eur. J. Biochem., 269:4202-4211 [2002]) or a swollenin-like protein. Expansins are implicated in loosening of the cell wall structure during plant cell growth. Expansins have been proposed to disrupt hydrogen bonding between cellulose and other cell wall polysaccharides without having hydrolytic activity. In this way, they are thought to allow the sliding of cellulose fibers and enlargement of the cell wall. Swollenin, an expansin-like protein contains an N-terminal Carbohydrate Binding Module Family 1 domain (CBD) and a C-terminal expansin-like domain. In some embodiments, an expansin-like protein or swollenin-like protein comprises one or both of such domains and/or disrupts the structure of cell walls (such as disrupting cellulose structure), optionally without producing detectable amounts of reducing sugars.

In some additional embodiments, the present invention provides EG1b and at least one polypeptide product of a cellulose integrating protein, scaffoldin or a scaffoldin-like protein, for example CipA or CipC from Clostridium thermocellum or Clostridium cellulolyticum respectively. Scaffoldins and cellulose integrating proteins are multi-functional integrating subunits which may organize cellulolytic subunits into a multi-enzyme complex. This is accomplished by the interaction of two complementary classes of domain (i.e. a cohesion domain on scaffoldin and a dockerin domain on each enzymatic unit). The scaffoldin subunit also bears a cellulose-binding module that mediates attachment of the cellulosome to its substrate. A scaffoldin or cellulose integrating protein for the purposes of this invention may comprise one or both of such domains.

In some additional embodiments, the present invention provides EG1b and at least one cellulose induced protein or modulating protein, for example as encoded by cip1 or cip2 gene or similar genes from T. reesei (See e.g., Foreman et al., J. Biol. Chem., 278:31988-31997 [2003]).

In some additional embodiments, the present invention provides EG1b and at least one member of each of the classes of the polypeptides described above, several members of one polypeptide class, or any combination of these polypeptide classes to provide enzyme mixtures suitable for various uses.

In some embodiments, the enzyme mixture comprises other types of cellulases, selected from but not limited to cellobiohydrolase, endoglucanase, beta-glucosidase, and glycoside hydrolase 61 protein (GH61) cellulases. These enzymes may be wild-type or recombinant enzymes. In some embodiments, the cellobiohydrolase is a type 1 cellobiohydrolase (e.g., a T. reesei cellobiohydrolase I). In some embodiments, the endoglucanase comprises a catalytic domain derived from the catalytic domain of a Streptomyces avermitilis endoglucanase (See e.g., US Pat. Appln. Pub. No. 2010/0267089, incorporated herein by reference). In some embodiments, the at least one cellulase is derived from Acidothermus cellulolyticus, Thermobifida fusca, Humicola grisea, Myceliophthora thermophila, Chaetomium thermophilum, Acremonium sp., Thielavia sp, Trichoderma reesei, Aspergillus sp., or a Chrysosporium sp. Cellulase enzymes of the cellulase mixture work together resulting in decrystallization and hydrolysis of the cellulose from a biomass substrate to yield fermentable sugars, such as but not limited to glucose.

Some cellulase mixtures for efficient enzymatic hydrolysis of cellulose are known (See e.g., Viikari et al., Adv. Biochem. Eng. Biotechnol., 108:121-45 [2007]; and US Pat. Appln. Publn. Nos. US 2009/0061484, US 2008/0057541, and US 2009/0209009, each of which is incorporated herein by reference in their entireties). In some embodiments, mixtures of purified naturally occurring or recombinant enzymes are combined with cellulosic feedstock or a product of cellulose hydrolysis. Alternatively or in addition, one or more cell populations, each producing one or more naturally occurring or recombinant cellulases, are combined with cellulosic feedstock or a product of cellulose hydrolysis.

In some embodiments, the enzyme mixture comprises commercially available purified cellulases. Commercial cellulases are known and available (e.g., C2730 cellulase from Trichoderma reesei ATCC No. 25921 available from Sigma-Aldrich, Inc.; and C9870 ACCELLERASE® 1500, available from Genencor).

In some embodiments, the enzyme mixture comprises an isolated EG1b as provided herein and at least one or more of an isolated cellobiohydrolase (e.g., CBH1a, and/or CBH2b); an isolated endoglucanase (EG) such as a type 2 endoglucanase (EG2), an isolated beta-glucosidase (Bgl), and/or an isolated glycoside hydrolase 61 protein (GH61). In some embodiments, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, or at least 50% of the enzyme mixture is EG1b. In some embodiments, the enzyme mixture further comprises a cellobiohydrolase type 1 (e.g., CBH1a), a cellobiohydrolase type 2 (e.g., CBH2b), and EG1b, wherein the enzymes together comprise at least 25%, at least 30%, at least 35%, at least 40%, at least 45%, at least 50%, at least 55%, at least 60%, at least 65%, at least 70%, at least 75%, or at least 80% of the enzyme mixture. In some embodiments, the enzyme mixture further comprises a beta-glucosidase (Bgl), EG1b, CBH1a, and CBH2b, wherein the four enzymes together comprise at least 30%, at least 35%, at least 40%, at least 45%, at least 50%, at least 55%, at least 60%, at least 65%, at least 70%, at least 75%, at least 80%, or at least 85% of the enzyme mixture. In some embodiments, the enzyme mixture further comprises another endoglucanase (e.g. EG2), EG1b, CBH2b, CBH1a, and Bgl, wherein the five enzymes together comprise at least 35%, at least 40%, at least 45%, at least 50%, at least 55%, at least 60%, at least 65%, at least 70%, at least 75%, at least 80%, at least 85%, or at least 90% of the enzyme mixture. In some embodiments, the enzyme mixture comprises EG1b, CBH2b, CBH1a, Bgl, EG2, and a glycoside hydrolase 61 protein (GH61), in any suitable proportion for the desired reaction. In some embodiments, the enzyme mixture composition comprises isolated cellulases in the following proportions by weight (wherein the total weight of the cellulases is 100%): about 20%-10% of EG1b, about 20%-10% of Bgl, about 30%-25% of CBH1a, about 10%-30% of GH61, and about 20%-25% of CBH2b. In some embodiments, the enzyme mixture composition comprises isolated cellulases in the following proportions by weight: about 20%-10% of EG1b, about 25%-15% of Bgl, about 20%-30% of CBH1a, about 10%-15% of GH61, and about 25%-30% of CBH2b. In some embodiments, the enzyme mixture composition comprises isolated cellulases in the following proportions by weight: about 10%-15% of EG1b, about 20%-25% of Bgl, about 30%-20% of CBH1a, about 15%-5% of GH61, and about 25%-35% of CBH2b. In some embodiments, the enzyme mixture composition comprises isolated cellulases in the following proportions by weight: about 15%-5% of EG1b, about 15%40% of Bgl, about 45%-30% of CBH1a, about 25%-5% of GH61, and about 40%-10% of CBH2b. In some embodiments, the enzyme mixture composition comprises isolated cellulases in the following proportions by weight: about 10% of EG1b, about 15% of Bgl, about 40% of CBH1a, about 25% of GH61, and about 10% of CBH2b. In some embodiments, the enzyme mixture comprises isolated cellulases in the following proportions by weight: about 12% EG1b, about 33% GH61, about 10% Bgl, about 22% CBH1a, about 23% CBH2b/EG2. In some other embodiments, the enzyme mixture comprises isolated cellulases in the following proportions by weight: about 9% EG1b, about 9% EG2, about 28% GH61, about 10% about BGL1, about 30% CBH1a, and about 14% CBH2b. It is not intended that the present invention be limited to any particular combinations nor proportions of cellulases in the enzyme mixture, as any suitable combinations of cellulases and/or proportions of cellulases find use in various embodiments of the invention.

By way of example, in some embodiments, the present invention provides various mixtures comprising at least four, at least five, or at least six of the following components, as well as any additional suitable components. In some embodiments, cellobiohydrolase 1 (CBH1) finds use; in some embodiments CBH1 is present at a concentration of about 0.14 to about 0.23 g/L (about 15% to about 25% of total protein). Exemplary CBH1 enzymes include, but are not limited to T. emersonii CBH1(wild-type; e.g., SEQ ID NO:125), M. thermophila CBH1a (wild-type; e.g., SEQ ID NO:128), and the variants CBH1a-983 (SEQ ID NO:134) and CBH1a-145 (SEQ ID NO:131). In some embodiments, cellobiohydrolase 2 (CBH2) finds use; in some embodiments, CBH2 is present at a concentration of about 0.14 to about 0.23 g/L (about 15% to about 25% of total protein). Exemplary CBH2 enzymes include but are not limited to CBH2b from M. thermophila (wild-type) (e.g., SEQ ID NO:137), as well as variants 196, 287 and 963 (SEQ ID NO:140, 143, and 146, respectively). In some embodiments, endoglucanase 2 (EG2) finds use; in some embodiments, EG2 is present at a concentration of 0 to about 0.05 g/L (0 to about 5% of total protein). Exemplary EGs include, but are not limited to M. thermophila EG2 (wild-type) (e.g., SEQ ID NO:113). In some embodiments, beta-glucosidase (BGL) finds use in the present invention; in some embodiments, BGL is present at a concentration of about 0.05 to about 0.09 g/L (about 5% to about 10% of total protein). Exemplary beta-glucosidases include, but are not limited to M. thermophila BGL1 (wild-type) (e.g., SEQ ID NO:116), variant BGL-900 (SEQ ID NO:122), and variant BGL-883 (SEQ ID NO:119). In some further embodiments, GH61 protein and/or protein variants find use; in some embodiments, GH61 enzymes are present at a concentration of about 0.23 to about 0.33 g/L (about 25% to about 35% of total protein). Exemplary GH61s include, but are not limited to M. thermophila GH61a wild-type (SEQ ID NO:5), Variant 1 (SEQ ID NO:8), Variant 5 (SEQ ID NO:11) and/or Variant 9 (SEQ ID NO:14), and/or any other GH61a variant proteins, as well as any of the other GH61 enzymes (e.g., GH61b, GH61c, GH61d, GH61e, GH61f, GH61g, GH61h, GH16i, GH61j, GH61k, GH61l, GH61m, GH61n, GH61o, GH61p, GH61q, GH61r, GH61s, GH61t, GH61u, GH61v, GH61w, GH61x, and/or GH61y) as provided herein.

In some embodiments, one, two or more than two enzymes are present in the mixtures of the present invention. In some embodiments, GH61p is present at a concentration of about 0.05 to about 0.14 g/L (e.g, about 1% to about 15% of total protein). Exemplary M. thermophila GH61p enzymes include those set forth in SEQ ID NOS:73 and 76. In some embodiments, GH61f is present at a concentration of about 0.05 to about 0.14 g/L (about 1% to about 15% of total protein). An exemplary M. thermophila GH61f is set forth in SEQ ID NO:32. In some additional embodiments, at least one additional GH61 enzyme provided herein (e.g., GH61b, GH61c, GH61d, GH61e, GH61g, GH61h, GH61i, GH61j, GH61k, GH61l, GH61m, GH61n, GH61n, GH61o, GH61q, GH61r, GH61s, GH61t, GH61u, GH61v, GH61w, GH61x, and/or GH61y, finds use at an appropriate concentration (e.g., about 0.05 to about 0.14 g/L [about 1% to about 15% of total protein]).

In some embodiments, at least one xylanase at a concentration of about 0.05 to about 0.14 g/L (about 1% to about 15% of total protein) finds use in the present invention. Exemplary xylanases include but are not limited to the M. thermophila xylanase-3 (SEQ ID NO:149), xylanase-2 (SEQ ID NO:152), xylanase-1 (SEQ ID NO:155), xylanase-6 (SEQ ID NO:158), and xylanase-5 (SEQ ID NO:161).

In some additional embodiments, at least one beta-xylosidase at a concentration of about 0.05 to about 0.14 g/L (e.g., about 1% to about 15% of total protein) finds use in the present invention. Exemplary beta-xylosidases include but are not limited to the M. thermophila beta-xylosidase (SEQ ID NO:164).

In still some additional embodiments, at least one acetyl xylan esterase at a concentration of about 0.05 to about 0.14 g/L (e.g., about 1% to about 15% of total protein) finds use in the present invention. Exemplary acetylxylan esterases include but are not limited to the M. thermophila acetylxylan esterase (SEQ ID NO:167).

In some further additional embodiments, at least one ferulic acid esterase at a concentration of about 0.05 to about 0.14 g/L (e.g., about 1% to about 15% of total protein) finds use in the present invention. Exemplary ferulic esterases include but are not limited to the M. thermophila ferulic acid esterase (SEQ ID NO:170).

In some embodiments, the enzyme mixtures comprise EG1b as provided herein and at least one cellulase, including but not limited to any of the enzymes described herein. In some embodiments, the enzyme mixtures comprise at least one EG1b protein and at least one non-cellulase enzyme. Indeed, it is intended that any combination of enzymes will find use in the enzyme compositions comprising the EG1b provided herein.

The concentrations listed above are appropriate for a final reaction volume with the biomass substrate in which all of the components listed (the “total protein”) is about 0.75 g/L, and the amount of glucan is about 93 g/L, subject to routine optimization. The user may empirically adjust the amount of each component and total protein for cellulosic substrates that have different characteristics and/or are processed at a different concentration. Any one or more of the components may be supplemented or substituted with variants with common structural and functional characteristics, as described below.

Without implying any limitation, the following mixtures further describe some embodiments of the present invention.

In some embodiments, the EG1b endoglucanase used in the mixtures of the present invention comprises at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to SEQ ID NO:2 or a fragment of SEQ ID NO:2 having endoglucanase activity.

Some mixtures comprise CBH1a within a range of about 15% to about 30% total protein, typically about 20% to about 25%; CBH2 within a range of about 15% to about 30%, typically about 17% to about 22%; EG2 within a range of about 1% to about 10%, typically about 2% to about 5%; BGL1 within a range of about 5% to about 15%, typically about 8% to about 12%; GH61a within a range of about 10% to about 40%, typically about 20% to about 30%; EG1b within a range of about 5% to about 25%, typically about 10% to about 18%; and GH61f within a range of 0% to about 30%; typically about 5% to about 20%.

In some mixtures, exemplary BGL1s include the BGL1 variant 900 (SEQ ID NO:122) and/or variant 883 (SEQ ID NO:119). In some embodiments, other enzymes are M. thermophila wild-type: CBH1a (SEQ ID NO:128), variant CBH1a (e.g., SEQ ID NOS: 131 and/or 134), CBH2b (SEQ ID NO:137), variant CHB2b (e.g., SEQ ID NOS: 140, 143, and/or 146), EG2 (SEQ ID NO:113), wildtype GH61a (SEQ ID NO:5), variant GH61a (e.g., SEQ ID NOS: 8, 11, and/or 14), and GH61f (SEQ ID NO:32), and/or T. emersonii CBH1a (e.g, SEQ ID NO:125). Any one or more of the components may be supplemented or substituted with variants having common structural and functional characteristics with the component being substituted or supplemented, as described below. In a saccharification reaction, the amount of glucan is generally about 50 to about 300 g/L, typically about 75 to about 150 g/L. The total protein is about 0.1 to about 10 g/L, typically about 0.5 to about 2 g/L, or about 0.75 g/L.

Some mixtures comprise CBH1 within a range of about 10% to about 30%, typically about 15% to about 25%; CBH2b within a range of about 10% to about 25%, typically about 15% to about 20%; EG2 within a range of about 1% to about 10%, typically about 2% to about 5%; EG1b within a range of about 2% to about 25%, typically about 6% to about 14%; GH61a within a range of about 5% to about 50%, typically about 10% to about 35%; and BGL1 within a range of about 2% to about 15%, typically about 5% to about 12%. In some embodiments, copper sulfate is also included, to generate a final concentration of Cu⁺⁺ of about 4 μM to about 200 μM, typically about 25 μM to about 60 μM. However, it is not intended that the added copper be limited to any particular concentration, as any suitable concentration finds use in the present invention and will be determined based on the reaction conditions.

In an additional mixture, an exemplary CBH1 is wild-type CBH1 from T. emersonii (SEQ ID NO:125), as well as wild-type M. thermophila CBH1a (SEQ ID NO:128), Variant 983 (SEQ ID NO:134), and Variant 145 (SEQ ID NO:131); exemplary CBH2 enzymes include the wild-type (SEQ ID NO:137), Variant 962 (SEQ ID NO:146), Variant 196 (SEQ ID NO:140), and Variant 287 (SEQ ID NO:143); an exemplary EG2 is the wild-type M. thermophila (SEQ ID NO:113);); exemplary GH61a enzymes include wild-type M. thermophila (SEQ ID NO:5), Variant 1 (SEQ ID NO:8), Variant 5 (SEQ ID NO:11), and Variant 9 (SEQ ID NO:14); and exemplary BGLs include wild-type M. thermophila BGL (SEQ ID NO:116), Variant 883 (SEQ ID NO:119), and Variant 900 (SEQ ID NO:122). In some embodiments, at least one non-GH61a enzyme is included in the mixtures. In some embodiments, multiple GH61 enzymes are included, either without the presence of wild-type GH61a and/or at least one variant GH61a or in combination with wild-type GH61a and/or at least one variant GH61a. Any one or more of the components may be supplemented or substituted with other variants having common structural and functional characteristics with the component being substituted or supplemented, as described below. In a saccharification reaction, the amount of glucan is generally about 50 to about 300 g/L, typically about 75 to about 150 g/L. The total protein is about 0.1 to about 10 g/L, typically about 0.5 to about 2 g/L, or about 0.75 g/L.

Any or all of the components listed in the mixtures referred to above may be supplemented or substituted with variant proteins that are structurally and functionally related, as described herein.

In some embodiments, the CBH1 cellobiohydrolase used in mixtures of the present invention comprises at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to either SEQ ID NO:128 (M. thermophila), SEQ ID NO:125 (T. emersonii), or a fragment of either SEQ ID NO:128 or SEQ ID NO:125 having cellobiohydrolase activity, as well as variants of M. thermophila CBH1a (e.g., SEQ ID NO:131 and/or SEQ ID NO:133), and variant fragment(s) having cellobiohydrolase activity. Exemplary CBH1 enzymes include, but are not limited to those described in US Pat. Appln. Publn. No. 2012/0003703 A1, which is hereby incorporated herein by reference in its entirety for all purposes.

In some embodiments, the CBH2b cellobiohydrolase used in the mixtures of the present invention comprises at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to SEQ ID NO:127 or a fragment of SEQ ID NO:127, as well as at least one variant M. thermophila CBH2b enzyme (e.g., SEQ ID NO:140, 143, and/or 146) and/or variant fragment(s) having cellobiohydrolase activity. Exemplary CBH2b enzymes are described in U.S. Patent Appln. Ser. No. 61/479,800, Ser. No. 13/459,038, both of which are hereby incorporated herein by reference in their entirety for all purposes.

In some embodiments, the EG2 endoglucanase used in the mixtures of the present invention comprises at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to SEQ ID NO:113 or a fragment of SEQ ID NO:113 having endoglucanase activity. Exemplary EG2 enzymes are described in U.S. patent application Ser. No. 13/332,114, and WO 2012/088159, both of which are hereby incorporated herein by reference in their entirety for all purposes.

In some embodiments, the BGL1 beta-glucosidase used the mixtures of the present invention comprises at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to SEQ ID NOS:116, 119, and/or 122, or a fragment of SEQ ID NOS:116, 119, and/or 122 having beta-glucosidase activity. Exemplary BGL1 enzymes include, but are not limited to those described in US Pat. Appln. Publ. No. 2011/0129881, WO 2011/041594, and US Pat. Appln. Publ. No. 2011/0124058 A1, all of which are hereby incorporated herein by reference in their entireties for all purposes.

In some embodiments, the GH61f protein used in the mixtures of the present invention comprises at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to SEQ ID NO:29, or a fragment of SEQ ID NO:29 having GH61 activity, assayed as described elsewhere in this disclosure.

In some embodiments, the GH61p protein used in the mixtures of the present invention comprises at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to SEQ ID NO:70, SEQ ID NO:73, or a fragment of such sequence having GH61p activity.

In some embodiments, the xylanase used in the mixtures of the present invention comprises at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to SEQ ID NO:149, SEQ ID NO:151, or a fragment of such sequence having xylanase activity.

In some embodiments, the enzyme component comprises more than one CBH2b, CBH1a, EG, Bgl, and/or GH61 enzyme (e.g., 2, 3 or 4 different variants), in any suitable combination with the EG 1b provided herein. In some embodiments, enzyme mixture compositions of the invention further comprise at least one additional protein and/or enzyme. In some embodiments, enzyme mixture compositions of the present invention further comprise at least one additional enzyme other than EG1b, Bgl, CBH1a, GH61, and/or CBH2b. In some embodiments, the enzyme mixture compositions of the invention further comprise at least one additional cellulase, other than the EG1b, EG2, Bgl, CBH1a, GH61, and/or CBH2b variant recited herein. In some embodiments, the EG1b polypeptide of the invention is also present in mixtures with non-cellulase enzymes that degrade cellulose, hemicellulose, pectin, and/or lignocellulose.

In some embodiments, the EG1b polypeptide of the present invention is used in combination with other optional ingredients such as at least one buffer, surfactant, and/or scouring agent. In some embodiments, at least one buffer is used with the EG1b polypeptide of the present invention (optionally combined with other enzymes) to maintain a desired pH within the solution in which the EG1b is employed. The exact concentration of buffer employed depends on several factors which the skilled artisan can determine. Suitable buffers are well known in the art. In some embodiments, at least one surfactant is used in with the EG1b of the present invention. Suitable surfactants include any surfactant compatible with the EG1b and, optionally, with any other enzymes being used in the mixture. Exemplary surfactants include an anionic, a non-ionic, and ampholytic surfactants. Suitable anionic surfactants include, but are not limited to, linear or branched alkylbenzenesulfonates; alkyl or alkenyl ether sulfates having linear or branched alkyl groups or alkenyl groups; alkyl or alkenyl sulfates; olefinsulfonates; alkanesulfonates, and the like. Suitable counter ions for anionic surfactants include, for example, alkali metal ions, such as sodium and potassium; alkaline earth metal ions, such as calcium and magnesium; ammonium ion; and alkanolamines having from 1 to 3 alkanol groups of carbon number 2 or 3 Ampholytic surfactants suitable for use in the practice of the present invention include, for example, quaternary ammonium salt sulfonates, betaine-type ampholytic surfactants, and the like. Suitable nonionic surfactants generally include polyoxalkylene ethers, as well as higher fatty acid alkanolamides or alkylene oxide adduct thereof, fatty acid glycerine monoesters, and the like. Mixtures of surfactants also find use in the present invention, as is known in the art.

The foregoing and other aspects of the invention may be better understood in connection with the following non-limiting examples.

### EXPERIMENTAL

The present invention is described in further detail in the following Examples, which are not in any way intended to limit the scope of the invention as claimed.

In the experimental disclosure below, the following abbreviations apply: ppm (parts per million); M (molar); mM (millimolar), uM and μM (micromolar); nM (nanomolar); mol (moles); gm and g (gram); mg (milligrams); ug and μg (micrograms); L and l (liter); ml and mL (milliliter); cm (centimeters); mm (millimeters); um and μm (micrometers); sec. (seconds); min(s) (minute(s)); h(s) and hr(s) (hour(s)); U (units); MW (molecular weight); rpm (rotations per minute); ° C. (degrees Centigrade); DNA (deoxyribonucleic acid); RNA (ribonucleic acid); HPLC (high pressure liquid chromatography); MES (2-N-morpholino ethanesulfonic acid); FIOPC (fold improvements over positive control); YPD (10 g/L yeast extract, 20 g/L peptone, and 20 g/L dextrose); SOE-PCR (splicing by overlapping extension PCR); ARS (ARS Culture Collection or NRRL Culture Collection, Peoria, Ill.); Axygen (Axygen, Inc., Union City, Calif.); Lallemand (Lallemand Ethanol Technology, Milwaukee, Wis.); Dual Biosystems (Dual Biosystems AG, Schlieven, Switzerland); Megazyme (Megazyme International Ireland, Ltd., Wicklow, Ireland); Sigma-Aldrich (Sigma-Aldrich, St. Louis, Mo.); Dasgip (Dasgip Biotools, LLC, Shrewsbury, Mass.); Difco (Difco Laboratories, BD Diagnostic Systems, Detroit, Mich.); PCRdiagnostics (PCRdiagnostics, by E coli SRO, Slovak Republic); Agilent (Agilent Technologies, Inc., Santa Clara, Calif.); Molecular Devices (Molecular Devices, Sunnyvale, Calif.); Symbio (Symbio, Inc., Menlo Park, Calif.); Newport (Newport Scientific, Australia); and Bio-Rad (Bio-Rad Laboratories, Hercules, Calif.).

The M. thermophila strains included in the development of the present invention included a “Strain CF-400” (Δcdh1), which is a derivative of C1 strain (“UV18#100fΔalplΔpyr5”), modified by deletion of cdh1, wherein cdh1 comprises the polynucleotide sequence of SEQ ID NO:5 of U.S. Pat. No. 8,236,551. “Strain CF-401” (Δcdh1Δcdh2) (ATCC No. PTA-12255), is a derivative of the C1 strain modified by deletion of both a cdh1 and a cdh2, wherein cdh2 comprises the polynucleotide sequence of SEQ ID NO:7 of U.S. Pat. No. 8,236,551. “Strain CF-404” is a derivative of the C1 strain further modified to overexpress bgl1 with a deletion of both cdh1 and cdh2, as described in U.S. Pat. No. 8,236,551, incorporated by reference herein.

The EG1b cDNA (SEQ ID NO:1) and amino acid (SEQ ID NO:2) sequences are provided below. The signal sequence is underlined in SEQ ID NO:2. SEQ ID NO:3 provides the sequence of EG1b, without the signal sequence.

(SEQ ID NO: 1)
ATGGGGCAGAAGACTCTCCAGGGGCTGGTGGCGGCGGCGGCACTGGCAGC
CTCGGTGGCGAACGCGCAGCAACCGGGCACCTTCACGCCCGAGGTGCATC
CGACGCTGCCGACGTGGAAGTGCACGACGAGCGGCGGGTGCGTCCAGCAG
GACACGTCGGTGGTGCTCGACTGGAACTACCGCTGGTTCCACACCGAGGA
CGGTAGCAAGTCGTGCATCACCTCTAGCGGCGTCGACCGGACCCTGTGCC
CGGACGAGGCGACGTGCGCCAAGAACTGCTTCGTCGAGGGCGTCAACTAC
ACGAGCAGCGGGGTCGAGACGTCCGGCAGCTCCCTCACCCTCCGCCAGTT
CTTCAAGGGCTCCGACGGCGCCATCAACAGCGTCTCCCCGCGCGTCTACC
TGCTCGGGGGAGACGGCAACTATGTCGTGCTCAAGCTCCTCGGCCAGGAG
CTGAGCTTCGACGTGGACGTATCGTCGCTCCCGTGCGGCGAGAACGCGGC
CCTGTACCTGTCCGAGATGGACGCGACGGGAGGACGGAACGAGTACAACA
CGGGCGGGGCCGAGTACGGGTCGGGCTACTGTGACGCCCAGTGCCCCGTG
CAGAACTGGAACAACGGGACGCTCAACACGGGCCGGGTGGGCTCGTGCTG
CAACGAGATGGACATCCTCGAGGCCAACTCCAAGGCCGAGGCCTTCACGC
CGCACCCCTGCATCGGCAACTCGTGCGACAAGAGCGGGTGCGGCTTCAAC
GCGTACGCGCGCGGTTACCACAACTACTGGGCCCCCGGCGGCACGCTCGA
CACGTCCCGGCCTTTCACCATGATCACCCGCTTCGTCACCGACGACGGCA
CCACCTCGGGCAAGCTCGCCCGCATCGAGCGCGTCTACGTCCAGGACGGC
AAGAAGGTGCCCAGCGCGGCGCCCGGGGGGGACGTCATCACGGCCGACGG
GTGCACCTCCGCGCAGCCCTACGGCGGCCTTTCCGGCATGGGCGACGCCC
TCGGCCGCGGCATGGTCCTGGCCCTGAGCATCTGGAACGACGCGTCCGGG
TACATGAACTGGCTCGACGCCGGCAGCAACGGCCCCTGCAGCGACACCGA
GGGTAACCCGTCCAACATCCTGGCCAACCACCCGGACGCCCACGTCGTGC
TCTCCAACATCCGCTGGGGCGACATCGGCTCCACCGTCGACACCGGCGAT
GGCGACAACAACGGCGGCGGCCCCAACCCGTCATCCACCACCACCGCTAC
CGCTACCACCACCTCCTCCGGCCCGGCCGAGCCTACCCAGACCCACTACG
GCCAGTGTGGAGGGAAAGGATGGACGGGCCCTACCCGCTGCGAGACGCCC
TACACCTGCAAGTACCAGAACGACTGGTACTCGCAGTGCCTGTAG
(SEQ ID NO: 2)
MGQKTLQGLVAAAALAASVANAQQPGTFTPEVHPTLPTWKCTTSGGCVQQ
DTSVVLDWNYRWFHTEDGSKSCITSSGVDRTLCPDEATCAKNCFVEGVNY
TSSGVETSGSSLTLRQFFKGSDGAINSVSPRVYLLGGDGNYVVLKLLGQE
LSFDVDVSSLPCGENAALYLSEMDATGGRNEYNTGGAEYGSGYCDAQCPV
QNWNNGTLNTGRVGSCCNEMDILEANSKAEAFTPHPCIGNSCDKSGCGFN
AYARGYHNYWAPGGTLDTSRPFTMITRFVTDDGTTSGKLARIERVYVQDG
KKVPSAAPGGDVITADGCTSAQPYGGLSGMGDALGRGMVLALSIWNDASG
YMNWLDAGSNGPCSDTEGNPSNILANHPDAHVVLSNIRWGDIGSTVDTGD
GDNNGGGPNPSSTTTATATTTSSGPAEPTQTHYGQCGGKGWTGPTRCETP
YTCKYQNDWYSQCL
(SEQ ID NO: 3)
QQPGTFTPEVHPTLPTWKCTTSGGCVQQDTSVVLDWNYRWFHTEDGSKSC
ITSSGVDRTLCPDEATCAKNCFVEGVNYTSSGVETSGSSLTLRQFFKGSD
GAINSVSPRVYLLGGDGNYVVLKLLGQELSFDVDVSSLPCGENAALYLSE
MDATGGRNEYNTGGAEYGSGYCDAQCPVQNWNNGTLNTGRVGSCCNEMDI
LEANSKAEAFTPHPCIGNSCDKSGCGFNAYARGYHNYWAPGGTLDTSRPF
TMITRFVTDDGTTSGKLARIERVYVQDGKKVPSAAPGGDVITADGCTSAQ
PYGGLSGMGDALGRGMVLALSIWNDASGYMNWLDAGSNGPCSDTEGNPSN
ILANHPDAHVVLSNIRWGDIGSTVDTGDGDNNGGGPNPSSTTTATATTTS
SGPAEPTQTHYGQCGGKGWTGPTRCETPYTCKYQNDWYSQCL

The wild-type M. thermophila C1 GH61a cDNA (SEQ ID NO:4) and amino acid (SEQ ID NO:5) sequences are provided below. The signal sequence is underlined in SEQ ID NO:5. SEQ ID NO:6 provides the GH61a sequence without the signal sequence.

(SEQ ID NO: 4)
ATGTCCAAGGCCTCTGCTCTCCTCGCTGGCCTGACGGGCGCGGCCCTCGT
CGCTGCACATGGCCACGTCAGCCACATCGTCGTCAACGGCGTCTACTACA
GGAACTACGACCCCACGACAGACTGGTACCAGCCCAACCCGCCAACAGTC
ATCGGCTGGACGGCAGCCGATCAGGATAATGGCTTCGTTGAACCCAACAG
CTTTGGCACGCCAGATATCATCTGCCACAAGAGCGCCACCCCCGGCGGCG
GCCACGCTACCGTTGCTGCCGGAGACAAGATCAACATCGTCTGGACCCCC
GAGTGGCCCGAATCCCACATCGGCCCCGTCATTGACTACCTAGCCGCCTG
CAACGGTGACTGCGAGACCGTCGACAAGTCGTCGCTGCGCTGGTTCAAGA
TTGACGGCGCCGGCTACGACAAGGCCGCCGGCCGCTGGGCCGCCGACGCT
CTGCGCGCCAACGGCAACAGCTGGCTCGTCCAGATCCCGTCGGATCTCAA
GGCCGGCAACTACGTCCTCCGCCACGAGATCATCGCCCTCCACGGTGCTC
AGAGCCCCAACGGCGCCCAGGCCTACCCGCAGTGCATCAACCTCCGCGTC
ACCGGCGGCGGCAGCAACCTGCCCAGCGGCGTCGCCGGCACCTCGCTGTA
CAAGGCGACCGACCCGGGCATCCTCTTCAACCCCTACGTCTCCTCCCCGG
ATTACACCGTCCCCGGCCCGGCCCTCATTGCCGGCGCCGCCAGCTCGATC
GCCCAGAGCACGTCGGTCGCCACTGCCACCGGCACGGCCACCGTTCCCGG
CGGCGGCGGCGCCAACCCTACCGCCACCACCACCGCCGCCACCTCCGCCG
CCCCGAGCACCACCCTGAGGACGACCACTACCTCGGCCGCGCAGACTACC
GCCCCGCCCTCCGGCGATGTGCAGACCAAGTACGGCCAGTGTGGTGGCAA
CGGATGGACGGGCCCGACGGTGTGCGCCCCCGGCTCGAGCTGCTCCGTCC
TCAACGAGTGGTACTCCCAGTGTTTGTAA
(SEQ ID NO: 5)
MSKASALLAGLTGAALVAAHGHVSHIVVNGVYYRNYDPTTDWYQPNPPTV
IGWTAADQDNGFVEPNSFGTPDIICHKSATPGGGHATVAAGDKINIVWTP
EWPESHIGPVIDYLAACNGDCETVDKSSLRWFKIDGAGYDKAAGRWAADA
LRANGNSWLVQIPSDLKAGNYVLRHEIIALHGAQSPNGAQAYPQCINLRV
TGGGSNLPSGVAGTSLYKATDPGILFNPYVSSPDYTVPGPALIAGAASSI
AQSTSVATATGTATVPGGGGANPTATTTAATSAAPSTTLRTTTTSAAQTT
APPSGDVQTKYGQCGGNGWTGPTVCAPGSSCSVLNEWYSQCL
(SEQ ID NO: 6)
HGHVSIHVVNGVYYRNYDPTTDWYQPNPPTVIGWTAADQDNGFVEPNSFG
TPDIICHKSATPGGGHATVAAGDKINIVWTPEWPESHIGPVIDYLAACNG
DCETVDKSSLRWFKIDGAGYDKAAGRWAADALRANGNSWLVQIPSDLKAG
NYVLRHEIIALHGAQSPNGAQAYPQCINLRVTGGGSNLPSGVAGTSLYKA
TDPGILFNPYVSSPDYTVPGPALIAGAASSIAQSTSVATATGTATVPGGG
GANPTATTTAATSAAPSTTLRTTTTSAAQTTAPPSGDVQTKYGQCGGNGW
TGPTVCAPGSSCSVLNEWYSQCL

The cDNA sequence of a M. thermophila GH61a variant (“Variant 1”) (SEQ ID NO:7) and amino acid (SEQ ID NO:8) sequence are provided below. The signal sequence is underlined in SEQ ID NO:8. SEQ ID NO:9 provides the GH61a Variant 1 sequence without the signal sequence.

(SEQ ID NO: 7)
ATGTCCAAGGCCTCTGCTCTCCTCGCTGGCCTGACGGGCGCGGCCCTCGT
CGCTGCACACGGCCACGTCAGCCACATCGTCGTCAACGGCGTCTACTACA
GGGGCTACGACCCCACGACAGACTGGTACCAGCCCAACCCGCCAACAGTC
ATCGGCTGGACGGCAGCCGATCAGGATAATGGCTTCGTTGAACCCAACAG
CTTTGGCACGCCAGATATCATCTGCCACAAGAGCGCCACCCCCGGCGGCG
GCCACGCTACCGTTGCTGCCGGAGACAAGATCAACATCGTCTGGACCCCC
GAGTGGCCCCACTCCCACATCGGCCCCGTCATTGACTACCTAGCCGCCTG
CAACGGTGACTGCGAGACCGTCGACAAGTCGTCGCTGCGCTGGTTCAAGA
TTGACGGCGCCGGCTACGACAAGGCCGCCGGCCGCTGGGCCGCCGACGCT
CTGCGCGCCAACGGCAACAGCTGGCTCGTCCAGATCCCGTCGGATCTCAA
GCCCGGCAACTACGTCCTCCGCCACGAGATCATCGCCCTCCACGGTGCTC
AGAGCCCCAACGGCGCCCAGGCGTACCCGCAGTGCATCAACCTCCGCGTC
ACCGGCGGCGGCAGCAACCTGCCCAGCGGCGTCGCCGGCACCTCGCTGTA
CAAGGCGACCGACCCGGGCATCCTCTTCAACCCCTACGTCTCCTCCCCGG
ATTACACCGTCCCCGGCCCGGCCCTCATTGCCGGCGCCGCCAGCTCGATC
GCCCAGAGCACGTCGGTCGCCACTGCCACCGGCACGGCCACCGTTCCCGG
CGGCGGCGGCGCCAACCCTACCGCCACCACCACCGCCGCCACCTCCGCCG
CCCCGAGCACCACCCTGAGGACGACCACTACCTCGGCCGCGCAGACTACC
GCCCCGCCCTCCGGCGATGTGCAGACCAAGTACGGCCAGTGTGGTGGCAA
CGGATGGACGGGCCCGACGGTGTGCGCCCCCGGCTCGAGCTGCTCCGTCC
TCAACGAGTGGTACTCCCAGTGTTTGTAA
(SEQ ID NO: 8)
MSKASALLAGLTGAALVAAHGHVSHIVVNGVYYRGYDPTTDWYQPNPPTV
IGWTAADQDNGFVEPNSFGTPDIICHKSATPGGGHATVAAGDKINIVWTP
EWPHSHIGPVIDYLAACNGDCETVDKSSLRWFKIDGAGYDKAAGRWAADA
LRANGNSWLVQIPSDLKPGNYVLRHEIIALHGAQSPNGAQAYPQCINLRV
TGGGSNLPSGVAGTSLYKATDPGILFNPYVSSPDYTVPGPALIAGAASSI
AQSTSVATATGTATVPGGGGANPTATTTAATSAAPSTTLRTTTTSAAQTT
APPSGDVQTKYGQCGGNGWTGPTVCAPGSSCSVLNEWYSQCL
(SEQ ID NO: 9)
HGHVSHIVVNGVYYRGYDPTTDWYQPNPPTVIGWTAADQDNGFVEPNSFG
TPDIICHKSATPGGGHATVAAGDKINIVWTPEWPHSHIGPVIDYLAACNG
DCETVDKSSLRWFKIDGAGYDKAAGRWAADALRANGNSWLVQIPSDLKPG
NYVLRHEIIALHGAQSPNGAQAYPQCINLRVTGGGSNLPSGVAGTSLYKA
TDPGILFNPYVSSPDYTVPGPALIAGAASSIAQSTSVATATGTATVPGGG
GANPTATTTAATSAAPSTTLRTTTTSAAQTTAPPSGDVQTKYGQCGGNGW
TGPTVCAPGSSCSVLNEWYSQCL

The cDNA sequence of a M. thermophila GH61a variant (“Variant 5”) (SEQ ID NO:10) and amino acid (SEQ ID NO:11) sequence are provided below. The signal sequence is underlined in SEQ ID NO:11. SEQ ID NO:12 provides the GH61a Variant 5 sequence without the signal sequence.

(SEQ ID NO: 10)
ACACAAATGTCCAAGGCCTCTGCTCTCCTCGCTGGCCTGACGGGCGCGGC
CCTCGTCGCTGCACACGGCCACGTCAGCCACATCGTCGTCAACGGCGTCT
ACTACAGGAACTACGACCCCACGACAGACTGGTACCAGCCCAACCCGCCA
ACAGTCATCGGCTGGACGGCAGCCGATCAGGATAATGGCTTCGITGAACC
CAACAGCTTTGGCACGCCAGATATCATCTGCCACAAGAGCGCCACCCCCG
GCGGCGGCCACGCTACCGTTGCTGCCGGAGACAAGATCAACATCGTATGG
ACCCCCGAGTGGCCCCACTCCCACATCGGCCCCGTCATTGACTACCTAGC
CGCCTGCAACGGTGACTGCGAGACCGTCGACAAGTCGTCGCTGCGCTGGT
TCAAGATTGACGGCGCCGGCTACGACAAGGCCGCCGGCCGCTGGGCCGCC
GACGCTCTGCGCGCCAACGGCAACAGCTGGCTCGTCCAGATCCCGTCGGA
TCTCGCGGCCGGCAACTACGTCCTCCGCCACGAGATCATCGCCCTCCACG
GTGCTCAGAGCCCCAACGGCGCCCAGGCGTACCCGCAGTGCATCAACCTC
CGCGTCACCGGCGGCGGCAGCAACCTGCCCAGCGGCGTCGCCGGCACCTC
GCTGTACAAGGCGACCGACCCGGGCATCCTCTTCAACCCCTACGTCTCCT
CCCCGGATTACACCGTCCCCGGCCCGGCCCTCATTGCCGGCGCCGCCAGC
TCGATCGCCCAGAGCACGTCGGTCGCCACTGCCACCGGCACGGCCACCGT
TCCCGGCGGCGGCGGCGCCAACCCTACCGCCACCACCACCGCCGCCACCT
CCGCCGCCCCGAGCACCACCCTGAGGACGACCACTACCTCGGCCGCGCAG
ACTACCGCCCCGCCCTCCGGCGATGTGCAGACCAAGTACGGCCAGTGTGG
TGGCAACGGATGGACGGGCCCGACGGTGTGCGCCCCCGGCTCGAGCTGCT
CCGTCCTCAACGAGTGGTACTCCCAGTGTTTGTAA
(SEQ ID NO: 11)
MSKASALLAGLTGAALVAAHGHVSHIVVNGVYYRNYDPTTDWYQPNPPTV
IGWTAADQDNGFVEPNSFGTPDIICHKSATPGGGHATVAAGDKINIVWTP
EWPHSHIGPVIDYLAACNGDCETVDKSSLRWFKIDGAGYDKAAGRWAADA
LRANGNSWLVQIPSDLAAGNYVLRHEIIALHGAQSPNGAQAYPQCINLRV
TGGGSNLPSGVAGTSLYKATDPGILFNPYVSSPDYTVPGPALIAGAASSI
AQSTSVATATGTATVPGGGGANPTATTTAATSAAPSTTLRTTTTSAAQTT
APPSGDVQTKYGQCGGNGWTGPTVCAPGSSCSVLNEWYSQCL
(SEQ ID NO: 12)
HGHVSHIVVNGVYYRNYDPTTDWYQPNPPTVIGWTAADQDNGFVEPNSFG
TPDIICHKSATPGGGHATVAAGDKINIVWTPEWPHSHIGPVIDYLAACNG
DCETVDKSSLRWFKIDGAGYDKAAGRWAADALRANGNSWLVQIPSDLAAG
NYVLRHEIIALHGAQSPNGAQAYPQCINLRVTGGGSNLPSGVAGTSLYKA
TDPGILFNPYVSSPDYTVPGPALIAGAASSIAQSTSVATATGTATVPGGG
GANPTATTTAATSAAPSTTLRTTTTSAAQTTAPPSGDVQTKYGQCGGNGW
TGPTVCAPGSSCSVLNEWYSQCL

The cDNA sequence of a M. thermophila GH61a variant (“Variant 9”) (SEQ ID NO:13) and amino acid (SEQ ID NO:14) sequence are provided below. The signal sequence is underlined in SEQ ID NO:14. SEQ ID NO:15 provides the GH61a Variant 9 sequence without the signal sequence.

(SEQ ID NO: 13)
ACAAACATGTCCAAGGCCTCTGCTCTCCTCGCTGGCCTGACGGGCGCGGC
CCTCGTCGCTGCACATGGCCACGTCAGCCACATCGTCGTCAACGGCGTCT
ACTACAGGAACTACGACCCCACGACAGACTGGTACCAGCCCAACCCGCCA
ACAGTCATCGGCTGGACGGCAGCCGATCAGGATAATGGCTTCGTTGAACC
CAACAGCTTTGGCACGCCAGATATCATCTGCCACAAGAGCGCCACCCCCG
GCGGCGGCCACGCTACCGTTGCTGCCGGAGACAAGATCAACATCCAGTGG
ACCCCCGAGTGGCCCGAATCCCACATCGGCCCCGTCATTGACTACCTAGC
CGCCTGCAACGGTGACTGCGAGACCGTCGACAAGTCGTCGCTGCGCTGGT
TCAAGATTGACGGCGCCGGCTACGACAAGGCCGCCGGCCGCTGGGCCGCC
GACGCTCTGCGCGCCAACGGCAACAGCTGGCTCGTCCAGATCCCGTCGGA
TCTCAAGGCCGGCAACTACGTCCTCCGCCACGAGATCATCGCCCTCCACG
GTGCTCAGAGCCCCAACGGCGCCCAGAACTACCCGCAGTGCATCAACCTC
CGCGTCACCGGCGGCGGCAGCAACCTGCCCAGCGGCGTCGCCGGCACCTC
GCTGTACAAGGCGACCGACCCGGGCATCCTCTTCAACCCCTACGTCTCCT
CCCCGGATTACACCGTCCCCGGCCCGGCCCTCATTGCCGGCGCCGCCAGC
TCGATCGCCCAGAGCACGTCGGTCGCCACTGCCACCGGCACGGCCACCGT
TCCCGGCGGCGGCGGCGCCAACCCTACCGCCACCACCACCGCCGCCACCT
CCGCCGCCCCGAGCACCACCCTGAGGACGACCACTACCTCGGCCGCGCAG
ACTACCGCCCCGCCCTCCGGCGATGTGCAGACCAAGTACGGCCAGTGTGG
TGGCAACGGATGGACGGGCCCGACGGTGTGCGCCCCCGGCTCGAGCTGCT
CCGTCCTCAACGAGTGGTACTCCCAGTGTTTGTAA
(SEQ ID NO: 14)
MSKASALLAGLTGAALVAAHGHVSHIVVNGVYYRNYDPTTDWYQPNPPTV
IGWTAADQDNGFVEPNSFGTPDIICHKSATPGGGHATVAAGDKINIQWTP
EWPESHIGPVIDYLAACNGDCETVDKSSLRWFKIDGAGYDKAAGRWAADA
LRANGNSWLVQLPSDLKAGNYVLRHEIIALHGAQSPNGAQNYPQCINLRV
TGGGSNLPSGVAGTSLYKATDPGILFNPYVSSPDYTVPGPALIAGAASSI
AQSTSVATATGTATVPGGGGANPTATTTAATSAAPSTTLRTTTTSAAQTT
APPSGDVQTKYGQCGGNGWTGPTVCAPGSSCSVLNEWYSQCL
(SEQ ID NO: 15)
HGHVSHIVVNGVYYRNYDPTTDWYQPNPPTVIGWTAADQDNGFVEPNSFG
TPDIICHKSATPGGGHATVAAGDKINIQWTPEWPESHIGPVIDYLAACNG
DCETVDKSSLRWFKIDGAGYDKAAGRWAADALRANGNSWLVQIPSDLKAG
NYVLRHEIIALHGAQSPNGAQNYPQCINLRVTGGGSNLPSGVAGTSLYKA
TDPGILFNPYVSSPDYTVPGPALIAGAASSIAQSTSVATATGTATVPGGG
GANPTATTTAATSAAPSTTLRTTTTSAAQTTAPPSGDVQTKYGQCGGNGW
TGPTVCAPGSSCSVLNEWYSQCL

The polynucleotide (SEQ ID NO:16) and amino acid (SEQ ID NO:17) sequences of an M. thermophila GH61b are provided below. The signal sequence is shown underlined in SEQ ID NO:17. SEQ ID NO:18 provides the sequence of this GH61b without the signal sequence.

(SEQ ID NO: 16)
ATGAAGCTCTCCCTCTTTTCCGTCCTGGCCACTGCCCTCACCGTCGAGGG
GCATGCCATCTTCCAGAAGGTCTCCGTCAACGGAGCGGACCAGGGCTCCC
TCACCGGCCTCCGCGCTCCCAACAACAACAACCCCGTGCAGAATGTCAAC
AGCCAGGACATGATCTGCGGCCAGTCGGGATCGACGTCGAACACTATCAT
CGAGGTCAAGGCCGGCGATAGGATCGGTGCCTGGTATCAGCATGTCATCG
GCGGTGCCCAGTTCCCCAACGACCCAGACAACCCGATTGCCAAGTCGCAC
AAGGGCCCCGTCATGGCCTACCTCGCCAAGGTTGACAATGCCGCAACCGC
CAGCAAGACGGGCCTGAAGTGGTTCAAGATTTGGGAGGATACCTTTAATC
CCAGCACCAAGACCTGGGGTGTCGACAACCTCATCAACAACAACGGCTGG
GTGTACTTCAACCTCCCGCAGTGCATCGCCGACGGCAACTACCTCCTCCG
CGTCGAGGTCCTCGCTCTGCACTCGGCCTACTCCCAGGGCCAGGCTCAGT
TCTACCAGTCCTGCGCCCAGATCAACGTATCCGGCGGCGGCTCCTTCACG
CCGGCGTCGACTGTCAGCTTCCCGGGTGCCTACAGCGCCAGCGACCCCGG
TATCCTGATCAACATCTACGGCGCCACCGGCCAGCCCGACAACAACGGCC
AGCCGTACACTGCCCCTGGGCCCGCGCCCATCTCCTGC
(SEQ ID NO: 17)
MKLSLFSVLATALTVEGHAIFQKVSVNGADQGSLTGLRAPNNNNPVQNVN
SQDMICGQSGSTSNTIIEVKAGDRIGAWYQHVIGGAQFPNDPDNPIAKSH
KGPVMAYLAKVDNAATASKTGLKWFKIWEDTFNPSTKTWGVDNLINNNGW
VYFNLPQCIADGNYLLRVEVLALHSAYSQGQAQFYQSCAQINVSGGGSFT
PASTVSFPGAYSASDPGILINIYGATGQPDNNGQPYTAPGPAPISC
(SEQ ID NO: 18)
IFQKVSVNGADQGSLTGLRAPNNNNPVQNVNSQDMICGQSGSTSNTIIEV
KAGDRIGAWYQHVIGGAQFPNDPDNPIAKSHKGPVMAYLAKVDNAATASK
TGLKWFKIWEDTFNPSTKTWGVDNLINNNGWVYFNLPQCIADGNYLLRVE
VLALHSAYSQGQAQFYQSCAQINVSGGGSFTPASTVSFPGAYSASDPGIL
INIYGATGQPDNNGQPYTAPGPAPISC

The polynucleotide (SEQ ID NO:19) and amino acid (SEQ ID NO:20) sequences of an M. thermophila GH61c are provided below. The signal sequence is shown underlined in SEQ ID NO:20. SEQ ID NO:21 provides the sequence of this GH61c without the signal sequence.

(SEQ ID NO: 19)
ATGGCCCTCCAGCTCTTGGCGAGCTTGGCCCTCCTCTCAGTGCCGGCCCT
TGCCCACGGTGGCTTGGCCAACTACACCGTCGGTGATACTTGGTACAGAG
GCTACGACCCAAACCTGCCGCCGGAGACGCAGCTCAACCAGACCTGGATG
ATCCAGCGGCAATGGGCCACCATCGACCCCGTCTTCACCGTGTCGGAGCC
GTACCTGGCCTGCAACAACCCGGGCGCGCCGCCGCCCTCGTACATCCCCA
TCCGCGCCGGTGACAAGATCACGGCCGTGTACTGGTACTGGCTGCACGCC
ATCGGGCCCATGAGCGTCTGGCTCGCGCGGTGCGGCGACACGCCCGCGGC
CGACTGCCGCGACGTCGACGTCAACCGGGTCGGCTGGTTCAAGATCTGGG
AGGGCGGCCTGCTGGAGGGTCCCAACCTGGCCGAGGGGCTCTGGTACCAA
AAGGACTTCCAGCGCTGGGACGGCTCCCCGTCCCTCTGGCCCGTCACGAT
CCCCAAGGGGCTCAAGAGCGGGACCTACATCATCCGGCACGAGATCCTGT
CGCTTCACGTCGCCCTCAAGCCCCAGTTTTACCCGGAGTGTGCGCATCTG
AATATTACTGGGGGCGGAGACTTGCTGCCACCCGAAGAGACTCTGGTGCG
GTTTCCGGGGGTTTACAAAGAGGACGATCCCTCTATCTTCATCGATGTCT
ACTCGGAGGAGAACGCGAACCGGACAGATTATACGGTTCCGGGAGGGCCA
ATCTGGGAAGGG
(SEQ ID NO: 20)
MALQLLASLALLSVPALAHGGLANYTVGDTWYRGYDPNLPPETQLNQTWM
IQRQWATIDPVFTVSEPYLACNNPGAPPPSYIPIRAGDKITAVYWYWLHA
IGPMSVWLARCGDTPAADCRDVDVNRVGWFKIWEGGLLEGPNLAEGLWYQ
KDFQRWDGSPSLWPVTIPKGLKSGTYIIRHEILSLHVALKPQFYPECAHL
NITGGGDLLPPEETLVRFPGVYKEDDPSIFIDVYSEENANRTDYTVPGGP
IWEG
(SEQ ID NO: 21)
NYTVGDTWYRGYDPNLPPETQLNQTWMIQRQWATIDPVFTVSEPYLACNN
PGAPPPSYIPIRAGDKITAVYWYWLHAIGPMSVWLARCGDTPAADCRDVD
VNRVGWFKIWEGGLLEGPNLAEGLWYQKDFQRWDGSPSLWPVTIPKGLKS
GTYIIRHEILSLHVALKPQFYPECAHLNITGGGDLLPPEETLVRFPGVYK
EDDPSIFIDVYSEENANRTDYTVPGGPIWEG

The polynucleotide (SEQ ID NO:22) and amino acid (SEQ ID NO:23) sequences of an M. thermophila GH61d are provided below. The signal sequence is shown underlined in SEQ ID NO:23. SEQ ID NO:24 provides the sequence of this GH61d without the signal sequence.

(SEQ ID NO: 22)
ATGAAGGCCCTCTCTCTCCTTGCGGCTGCCGGGGCAGTCTCTGCGCATAC
CATCTTCGTCCAGCTCGAAGCAGACGGCACGAGGTACCCGGTTTCGTACG
GGATCCGGGACCCAACCTACGACGGCCCCATCACCGACGTCACATCCAAC
GACGTTGCTTGCAACGGCGGTCCGAACCCGACGACCCCCTCCAGCGACGT
CATCACCGTCACCGCGGGCACCACCGTCAAGGCCATCTGGAGGCACACCC
TCCAATCCGGCCCGGACGATGTCATGGACGCCAGCCACAAGGGCCCGACC
CTGGCCTACATCAAGAAGGTCGGCGATGCCACCAAGGACTCGGGCGTCGG
CGGTGGCTGGTTCAAGATCCAGGAGGACGGTTACAACAACGGCCAGTGGG
GCACCAGCACCGTTATCTCCAACGGCGGCGAGCACTACATTGACATCCCG
GCCTGCATCCCCGAGGGTCAGTACCTCCTCCGCGCCGAGATGATCGCCCT
CCACGCGGCCGGGTCCCCCGGCGGCGCTCAGCTCTACATGGAATGTGCCC
AGATCAACATCGTCGGCGGCTCCGGCTCGGTGCCCAGCTCGACGGTCAGC
TTCCCCGGCGCGTATAGCCCCAACGACCCGGGTCTCCTCATCAACATCTA
TTCCATGTCGCCCTCGAGCTCGTACACCATCCCGGGCCCGCCCGTTTTCA
AGTGC
(SEQ ID NO: 23)
MKALSLLAAAGAVSAHTIFVQLEADGTRYPVSYGIRDPTYDGPITDVTSN
DVACNGGPNPTTPSSDVITVTAGTTVKAIWRHTLQSGPDDVMDASHKGPT
LAYIKKVGDATKDSGVGGGWFKIQEDGYNNGQWGTSTVISNGGEHYIDIP
ACIPEGQYLLRAEMIALHAAGSPGGAQLYMECAQINIVGGSGSVPSSTVS
FPGAYSPNDPGLLINIYSMSPSSSYTIPGPPVFKC
(SEQ ID NO: 24)
HTIFVQLEADGTRYPVSYGIRDPTYDGPITDVTSNDVACNGGPNPTTPSS
DVITVTAGTTVKAIWRHTLQSGPDDVMDASHKGPTLAYIKKVGDATKDSG
VGGGWFKIQEDGYNNGQWGTSTVISNGGEHYIDIPACIPEGQYLLRAEMI
ALHAAGSPGGAQLYMECAQINIVGGSGSVPSSTVSFPGAYSPNDPGLLIN
IYSMSPSSSYTIPGPPVFKC

The polynucleotide (SEQ ID NO:25) and amino acid (SEQ ID NO:26) sequences of an M. thermophila GH61e are provided below. The signal sequence is shown underlined in SEQ ID NO:26. SEQ ID NO:27 provides the sequence of this GH61d without the signal sequence.

(SEQ ID NO: 25)
ATGAAGTCGTCTACCCCGGCCTTGTTCGCCGCTGGGCTCCTTGCTCAGCA
TGCTGCGGCCCACTCCATCTTCCAGCAGGCGAGCAGCGGCTCGACCGACT
TTGATACGCTGTGCACCCGGATGCCGCCCAACAATAGCCCCGTCACTAGT
GTGACCAGCGGCGACATGACCTGCAAAGTCGGCGGCACCAAGGGGGTGTC
CGGCTTCTGCGAGGTGAACGCCGGCGACGAGTTCACGGTTGAGATGCACG
CGCAGCCCGGCGACCGCTCGTGCGCCAACGAGGCCATCGGCGGGAACCAC
TTCGGCCCGGTCCTCATCTACATGAGCAAGGTCGACGACGCCTCCACCGC
CGACGGGTCCGGCGACTGGTTCAAGGTGGACGAGTTCGGCTACGACGCAA
GCACCAAGACCTGGGGCACCGACAAGCTCAACGAGAACTGCGGCAAGCGC
ACCTTCAACATCCCCAGCCACATCCCCGCGGGCGACTATCTCGTCCGGGC
CGAGGCTATCGCGCTACACACTGCCAACCAGCCAGGCGGCGCGCAGTTCT
ACATGAGCTGCTATCAAGTCAGGATTTCCGGCGGCGAAGGGGGCCAGCTG
CCTGCCGGAGTCAAGATCCCGGGCGCGTACAGTGCCAACGACCCCGGCAT
CCTTGTCGACATCTGGGGTAACGATTTCAACGACCCTCCAGGACACTCGG
CCCGTCACGCCATCATCATCATCAGCAGCAGCAGCAACAACAGCGGCGCC
AAGATGACCAAGAAGATCCAGGAGCCCACCATCACATCGGTCACGGACCT
CCCCACCGACGAGGCCAAGTGGATCGCGCTCCAAAAGATCTCGTACGTGG
ACCAGACGGGCACGGCGCGGACATACGAGCCGGCGTCGCGCAAGACGCGG
TCGCCAAGAGTCTAG
(SEQ ID NO: 26)
MKSSTPALFAAGLLAQHAAAHSIFQQASSGSTDFDTLCTRMPPNNSPVTS
VTSGDMTCKVGGTKGVSGFCEVNAGDEFTVEMHAQPGDRSCANEAIGGNH
FGPVLIYMSKVDDASTADGSGDWFKVDEFGYDASTKTWGTDKLNENCGKR
TFNIPSHIPAGDYLVRAEAIALHTANQPGGAQFYMSCYQVRISGGEGGQL
PAGVKIPGAYSANDPGILVDIWGNDFNDPPGHSARHAIIIISSSSNNSGA
KMTKKIQEPTITSVTDLPTDEAKWIALQKISYVDQTGTARTYEPASRKTR
SPRV
(SEQ ID NO: 27)
HSIFQQASSGSTDFDTLCTRMPPNNSPVTSVTSGDMTCKVGGTKGVSGFC
EVNAGDEFTVEMHAQPGDRSCANEAIGGNHFGPVLIYMSKVDDASTADGS
GDWFKVDEFGYDASTKTWGTDKLNENCGKRTFNIPSHIPAGDYLVRAEAI
ALHTANQPGGAQFYMSCYQVRISGGEGGQLPAGVKIPGAYSANDPGILVD
IWGNDFNDPPGHSARHAIIIISSSSNNSGAKMTKKIQEPTITSVTDLPTD
EAKWIALQKISYVDQTGTARTYEPASRKTRSPRV

The polynucleotide (SEQ ID NO:28) and amino acid (SEQ ID NO:29) sequences of an alternative M. thermophila GH61e are provided below. The signal sequence is shown underlined in SEQ ID NO:29. SEQ ID NO:30 provides the sequence of this GH61e without the signal sequence.

(SEQ ID NO: 28)
ATGAAGTCGTCTACCCCGGCCTTGTTCGCCGCTGGGCTCCTTGCTCAGCA
TGCTGCGGCCCACTCCATCTTCCAGCAGGCGAGCAGCGGCTCGACCGACT
TTGATACGCTGTGCACCCGGATGCCGCCCAACAATAGCCCCGTCACTAGT
GTGACCAGCGGCGACATGACCTGCAACGTCGGCGGCACCAAGGGGGTGTC
GGGCTTCTGCGAGGTGAACGCCGGCGACGAGTTCACGGTTGAGATGCACG
CGCAGCCCGGCGACCGCTCGTGCGCCAACGAGGCCATCGGCGGGAACCAC
TTCGGCCCGGTCCTCATCTACATGAGCAAGGTCGACGACGCCTCCACTGC
CGACGGGTCCGGCGACTGGTTCAAGGTGGACGAGTTCGGCTACGACGCAA
GCACCAAGACCTGGGGCACCGACAAGCTCAACGAGAACTGCGGCAAGCGC
ACCTTCAACATCCCCAGCCACATCCCCGCGGGCGACTATCTCGTCCGGGC
CGAGGCTATCGCGCTACACACTGCCAACCAGCCAGGCGGCGCGCAGTTCT
ACATGAGCTGCTATCAAGTCAGGATTTCCGGCGGCGAAGGGGGCCAGCTG
CCTGCCGGAGTCAAGATCCCGGGCGCGTACAGTGCCAACGACCCCGGCAT
CCTTGTCGACATCTGGGGTAACGATTTCAACGAGTACGTTATTCCGGGCC
CCCCGGTCATCGACAGCAGCTACTTC
(SEQ ID NO: 29)
MKSSTPALFAAGLLAQHAAAHSIFQQASSGSTDFDTLCTRMPPNNSPVTS
VTSGDMTCNVGGTKGVSGFCEVNAGDEFTVEMHAQPGDRSCANEAIGGNH
FGPVLIYMSKVDDASTADGSGDWFKVDEFGYDASTKTWGTDKLNENCGKR
TFNIPSHIPAGDYLVRAEAIALHTANQPGGAQFYMSCYQVRISGGEGGQL
PAGVKIPGAYSANDPGILVDIWGNDFNEYVIPGPPVIDSSYF
(SEQ ID NO: 30)
HSIFQQASSGSTDFDTLCTRMPPNNSPVTSVTSGDMTCNVGGTKGVSGFC
EVNAGDEFTVEMHAQPGDRSCANEAIGGNHFGPVLIYMSKVDDASTADGS
GDWFKVDEFGYDASTKTWGTDKLNENCGKRTFNIPSHIPAGDYLVRAEAI
ALHTANQPGGAQFYMSCYQVRISGGEGGQLPAGVKIPGAYSANDPGILVD
IWGNDFNEYVIPGPPVIDSSYF

The polynucleotide (SEQ ID NO:31) and amino acid (SEQ ID NO:32) sequences of a M. thermophila GH61f are provided below. The signal sequence is shown underlined in SEQ ID NO:32. SEQ ID NO:33 provides the sequence of this GH61f without the signal sequence.

(SEQ ID NO: 31)
ATGAAGTCCTTCACCCTCACCACTCTGGCCGCCCTGGCTGGCAACGCCGC
CGCTCACGCGACCTTCCAGGCCCTCTGGGTCGACGGCGTCGACTACGGCG
CGCAGTGTGCCCGTCTGCCCGCGTCCAACTCGCCGGTCACCGACGTGACC
TCCAACGCGATCCGCTGCAACGCCAACCCCTCGCCCGCTCGGGGCAAGTG
CCCGGTCAAGGCCGGCTCGACCGTTACGGTCGAGATGCATCAGCAACCCG
GTGACCGCTCGTGCAGCAGCGAGGCGATCGGCGGGGCGCACTACGGCCCC
GTGATGGTGTACATGTCCAAGGTGTCGGACGCGGCGTCGGCGGACGGGTC
GTCGGGCTGGTTCAAGGTGTTCGAGGACGGCTGGGCCAAGAACCCGTCCG
GCGGGTCGGGCGACGACGACTACTGGGGCACCAAGGACCTGAACTCGTGC
TGCGGGAAGATGAACGTCAAGATCCCCGCCGACCTGCCCTCGGGCGACTA
CCTGCTCCGGGCCGAGGCCCTCGCGCTGCACACGGCCGGCAGCGCGGGCG
GCGCCCAGTTCTACATGACCTGCTACCAGCTCACCGTGACCGGCTCCGGC
AGCGCCAGCCCGCCCACCGTCTCCTTCCCGGGCGCCTACAAGGCCACCGA
CCCGGGCATCCTCGTCAACATCCACGCCCCGCTGTCCGGCTACACCGTGC
CCGGCCCGGCCGTCTACTCGGGCGGCTCCACCAAGAAGGCCGGCAGCGCC
TGCACCGGCTGCGAGTCCACTTGCGCCGTCGGCTCCGGCCCCACCGCCAC
CGTCTCCCAGTCGCCCGGTTCCACCGCCACCTCGGCCCCCGGCGGCGGCG
GCGGCTGCACCGTCCAGAAGTACCAGCAGTGCGGCGGCCAGGGCTACACC
GGCTGCACCAACTGCGCGTCCGGCTCCACCTGCAGCGCGGTCTCGCCGCC
CTACTACTCGCAGTGCGTC
(SEQ ID NO: 32)
MKSFTLTTLAALAGNAAAHATFQALWVDGVDYGAQCARLPASNSPVTDVT
SNAIRCNANPSPARGKCPVKAGSTVTVEMHQQPGDRSCSSEAIGGAHYGP
VMVYMSKVSDAASADGSSGWFKVFEDGWAKNPSGGSGDDDYWGTKDLNSC
CGKMNVKIPADLPSGDYLLRAEALALHTAGSAGGAQFYMTCYQLTVTGSG
SASPPTVSFPGAYKATDPGALVNIHAPLSGYTVPGPAVYSGGSTKKAGSA
CTGCESTCAVGSGPTATVSQSPGSTATSAPGGGGGCTVQKYQQCGGQGYT
GCTNCASGSTCSAVSPPYYSQCV
(SEQ ID NO: 33)
HATFQALWVDGVDYGAQCARLPASNSPVTDVTSNAIRCNANPSPARGKCP
VKAGSTVTVEMHQQPGDRSCSSEAIGGAHYGPVMVYMSKVSDAASADGSS
GWFKVFEDGWAKNPSGGSGDDDYWGTKDLNSCCGKMNVKIPADLPSGDYL
LRAEALALHTAGSAGGAQFYMTCYQLTVTGSGSASPPTVSFPGAYKATDP
GILVNIHAPLSGYTVPGPAVYSGGSTKKAGSACTGCESTCAVGSGPTATV
SQSPGSTATSAPGGGGGCTVQKYQQCGGQGYTGCTNCASGSTCSAVSPPY
YSQCV

The polynucleotide (SEQ ID NO:34) and amino acid (SEQ ID NO:35) sequences of an M. thermophila GH61g are provided below. The signal sequence is shown underlined in SEQ ID NO:35. SEQ ID NO:36 provides the sequence of this GH61g without the signal sequence.

(SEQ ID NO: 34)
ATGAAGGGACTCCTCGGCGCCGCCGCCCTCTCGCTGGCCGTCAGCGATGT
CTCGGCCCACTACATCTTTCAGCAGCTGACGACGGGCGGCGTCAAGCACG
CTGTGTACCAGTACATCCGCAAGAACACCAACTATAACTCGCCCGTGACC
GATCTGACGTCCAACGACCTCCGCTGCAATGTGGGTGCTACCGGTGCGGG
CACCGATACCGTCACGGTGCGCGCCGGCGATTCGTTCACCTTCACGACCG
ATACGCCCGTTTACCACCAGGGCCCGACCTCGATCTACATGTCCAAGGCC
CCCGGCAGCGCGTCCGACTACGACGGCAGCGGCGGCTGGTTCAAGATCAA
GGACTGGGCTGACTACACCGCCACGATTCCGGAATGTATTCCCCCCGGCG
ACTACCTGCTTCGCATCCAGCAACTCGGCATCCACAACCCTTGGCCCGCG
GGCATCCCCCAGTTCTACATCTCTTGTGCCCAGATCACCGTGACTGGTGG
CGGCAGTGCCAACCCCGGCCCGACCGTCTCCATCCCAGGCGCCTTCAAGG
AGACCGACCCGGGCTACACTGTCAACATCTACAACAACTTCCACAACTAC
ACCGTCCCTGGCCCAGCCGTCTTCACCTGCAACGGTAGCGGCGGCAACAA
CGGCGGCGGCTCCAACCCAGTCACCACCACCACCACCACCACCACCAGGC
CGTCCACCAGCACCGCCCAGTCCCAGCCGTCGTCGAGCCCGACCAGCCCC
TCCAGCTGCACCGTCGCGAAGTGGGGCCAGTGCGGAGGACAGGGTTACAG
CGGCTGCACCGTGTGCGCGGCCGGGTCGACCTGCCAGAAGACCAACGACT
ACTACAGCCAGTGCTTGTAG
(SEQ ID NO: 35)
MKGLLGAAALSLAVSDVSAHYIFQQLTTGGVKHAVYQYIRKNTNYNSPVT
DLTSNDLRCNVGATGAGTDTVTVRAGDSFTFTTDTPVYHQGPTSIYMSKA
PGSASDYDGSGGWFKIKDWADYTATIPECIPPGDYLLRIQQLGIHNPWPA
GIPQFYISCAQITVTGGGSANPGPTVSIPGAFKETDPGYTVNIYNNFHNY
TVPGPAVFTCNGSGGNNGGGSNPVTTTTTTTTRPSTSTAQSQPSSSPTSP
SSCTVAKWGQCGGQGYSGCTVCAAGSTCQKTNDYYSQCL
(SEQ ID NO: 36)
HYIFQQLTTGGVKHAVYQYIRKNTNYNSPVTDLTSNDLRCNVGATGAGTD
TVTVRAGDSFTFTTDTPVYHQGPTSIYMSKAPGSASDYDGSGGWFKIKDW
ADYTATIPECIPPGDYLLRIQQLGIHNPWPAGIPQFYISCAQITVTGGGS
ANPGPTVSIPGAFKETDPGYTVNIYNNFHNYTVPGPAVFTCNGSGGNNGG
GSNPVTTTTTTTTRPSTSTAQSQPSSSPTSPSSCTVAKWGQCGGQGYSGC
TVCAAGSTCQKTNDYYSQCL

The polynucleotide (SEQ ID NO:37) and amino acid (SEQ ID NO:38) sequences of an alternative M. thermophila GH61g are provided below. The signal sequence is shown underlined in SEQ ID NO:38. SEQ ID NO:39 provides the sequence of this GH61g without the signal sequence.

(SEQ ID NO: 37)
CTGACGACGGGCGGCGTCAAGCACGCTGTGTACCAGTACATCCGCAAGAA
CACCAACTATAACTCGCCCGTGACCGATCTGACGTCCAACGACCTCCGCT
GCAATGTGGGTGCTACCGGTGCGGGCACCGATACCGTCACGGTGCGCGCC
GGCGATTCGTTCACCTTCACGACCGATACGCCCGTTTACCACCAGGGCCC
GACCTCGATCTACATGTCCAAGGCCCCCGGCAGCGCGTCCGACTACGACG
GCAGCGGCGGCTGGTTCAAGATCAAGGACTGGGGTGCCGACTTTAGCAGC
GGCCAGGCCACCTGGACCTTGGCGTCTGACTACACCGCCACGATTCCGGA
ATGTATTCCCCCCGGCGACTACCTGCTTCGCATCCAGCAACTCGGCATCC
ACAACCCTTGGCCCGCGGGCATCCCCCAGTTCTACATCTCTTGTGCCCAG
ATCACCGTGACTGGTGGCGGCAGTGCCAACCCCGGCCCGACCGTCTCCAT
CCCAGGCGCCTTCAAGGAGACCGACCCGGGCTACACTGTCAACATCTACA
ACAACTTCCACAACTACACCGTCCCTGGCCCAGCCGTCTTCACCTGCAAC
GGTAGCGGCGGCAACAACGGCGGCGGCTCCAACCCAGTCACCACCACCAC
CACCACCACCACCAGGCCGTCCACCAGCACCGCCCAGTCCCAGCCGTCGT
CGAGCCCGACCAGCCCCTCCAGCTGCACCGTCGCGAAGTGGGGCCAGTGC
GGAGGACAGGGTTACAGCGGCTGCACCGTGTGCGCGGCCGGGTCGACCTG
CCAGAAGACCAACGACTACTACAGCCAGTGCTTG
(SEQ ID NO: 38)
MKGLLGAAALSLAVSDVSAHYIFQQLTTGGVKHAVYQYIRKNTNYNSPVT
DLTSNDLRCNVGATGAGTDTVTVRAGDSFTFTTDTPVYHQGPTSIYMSKA
PGSASDYDGSGGWFKIKDWGADFSSGQATWTLASDYTATIPECIPPGDYL
LRIQQLGIHNPWPAGIPQFYISCAQITVTGGGSANPGPTVSIPGAFKETD
PGYTVNIYNNFHNYTVPGPAVFTCNGSGGNNGGGSNPVTTTTTTTTRPST
STAQSQPSSSPTSPSSCTVAKWGQCGGQGYSGCTVCAAGSTCQKTNDYYS
QCL
(SEQ ID NO: 39)
HYIFQQLTTGGVKHAVYQYIRKNTNYNSPVTDLTSNDLRCNVGATGAGTD
TVTVRAGDSFTFTTDTPVYHQGPTSIYMSKAPGSASDYDGSGGWFKIKDW
GADFSSGQATWTLASDYTATIPECIPPGDYLLRIQQLGIHNPWPAGIPQF
YISCAQITVTGGGSANPGPTVSIPGAFKETDPGYTVNIYNNFHNYTVPGP
AVFTCNGSGGNNGGGSNPVTTTTTTTTRPSTSTAQSQPSSSPTSPSSCTV
AKWGQCGGQGYSGCTVCAAGSTCQKTNDYYSQCL

The polynucleotide (SEQ ID NO:40) and amino acid (SEQ ID NO:41) sequences of an M. thermophile GH61h are provided below. The signal sequence is shown underlined in SEQ ID NO:41. SEQ ID NO:42 provides the sequence of this GH61h without the signal sequence.

(SEQ ID NO: 40)
ATGTCTTCCTTCACCTCCAAGGGTCTCCTTTCCGCCCTCATGGGCGCGGC
AACGGTTGCCGCCCACGGTCACGTCACCAACATCGTCATCAACGGCGTCT
CATACCAGAACTTCGACCCATTCACGCACCCTTATATGCAGAACCCTCCG
ACGGTTGTCGGCTGGACCGCGAGCAACACGGACAACGGCTTCGTCGGCCC
CGAGTCCTTCTCTAGCCCGGACATCATCTGCCACAAGTCCGCCACCAACG
CTGGCGGCCATGCCGTCGTCGCGGCCGGCGATAAGGTCTTCATCCAGTGG
GACACCTGGCCCGAGTCGCACCACGGTCCGGTCATCGACTATCTCGCCGA
CTGCGGCGACGCGGGCTGCGAGAAGGTCGACAAGACCACGCTCAAGTTCT
TCAAGATCAGCGAGTCCGGCCTGCTCGACGGCACTAACGCCCCCGGCAAG
TGGGCGTCCGACACGCTGATCGCCAACAACAACTCGTGGCTGGTCCAGAT
CCCGCCCAACATCGCCCCGGGCAACTACGTCCTGCGCCACGAGATCATCG
CCCTGCACAGCGCCGGCCAGCAGAACGGCGCCCAGAACTACCCTCAGTGC
TTCAACCTGCAGGTCACCGGCTCCGGCACTCAGAAGCCCTCCGGCGTCCT
CGGCACCGAGCTCTACAAGGCCACCGACGCCGGCATCCTGGCCAACATCT
ACACCTCGCCCGTCACCTACCAGATCCCCGGCCCGGCCATCATCTCGGGC
GCCTCCGCCGTCCAGCAGACCACCTCGGCCATCACCGCCTCTGCTAGCGC
CATCACCGGCTCCGCTACCGCCGCGCCCACGGCTGCCACCACCACCGCCG
CCGCCGCCGCCACCACTACCACCACCGCTGGCTCCGGTGCTACCGCCACG
CCCTCGACCGGCGGCTCTCCTTCTTCCGCCCAGCCTGCTCCTACCACCGC
TGCCGCTACCTCCAGCCCTGCTCGCCCGACCCGCTGCGCTGGTCTGAAGA
AGCGCCGTCGCCACGCCCGTGACGTCAAGGTTGCCCTC
(SEQ ID NO: 41)
MSSFTSKGLLSALMGAATVAAHGHVTNIVINGVSYQNFDPFTHPYMQNPP
TVVGWTASNTDNGFVGPESFSSPDIICHKSATNAGGHAVVAAGDKVFIQW
DTWPESHHGPVIDYLADCGDAGCEKVDKTTLKFFKISESGLLDGTNAPGK
WASDTLIANNNSWLVQIPPNIAPGNYVLRHEIIALHSAGQQNGAQNYPQC
FNLQVTGSGTQKPSGVLGTELYKATDAGILANIYTSPVTYQIPGPAIISG
ASAVQQTTSAITASASAITGSATAAPTAATTTAAAAATTTTTAGSGATAT
YSTGGSPSSAQPAPTTAAATSSPARPTRCAGLKKRRRHARDVKVAL
(SEQ ID NO: 42)
AHGHVTNIVINGVSYQNFDPFTHPYMQNPPTVVGWTASNTDNGFVGPESF
SSPDIICHKSATNAGGHAVVAAGDKVFIQWDTWPESHHGPVIDYLADCGD
AGCEKVDKTTLKFFKISESGLLDGTNAPGKWASDTLIANNNSWLVQIPPN
IAPGNYVLRHEIIALHSAGQQNGAQNYPQCFNLQVTGSGTQKPSGVLGTE
LYKATDAGILANIYTSPVTYQTPGPAIISGASAVQQTTSAITASASAITG
SATAAPTAATTTAAAAATTTTTAGSGATATPSTGGSPSSAQPAPTTAAAT
SSPARPTRCAGLKKRRRHARDVKVAL

The polynucleotide (SEQ ID NO:43) and amino acid (SEQ ID NO:44) sequences of an M. thermophila GH61i are provided below. The signal sequence is shown underlined in SEQ ID NO:44. SEQ ID NO:45 provides the sequence of this GH61i without the signal sequence.

(SEQ ID NO: 43)
ATGAAGACGCTCGCCGCCCTCGTGGTCTCGGCCGCCCTCGTGGCCGCGCA
CGGCTATGTTGACCACGCCACGATCGGTGGCAAGGATTATCAGTTCTACC
AGCCGTACCAGGACCCTTACATGGGCGACAACAAGCCCGATAGGGTTTCC
CGCTCCATCCCGGGCAACGGCCCCGTGGAGGACGTCAACTCCATCGACCT
CCAGTGCCACGCCGGTGCCGAACCGGCCAAGCTCCACGCCCCCGCCGCCG
CCGGCTCGACCGTGACGCTCTACTGGACCCTCTGGCCCGACTCCCACGTC
GGCCCCGTCATCACCTACATGGCTCGCTGCCCCGACACCGGCTGCCAGGA
CTGGTCCCCGGGAACTAAGCCCGTTTGGTTCAAGATCAAGGAAGGCGGCC
GTGAGGGCACCTCCAATACCCCGCTCATGACGGCCCCCTCCGCCTACACC
TACACGATCCCGTCCTGCCTCAAGAGCGGCTACTACCTCGTCCGCCACGA
GATCATCGCCCTGCACTCGGCCTGGCAGTACCCCGGCGCCCAGTTCTACC
CGGGCTGCCACCAGCTCCAGGTCACCGGCGGCGGCTCCACCGTGCCCTCT
ACCAACCTGGTCTCCTTCCCCGGCGCCTACAAGGGGAGCGACCCCGGCAT
CACCTACGACGCTTACAAGGCGCAACCTTACACCATCCCTGGCCCGGCCG
TGTTTACCTGCTGA
(SEQ ID NO: 44)
MKTLAALVVSAALVAAHGYVDHATIGGKDYQFYQPYQDPYMGDNKPDRVS
RSIPGNGPVEDVNSIDLQCHAGAEPAKLHAPAAAGSTVTLYWTLWPDSHV
GPVITYMARCPDTGCQDWSPGTKPVWFKIKEGGREGTSNTPLMTAPSAYT
YTIPSCLKSGYYLVRHEIIALHSAWQYPGAQFYPGCHQLQVTGGGSTVPS
TNLVSFPGAYKGSDPGITYDAYKAQPYTIPGPAVFTC
(SEQ ID NO: 45)
YVDHATIGGKDYQFYQPYQDPYMGDNKPDRVSRSIPGNGPVEDVNSIDLQ
CHAGAEPAKLHAPAAAGSTVTLYWTLWPDSHVGPVITYMARCPDTGCQDW
SPGTKPVWFKIKEGGREGTSNTPLMTAPSAYTYTIPSCLKSGYYLVRHEI
IALHSAWQYPGAQFYPGCHQLQVTGGGSTVPSTNLVSFPGAYKGSDPGIT
YDAYKAQPYTIPGPAVFTC

The polynucleotide (SEQ ID NO:46) and amino acid (SEQ ID NO:47) sequences of an alternative M. thermophila GH61i are provided below. The signal sequence is shown underlined in SEQ ID NO:47. SEQ ID NO:48 provides the sequence of this GH61i without the signal sequence.

(SEQ ID NO: 46)
ATGAAGACGCTCGCCGCCCTCGTGGTCTCGGCCGCCCTCGTGGCCGCGCA
CGGCTATGTTGACCACGCCACGATCGGTGGCAAGGATTATCAGTTCTACC
AGCCGTACCAGGACCCTTACATGGGCGACAACAAGCCCGATAGGGTTTCC
CGCTCCATCCCGGGCAACGGCCCCGTGGAGGACGTCAACTCCATCGACCT
CCAGTGCCACGCCGGTGCCGAACCGGCCAAGCTCCACGCCCCCGCCGCCG
CCGGCTCGACCGTGACGCTCTACTGGACCCTCTGGCCCGACTCCCACGTC
GGCCCCGTCATCACCTACATGGCTCGCTGCCCCGACACCGGCTGCCAGGA
CTGGTCCCCGGGAACTAAGCCCGTTTGGTTCAAGATCAAGGAAGGCGGCC
GTGAGGGCACCTCCAATGTCTGGGCTGCTACCCCGCTCATGACGGCCCCC
TCCGCCTACACCTACACGATCCCGTCCTGCCTCAAGAGCGGCTACTACCT
CGTCCGCCACGAGATCATCGCCCTGCACTCGGCCTGGCAGTACCCCGGCG
CCCAGTTCTACCCGGGCTGCCACCAGCTCCAGGTCACCGGCGGCGGCTCC
ACCGTGCCCTCTACCAACCTGGTCTCCTTCCCCGGCGCCTACAAGGGGAG
CGACCCCGGCATCACCTACGACGCTTACAAGGCGCAACCTTACACCATCC
CTGGCCCGGCCGTGTTTACCTGC
(SEQ ID NO: 47)
MKTLAALVVSAALVAAHGYVDHATIGGKDYQFYQPYQDPYMGDNKPDRVS
RSIPGNGPVEDVNSIDLQCHAGAEPAKLHAPAAAGSTVTLYWTLWPDSHV
GPVITYMARCPDTGCQDWSPGTKPVWFKIKEGGREGTSNVWAATPLMTAP
SAYTYTIPSCLKSGYYLVRHEIIALHSAWQYPGAQFYPGCHQLQVTGGGS
TVPSTNLVSFPGAYKGSDPGITYDAYKAQPYTIPGPAVFTC
(SEQ ID NO: 48)
YVDHATIGGKDYQFYQPYQDPYMGDNKPDRVSRSIPGNGPVEDVNSIDLQ
CHAGAEPAKLHAPAAAGSTVTLYWTLWPDSHVGPVITYMARCPDTGCQDW
SPGTKPVWFKIKEGGREGTSNVWAATPLMTAPSAYTYTIPSCLKSGYYLV
RHEIIALHSAWQYPGAQFYPGCHQLQVTGGGSTVPSTNLVSFPGAYKGSD
PGITYDAYKAQPYTIPGPAVFTC

The polynucleotide (SEQ ID NO:49) and amino acid (SEQ ID NO:50) sequences of an M. thermophila GH61j are provided below. The signal sequence is shown underlined in SEQ ID NO:50. SEQ ID NO:51 provides the sequence of this GH61 j without the signal sequence.

(SEQ ID NO: 49)
ATGAGATACTTCCTCCAGCTCGCTGCGGCCGCGGCCTTTGCCGTGAACAG
CGCGGCGGGTCACTACATCTTCCAGCAGTTCGCGACGGGCGGGTCCAAGT
ACCCGCCCTGGAAGTACATCCGGCGCAACACCAACCCGGACTGGCTGCAG
AACGGGCCGGTGACGGACCTGTCGTCGACCGACCTGCGCTGCAACGTGGG
CGGGCAGGTCAGCAACGGGACCGAGACCATCACCTTGAACGCCGGCGACG
AGTTCAGCTTCATCCTCGACACGCCCGTCTACCATGCCGGCCCCACCTCG
CTCTACATGTCCAAGGCGCCCGGAGCTGTGGCCGACTACGACGGCGGCGG
GGCCTGGTTCAAGATCTACGACTGGGGTCCGTCGGGGACGAGCTGGACGT
TGAGTGGCACGTACACTCAGAGAATTCCCAAGTGCATCCCTGACGGCGAG
TACCTCCTCCGCATCCAGCAGATCGGGCTCCACAACCCCGGCGCCGCGCC
ACAGTTCTACATCAGCTGCGCTCAAGTCAAGGTCGTCGATGGCGGCAGCA
CCAATCCGACCCCGACCGCCCAGATTCCGGGAGCCTTCCACAGCAACGAC
CCTGGCTTGACTGTCAATATCTACAACGACCCTCTCACCAACTACGTCGT
CCCGGGACCTAGAGTTTCGCACTGG
(SEQ ID NO: 50)
MRYFLQLAAAAAFAVNSAAGHYIFQQFATGGSKYPPWKYIRRNTNPDWLQ
NGPVTDLSSTDLRCNVGGQVSNGTETITLNAGDEFSFILDTPVYHAGPTS
LYMSKAPGAVADYDGGGAWFKIYDWGPSGTSWTLSGTYTQRIPKCIPDGE
YLLRIQQIGLHNPGAAPQFYISCAQVKVVDGGSTNPTPTAQIPGAFHSND
PGLTVNIYNDPLTNYVVPGPRVSHW
(SEQ ID NO: 51)
HYIFQQFATGGSKYPPWKYIRRNTNPDWLQNGPVTDLSSTDLRCNVGGQV
SNGTETITLNAGDEFSFILDTPVYHAGPTSIYMSKAPGAVADYDGGGAWF
KIYDWGPSGTSWTLSGTYTQRIPKCIPDGEYLLRIQQIGLHNPGAAPQFY
ISCAQVKVVDGGSTNPTPTAQIPGAFHSNDPGLTVNIYNDPLTNYVVPGP
RVSHW

The polynucleotide (SEQ ID NO:52) and amino acid (SEQ ID NO:53) sequences of an M. thermophila GH61k are provided below. The signal sequence is shown underlined in SEQ ID NO:53. SEQ ID NO:54 provides the sequence of this GH61k without the signal sequence.

(SEQ ID NO: 52)
ATGCACCCCTCCCTTCTTTTCACGCTTGGGCTGGCGAGCGTGCTTGTCCC
CCTCTCGTCTGCACACACTACCTTCACGACCCTCTTCGTCAACGATGTCA
ACCAAGGTGATGGTACCTGCATTCGCATGGCGAAGAAGGGCAATGTCGCC
ACCCATCCTCTCGCAGGCGGTCTCGACTCCGAAGACATGGCCTGTGGTCG
GGATGGTCAAGAACCCGTGGCATTTACGTGTCCGGCCCCAGCTGGTGCCA
AGTTGACTCTCGAGTTTCGCATGTGGGCCGATGCTTCGCAGTCCGGATCG
ATCGATCCATCCCACCTTGGCGTCATGGCCATCTACCTCAAGAAGGTTTC
CGACATGAAATCTGACGCGGCCGCTGGCCCGGGCTGGTTCAAGATTTGGG
ACCAAGGCTACGACTTGGCGGCCAAGAAGTGGGCCACCGAGAAGCTCATC
GACAACAACGGCCTCCTGAGCGTCAACCTTCCAACCGGCTTACCAACCGG
CTACTACCTCGCCCGCCAGGAGATCATCACGCTCCAAAACGTTACCAATG
ACAGGCCAGAGCCCCAGTTCTACGTCGGCTGCGCACAGCTCTACGTCGAG
GGCACCTCGGACTCACCCATCCCCTCGGACAAGACGGTCTCCATTCCCGG
CCACATCAGCGACCCGGCCGACCCGGGCCTGACCTTCAACGTCTACACGG
GCGACGCATCCACCTACAAGCCGCCCGGCCCCGAGGTTTACTTCCCCACC
ACCACCACCACCACCTCCTCCTCCTCCTCCGGAAGCAGCGACAACAAGGG
AGCCAGGCGCCAGCAAACCCCCGACGACAAGCAGGCCGACGGCCTCGTTC
CAGCCGACTGCCTCGTCAAGAACGCGAACTGGTGCGCCGCTGCCCTGCCG
CCGTACACCGACGAGGCCGGCTGCTGGGCCGCCGCCGAGGACTGCAACAA
GCAGCTGGACGCGTGCTACACCAGCGCACCCCCCTCGGGCAGCAAGGGGT
GCAAGGTCTGGGAGGAGCAGGTGTGCACCGTCGTCTCGCAGAAGTGCGAG
GCCGGGGATTTCAAGGGGCCCCCGCAGCTCGGGAAGGAGCTCGGCGAGGG
GATCGATGAGCCTATTCCGGGGGGAAAGCTGCCCCCGGCGGTCAACGCGG
GAGAGAACGGGAATCATGGCGGAGGTGGTGGTGATGATGGTGATGATGAT
AATGATGAGGCCGGGGCTGGGGCAGCGTCGACTCCGACTTTTGCTGCTCC
TGGTGCGGCCAAGACTCCCCAACCAAACTCCGAGAGGGCCCGGCGCCGTG
AGGCGCATTGGCGGCGACTGGAATCTGCTGAG
(SEQ ID NO: 53)
MHPSLLFTLGLASVLVPLSSAHTTFTTLFVNDVNQGDGTCIRMAKKGNVA
THPLAGGLDSEDMACGRDGQEPVAFTCPAPAGAKLTLEFRMWADASQSGS
IDPSHLGVMAIYLKKVSDMKSDAAAGPGWFKIWDQGYDLAAKKWATEKLI
DNNGLLSVNLPTGLPTGYYLARQEIITLQNVTNDRPEPQFYVGCAQLYVE
GTSDSPIPSDKTVSIPGHISDPADPGLTFNVYTGDASTYKPPGPEVYFPT
TTTTTSSSSSGSSDNKGARRQQTPDDKQADGLVPADCLVKNANWCAAALP
PYTDEAGCWAAAEDCNKQLDACYTSAPPSGSKGCKVWEEQVCTVVSQKCE
AGDFKGPPQLGKELGEGIDEPIPGGKLPPAVNAGENGNHGGGGGDDGDDD
NDEAGAGAASTPTFAAPGAAKTPQPNSERARRREAHWRRLESAE
(SEQ ID NO: 54)
HTTFTTLFVNDVNQGDGTCIRMAKKGNVATHPLAGGLDSEDMACGRDGQE
PVAFTCPAPAGAKLTLEFRMWADASQSGSIDPSHLGVMAIYLKKVSDMKS
DAAAGPGWFKIWDQGYDLAAKKWATEKLIDNNGLLSVNLPTGLPTGYYLA
RQEIITLQNVTNDRPEPQFYVGCAQLYVEGTSDSPIPSDKTVSIPGHISD
PADPGLTFNVYTGDASTYKPPGPEVYFPTTTTTTSSSSSGSSDNKGARRQ
QTPDDKQADGLVPADCLVKNANWCAAALPPYTDEAGCWAAAEDCNKQLDA
CYTSAPPSGSKGCKVWEEQVCTVVSQKCEAGDFKGPPQLGKELGEGIDEP
IPGGKLPPAVNAGENGNHGGGGGDDGDDDNDEAGAGAASTPTFAAPGAAK
TPQPNSERARRREAHWRRLESAE

The polynucleotide (SEQ ID NO:55) and amino acid (SEQ ID NO:56) sequences of a M. thermophila GH61l are provided below. The signal sequence is shown underlined in SEQ ID NO:56. SEQ ID NO:57 provides the sequence of this GH61l without the signal sequence.

(SEQ ID NO: 55)
ATGTTTTCTCTCAAGTTCTTTATCTTGGCCGGTGGGCTTGCTGTCCTCAC
CGAGGCTCACATAAGACTAGTGTCGCCCGCCCCTTTTACCAACCCTGACC
AGGGCCCCAGCCCACTCCTAGAGGCTGGCAGCGACTATCCCTGCCACAAC
GGCAATGGGGGCGGTTATCAGGGAACGCCAACCCAGATGGCAAAGGGTTC
TAAGCAGCAGCTAGCCTTCCAGGGGTCTGCCGTTCATGGGGGTGGCTCCT
GCCAAGTGTCCATCACCTACGACGAAAACCCGACCGCTCAGAGCTCCTTC
AAGGTCATTCACTCGATTCAAGGTGGCTGCCCCGCCAGGGCCGAGACGAT
CCCGGATTGCAGCGCACAAAATATCAACGCCTGCAATATAAAGCCCGATA
ATGCCCAGATGGACACCCCGGATAAGTATGAGTTCACGATCCCGGAGGAT
CTCCCCAGTGGCAAGGCCACCCTCGCCTGGACATGGATCAACACTATCGG
CAACCGCGAGTTTTATATGGCATGCGCCCCGGTTGAGATCACCGGCGACG
GCGGTAGCGAGTCGGCTCTGGCTGCGCTGCCCGACATGGTCATTGCCAAC
ATCCCGTCCATCGGAGGAACCTGCGCGACCGAGGAGGGGAAGTACTACGA
ATATCCCAACCCCGGTAAGTCGGTCGAAACCATCCCGGGCTGGACCGATT
TGGTTCCCCTGCAAGGCGAATGCGGTGCTGCCTCCGGTGTCTCGGGCTCC
GGCGGAAACGCCAGCAGTGCTACCCCTGCCGCAGGGGCCGCCCCGACTCC
TGCTGTCCGCGGCCGCCGTCCCACCTGGAACGCC
(SEQ ID NO: 56)
MFSLKFFILAGGLAVLTEAHIRLVSPAPFTNPDQGPSPLLEAGSDYPCHN
GNGGGYQGTPTQMAKGSKQQLAFQGSAVHGGGSCQVSITYDENPTAQSSF
KVIHSIQGGCPARAETIPDCSAQNINACNIKPDNAQMDTPDKYEFTIPED
LPSGKATLAWTWINTIGNREFYMACAPVEITGDGGSESALAALPDMVIAN
IPSIGGTCATEEGKYYEYPNPGKSVETIPGWTDLVPLQGECGAASGVSGS
GGNASSATPAAGAAPTPAVRGRRPTWNA
(SEQ ID NO: 57)
HIRLVSPAPFTNPDQGPSPLLEAGSDYPCHNGNGGGYQGTPTQMAKGSKQ
QLAFQGSAVHGGGSCQVSITYDENPTAQSSFKVIHSIQGGCPARAETIPD
CSAQNINACNIKPDNAQMDTPDKYEFTIPEDLPSGKATLAWTWINTIGNR
EFYMACAPVEITGDGGSESALAALPDMVIANIPSIGGTCATEEGKYYEYP
NPGKSVETIPGWTDLVPLQGECGAASGVSGSGGNASSATPAAGAAPTPAV
RGRRPTWNA

The polynucleotide (SEQ ID NO:58) and amino acid (SEQ ID NO:59) sequences of a M. thermophila GH61m are provided below. The signal sequence is shown underlined in SEQ ID NO:59. SEQ ID NO:60 provides the sequence of this GH61m without the signal sequence.

(SEQ ID NO: 58)
ATGAAGCTCGCCACGCTCCTCGCCGCCCTCACCCTCGGGGTGGCCGACCA
GCTCAGCGTCGGGTCCAGAAAGTTTGGCGTGTACGAGCACATTCGCAAGA
ACACGAACTACAACTCGCCCGTTACCGACCTGTCGGACACCAACCTGCGC
TGCAACGTCGGCGGGGGCTCGGGCACCAGCACCACCGTGCTCGACGTCAA
GGCCGGAGACTCGTTCACCTTCTTCAGCGACGTTGCCGTCTACCACCAGG
GGCCCATCTCGCTGTGCGTGGACCGGACCAGTGCAGAGAGCATGGATGGA
CGGGAACCGGACATGCGCTGCCGAACTGGCTCACAAGCTGGCTACCTGGC
GGTGACTGACTACGACGGGTCCGGTGACTGTTTCAAGATCTATGACTGGG
GACCGACGTTCAACGGGGGCCAGGCGTCGTGGCCGACGAGGAATTCGTAC
GAGTACAGCATCCTCAAGTGCATCAGGGACGGCGAATACCTACTGCGGAT
TCAGTCCCTGGCCATCCATAACCCAGGTGCCCTTCCGCAGTTCTACATCA
GCTGCGCCCAGGTGAATGTGACGGGCGGAGGCACCGTCACCCCGAGATCA
AGGCGACCGATCCTGATCTATTTCAACTTCCACTCGTATATCGTCCCTGG
GCCGGCAGTGTTCAAGTGCTAG
(SEQ ID NO: 59)
MKLATLLAALTLGVADQLSVGSRKFGVYEHIRKNTNYNSPVTDLSDTNLR
CNVGGGSGTSTTVLDVKAGDSFTFFSDVAVYHQGPISLCVDRTSAESMDG
REPDMRCRTGSQAGYLAVTDYDGSGDCFKIYDWGPTFNGGQASWPTRNSY
EYSILKCIRDGEYLLRIQSLAIHNPGALPQFYISCAQVNVTGGGTVTPRS
RRPILIYFNFHSYIVPGPAVFKC
(SEQ ID NO: 60)
DQLSVGSRKFGVYEHIRKNTNYNSPVTDLSDTNLRCNVGGGSGTSTTVLD
VKAGDSFTFFSDVAVYHQGPISLCVDRTSAESMDGREPDMRCRTGSQAGY
LAVTDYDGSGDCFKIYDWGPTFNGGQASWPTRNSYEYSILKCIRDGEYLL
RIQSLAIHNPGALPQFYISCAQVNVTGGGTVTPRSRRPILIYFNFHSYIV
PGPAVFKC

The polynucleotide (SEQ ID NO:61) and amino acid (SEQ ID NO:62) sequences of an alternative M. thermophila GH61m are provided below. The signal sequence is shown underlined in SEQ ID NO:62. SEQ ID NO:63 provides the sequence of this GH61m without the signal sequence.

(SEQ ID NO: 61)
ATGAAGCTCGCCACGCTCCTCGCCGCCCTCACCCTCGGGCTCAGCGTCGG
GTCCAGAAAGTTTGGCGTGTACGAGCACATTCGCAAGAACACGAACTACA
ACTCGCCCGTTACCGACCTGTCGGACACCAACCTGCGCTGCAACGTCGGC
GGGGGCTCGGGCACCAGCACCACCGTGCTCGACGTCAAGGCCGGAGACTC
GTTCACCTTCTTCAGCGACGTTGCCGTCTACCACCAGGGGCCCATCTCGC
TGTGCGTGGACCGGACCAGTGCAGAGAGCATGGATGGACGGGAACCGGAC
ATGCGCTGCCGAACTGGCTCACAAGCTGGCTACCTGGCGGTGACTGTGAT
GACTGTGACTGACTACGACGGGTCCGGTGACTGTTTCAAGATCTATGACT
GGGGACCGACGTTCAACGGGGGCCAGGCGTCGTGGCCGACGAGGAATTCG
TACGAGTACAGCATCCTCAAGTGCATCAGGGACGGCGAATACCTACTGCG
GATTCAGTCCCTGGCCATCCATAACCCAGGTGCCCTTCCGCAGTTCTACA
TCAGCTGCGCCCAGGTGAATGTGACGGGCGGAGGCACCATCTATTTCAAC
TTCCACTCGTATATCGTCCCTGGGCCGGCAGTGTTCAAGTGC
(SEQ ID NO: 62)
MKLATLLAALTLGLSVGSRKFGVYEHIRKNTNYNSPVTDLSDTNLRCNVG
GGSGTSTTVLDVKAGDSFTFFSDVAVYHQGPISLCVDRTSAESMDGREPD
MRCRTGSQAGYLAVTVMTVTDYDGSGDCFKIYDWGPTFNGGQASWPTRNS
YEYSILKCIRDGEYLLRIQSLAIHNPGALPQFYISCAQVNVTGGGTIYFN
FHSYIVPGPAVFKC
(SEQ ID NO: 63)
RKFGVYEHIRKNTNYNSPVTDLSDTNLRCNVGGGSGTSTTVLDVKAGDSF
TFFSDVAVYHQGPISLCVDRTSAESMDGREPDMRCRTGSQAGYLAVTVMT
VTDYDGSGDCFKIYDWGPTFNGGQASWPTRNSYEYSILKCIRDGEYLLRI
QSLAIHNPGALPQFYISCAQVNVTGGGTIYFNFHSYTVPGPAVFKC

The polynucleotide (SEQ ID NO:64) and amino acid (SEQ ID NO:65) sequences of a M. thermophila GH61n are provided below.

(SEQ ID NO: 64)
ATGACCAAGAATGCGCAGAGCAAGCAGGGCGTTGAGAACCCAACAAGCGG
CGACATCCGCTGCTACACCTCGCAGACGGCGGCCAACGTCGTGACCGTGC
CGGCCGGCTCGACCATTCACTACATCTCGACCCAGCAGATCAACCACCCC
GGCCCGACTCAGTACTACCTGGCCAAGGTACCCCCCGGCTCGTCGGCCAA
GACCTTTGACGGGTCCGGCGCCGTCTGGTTCAAGATCTCGACCACGATGC
CTACCGTGGACAGCAACAAGCAGATGTTCTGGCCAGGGCAGAACACTTAT
GAGACCTCAAACACCACCATTCCCGCCAACACCCCGGACGGCGAGTACCT
CCTTCGCGTCAAGCAGATCGCCCTCCACATGGCGTCTCAGCCCAACAAGG
TCCAGTTCTACCTCGCCTGCACCCAGATCAAGATCACCGGTGGTCGCAAC
GGCACCCCCAGCCCGCTGGTCGCGCTGCCCGGAGCCTACAAGAGCACCGA
CCCCGGCATCCTGGTCGACATCTACTCCATGAAGCCCGAATCGTACCAGC
CTCCCGGGCCGCCCGTCTGGCGCGGCTAA
(SEQ ID NO: 65)
MTKNAQSKQGVENPTSGD1RCYTSQTAANVVTVPAGSTIHYISTQQINHP
GPTQYYLAKVPPGSSAKTFDGSGAVWFKISTTMPTVDSNKQMFWPGQNTY
ETSNTTIPANTPDGEYLLRVKQIALHMASQPNKVQFYLACTQIKITGGRN
GTPSPLVALPGAYKSTDPGILVDTYSMKPESYQPPGPPVWRG

The polynucleotide (SEQ ID NO:66) and amino acid (SEQ ID NO:67) sequences of an alternative M. thermophila GH61n are provided below. The signal sequence is shown underlined in SEQ ID NO:67. SEQ ID NO:68 provides the sequence of this GH61n without the signal sequence.

(SEQ ID NO: 66)
ATGAGGCTTCTCGCAAGCTTGTTGCTCGCAGCTACGGCTGTTCAAGCTCA
CTTTGTTAACGGACAGCCCGAAGAGAGTGACTGGTCAGCCACGCGCATGA
CCAAGAATGCGCAGAGCAAGCAGGGCGTTGAGAACCCAACAAGCGGCGAC
ATCCGCTGCTACACCTCGCAGACGGCGGCCAACGTCGTGACCGTGCCGGC
CGGCTCGACCATTCACTACATCTCGACCCAGCAGATCAACCACCCCGGCC
CGACTCAGTACTACCTGGCCAAGGTACCCCCCGGCTCGTCGGCCAAGACC
TTTGACGGGTCCGGCGCCGTCTGGTTCAAGATCTCGACCACGATGCCTAC
CGTGGACAGCAACAAGCAGATGTTCTGGCCAGGGCAGAACACTTATGAGA
CCTCAAACACCACCATTCCCGCCAACACCCCGGACGGCGAGTACCTCCTT
CGCGTCAAGCAGATCGCCCTCCACATGGCGTCTCAGCCCAACAAGGTCCA
GTTCTACCTCGCCTGCACCCAGATCAAGATCACCGGTGGTCGCAACGGCA
CCCCCAGCCCGCTGGTCGCGCTGCCCGGAGCCTACAAGAGCACCGACCCC
GGCATCCTGGTCGACATCTACTCCATGAAGCCCGAATCGTACCAGCCTCC
CGGGCCGCCCGTCTGGCGCGGC
(SEQ ID NO: 67)
MRLLASLLLAATAVQAHFVNGQPEESDWSATRMTKNAQSKQGVENPTSGD
IRCYTSQTAANVVTVPAGSTIHYISTQQINHPGPTQYYLAKVPPGSSAKT
FDGSGAVWFKISTTMPTVDSNKQMFWPGQNTYETSNTTIPANTPDGEYLL
RVKQIALHMASQPNKVQFYLACTQIKITGGRNGTPSPLVALPGAYKSTDP
GILVDIYSMKPESYQPPGPPVWRG
(SEQ ID NO: 68)
HFVNGQPEESDWSATRMTKNAQSKQGVENPTSGDIRCYTSQTAANVVTVP
AGSTIHYISTQQINHPGPTQYYLAKVPPGSSAKTFDGSGAVWFKISTTMP
TVDSNKQMFWPGQNTYETSNTTIPANTPDGEYLLRVKQIALHMASQPNKV
QFYLACTQIKITGGRNGTPSPLVALPGAYKSTDPGILVDIYSMKPESYQP
PGPPVWRG

The polynucleotide (SEQ ID NO:69) and amino acid (SEQ ID NO:70) sequences of an alternative M. thermophila GH61o are provided below. The signal sequence is shown underlined in SEQ ID NO:70. SEQ ID NO:71 provides the sequence of this GH61o without the signal sequence.

(SEQ ID NO: 69)
ATGAAGCCCTTTAGCCTCGTCGCCCTGGCGACTGCCGTGAGCGGCCATGC
CATCTTCCAGCGGGTGTCGGTCAACGGGCAGGACCAGGGCCAGCTCAAGG
GGGTGCGGGCGCCGTCGAGCAACTCCCCGATCCAGAACGTCAACGATGCC
AACATGGCCTGCAACGCCAACATTGTGTACCACGACAACACCATCATCAA
GGTGCCCGCGGGAGCCCGCGTCGGCGCGTGGTGGCAGCACGTCATCGGCG
GGCCGCAGGGCGCCAACGACCCGGACAACCCGATCGCCGCCTCCCACAAG
GGCCCCATCCAGGTCTACCTGGCCAAGGTGGACAACGCGGCGACGGCGTC
GCCGTCGGGCCTCAAGTGGTTCAAGGTGGCCGAGCGCGGCCTGAACAACG
GCGTGTGGGCCTACCTGATGCGCGTCGAGCTGCTCGCCCTGCACAGCGCC
TCGAGCCCCGGCGGCGCCCAGTTCTACATGGGCTGTGCACAGATCGAAGT
CACTGGCTCCGGCACCAACTCGGGCTCCGACTTTGTCTCGTTCCCCGGCG
CCTACTCGGCCAACGACCCGGGCATCTTGCTGAGCATCTACGACAGCTCG
GGCAAGCCCAACAATGGCGGGCGCTCGTACCCGATCCCCGGCCCGCGCCC
CATCTCCTGCTCCGGCAGCGGCGGCGGCGGCAACAACGGCGGCGACGGCG
GCGACGACAACAACGGTGGTGGCAACAACAACGGCGGCGGCAGCGTCCCC
CTGTACGGGCAGTGCGGCGGCATCGGCTACACGGGCCCGACCACCTGTGC
CCAGGGAACTTGCAAGGTGTCGAACGAATACTACAGCCAGTGCCTCCCC
(SEQ ID NO: 70)
MKPFSLVALATAVSGHAIFQRVSVNGQDQGQLKGVRAPSSNSPIQNVNDA
NMACNANIVYHDNTIIKVPAGARVGAWWQHVIGGPQGANDPDNPIAASHK
GPIQVYLAKVDNAATASPSGLKWFKVAERGLNNGVWAYLMRVELLALHSA
SSPGGAQFYMGCAQIEVTGSGTNSGSDFVSFPGAYSANDPGILLSIYDSS
GKPNNGGRSYPIPGPRPISCSGSGGGGNNGGDGGDDNNGGGNNNGGGSVP
LYGQCGGIGYTGPTTCAQGTCKVSNEYYSQCLP
(SEQ ID NO: 71)
HAIFQRVSVNGQDQGQLKGVRAPSSNSPIQNVNDANMACNANIVYHDNTI
IKVPAGARVGAWWQHVIGGPQGANDPDNPIAASHKGPIQVYLAKVDNAAT
ASPSGLKWFKVAERGLNNGVWAYLMRVELLALHSASSPGGAQFYMGCAQI
EVTGSGTNSGSDFVSFPGAYSANDPGILLSIYDSSGKPNNGGRSYPIPGP
RPISCSGSGGGGNNGGDGGDDNNGGGNNNGGGSVPLYGQCGGIGYTGPTT
CAQGTCKVSNEYYSQCLP

The polynucleotide (SEQ ID NO:72) and amino acid (SEQ ID NO:73) sequences of a M. thermophila GH61p are provided below. The signal sequence is shown underlined in SEQ ID NO:73. SEQ ID NO:74 provides the sequence of this GH61p without the signal sequence.

(SEQ ID NO: 72)
ATGAAGCTCACCTCGTCCCTCGCTGTCCTGGCCGCTGCCGGCGCCCAGGC
TCACTATACCTTCCCTAGGGCCGGCACTGGTGGTTCGCTCTCTGGCGAGT
GGGAGGTGGTCCGCATGACCGAGAACCATTACTCGCACGGCCCGGTCACC
GATGTCACCAGCCCCGAGATGACCTGCTATCAGTCCGGCGTGCAGGGTGC
GCCCCAGACCGTCCAGGTCAAGGCGGGCTCCCAATTCACCTTCAGCGTGG
ATCCCTCCATCGGCCACCCCGGCCCTCTCCAGTTCTACATGGCTAAGGTG
CCGTCGGGCCAGACGGCCGCCACCTTTGACGGCACGGGAGCCGTGTGGTT
CAAGATCTACCAAGACGGCCCGAACGGCCTCGGCACCGACAGCATTACCT
GGCCCAGCGCCGGCAAAACCGAGGTCTCGGTCACCATCCCCAGCTGCATC
GAGGATGGCGAGTACCTGCTCCGGGTCGAGCACACCCCCCTCCCTACAGC
GCCAGCAGCGCAAAACCGAGCTCGCTCGTCACCATCCCCAGCTGCATACA
AGGCCACCGACCCGGGCATCCTCTTCCAGCTCTACTGGCCCATCCCGACC
GAGTACATCAACCCCGGCCCGGCCCCCGTCTCTTGCTAA
(SEQ ID NO: 73)
MKLTSSLAVLAAAGAQAHYTFPRAGTGGSLSGEWEVVRMTENHYSHGPVT
DVTSPEMTCYQSGVQGAPQTVQVKAGSQFTFSVDPSIGHPGPLQFYMAKV
PSGQTAATFDGTGAVWFKIYQDGPNGLGTDSITWPSAGKTEVSVTIPSCI
EDGEYLLRVEHTPLPTAPAAQNRARSSPSPAAYKATDPGILFQLYWPIPT
EYINPGPAPVSC
(SEQ ID NO: 74)
HYTFPRAGTGGSLSGEWEVVRMTENHYSHGPVTDVTSPEMTCYQSGVQGA
PQTVQVKAGSQFTFSVDPSIGHPGPLQFYMAKVPSGQTAATFDGTGAVWF
KIYQDGPNGLGTDSITWPSAGKTEVSVTIPSCIEDGEYLLRVEHTPLPTA
PAAQNRARSSPSPAAYKATDPGILFQLYWPIPTEYINPGPAPVSC

The polynucleotide (SEQ ID NO:75) and amino acid (SEQ ID NO:76) sequences of an alternative M. thermophila GH61p are provided below. The signal sequence is shown underlined in SEQ ID NO:76. SEQ ID NO:77 provides the sequence of this GH61p without the signal sequence.

(SEQ ID NO: 75)
ATGAAGCTCACCTCGTCCCTCGCTGTCCTGGCCGCTGCCGGCGCCCAGGC
TCACTATACCTTCCCTAGGGCCGGCACTGGTGGTTCGCTCTCTGGCGAGT
GGGAGGTGGTCCGCATGACCGAGACCATTACTCGCACGGCCCGGTCACCG
ATGTCACCAGCCCCGAGATGACCTGCTATCAGTCCGGCGTGCAGGGTGCG
CCCCAGACCGTCCAGGTCAAGGCGGGCTCCCAATTCACCTTCAGCGTGGA
TCCCTCCATCGGCCACCCCGGCCCTCTCCAGTTCTACATGGCTAAGGTGC
CGTCGGGCCAGACGGCCGCCACCTTTGACGGCACGGGAGCCGTGTGGTTC
AAGATCTACCAAGACGGCCCGAACGGCCTCGGCACCGACAGCATTACCTG
GCCCAGCGCCGGCAAAACCGAGGTCTCGGTCACCATCCCCAGCTGCATCG
AGGATGGCGAGTACCTGCTCCGGGTCGAGCACATCGCGCTCCACAGCGCC
AGCAGCGTGGGCGGCGCCCAGTTCTACATCGCCTGCGCCCAGCTCTCCGT
CACCGGCGGCTCCGGCACCCTCAACACGGGCTCGCTCGTCTCCCTGCCCG
GCGCCTACAAGGCCACCGACCCGGGCATCCTCTTCCAGCTCTACTGGCCC
ATCCCGACCGAGTACATCAACCCCGGCCCGGCCCCCGTCTCTTGC
(SEQ ID NO: 76)
MKLTSSLAVLAAAGAQAHYTFPRAGTGGSLSGEWEVVRMTENHYSHGPVT
DVTSPEMTCYQSGVQGAPQTVQVKAGSQFTFSVDPSIGHPGPLQFYMAKV
PSGQTAATFDGTGAVWFKIYQDGPNGLGTDSITWPSAGKTEVSVTIPSCI
EDGEYLLRVEHIALHSASSVGGAQFYIACAQLSVTGGSGTLNTGSLVSLP
GAYKATDPGILFQLYWPIPTEYINPGPAPVSC
(SEQ ID NO: 77)
HYTFPRAGTGGSLSGEWEVVRMTENHYSHGPVTDVTSPEMTCYQSGVQGA
PQTVQVKAGSQFTFSVDPSIGHPGPLQFYMAKVPSGQTAATFDGTGAVWF
KIYQDGPNGLGTDSITWPSAGKTEVSVTIPSCIEDGEYLLRVEHIALHSA
SSVGGAQFYIACAQLSVTGGSGTLNTGSLVSLPGAYKATDPGILFQLYWP
IPTEYINPGPAPVSC

The polynucleotide (SEQ ID NO:78) and amino acid (SEQ ID NO:79) sequences of an alternative M. thermophila GH61q are provided below. The signal sequence is shown underlined in SEQ ID NO:79. SEQ ID NO:80 provides the sequence of this GH61q without the signal sequence.

(SEQ ID NO: 78)
ATGCCGCCACCACGACTGAGCACCCTCCTTCCCCTCCTAGCCTTAATAGC
CCCCACCGCCCTGGGGCACTCCCACCTCGGGTACATCATCATCAACGGCG
AGGTATACCAAGGATTCGACCCGCGGCCGGAGCAGGCGAACTCGCCGTTG
CGCGTGGGCTGGTCGACGGGGGCAATCGACGACGGGTTCGTGGCGCCGGC
CAACTACTCGTCGCCCGACATCATCTGCCACATCGAGGGGGCCAGCCCGC
CGGCGCACGCGCCCGTCCGGGCGGGCGACCGGGTGCACGTGCAATGGAAC
GGCTGGCCGCTCGGACACGTGGGGCCGGTGCTGTCGTACCTGGCGCCCTG
CGGCGGGCTGGAGGGGTCCGAGAGCGGGTGCGCCGGGGTGGACAAGCGGC
AGCTGCGGTGGACCAAGGTGGACGACTCGCTGCCGGCGATGGAGCTG
(SEQ ID NO: 79)
MPPPRLSTLLPLLALIAPTALGHSHLGYIIINGEVYQGFDPRPEQANSPL
RVGWSTGAIDDGFVAPANYSSPDIICHIEGASPPAHAPVRAGDRVHVQWN
GWPLGHVGPVLSYLAPCGGLEGSESGCAGVDKRQLRWTKVDDSLPAMEL
(SEQ ID NO: 80)
HSHLGYIIINGEVYQGFDPRPEQANSPLRVGWSTGAIDDGFVAPANYSSP
DIICHIEGASPPAHAPVRAGDRVHVQWNGWPLGHVGPVLSYLAPCGGLEG
SESGCAGVDKRQLRWTKVDDSLPAMEL

The polynucleotide (SEQ ID NO:81) and amino acid (SEQ ID NO:82) sequences of an alternative M. thermophila GH61q are provided below. The signal sequence is shown underlined in SEQ ID NO:82. SEQ ID NO:83 provides the sequence of this GH61q without the signal sequence.

(SEQ ID NO: 81)
ATGCCGCCACCACGACTGAGCACCCTCCTTCCCCTCCTAGCCTTAATAGC
CCCCACCGCCCTGGGGCACTCCCACCTCGGGTACATCATCATCAACGGCG
AGGTATACCAAGGATTCGACCCGCGGCCGGAGCAGGCGAACTCGCCGTTG
CGCGTGGGCTGGTCGACGGGGGCAATCGACGACGGGTTCGTGGCGCCGGC
CAACTACTCGTCGCCCGACATCATCTGCCACATCGAGGGGGCCAGCCCGC
CGGCGCACGCGCCCGTCCGGGCGGGCGACCGGGTGCACGTGCAATGGAAA
CGGCTGGCCGCTCGGACACGTGGGGCCGGTGCTGTCGTACCTGGCGCCCT
GCGGCGGGCTGGAGGGGTCCGAGAGCGGGTGGACGACTCGCTGCCGGCGA
TGGAGCTGGTCGGGGCCGCGGGGGGCGCGGGGGGCGAGGACGACGGCAGC
GGCAGCGACGGCAGCGGCAGCGGCGGCAGCGGACGCGTCGGCGTGCCCGG
GCAGCGCTGGGCCACCGACGTGTTGATCGCGGCCAACAACAGCTGGCAGG
TCGAGATCCCGCGCGGGCTGCGGGACGGGCCGTACGTGCTGCGCCACGAG
ATCGTCGCGCTGCACTACGCGGCCGAGCCCGGCGGCGCGCAGAACTACCC
GCTCTGCGTCAACCTGTGGGTCGAGGGCGGCGACGGCAGCATGGAGCTGG
ACCACTTCGACGCCACCCAGTTCTACCGGCCCGACGACCCGGGCATCCTG
CTCAACGTGACGGCCGGCCTGCGCTCATACGCCGTGCCGGGCCCGACGCT
GGCCGCGGGGGCGACGCCGGTGCCGTACGCGCAGCAGAACATCAGCTCGG
CGAGGGCGGATGGAACCCCCGTGATTGTCACCAGGAGCACGGAGACGGTG
CCCTTCACCGCGGCACCCACGCCAGCCGAGACGGCAGAAGCCAAAGGGGG
GAGGTATGATGACCAAACCCGAACTAAAGACCTAAATGAACGCTTCTTTT
ATAGTAGCCGGCCAGAACAGAAGAGGCTGACAGCGACCTCAAGAAGGGAA
CTAGTTGATCATCGTACCCGGTACCTCTCCGTAGCTGTCTGCGCAGATTT
CGGCGCTCATAAGGCAGCAGAAACCAACCACGAAGCTTTGAGAGGCGGCA
ATAAGCACCATGGCGGTGTTTCAGAG
(SEQ ID NO: 82)
MPPPRLSTLLPLLALIAPTALGHSHLGYIIINGEVYQGFDPRPEQANSPL
RVGWSTGAIDDGFVAPANYSSPDIICHIEGASPPAHAPVRAGDRVHVQWK
RLAARTRGAGAVVPGALRRAGGVRERVDDSLPAMELVGAAGGAGGEDDGS
GSDGSGSGGSGRVGVPGQRWATDVLIAANNSWQVEIPRGLRDGPYVLRHE
IVALHYAAEPGGAQNYPLCVNLWVEGGDGSMELDHFDATQFYRPDDPGIL
LNVTAGLRSYAVPGPTLAAGATPVPYAQQNISSARADGTPVIVTRSTETV
PFTAAPTPAETAEAKGGRYDDQTRTKDLNERFFYSSRPEQKRLTATSRRE
LVDHRTRYLSVAVCADFGAHKAAETNHEALRGGNKHHGGVSE
(SEQ ID NO: 83)
HSHLGYIIINGEVYQGFDPRPEQANSPLRVGWSTGAIDDGFVAPANYSSP
DIICHIEGASPPAHAPVRAGDRVHVQWKRLAARTRGAGAVVPGALRRAGG
VRERVDDSLPAMELVGAAGGAGGEDDGSGSDGSGSGGSGRVGVPGQRWAT
DVLIAANNSWQVEIPRGLRDGPYVLRHEIVALHYAAEPGGAQNYPLCVNL
WVEGGDGSMELDHFDATQFYRPDDPGILLNVTAGLRSYAVPGPTLAAGAT
PVPYAQQNISSARADGTPVIVTRSTETVPFTAAPTPAETAEAKGGRYDDQ
TRTKDLNERFFYSSRPEQKRLTATSRRELVDHRTRYLSVAVCADFGAHKA
AETNHEALRGGNKHHGGVSE

The polynucleotide (SEQ ID NO:84) and amino acid (SEQ ID NO:85) sequences of an M. thermophila GH61r are provided below. The signal sequence is shown underlined in SEQ ID NO:85. SEQ ID NO:86 provides the sequence of this GH61r without the signal sequence.

(SEQ ID NO: 84)
ATGAGGTCGACATTGGCCGGTGCCCTGGCAGCCATCGCTGCTCAGAAAGT
AGCCGGCCACGCCACGTTTCAGCAGCTCTGGCACGGCTCCTCCTGTGTCC
GCCTTCCGGCTAGCAACTCACCCGTCACCAATGTGGGAAGCAGAGACTTC
GTCTGCAACGCTGGCACCCGCCCCGTCAGTGGCAAGTGCCCCGTGAAGGC
TGGCGGCACCGTCACCATCGAGATGCACCAGCAACCCGGCGACCGCAGCT
GCAACAACGAAGCCATCGGAGGGGCGCATTGGGGCCCCGTCCAGGTGTAC
CTGACCAAGGTTCAGGACGCCGCGACGGCCGACGGCTCGACGGGCTGGTT
CAAGATCTTCTCCGACTCGTGGTCCAAGAAGCCCGGGGGCAACTTGGGCG
ACGACGACAACTGGGGCACGCGCGACCTGAACGCCTGCTGCGGGAAGATG
GAC
(SEQ ID NO: 85)
MRSTLAGALAAIAAQKVAGHATFQQLWHGSSCVRLPASNSPVTNVGSRDF
VCNAGTRPVSGKCPVKAGGTVTIEMHQQPGDRSCNNEAIGGAHWGPVQVY
LTKVQDAATADGSTGWFKIFSDSWSKKPGGNLGDDDNWGTRDLNACCGKM
D
(SEQ ID NO: 86)
HATFQQLWHGSSCVRLPASNSPVTNVGSRDFVCNAGTRPVSGKCPVKAGG
TVTIEMHQQPGDRSCNNEAIGGAHWGPVQVYLTKVQDAATADGSTGWFKI
FSDSWSKKPGGNLGDDDNWGTRDLNACCGKMD

The polynucleotide (SEQ ID NO:87) and amino acid (SEQ ID NO:88) sequences of an alternative M. thermophila GH61r are provided below. The signal sequence is shown underlined in SEQ ID NO:88. SEQ ID NO:89 provides the sequence of this GH61r without the signal sequence.

(SEQ ID NO: 87)
ATGAGGTCGACATTGGCCGGTGCCCTGGCAGCCATCGCTGCTCAGAAAGT
AGCCGGCCACGCCACGTTTCAGCAGCTCTGGCACGGCTCCTCCTGTGTCC
GCCTTCCGGCTAGCAACTCACCCGTCACCAATGTGGGAAGCAGAGACTTC
GTCTGCAACGCTGGCACCCGCCCCGTCAGTGGCAAGTGCCCCGTGAAGGC
TGGCGGCACCGTCACCATCGAGATGCACCAGCAACCCGGCGACCGCAGCT
GCAACAACGAAGCCATCGGAGGGGCGCATTGGGGCCCCGTCCAGGTGTAC
CTGACCAAGGTTCAGGACGCCGCGACGGCCGACGGCTCGACGGGCTGGTT
CAAGATCTTCTCCGACTCGTGGTCCAAGAAGCCCGGGGGCAACTCGGGCG
ACGACGACAACTGGGGCACGCGCGACCTGAACGCCTGCTGCGGGAAGATG
GACGTGGCCATCCCGGCCGACATCGCGTCGGGCGACTACCTGCTGCGGGC
CGAGGCGCTGGCCCTGCACACGGCCGGACAGGCCGGCGGCGCCCAGTTCT
ACATGAGCTGCTACCAGATGACGGTCGAGGGCGGCTCCGGGACCGCCAAC
CCGCCCACCGTCAAGTTCCCGGGCGCCTACAGCGCCAACGACCCGGGCAT
CCTCGTCAACATCCACGCCCCCCTTTCCAGCTACACCGCGCCCGGCCCGG
CCGTCTACGCGGGCGGCACCATCCGCGAGGCCGGCTCCGCCTGCACCGGC
TGCGCGCAGACCTGCAAGGTCGGGTCGTCCCCGAGCGCCGTTGCCCCCGG
CAGCGGCGCGGGCAACGGCGGCGGGTTCCAACCCCGA
(SEQ ID NO: 88)
MRSTLAGALAAIAAQKVAGHATFQQLWHGSSCVRLPASNSPVTNVGSRDF
VCNAGTRPVSGKCPVKAGGTVTIEMHQQPGDRSCNNEAIGGAHWGPVQVY
LTKVQDAATADGSTGWFKIFSDSWSKKPGGNSGDDDNWGTRDLNACCGKM
DVAIPADIASGDYLLRAEALALHTAGQAGGAQFYMSCYQMTVEGGSGTAN
PPTVKFPGAYSANDPGILVNIHAPLSSYTAPGPAVYAGGTIREAGSACTG
CAQTCKVGSSPSAVAPGSGAGNGGGFQPR
(SEQ ID NO: 89)
HATFQQLWHGSSCVRLPASNSPVTNVGSRDFVCNAGTRPVSGKCPVKAGG
TVTIEMHQQPGDRSCNNEAIGGAHWGPVQVYLIKVQDAATADGSTGWFKI
FSDSWSKKPGGNSGDDDNWGTRDLNACCGKMDVAIPADIASGDYLLRAEA
LALHTAGQAGGAQFYMSCYQMTVEGGSGTANPPTVKFPGAYSANDPGILV
NIHAPLSSYTAPGPAVYAGGTIREAGSACTGCAQTCKVGSSPSAVAPGSG
AGNGGGFQPR

The polynucleotide (SEQ ID NO:90) and amino acid (SEQ ID NO:91) sequences of an M. thermophila GH61s are provided below. The signal sequence is shown underlined in SEQ ID NO:91. SEQ ID NO:92 provides the sequence of this GH61s without the signal sequence.

(SEQ ID NO: 90)
ATGCTCCTCCTCACCCTAGCCACACTCGTCACCCTCCTGGCGCGCCACGT
CTCGGCTCACGCCCGGCTGTTCCGCGTCTCTGTCGACGGGAAAGACCAGG
GCGACGGGCTGAACAAGTACATCCGCTCGCCGGCGACCAACGACCCCGTG
CGCGACCTCTCGAGCGCCGCCATCGTGTGCAACACCCAGGGGTCCAAGGC
CGCCCCGGACTTCGTCAGGGCCGCGGCCGGCGACAAGCTGACCTTCCTCT
GGGCGCACGACAACCCGGACGACCCGGTCGACTACGTCCTCGACCCGTCC
CACAAGGGCGCCATCCTGACCTACGTCGCCGCCTACCCCTCCGGGGACCC
GACCGGCCCCATCTGGAGCAAGCTTGCCGAGGAAGGATTCACCGGCGGGC
AGTGGGCGACCATCAAGATGATCGACAACGGCGGCAAGGTCGACGTGACG
CTGCCCGAGGCCCTTGCGCCGGGAAAGTACCTGATCCGCCAGGAGCTGCT
GGCCCTGCACCGGGCCGACTTTGCCTGCGACGACCCGGCCCACCCCAACC
GCGGCGCCGAGTCGTACCCCAACTGCGTCCAGGTGGAGGTGTCGGGCAGC
GGCGACAAGAAGCCGGACCAGAACTTTGACTTCAACAAGGGCTATACCTG
CGATAACAAAGGACTCCACTTTAAGATCTACATCGGTCAGGACAGCCAGT
ATGTGGCCCCGGGGCCGCGGCCTTGGAATGGGAGC
(SEQ ID NO: 91)
MLLLTLATLVTLLARHVSAHARLFRVSVDGKDQGDGLNKYIRSPATNDPV
RDLSSAAIVCNTQGSKAAPDFVRAAAGDKLTFLWAHDNPDDPVDYVLDPS
HKGAILTYVAAYPSGDPTGPIWSKLAEEGFTGGQWATIKMIDNGGKVDVT
LPEALAPGKYLIRQELLALHRADFACDDPAHPNRGAESYPNCVQVEVSGS
GDKKPDQNFDFNKGYTCDNKGLHFKIYIGQDSQYVAPGPRPWNGS
(SEQ ID NO: 92)
HARLFRVSVDGKDQGDGLNKYIRSPATNDPVRDLSSAAIVCNTQGSKAAP
DFVRAAAGDKLTFLWAHDNPDDPVDYVLDPSHKGAILTYVAAYPSGDPTG
PIWSKLAEEGFTGGQWATIKMIDNGGKVDVTLPEALAPGKYLIRQELLAL
HRADFACDDPAHPNRGAESYPNCVQVEVSGSGDKKPDQNFDFNKGYTCDN
KGLHFKIYIGQDSQYVAPGPRPWNGS

The polynucleotide (SEQ ID NO:93) and amino acid (SEQ ID NO:94) sequences of an M. thermophila GH61t are provided below.

(SEQ ID NO: 93)
ATGTTCACTTCGCTTTGCATCACAGATCATTGGAGGACTCTTAGCAGCCA
CTCTGGGCCAGTCATGAACTATCTCGCCCATTGCACCAATGACGACTGCA
AGTCTTTCAAGGGCGACAGCGGCAACGTCTGGGTCAAGATCGAGCAGCTC
GCGTACAACCCGTCAGCCAACCCCCCCTGGGCGTCTGACCTCCTCCGTGA
GCACGGTGCCAAGTGGAAGGTGACGATCCCGCCCAGTCTTGTCCCCGGCG
AATATCTGCTGCGGCACGAGATCCTGGGGTTGCACGTCGCAGGAACCGTG
ATGGGCGCCCAGTTCTACCCCGGCTGCACCCAGATCAGGGTCACCGAAGG
CGGGAGCACGCAGCTGCCCTCGGGTATTGCGCTCCCAGGCGCTTACGGCC
CACAAGACGAGGGTATCTTGGTCGACTTGTGGAGGGTTAACCAGGGCCAG
GTCAACTACACGGCGCCTGGAGGACCCGTTTGGAGCGAAGCGTGGGACAC
CGAGTTTGGCGGGTCCAACACGACCGAGTGCGCCACCATGCTCGACGACC
TGCTCGACTACATGGCGGCCAACGACGAGTGGATCGGCTGGACGGCCTAG
(SEQ ID NO: 94)
MFTSLCITDHWRTLSSHSGPVMNYLAHCTNDDCKSFKGDSGNVWVKIEQL
AYNPSANPPWASDLLREHGAKWKVTIPPSLVPGEYLLRHEILGLHVAGTV
MGAQFYPGCTQIRVTEGGSTQLPSGIALPGAYGPQDEGILVDLWRVNQGQ
VNYTAPGGPVWSEAWDTEFGGSNTTECATMLDDLLDYMAANDEWIGWTA

The polynucleotide (SEQ ID NO:95) and amino acid (SEQ ID NO:96) sequences of an alternative M. thermophila GH61t are provided below.

(SEQ ID NO: 95)
ATGAACTATCTCGCCCATTGCACCAATGACGACTGCAAGTCTTTCAAGGG
CGACAGCGGCAACGTCTGGGTCAAGATCGAGCAGCTCGCGTACAACCCGT
CAGCCAACCCCCCCTGGGCGTCTGACCTCCTCCGTGAGCACGGTGCCAAG
TGGAAGGTGACGATCCCGCCCAGTCTTGTCCCCGGCGAATATCTGCTGCG
GCACGAGATCCTGGGGTTGCACGTCGCAGGAACCGTGATGGGCGCCCAGT
TCTACCCCGGCTGCACCCAGATCAGGGTCACCGAAGGCGGGAGCACGCAG
CTGCCCTCGGGTATTGCGCTCCCAGGCGCTTACGGCCCACAAGACGAGGG
TATCTTGGTCGACTTGTGGAGGGTTAACCAGGGCCAGGTCAACTACACGG
CGCCTGGAGGACCCGTTTGGAGCGAAGCGTGGGACACCGAGTTTGGCGGG
TCCAACACGACCGAGTGCGCCACCATGCTCGACGACCTGCTCGACTACAT
GGCGGCCAACGACGACCCATGCTGCACCGACCAGAACCAGTTCGGGAGTC
TCGAGCCGGGGAGCAAGGCGGCCGGCGGCTCGCCGAGCCTGTACGATACC
GTCTTGGTCCCCGTTCTCCAGAAGAAAGTGCCGACAAAGCTGCAGTGGAG
CGGACCGGCGAGCGTCAACGGGGATGAGTTGACAGAGAGGCCC
(SEQ ID NO: 96)
MNYLAHCTNDDCKSFKGDSGNVWVKIEQLAYNPSANPPWASDLLREHGAK
WKVTIPPSLVPGEYLLRHEILGLHVAGTVMGAQFYPGCTQIRVTEGGSTQ
LPSGIALPGAYGPQDEGILVDLWRVNQGQVNYTAPGGPVWSEAWDTEFGG
SNTTECATMIDDLLDYMAANDDPCCTDQNQFGSLEPGSKAAGGSPSLYDT
VLVPVLQKKVPTKLQWSGPASVNGDELTERP

The polynucleotide (SEQ ID NO:97) and amino acid (SEQ ID NO:98) sequences of an M. thermophila GH61u are provided below. The signal sequence is shown underlined in SEQ ID NO:98. SEQ ID NO:99 provides the sequence of this GH61u without the signal sequence.

(SEQ ID NO: 97)
ATGAAGCTGAGCGCTGCCATCGCCGTGCTCGCGGCCGCCCTTGCCGAGGG
GCACTATACCTTCCCCAGCATCGCCAACACGGCCGACTGGCAATATGTGC
GCATCACGACCAACTTCCAGAGCAACGGCCCCGTGACGGACGTCAACTCG
GACCAGATCCGGTGCTACGAGCGCAACCCGGGCACCGGCGCCCCCGGCAT
CTACAACGTCACGGCCGGCACAACCATCAACTACAACGCCAAGTCGTCCA
TCTCCCACCCGGGACCCATGGCCTTCTACATTGCCAAGGTTCCCGCCGGC
CAGTCGGCCGCCACCTGGGACGGTAAGGGCGCCGTCTGGTCCAAGATCCA
CCAGGAGATGCCGCACTTTGGCACCAGCCTCACCTGGGACTCCAACGGCC
GCACCTCCATGCCCGTCACCATCCCCCGCTGTCTGCAGGACGGCGAGTAT
CTGCTGCGTGCAGAGCACATTGCCCTCCACAGCGCCGGCAGCCCCGGCGG
CGCCCAGTTCTACATTTCTTGTGCCCAGCTCTCAGTCACCGGCGGCAGCG
GGACCTGGAACCCCAGGAACAAGGTGTCGTTCCCCGGCGCCTACAAGGCC
ACTGACCCGGGCATCCTGATCAACATCTACTACCCCGTCCCGACTAGCTA
CACTCCCGCTGGTCCCCCCGTCGACACCTGC
(SEQ ID NO: 98)
MKLSAAIAVLAAALAEGHYTFPSIANTADWQYVRITTNFQSNGPVTDVNS
DQIRCYERNPGTGAPGIYNVTAGTTINYNAKSSISHPGPMAFYIAKVPAG
QSAATWDGKGAVWSKIHQEMPHFGTSLTWDSNGRTSMPVTIPRCLQDGEY
LLRAEHIALHSAGSPGGAQFYISCAQLSVTGGSGTWNPRNKVSFPGAYKA
TDPGILINIYYPVPTSYTPAGPPVDTC
(SEQ ID NO: 99)
HYTFPSIANTADWQYVRITTNFQSNGPVTDVNSDQIRCYERNPGTGAPGI
YNVTAGTTINYNAKSSISHPGPMAFYIAKVPAGQSAATWDGKGAVWSKIH
QEMPHFGTSLTWDSNGRTSMPVTIPRCLQDGEYLLRAEHIALHSAGSPGG
AQFYISCAQLSVTGGSGTWNPRNKVSFPGAYKATDPGILINIYYPVPTSY
TPAGPPVDTC

The polynucleotide (SEQ ID NO:100) and amino acid (SEQ ID NO:101) sequences of an M. thermophila GH61v are provided below. The signal sequence is shown underlined in SEQ ID NO:101. SEQ ID NO:102 provides the sequence of this GH61v without the signal sequence.

(SEQ ID NO: 100)
ATGTACCGCACGCTCGGTTCCATTGCCCTGCTCGCGGGGGGCGCTGCCGC
CCACGGCGCCGTGACCAGCTACAACATTGCGGGCAAGGACTACCCTGGAT
ACTCGGGCTTCGCCCCTACCGGCCAGGATGTCATCCAGTGGCAATGGCCC
GACTATAACCCCGTGCTGTCCGCCAGCGACCCCAAGCTCCGCTGCAACGG
CGGCACCGGGGCGGCGCTGTATGCCGAGGCGGCCCCCGGCGACACCATCA
CGGCCACCTGGGCCCAGTGGACGCACTCCCAGGGCCCGATCCTGGTGTGG
ATGTACAAGTGCCCCGGCGACTTCAGCTCCTGCGACGGCTCCGGCGCGGG
TTGGTTCAAGATCGACGAGGCCGGCTTCCACGGCGACGGCACGACCGTCT
TCCTCGACACCGAGACCCCCTCGGGCTGGGACATTGCCAAGCTGGTCGGC
GGCAACAAGTCGTGGAGCAGCAAGATCCCTGACGGCCTCGCCCCGGGCAA
TTACCTGGTCCGCCACGAGCTCATCGCCCTGCACCAGGCCAACAACCCGC
AATTCTACCCCGAGTGCGCCCAGATCAAGGTCACCGGCTCTGGCACCGCC
GAGCCCGCCGCCTCCTACAAGGCCGCCATCCCCGGCTACTGCCAGCAGAG
CGACCCCAACATTTCGTTCAACATCAACGACCACTCCCTCCCGCAGGAGT
ACAAGATCCCCGGTCCCCCGGTCTTCAAGGGCACCGCCTCCGCCAAGGCT
CGCGCTTTCCAGGCC
(SEQ ID NO: 101)
MYRTLGSIALLAGGAAAHGAVTSYNIAGKDYPGYSGFAPTGQDVIQWQWP
DYNPVLSASDPKLRCNGGTGAALYAEAAPGDTITATWAQWTHSQGPILVW
MYKCPGDFSSCDGSGAGWFKIDEAGFHGDGTTVFLDTETPSGWDIAKLVG
GNKSWSSKIPDGLAPGNYLVRHELIALHQANNPQFYPECAQIKVTGSGTA
EPAASYKAAIPGYCQQSDPNISFNINDHSLPQEYKIPGPPVFKGTASAKA
RAFQA
(SEQ ID NO: 102)
AVTSYNIAGKDYPGYSGFAPTGQDVIQWQWPDYNPVLSASDPKLRCNGGT
GAALYAEAAPGDTITATWAQWTHSQGPILVWMYKCPGDFSSCDGSGAGWF
KIDEAGFHGDGTTVFLDTETPSGWDIAKLVGGNKSWSSKIPDGLAPGNYL
VRHELIALHQANNPQFYPECAQIKVTGSGTAEPAASYKAAIPGYCQQSDP
NISFNINDHSLPQEYKIPGPPVFKGTASAKARAFQA

The polynucleotide (SEQ ID NO:103) and amino acid (SEQ ID NO:104) sequences of an M. thermophila GH61w are provided below. The signal sequence is shown underlined in SEQ ID NO:104. SEQ ID NO:105 provides the sequence of this GH61w without the signal sequence.

(SEQ ID NO: 103)
ATGCTGACAACAACCTTCGCCCTCCTGACGGCCGCTCTCGGCGTCAGCGC
CCATTATACCCTCCCCAGGGTCGGGACCGGTTCCGACTGGCAGCACGTGC
GGCGGGCTGACAACTGGCAAAACAACGGCTTCGTCGGCGACGTCAACTCG
GAGCAGATCAGGTGCTTCCAGGCGACCCCTGCCGGCGCCCAAGACGTCTA
CACTGTTCAGGCGGGATCGACCGTGACCTACCACGCCAACCCCAGTATCT
ACCACCCCGGCCCCATGCAGTTCTACCTGGCCCGCGTTCCGGACGGACAG
GACGTCAAGTCGTGGACCGGCGAGGGTGCCGTGTGGTTCAAGGTGTACGA
GGAGCAGCCTCAATTTGGCGCCCAGCTGACCTGGCCTAGCAACGGCAAGA
GCTCGTTCGAGGTTCCTATCCCCAGCTGCATTCGGGCGGGCAACTACCTC
CTCCGCGCTGAGCACATCGCCCTGCACGTTGCCCAAAGCCAGGGCGGCGC
CCAGTTCTACATCTCGTGCGCCCAGCTCCAGGTCACTGGTGGCGGCAGCA
CCGAGCCTTCTCAGAAGGTTTCCTTCCCGGGTGCCTACAAGTCCACCGAC
CCCGGCATTCTTATCAACATCAACTACCCCGTCCCTACCTCGTACCAGAA
TCCGGGTCCGGCTGTCTTCCGTTGC
(SEQ ID NO: 104)
MLTTTFALLTAALGVSAHYTLPRVGTGSDWQHVRRADNWQNNGFVGDVNS
EQIRCFQATPAGAQDVYTVQAGSTVTYHANPSIYHPGPMQFYLARVPDGQ
DVKSWTGEGAVWFKVYEEQPQFGAQLTWPSNGKSSFEVPIPSCIRAGNYL
LRAEHIALHVAQSQGGAQFYISCAQLQVTGGGSTEPSQKVSFPGAYKSTD
PGILININYPVPTSYQNPGPAVFRC
(SEQ ID NO: 105)
HYTLPRVGTGSDWQHVRRADNWQNNGFVGDVNSEQIRCFQATPAGAQDVY
TVQAGSTVTYHANPSIYHPGPMQFYLARVPDGQDVKSWTGEGAVWFKVYE
EQPQFGAQLTWPSNGKSSFEVPIPSCIRAGNYLLRAEHIALHVAQSQGGA
QFYISCAQLQVTGGGSTEPSQKVSFPGAYKSTDPGILININYPVPTSYQN
PGPAVFRC

The polynucleotide (SEQ ID NO:106) and amino acid (SEQ ID NO:107) sequences of a M. thermophila GH61x are provided below. The signal sequence is shown underlined in SEQ ID NO:107. SEQ ID NO:108 provides the sequence of this GH61x without the signal sequence.

(SEQ ID NO: 106)
ATGAAGGTTCTCGCGCCCCTGATTCTGGCCGGTGCCGCCAGCGCCCACAC
CATCTTCTCATCCCTCGAGGTGGGCGGCGTCAACCAGGGCATCGGGCAGG
GTGTCCGCGTGCCGTCGTACAACGGTCCGATCGAGGACGTGACGTCCAAC
TCGATCGCCTGCAACGGGCCCCCCAACCCGACGACGCCGACCAACAAGGT
CATCACGGTCCGGGCCGGCGAGACGGTGACGGCCGTCTGGCGGTACATGC
TGAGCACCACCGGCTCGGCCCCCAACGACATCATGGACAGCAGCCACAAG
GGCCCGACCATGGCCTACCTCAAGAAGGTCGACAACGCCACCACCGACTC
GGGCGTCGGCGGCGGCTGGTTCAAGATCCAGGAGGACGGCCTTACCAACG
GCGTCTGGGGCACCGAGCGCGTCATCAACGGCCAGGGCCGCCACAACATC
AAGATCCCCGAGTGCATCGCCCCCGGCCAGTACCTCCTCCGCGCCGAGAT
GCTTGCCCTGCACGGAGCTTCCAACTACCCCGGCGCTCAGTTCTACATGG
AGTGCGCCCAGCTCAATATCGTCGGCGGCACCGGCAGCAAGACGCCGTCC
ACCGTCAGCTTCCCGGGCGCTTACAAGGGTACCGACCCCGGAGTCAAGAT
CAACATCTACTGGCCCCCCGTCACCAGCTACCAGATTCCCGGCCCCGGCG
TGTTCACCTGC
(SEQ ID NO: 107)
MKVLAPLILAGAASAHTIFSSLEVGGVNQGIGQGVRVPSYNGPIEDVTSN
SIACNGPPNPTTPTNKVITVRAGETVTAVWRYMLSTTGSAPNDIMDSSHK
GPTMAYLKKVDNATTDSGVGGGWFKIQEDGLTNGVWGTERVINGQGRHNI
KIPECIAPGQYLLRAEMLALHGASNYPGAQFYMECAQLNIVGGTGSKTPS
TVSFPGAYKGTDPGVKINIYWPPVTSYQIPGPGVFTC
(SEQ ID NO: 108)
HTIFSSLEVGGVNQGIGQGVRVPSYNGPIEDVTSNSIACNGPPNPTTPTN
KVITVRAGETVTAVWRYMLSTTGSAPNDIMDSSHKGPTMAYLKKVDNATT
DSGVGGGWFKIQEDGLTNGVWGTERVINGQGRHNIKTPECIAPGQYLLRA
EMLALHGASNYPGAQFYMECAQLNIVGGTGSKTPSTVSFPGAYKGTDPGV
KINIYWPPVTSYQIPGPGVFTC

The polynucleotide (SEQ ID NO:109) and amino acid (SEQ ID NO:110) sequences of an M. thermophila GH61y are provided below. The signal sequence is underlined in SEQ ID NO:110. SEQ ID NO:111 provides the sequence of GH61y, without the signal sequence.

(SEQ ID NO: 109)
ATGATCGACAACCTCCCTGATGACTCCCTACAACCCGCCTGCCTCCGCCC
GGGCCACTACCTCGTCCGCCACGAGATCATCGCGCTGCACTCGGCCTGGG
CCGAGGGCGAGGCCCAGTTCTACCCCTTCCCCCTTTTTCCTTTTTTTCCC
TCCCTTCTTTTGTCCGGTAACTACACGATTCCCGGTCCCGCGATCTGGAA
GTGCCCAGAGGCACAGCAGAACGAG
(SEQ ID NO: 110)
MIDNLPDDSLQPACLRPGHYLVRHEIIALHSAWAEGEAQFYPFPLFPFFP
SLLLSGNYTIPGPAIWKCPEAQQNE
(SEQ ID NO: 111)
HYLVRHEIIALHSAWAEGEAQFYPFPLFPFFPSLLLSGNYTIPGPAIWKC
PEAQQNE

Wild-type M. thermophila EG2 polynucleotide (SEQ ID NO:112) and amino acid (SEQ ID NO:113) sequences are provided below. The signal sequence is underlined in SEQ ID NO:113. SEQ ID NO:114 provides the sequence of EG2, without the signal sequence.

(SEQ ID NO: 112)
ATGAAGTCCTCCATCCTCGCCAGCGTCTTCGCCACGGGCGCCGTGGCTCA
AAGTGGTCCGTGGCAGCAATGTGGTGGCATCGGATGGCAAGGATCGACCG
ACTGTGTGTCGGGTTACCACTGCGTCTACCAGAACGATTGGTACAGCCAG
TGCGTGCCTGGCGCGGCGTCGACAACGCTCCAGACATCTACCACGTCCAG
GCCCACCGCCACCAGCACCGCCCCTCCGTCGTCCACCACCTCGCCTAGCA
AGGGCAAGCTCAAGTGGCTCGGCAGCAACGAGTCGGGCGCCGAGTTCGGG
GAGGGCAACTACCCCGGCCTCTGGGGCAAGCACTTCATCTTCCCGTCGAC
TTCGGCGATTCAGACGCTCATCAATGATGGATACAACATCTTCCGGATCG
ACTTCTCGATGGAGCGTCTGGTGCCCAACCAGTTGACGTCGTCCTTCGAC
GAGGGCTACCTCCGCAACCTGACCGAGGTGGTCAACTTCGTGACGAACGC
GGGCAAGTACGCCGTCCTGGACCCGCACAACTACGGCCGGTACTACGGCA
ACGTCATCACGGACACGAACGCGTTCCGGACCTTCTGGACCAACCTGGCC
AAGCAGTTCGCCTCCAACTCGCTCGTCATCTTCGACACCAACAACGAGTA
CAACACGATGGACCAGACCCTGGTGCTCAACCTCAACCAGGCCGCCATCG
ACGGCATCCGGGCCGCCGGCGCGACCTCGCAGTACATCTTCGTCGAGGGC
AACGCGTGGAGCGGGGCCTGGAGCTGGAACACGACCAACACCAACATGGC
CGCCCTGACGGACCCGCAGAACAAGATCGTGTACGAGATGCACCAGTACC
TCGACTCGGACAGCTCGGGCACCCACGCCGAGTGCGTCAGCAGCAACATC
GGCGCCCAGCGCGTCGTCGGAGCCACCCAGTGGCTCCGCGCCAACGGCAA
GCTCGGCGTCCTCGGCGAGTTCGCCGGCGGCGCCAACGCCGTCTGCCAGC
AGGCCGTCACCGGCCTCCTCGACCACCTCCAGGACAACAGCGACGTCTGG
CTGGGTGCCCTCTGGTGGGCCGCCGGTCCCTGGTGGGGCGACTACATGTA
CTCGTTCGAGCCTCCTTCGGGCACCGGCTATGTCAACTACAACTCGATCC
TAAAGAAGTACTTGCCGTAA
(SEQ ID NO: 113)
MKSSILASVFATGAVAQSGPWQQCGGIGWQGSTDCVSGYHCVYQNDWYSQ
CVPGAASTTLQTSTTSRPTATSTAPPSSTTSPSKGKLKWLGSNESGAEFG
EGNYPGLWGKHFIFPSTSAIQTLINDGYNIFRIDFSMERLVPNQLTSSFD
EGYLRNLTEVVNFVTNAGKYAVLDPHNYGRYYGNVITDTNAFRTFWTNLA
KQFASNSLVIFDTNNEYNTMDQTLVLNLNQAAIDGIRAAGATSQYIFVEG
NAWSGAWSWNTTNTNMAALTDPQNKIVYEMHQYLDSDSSGTHAECVSSNI
GAQRVVGATQWLRANGKLGVLGEFAGGANAVCQQAVTGLLDHLQDNSEVW
LGALWWAAGPWWGDYMYSFEPPSGTGYVNYNSILKKYLP
(SEQ ID NO: 114)
QSGPWQQCGGIGWQGSTDCVSGYHCVYQNDWYSQCVPGAASTTLQTSTTS
RPTATSTAPPSSTTSPSKGKLKWLGSNESGAEFGEGNYPGLWGKHFIFPS
TSAIQTLINDGYNIFRIDFSMERLVPNQLTSSFDEGYLRNLTEVVNFVTN
AGKYAVLDPHNYGRYYGNVITDTNAFRTFWTNLAKQFASNSLVIFDTNNE
YNTMDQTLVLNLNQAAIDGIRAAGATSQYIFVEGNAWSGAWSWNTTNTNM
AALTDPQNKIVYEMHQYLDSDSSGTHAECVSSNIGAQRVVGATQWLRANG
KLGVLGEFAGGANAVCQQAVTGLLDHLQDNSEVWLGALWWAAGPWWGDYM
YSFEPPSGTGYVNYNSILKKYLP

The polynucleotide (SEQ ID NO:115) and amino acid (SEQ ID NO:116) sequences of a wild-type BGL are provided below. The signal sequence is underlined in SEQ ID NO:116. SEQ ID NO:117 provides the polypeptide sequence without the signal sequence.

(SEQ ID NO: 115)
ATGAAGGCTGCTGCGCTTTCCTGCCTCTTCGGCAGTACCCTTGCCGTTGC
AGGCGCCATTGAATCGAGAAAGGTTCACCAGAAGCCCCTCGCGAGATCTG
AACCTTTTTACCCGTCGCCATGGATGAATCCCAACGCCGACGGCTGGGCG
GAGGCCTATGCCCAGGCCAAGTCCTTTGTCTCCCAAATGACTCTGCTAGA
GAAGGTCAACTTGACCACGGGAGTCGGCTGGGGGGCTGAGCAGTGCGTCG
GCCAAGTGGGCGCGATCCCTCGCCTTGGACTTCGCAGTCTGTGCATGCAT
GACTCCCCTCTCGGCATCCGAGGAGCCGACTACAACTCAGCGTTCCCCTC
TGGCCAGACCGTTGCTGCTACCTGGGATCGCGGTCTGATGTACCGTCGCG
GCTACGCAATGGGCCAGGAGGCCAAAGGCAAGGGCATCAATGTCCTTCTC
GGACCAGTCGCCGGCCCCCTTGGCCGCATGCCCGAGGGCGGTCGTAACTG
GGAAGGCTTCGCTCCGGATCCCGTCCTTACCGGCATCGGCATGTCCGAGA
CGATCAAGGGCATTCAGGATGCTGGCGTCATCGCTTGTGCGAAGCACTTT
ATTGGAAACGAGCAGGAGCACTTCAGACAGGTGCCAGAAGCCCAGGGATA
CGGTTACAACATCAGCGAAACCCTCTCCTCCAACATTGACGACAAGACCA
TGCACGAGCTCTACCTTTGGCCGTTTGCCGATGCCGTCCGGGCCGGCGTC
GGCTCTGTCATGTGCTCGTACCAGCAGGTCAACAACTCGTACGCCTGCCA
GAACTCGAAGCTGCTGAACGACCTCCTCAAGAACGAGCTTGGGTTTCAGG
GCTTCGTCATGAGCGACTGGCAGGCACAGCACACTGGCGCAGCAAGCGCC
GTGGCTGGTCTCGATATGTCCATGCCGGGCGACACCCAGTTCAACACTGG
CGTCAGTTTCTGGGGCGCCAATCTCACCCTCGCCGTCCTCAACGGCACAG
TCCCTGCCTACCGTCTCGACGACATGGCCATGCGCATCATGGCCGCCCTC
TTCAAGGTCACCAAGACCACCGACCTGGAACCGATCAACTTCTCCTTCTG
GACCGACGACACTTATGGCCCGATCCACTGGGCCGCCAAGCAGGGCTACC
AGGAGATTAATTCCCACGTTGACGTCCGCGCCGACCACGGCAACCTCATC
CGGGAGATTGCCGCCAAGGGTACGGTGCTGCTGAAGAATACCGGCTCTCT
ACCCCTGAACAAGCCAAAGTTCGTGGCCGTCATCGGCGAGGATGCTGGGT
CGAGCCCCAACGGGCCCAACGGCTGCAGCGACCGCGGCTGTAACGAAGGC
ACGCTCGCCATGGGCTGGGGATCCGGCACAGCCAACTATCCGTACCTCGT
TTCCCCCGACGCCGCGCTCCAGGCCCGGGCCATCCAGGACGGCACGAGGT
ACGAGAGCGTCCTGTCCAACTACGCCGAGGAAAAGACAAAGGCTCTGGTC
TCGCAGGCCAATGCAACCGCCATCGTCTTCGTCAATGCCGACTCAGGCGA
GGGCTACATCAACGTGGACGGTAACGAGGGCGACCGTAAGAACCTGACTC
TCTGGAACAACGGTGATACTCTGGTCAAGAACGTCTCGAGCTGGTGCAGC
AACACCATCGTCGTCATCCACTCGGTCGGCCCGGTCCTCCTGACCGATTG
GTACGACAACCCCAACATCACGGCCATTCTCTGGGCTGGTCTTCCGGGCC
AGGAGTCGGGCAACTCCATCACCGACGTGCTTTACGGCAAGGTCAACCCC
GCCGCCCGCTCGCCCTTCACTTGGGGCAAGACCCGCGAAAGCTATGGCGC
GGACGTCCTGTACAAGCCGAATAATGGCAATGGTGCGCCCCAACAGGACT
TCACCGAGGGCGTCTTCATCGACTACCGCTACTTCGACAAGGTTGACGAT
GACTCGGTCATCTACGAGTTCGGCCACGGCCTGAGCTACACCACCTTCGA
GTACAGCAACATCCGCGTCGTCAAGTCCAACGTCAGCGAGTACCGGCCCA
CGACGGGCACCACGGCCCAGGCCCCGACGTTTGGCAACTTCTCCACCGAC
CTCGAGGACTATCTCTTCCCCAAGGACGAGTTCCCCTACATCTACCAGTA
CATCTACCCGTACCTCAACACGACCGACCCCCGGAGGGCCTCGGCCGATC
CCCACTACGGCCAGACCGCCGAGGAGTTCCTCCCGCCCCACGCCACCGAT
GACGACCCCCAGCCGCTCCTCCGGTCCTCGGGCGGAAACTCCCCCGGCGG
CAACCGCCAGCTGTACGACATTGTCTACACAATCACGGCCGACATCACGA
ATACGGGCTCCGTTGTAGGCGAGGAGGTACCGCAGCTCTACGTCTCGCTG
GGCGGTCCCGAGGATCCCAAGGTGCAGCTGCGCGACTTTGACAGGATGCG
GATCGAACCCGGCGAGACGAGGCAGTTCACCGGCCGCCTGACGCGCAGAG
ATCTGAGCAACTGGGACGTCACGGTGCAGGACTGGGTCATCAGCAGGTAT
CCCAAGACGGCATATGTTGGGAGGAGCAGCCGGAAGTTGGATCTCAAGAT
TGAGCTTCCTTGA
(SEQ ID NO: 116)
MKAAALSCLFGSTLAVAGAIESRKVHQKPLARSEPFYPSPWMNPNADGWA
EAYAQAKSFVSQMTLLEKVNLTTGVGWGAEQCVGQVGAIPRLGLRSLCMH
DSPLGIRGADYNSAFPSGQTVAATWDRGLMYRRGYAMGQEAKGKGINVLL
GPVAGPLGRMPEGGRNWEGFAPDPVLTGIGMSETIKGIQDAGVIACAKHF
IGNEQEHFRQVPEAQGYGYNISETLSSNIDDKTMHELYLWPFADAVRAGV
GSVMCSYQQVNNSYACQNSKLLNDLLKNELGFQGFVMSDWQAQHTGAASA
VAGLDMSMPGDTQFNTGVSFWGANLTLAVLNGTVPAYRLDDMAMRIMAAL
FKVTKTTDLEPINFSFWTDDTYGPIHWAAKQGYQEINSHVDVRADHGNLI
REIAAKGTVLLKNTGSLPLNKPKFVAVIGEDAGSSPNGPNGCSDRGCNEG
TLAMGWGSGTANYPYLVSPDAALQARAIQDGTRYESVLSNYAEEKTKALV
SQANATAFVFVNADSGEGYINVDGNEGDRKNLTLWNNGDTLVKNVSSWCS
NTIVVIHSVGPVLLTDWYDNPNITAILWAGLPGQESGNSITDVLYGKVNP
AARSPFTWGKTRESYGADVLYKPNNGNGAPQQDFTEGVFIDYRYFDKVDD
DSVIYEFGHGLSYTTFEYSNIRVVKSNVSEYRPTTGTTAQAPTFGNFSTD
LEDYLFPKDEFPYIYQYIYPYLNTTDPRRASADPHYGQTAEEFLPPHATD
DDPQPLLRSSGGNSPGGNRQLYDIVYTITADITNTGSVVGEEVPQLYVSL
GGPEDPKVQLRDFDRMRIEPGETRQFTGRLTRRDLSNWDVTVQDWVISRY
PKTAYVGRSSRKLDLKIELP
(SEQ ID NO: 117)
IESRKVHQKPLARSEPFYPSPWMNPNADGWAEAYAQAKSFVSQMTLLEKV
NLTTGVGWGAEQCVGQVGAIPRLGLRSLCMHDSPLGIRGADYNSAFPSGQ
TVAATWDRGLMYRRGYAMGQEAKGKGINVLLGPVAGPLGRMPEGGRNWEG
FAPDPVLTGIGMSETIKGIQDAGVIACAKHFIGNEQEHFRQVPEAQGYGY
NISETLSSNIDDKTMHELYLWPFADAVRAGVGSVMCSYQQVNNSYACQNS
KLLNDLLKNELGFQGFVMSDWQAQHTGAASAVAGLDMSMPGDTQFNTGVS
FWGANLTLAVLNGTVPAYRLDDMAMRIMAALFKVTKTTDLEPINFSFWTD
DTYGPIHWAAKQGYQEINSHVDVRADHGNLIREIAAKGTVLLKNTGSLPL
NKPKFVAVIGEDAGSSPNGPNGCSDRGCNEGTLAMGWGSGTANYPYLVSP
DAALQARAIQDGTRYESVLSNYAEEKTKALVSQANATAIVFVNADSGEGY
INVDGNEGDRKNLTLWNNGDTLVKNVSSWCSNTIVVIHSVGPVLLTDWYD
NPNITAILWAGLPGQESGNSITDVLYGKVNPAARSPFTWGKTRESYGADV
LYKPNNGNGAPQQDFTEGVFIDYRYFDKVDDDSVIYEFGHGLSYTTFEYS
NIRVVKSNVSEYRPTTGTTAQAPTFGNFSTDLEDYLFPKDEFPYIYQYIY
PYLNTTDPRRASADPHYGQTAEEFLPPHATDDDPQPLLRSSGGNSPGGNR
QLYDIVYTITADITNTGSVVGEEVPQLYVSLGGPEDPKVQLRDFDRMRIE
PGETRQFTGRLTRRDLSNWDVTVQDWVISRYPKTAYVGRSSRKLDLKIEL
P

The polynucleotide (SEQ ID NO:118) and amino acid (SEQ ID NO:119) sequences of a BGL variant (“Variant 883”) are provided below. The signal sequence is underlined in SEQ ID NO:119. SEQ ID NO:120 provides the sequence of this BGL variant, without the signal sequence.

(SEQ ID NO: 118)
ATGAAGGCTGCTGCGCTTTCCTGCCTCTTCGGCAGTACCCTTGCCGTTGC
AGGCGCCATTGAATCGAGAAAGGTTCACCAGAAGCCCCTCGCGAGATCTG
AACCTTTTTACCCGTCGCCATGGATGAATCCCAACGCCGACGGCTGGGCG
GAGGCCTATGCCCAGGCCAAGTCCTTTGTCTCCCAAATGACTCTGCTAGA
GAAGGTCAACTTGACCACGGGAGTCGGCTGGGGGGCTGAGCAGTGCGTCG
GCCAAGTGGGCGCGATCCCTCGCCTTGGACTTCGCAGTCTGTGCATGCAT
GACTCCCCTCTCGGCATCCGAGGAGCCGACTACAACTCAGCGTTCCCCTC
TGGCCAGACCGTTGCTGCTACCTGGGATCGCGGTCTGATGTACCGTCGCG
GCTACGCAATGGGCCAGGAGGCCAAAGGCAAGGGCATCAATGTCCTTCTC
GGACCAGTCGCCGGCCCCCTTGGCCGCATGCCCGAGGGCGGTCGTAACTG
GGAAGGCTTCGCTCCGGATCCCGTCCTTACCGGCATCGGCATGTCCGAGA
CGATCAAGGGCATTCAGGATGCTGGCGTCATCGCTTGTGCGAAGCACTTT
ATTGGAAACGAGCAGGAGCACTTCAGACAGGTGCCAGAAGCCCAGGGATA
CGGTTACAACATCAGCGAAACCCTCTCCTCCAACATTGACGACAAGACCA
TGCACGAGCTCTACCTTTGGCCGTTTGCCGATGCCGTCCGGGCCGGCGTC
GGCTCTGTCATGTGCTCGTACAACCAGGTCAACAACTCGTACGCCTGCCA
GAACTCGAAGCTGCTGAACGACCTCCTCAAGAACGAGCTTGGGTTTCAGG
GCTTCGTCATGAGCGACTGGTGGGCACAGCACACTGGCGCAGCAAGCGCC
GTGGCTGGTCTCGATATGTCCATGCCGGGCGACACCATGTTCAACACTGG
CGTCAGTTTCTGGGGCGCCAATCTCACCCTCGCCGTCCTCAACGGCACAG
TCCCTGCCTACCGTCTCGACGACATGGCCATGCGCATCATGGCCGCCCTC
TTCAAGGTCACCAAGACCACCGACCTGGAACCGATCAACTTCTCCTTCTG
GACCCGCGACACTTATGGCCCGATCCACTGGGCCGCCAAGCAGGGCTACC
AGGAGATTAATTCCCACGTTGACGTCCGCGCCGACCACGGCAACCTCATC
CGGAACATTGCCGCCAAGGGTACGGTGCTGCTGAAGAATACCGGCTCTCT
ACCCCTGAACAAGCCAAAGTTCGTGGCCGTCATCGGCGAGGATGCTGGGC
CGAGCCCCAACGGGCCCAACGGCTGCAGCGACCGCGGCTGTAACGAAGGC
ACGCTCGCCATGGGCTGGGGATCCGGCACAGCCAACTATCCGTACCTCGT
TTCCCCCGACGCCGCGCTCCAGTTGCGGGCCATCCAGGACGGCACGAGGT
ACGAGAGCGTCCTGTCCAACTACGCCGAGGAAAATACAAAGGCTCTGGTC
TCGCAGGCCAATGCAACCGCCATCGTCTTCGTCAATGCCGACTCAGGCGA
GGGCTACATCAACGTGGACGGTAACGAGGGCGACCGTAAGAACCTGACTC
TCTGGAACAACGGTGATACTCTGGTCAAGAACGTCTCGAGCTGGTGCAGC
AACACCATCGTCGTCATCCACTCGGTCGGCCCGGTCCTCCTGACCGATTG
GTACGACAACCCCAACATCACGGCCATTCTCTGGGCTGGTCTTCCGGGCC
AGGAGTCGGGCAACTCCATCACCGACGTGCTTTACGGCAAGGTCAACCCC
GCCGCCCGCTCGCCCTTCACTTGGGGCAAGACCCGCGAAAGCTATGGCGC
GGACGTCCTGTACAAGCCGAATAATGGCAATTGGGCGCCCCAACAGGACT
TCACCGAGGGCGTCTTCATCGACTACCGCTACTTCGACAAGGTTGACGAT
GACTCGGTCATCTACGAGTTCGGCCACGGCCTGAGCTACACCACCTTCGA
GTACAGCAACATCCGCGTCGTCAAGTCCAACGTCAGCGAGTACCGGCCCA
CGACGGGCACCACGATTCAGGCCCCGACGTTTGGCAACTTCTCCACCGAC
CTCGAGGACTATCTCTTCCCCAAGGACGAGTTCCCCTACATCCCGCAGTA
CATCTACCCGTACCTCAACACGACCGACCCCCGGAGGGCCTCGGCCGATC
CCCACTACGGCCAGACCGCCGAGGAGTTCCTCCCGCCCCACGCCACCGAT
GACGACCCCCAGCCGCTCCTCCGGTCCTCGGGCGGAAACTCCCCCGGCGG
CAACCGCCAGCTGTACGACATTGTCTACACAATCACGGCCGACATCACGA
ATACGGGCTCCGTTGTAGGCGAGGAGGTACCGCAGCTCTACGTCTCGCTG
GGCGGTCCCGAGGATCCCAAGGTGCAGCTGCGCGACTTTGACAGGATGCG
GATCGAACCCGGCGAGACGAGGCAGTTCACCGGCCGCCTGACGCGCAGAG
ATCTGAGCAACTGGGACGTCACGGTGCAGGACTGGGTCATCAGCAGGTAT
CCCAAGACGGCATATGTTGGGAGGAGCAGCCGGAAGTTGGATCTCAAGAT
TGAGCTTCCTTGA
(SEQ ID NO: 119)
MKAAALSCLFGSTLAVAGAIESRKVHQKPLARSEPFYPSPWMNPNADGWA
EAYAQAKSFVSQMTLLEKVNLTTGVGWGAEQCVGQVGAIPRLGLRSLCMH
DSPLGIRGADYNSAFPSGQTVAATWDRGLMYRRGYAMGQEAKGKGINVLL
GPVAGPLGRMPEGGRNWEGFAPDPVLTGIGMSETIKGIQDAGVIACAKHF
IGNEQEHFRQVPEAQGYGYNISETLSSNIDDKTMHELYLWPFADAVRAGV
GSVMCSYNQVNNSYACQNSKLLNDLLKNELGFQGFVMSDWWAQHTGAASA
VAGLDMSMPGDTMFNTGVSFWGANLTLAVLNGTVPAYRLDDMAMRIMAAL
FKVTKTTDLEPINFSFWTRDTYGPIHWAAKQGYQEINSHVDVRADHGNLI
RNIAAKGTVLLKNTGSLPLNKPKFVAVIGEDAGPSPNGPNGCSDRGCNEG
TLAMGWGSGTANYPYLVSPDAALQLRAIQDGTRYESVLSNYAEENTKALV
SQANATAIVFVNADSGEGYINVDGNEGDRKNLTLWNNGDTLVKNVSSWCS
NTIVVIHSVGPVLLTDWYDNPNITAILWAGLPGQESGNSITDVLYGKVNP
AARSPFTWGKTRESYGADVLYKPNNGNWAPQQDFTEGVFIDYRYFDKVDD
DSVIYEFGHGLSYTTFEYSNIRVVKSNVSEYRPTTGTTIQAPTFGNFSTD
LEDYLFPKDEFPYIPQYTYPYLNTTDPRRASADPHYGQTAEEFLPPHATD
DDPQPLLRSSGGNSPGGNRQLYDIVYTITADITNTGSVVGEEVPQLYVSL
GGPEDPKVQLRDFDRMRIEPGETRQFTGRLTRRDLSNWDVTVQDWVISRY
PKTAYVGRSSRKLDLKIELP
(SEQ ID NO: 120)
IESRKVHQKPLARSEPFYPSPWMNPNADGWAEAYAQAKSFVSQMTLLEKV
NLYTGVGWGAEQCVGQVGAIPRLGLRSLCMHDSPLGIRGADYNSAFPSGQ
TVAATWDRGLMYRRGYAMGQEAKGKGINVLLGPVAGPLGRMPEGGRNWEG
FAPDPVLTGIGMSETIKGIQDAGVIACAKHFIGNEQEHFRQVPEAQGYGY
NISETLSSNIDDKTMHELYLWPFADAVRAGVGSVMCSYNQVNNSYACQNS
KLLNDLLKNELGFQGFVMSDWWAQHTGAASAVAGLDMSMPGDTMFNTGVS
FWGANLTLAVLNGTVPAYRLDDMAMRIMAALFKVTKTTDLEPINFSFWTR
DTYGPIHWAAKQGYQEINSHVDVRADHGNLIRNIAAKGTVLLKNTGSLPL
NKPKFVAVIGEDAGPSPNGPNGCSDRGCNEGTLAMGWGSGTANYPYLVSP
DAALQLRAIQDGTRYESVLSNYAEENTKALVSQANATAIVFVNADSGEGY
INVDGNEGDRKNLTKWNNGDTLVKNVSSWCSNTIVVIHSVGPVLLTDWYD
NPNITAILWAGLPGQESGNSITDVLYGKVNPAARSPFTWGKTRESYGADV
LYKPNNGNWAPQQDFTEGVFIDYRYFDKVDDDSVIYEFGHGLSYTTFEYS
NIRVVKSNVSEYRPTTGTTIQAPTFGNFSTDLEDYLFPKDEFPYIPQYIY
PYLNTTDPRRASADPHYGQTAEEFLPPHATDDDPQPLLRSSGGNSPGGNR
QLYDIVYTITADITNTGSVVGEEVPQLYVSLGGPEDPKVQLRDFDRMRIE
PGETRQFTGRLTRRDLSNWDVTVQDWVISRYPKTAYVGRSSRKLDLKIEL
P

The polynucleotide (SEQ ID NO:121) and amino acid (SEQ ID NO:122) sequences of a BGL variant (“Variant 900”) are provided below. The signal sequence is underlined in SEQ ID NO:122. SEQ ID NO:123 provides the sequence of this BGL variant, without the signal sequence.

(SEQ ID NO: 121)
ATGAAGGCTGCTGCGCTTTCCTGCCTCTTCGGCAGTACCCTTGCCGTTGC
AGGCGCCATTGAATCGAGAAAGGTTCACCAGAAGCCCCTCGCGAGATCTG
AACCTTTTTACCCGTCGCCATGGATGAATCCCAACGCCATCGGCTGGGCG
GAGGCCTATGCCCAGGCCAAGTCCTTTGTCTCCCAAATGACTCTGCTAGA
GAAGGTCAACTTGACCACGGGAGTCGGCTGGGGGGAGGAGCAGTGCGTCG
GCAACGTGGGCGCGATCCCTCGCCTTGGACTTCGCAGTCTGTGCATGCAT
GACTCCCCTCTCGGCGTGCGAGGAACCGACTACAACTCAGCGTTCCCCTC
TGGCCAGACCGTTGCTGCTACCTGGGATCGCGGTCTGATGTACCGTCGCG
GCTACGCAATGGGCCAGGAGGCCAAAGGCAAGGGCATCAATGTCCTTCTC
GGACCAGTCGCCGGCCCCCTTGGCCGCATGCCCGAGGGCGGTCGTAACTG
GGAAGGCTTCGCTCCGGATCCCGTCCTTACCGGCATCGGCATGTCCGAGA
CGATCAAGGGCATTCAGGATGCTGGCGTCATCGCTTGTGCGAAGCACTTT
ATTGGAAACGAGCAGGAGCACTTCAGACAGGTGCCAGAAGCCCAGGGATA
CGGTTACAACATCAGCGAAACCCTCTCCTCCAACATTGACGACAAGACCA
TGCACGAGCTCTACCTTTGGCCGTTTGCCGATGCCGTCCGGGCCGGCGTC
GGCTCTGTCATGTGCTCGTACAACCAGGGCAACAACTCGTACGCCTGCCA
GAACTCGAAGCTGCTGAACGACCTCCTCAAGAACGAGCTTGGGTTTCAGG
GCTTCGTCATGAGCGACTGGTGGGCACAGCACACTGGCGCAGCAAGCGCC
GTGGCTGGTCTCGATATGTCCATGCCGGGCGACACCATGGTCAACACTGG
CGTCAGTTTCTGGGGCGCCAATCTCACCCTCGCCGTCCTCAACGGCACAG
TCCCTGCCTACCGTCTCGACGACATGTGCATGCGCATCATGGCCGCCCTC
TTCAAGGTCACCAAGACCACCGACCTGGAACCGATCAACTTCTCCTTCTG
GACCCGCGACACTTATGGCCCGATCCACTGGGCCGCCAAGCAGGGCTACC
AGGAGATTAATTCCCACGTTGACGTCCGCGCCGACCACGGCAACCTCATC
CGGAACATTGCCGCCAAGGGTACGGTGCTGCTGAAGAATACCGGCTCTCT
ACCCCTGAACAAGCCAAAGTTCGTGGCCGTCATCGGCGAGGATGCTGGGC
CGAGCCCCAACGGGCCCAACGGCTGCAGCGACCGCGGCTGTAACGAAGGC
ACGCTCGCCATGGGCTGGGGATCCGGCACAGCCAACTATCCGTACCTCGT
TTCCCCCGACGCCGCGCTCCAGGCGCGGGCCATCCAGGACGGCACGAGGT
ACGAGAGCGTCCTGTCCAACTACGCCGAGGAAAATACAAAGGCTCTGGTC
TCGCAGGCCAATGCAACCGCCATCGTCTTCGTCAATGCCGACTCAGGCGA
GGGCTACATCAACGTGGACGGTAACGAGGGCGACCGTAAGAACCTGACTC
TCTGGAACAACGGTGATACTCTGGTCAAGAACGTCTCGAGCTGGTGCAGC
AACACCATCGTCGTCATCCACTCGGTCGGCCCGGTCCTCCTGACCGATTG
GTACGACAACCCCAACATCACGGCCATTCTCTGGGCTGGTCTTCCGGGCC
AGGAGTCGGGCAACTCCATCACCGACGTGCTTTACGGCAAGGTCAACCCC
GCCGCCCGCTCGCCCTTCACTTGGGGCAAGACCCGCGAAAGCTATGGCGC
GGACGTCCTGTACAAGCCGAATAATGGCAATTGGGCGCCCCAACAGGACT
TCACCGAGGGCGTCTTCATCGACTACCGCTACTTCGACAAGGTTGACGAT
GACTCGGTCATCTACGAGTTCGGCCACGGCCTGAGCTACACCACCTTCGA
GTACAGCAACATCCGCGTCGTCAAGTCCAACGTCAGCGAGTACCGGCCCA
CGACGGGCACCACGATTCAGGCCCCGACGTTTGGCAACTTCTCCACCGAC
CTCGAGGACTATCTCTTCCCCAAGGACGAGTTCCCCTACATCCCGCAGTA
CATCTACCCGTACCTCAACACGACCGACCCCCGGAGGGCCTCGGGCGATC
CCCACTACGGCCAGACCGCCGAGGAGTTCCTCCCGCCCCACGCCACCGAT
GACGACCCCCAGCCGCTCCTCCGGTCCTCGGGCGGAAACTCCCCCGGCGG
CAACCGCCAGCTGTACGACATTGTCTACACAATCACGGCCGACATCACGA
ATACGGGCTCCGTTGTAGGCGAGGAGGTACCGCAGCTCTACGTCTCGCTG
GGCGGTCCCGAGGATCCCAAGGTGCAGCTGCGCGACTTTGACAGGATGCG
GATCGAACCCGGCGAGACGAGGCAGTTCACCGGCCGCCTGACGCGCAGAG
ATCTGAGCAACTGGGACGTCACGGTGCAGGACTGGGTCATCAGCAGGTAT
CCCAAGACGGCATATGTTGGGAGGAGCAGCCGGAAGTTGGATCTCAAGAT
TGAGCTTCCTTGA
(SEQ ID NO: 122)
MKAAALSCLFGSTLAVAGAIESRKVHQKPLARSEPFYPSPWMNPNAIGWA
EAYAQAKSFVSQMTLLEKVNLTTGVGWGEEQCVGNVGAIPRLGLRSLCMH
DSPLGVRGTDYNSAFPSGQTVAATWDRGLMYRRGYAMGQEAKGKGINVLL
GPVAGPLGRMPEGGRNWEGFAPDPVLTGIGMSETIKGIQDAGVIACAKHF
IGNEQEHFRQVPEAQGYGYNISETLSSNIDDKTMHELYLWPFADAVRAGV
GSVMCSYNQGNNSYACQNSKLLNDLLKNELGFQGFVMSDWWAQHTGAASA
VAGLDMSMPGDTMVNTGVSFWGANLTLAVLNGTVPAYRLDDMCMRIMAAL
FKVTKTTDLEPINFSFWTRDTYGPIHWAAKQGYQEINSHVDVRADHGNLI
RNIAAKGTVLLKNTGSLPLNKPKFVAVIGEDAGPSPNGPNGCSDRGCNEG
TLAMGWGSGTANYPYLVSPDAALQARAIQDGTRYESVLSNYAEENTKALV
SQANATAIVFVNADSGEGYINVDGNEGDRKNLTLWNNGDTLVKNVSSWCS
NTIVVIHSVGPVLLTDWYDNPNITAILWAGLPGQESGNSITDVLYGKVNP
AARSPFTWGKTRESYGADVLYKPNNGNWAPQQDFTEGVFIDYRYFDKVDD
DSVIYEFGHGLSYTTFEYSNIRVVKSNVSEYRPTTGTTIQAPTFGNFSTD
LEDYLFPKDEFPYIPQYIYPYLNTTDPRRASGDPHYGQTAEEFLPPHATD
DDPQPLLRSSGGNSPGGNRQLYDIVYTITADITNTGSVVGEEVPQLYVSL
GGPEDPKVQLRDFDRMRIEPGETRQFTGRLTRRDLSNWDVTVQDWVISRY
PKTAYVGRSSRKLDLKIELP
(SEQ ID NO: 123)
IESRKVHQKPLARSEPFYPSPWMNPNAIGWAEAYAQAKSFVSQMTLLEKV
NLTTGVGWGEEQCVGNVGAIPRLGLRSLCMHDSPLGVRGTDYNSAFPSGQ
TVAATWDRGLMYRRGYAMGQEAKGKGINVLLGPVAGPLGRMPEGGRNWEG
FAPDPVLTGIGMSETIKGIQDAGVIACAKHFIGNEQEHFRQVPEAQGYGY
NISETLSSNIDDKTMHELYLWPFADAVRAGVGSVMCSYNQGNNSYACQNS
KLLNDLLKNELGFQGFVMSDWWAQHTGAASAVAGLDMSMPGDTMVNTGVS
FWGANLTLAVLNGTVPAYRLDDMCMRIMAALFKVTKTTDLEPINFSFWTR
DTYGPIHWAAKQGYQEINSHVDVRADHGNLIRNIAAKGTVLLKNTGSLPL
NKPKFVAVIGEDAGPSPNGPNGCSDRGCNEGTLAMGWGSGTANYPYLVSP
DAALQARAIQDGTRYESVLSNYAEENTKALVSQANATAIVFVNADSGEGY
INVDGNEGDRKNLTLWNNGDTLVKNVSSWCSNTIVVIHSVGPVLLTDWYD
NPNITAILWAGLPGQESGNSITDVLYGKVNPAARSPFTWGKTRESYGADV
LYKPNNGNWAPQQDFTEGVFIDYRYFDKVDDDSVIYEFGHGLSYTTFEYS
NIRVVKSNVSEYRPTTGTTIQAPTFGNFSTDLEDYLFPKDEFPYIPQYIY
PYLNTTDPRRASGDPHYGQTAEEFLPPHATDDDPQPLLRSSGGNSPGGNR
QLYDIVYTITADITNTGSVVGEEVPQLYVSLGGPEDPKVQLRDFDRMRIE
PGETRQFTGRLTRRDLSNWDVTVQDWVISRYPKTAYVGRSSRKLDLKIEL
P

The polynucleotide (SEQ ID NO:124) and amino acid (SEQ ID NO:125) sequences of wild-type Talaromyces emersonii CBH1 are provided below. The signal sequence is shown underlined in SEQ ID NO:125. SEQ ID NO:126 provides the sequence of this CBH1, without the signal sequence.

(SEQ ID NO: 124)
ATGCTTCGACGGGCTCTTCTTCTATCCTCTTCCGCCATCCTTGCTGTCAA
GGCACAGCAGGCCGGCACGGCGACGGCAGAGAACCACCCGCCCCTGACAT
GGCAGGAATGCACCGCCCCTGGGAGCTGCACCACCCAGAACGGGGCGGTC
GTTCTTGATGCGAACTGGCGTTGGGTGCACGATGTGAACGGATACACCAA
CTGCTACACGGGCAATACCTGGGACCCCACGTACTGCCCTGACGACGAAA
CCTGCGCCCAGAACTGTGCGCTGGACGGCGCGGATTACGAGGGCACCTAC
GGCGTGACTTCGTCGGGCAGCTCCTTGAAACTCAATTTCGTCACCGGGTC
GAACGTCGGATCCCGTCTCTACCTGCTGCAGGACGACTCGACCTATCAGA
TCTTCAAGCTTCTGAACCGCGAGTTCAGCTTTGACGTCGATGTCTCCAAT
CTTCCGTGCGGATTGAACGGCGCTCTGTACTTTGTCGCCATGGACGCCGA
CGGCGGCGTGTCCAAGTACCCGAACAACAAGGCTGGTGCCAAGTACGGAA
CCGGGTATTGCGACTCCCAATGCCCACGGGACCTCAAGTTCATCGACGGC
GAGGCCAACGTCGAGGGCTGGCAGCCGTCTTCGAACAACGCCAACACCGG
AATTGGCGACCACGGCTCCTGCTGTGCGGAGATGGATGTCTGGGAAGCAA
ACAGCATCTCCAATGCGGTCACTCCGCACCCGTGCGACACGCCAGGCCAG
ACGATGTGCTCTGGAGATGACTGCGGTGGCACATACTCTAACGATCGCTA
CGCGGGAACCTGCGATCCTGACGGCTGTGACTTCAACCCTTACCGCATGG
GCAACACTTCTTTCTACGGGCCTGGCAAGATCATCGATACCACCAAGCCC
TTCACTGTCGTGACGCAGTTCCTCACTGATGATGGTACGGATACTGGAAC
TCTCAGCGAGATCAAGCGCTTCTACATCCAGAACAGCAACGTCATTCCGC
AGCCCAACTCGGACATCAGTGGCGTGACCGGCAACTCGATCACGACGGAG
TTCTGCACTGCTCAGAAGCAGGCCTTTGGCGACACGGACGACTTCTCTCA
GCACGGTGGCCTGGCCAAGATGGGAGCGGCCATGCAGCAGGGTATGGTCC
TGGTGATGAGTTTGTGGGACGACTACGCCGCGCAGATGCTGTGGTTGGAT
TCCGACTACCCGACGGATGCGGACCCCACGACCCCTGGTATTGCCCGTGG
AACGTGTCCGACGGACTCGGGCGTCCCATCGGATGTCGAGTCGCAGAGCC
CCAACTCCTACGTGACCTACTCGAACATTAAGTTTGGTCCGATCAACTCG
ACCTTCACCGCTTCGTGA
(SEQ ID NO: 125)
MLRRALLLSSSAILAVKAQQAGTATAENHPPLTWQECTAPGSCTTQNGAV
VLDANWRWVHDVNGYTNCYTGNTWDPTYCPDDETCAQNCALDGADYEGTY
GVTSSGSSLKLNFVTGSNVGSRLYLLQDDSTYQIFKLLNREFSFDVDVSN
LPCGLNGALYFVAMDADGGVSKYPNNKAGAKYGTGYCDSQCPRDLKFIDG
EANVEGWQPSSNNANTGIGDHGSCCAEMDVWEANSISNAVTPHPCDTPGQ
TMCSGDDCGGTYSNDRYAGTCDPDGCDFNPYRMGNTSFYGPGKIIDTTKP
FTVVTQFLTDDGTDTGTLSEIKRFYIQNSNVIPQPNSDISGVTGNSITTE
FCTAQKQAFGDTDDFSQHGGLAKMGAAMQQGMVLVMSLWDDYAAQMLWLD
SDYPTDADPTTPGIARGTCPTDSGVPSDVESQSPNSYVTYSNIKFGPINS
TFTAS
(SEQ ID NO: 126)
QQAGTATAENHPPLTWQECTAPGSCTTQNGAVVLDANWRWVHDVNGYTNC
YTGNTWDPTYCPDDETCAQNCALDGADYEGTYGVTSSGSSLKLNFVTGSN
VGSRLYLLQDDSTYQIFKLLNREFSFDVDVSNLPCGLNGALYFVAMDADG
GVSKYPNNKAGAKYGTGYCDSQCPRDLKFIDGEANVEGWQPSSNNANTGI
GDHGSCCAEMDVWEANSISNAVTPHPCDTPGQTMCSGDDCGGTYSNDRYA
GTCDPDGCDFNPYRMGNTSFYGPGKIIDTTKPFTVVTQFLTDDGTDTGTL
SEIKRFYIQNSNVIPQPNSDISGVTGNSITTEFCTAQKQAFGDTDDFSQH
GGLAKMGAAMQQGMVLVMSLWDDYAAQMLWLDSDYPTDADPTTPGIARGT
CPTDSGVPSDVESQSPNSYVTYSNIKFGPINSTFTAS

The polynucleotide (SEQ ID NO:127) and amino acid (SEQ ID NO:128) sequences of wild-type M. thermophila CBH1a are provided below. The signal sequence is shown underlined in SEQ ID NO:128. SEQ ID NO:129 provides the sequence of this CBH1a, without the signal sequence.

(SEQ ID NO: 127)
ATGTACGCCAAGTTCGCGACCCTCGCCGCCCTTGTGGCTGGCGCCGCTGC
TCAGAACGCCTGCACTCTGACCGCTGAGAACCACCCCTCGCTGACGTGGT
CCAAGTGCACGTCTGGCGGCAGCTGCACCAGCGTCCAGGGTTCCATCACC
ATCGACGCCAACTGGCGGTGGACTCACCGGACCGATAGCGCCACCAACTG
CTACGAGGGCAACAAGTGGGATACTTCGTACTGCAGCGATGGTCCTTCTT
GCGCCTCCAAGTGCTGCATCGACGGCGCTGACTACTCGAGCACCTATGGC
ATCACCACGAGCGGTAACTCCCTGAACCTCAAGTTCGTCACCAAGGGCCA
GTACTCGACCAACATCGGCTCGCGTACCTACCTGATGGAGAGCGACACCA
AGTACCAGATGTTCCAGCTCCTCGGCAACGAGTTCACCTTCGATGTCGAC
GTCTCCAACCTCGGCTGCGGCCTCAATGGCGCCCTCTACTTCGTGTCCAT
GGATGCCGATGGTGGCATGTCCAAGTACTCGGGCAACAAGGCAGGTGCCA
AGTACGGTACCGGCTACTGTGATTCTCAGTGCCCCCGCGACCTCAAGTTC
ATCAACGGCGAGGCCAACGTAGAGAACTGGCAGAGCTCGACCAACGATGC
CAACGCCGGCACGGGCAAGTACGGCAGCTGCTGCTCCGAGATGGACGTCT
GGGAGGCCAACAACATGGCCGCCGCCTTCACTCCCCACCCTTGCACCGTG
ATCGGCCAGTCGCGCTGCGAGGGCGACTCGTGCGGCGGTACCTACAGCAC
CGACCGCTATGCCGGCATCTGCGACCCCGACGGATGCGACTTCAACTCGT
ACCGCCAGGGCAACAAGACCTTCTACGGCAAGGGCATGACGGTCGACACG
ACCAAGAAGATCACGGTCGTCACCCAGTTCCTCAAGAACTCGGCCGGCGA
GCTCTCCGAGATCAAGCGGITCTACGTCCAGAACGGCAAGGTCATCCCCA
ACTCCGAGTCCACCATCCCGGGCGTCGAGGGCAACTCCATCACCCAGGAC
TGGTGCGACCGCCAGAAGGCCGCCTTCGGCGACGTGACCGACTTCCAGGA
CAAGGGCGGCATGGTCCAGATGGGCAAGGCCCTCGCGGGGCCCATGGTCC
TCGTCATGTCCATCTGGGACGACCACGCCGTCAACATGCTCTGGCTCGAC
TCCACCTGGCCCATCGACGGCGCCGGCAAGCCGGGCGCCGAGCGCGGTGC
CTGCCCCACCACCTCGGGCGTCCCCGCTGAGGTCGAGGCCGAGGCCCCCA
ACTCCAACGTCATCTTCTCCAACATCCGCTTCGGCCCCATCGGCTCCACC
GTCTCCGGCCTGCCCGACGGCGGCAGCGGCAACCCCAACCCGCCCGTCAG
CTCGTCCACCCCGGTCCCCTCCTCGTCCACCACATCCTCCGGTTCCTCCG
GCCCGACTGGCGGCACGGGTGTCGCTAAGCACTATGAGCAATGCGGAGGA
ATCGGGTTCACTGGCCCTACCCAGTGCGAGAGCCCCTACACTTGCACCAA
GCTGAATGACTGGTACTCGCAGTGCCTGTAA
(SEQ ID NO: 128)
MYAKFATLAALVAGAAAQNACTLTAENHPSLTYSKCTSGGSCTSVQGSIT
IDANWRWTHRTDSATNCYEGNKWDTSWCSDGPSCASKCCIDGADYSSTYG
ITTSGNSLNLKFVTKGQYSTNIGSRTYLMESDTKYQMFQLLGNEFTFDVD
VSNLGCGLNGALYFVSMDADGGMSKYSGNKAGAKYGTGYCDSQCPRDLKF
INGEANVENWQSSTNDANAGTGKYGSCCSEMDVWEANNMAAAFTPHPCTV
IGQSRCEGDSCGGTYSTDRYAGICDPDGCDFNSYRQGNKTFYGKGMTVDT
TKKITVVTQFLKNSAGELSEIKRFYVQNGKVIPNSESTIPGVEGNSITQD
WCDRQKAAFGDVTDFQDKGGMVQMGKALAGPMVLVMSIWDDHAVNMLWLD
STWPIDGAGKPGAERGACPTTSGVPAEVEAEAPNSNVIFSNIRFGPIGST
VSGLPDGGSGNPNPPVSSSTPVPSSSTTSSGSSGPTGGTGVAKHYEQCGG
IGFTGPTQCESPYTCTKLNDWYSQCL
(SEQ ID NO: 129)
QNACTLTAENHPSLTYSKCTSGGSCTSVQGSITIDANWRWTHRTDSATNC
YEGNKWDTSWCSDGPSCASKCCIDGADYSSTYGITTSGNSLNLKFVTKGQ
YSTNIGSRTYLMESDTKYQMFQLLGNEFTFDVDVSNLGCGLNGALYFVSM
DADGGMSKYSGNKAGAKYGTGYCDSQCPRDLKFINGEANVENWQSSTNDA
NAGTGKYGSCCSEMDVWEANNMAAAFTPHPCTVIGQSRCEGDSCGGTYST
DRYAGICDPDGCDFNSYRQGNKTFYGKGMTVDTTKKITVVTQFLKNSAGE
LSEIKRFYVQNGKVIPNSESTIPGVEGNSITQDWCDRQKAAFGDVTDFQD
KGGMVQMGKALAGPMVLVMSIWDDHAVNMLWLDSTWPIDGAGKPGAERGA
CPTTSGVPAEVEAEAPNSNVIFSNIRFGPIGSTVSGLPDGGSGNPNPPVS
SSTPVPSSSTTSSGSSGPTGGTGVAKHYEQCGGIGFTGPTQCESPYTCTK
LNDWYSQCL

The polynucleotide (SEQ ID NO:130) and amino acid (SEQ ID NO:131) sequences of a M. thermophila CBH1a variant (“Variant 145”) are provided below. The signal sequence is shown underlined in SEQ ID NO:131. SEQ ID NO:132 provides the sequence of this CBH1a, without the signal sequence.

(SEQ ID NO: 130)
ATGTACGCCAAGTTCGCGACCCTCGCCGCCCTTGTGGCTGGCGCCGCTGC
TCAGAACGCCTGCACTCTGACCGCTGAGAACCACCCCTCGCTGACGTGGT
CCAAGTGCACGTCTGGCGGCAGCTGCACCAGCGTCCAGGGTTCCATCACC
ATCGACGCCAACTGGCGGTGGACTCACCGGACCGATAGCGCCACCAACTG
CTACGAGGGCAACAAGTGGGATACTTCGTGGTGCAGCGATGGTCCTTCTT
GCGCCTCCAAGTGCTGCATCGACGGCGCTGACTACTCGAGCACCTATGGC
ATCACCACGAGCGGTAACTCCCTGAACCTCAAGTTCGTCACCAAGGGCCA
GTACTCGACCAACATCGGCTCGCGTACCTACCTGATGGAGAGCGACACCA
AGTACCAGATGTTCCAGCTCCTCGGCAACGAGTTCACCTTCGATGTCGAC
GTCTCCAACCTCGGCTGCGGCCTCAATGGCGCCCTCTACTTCGTGTCCAT
GGATGCCGATGGTGGCATGTCCAAGTACTCGGGCAACAAGGCAGGTGCCA
AGTACGGTACCGGCTACTGTGATTCTCAGTGCCCCCGCGACCTCAAGTTC
ATCAACGGCGAGGCCAACGTAGAGAACTGGCAGAGCTCGACCAACGATGC
CAACGCCGGCACGGGCAAGTACGGCAGCTGCTGCTCCGAGATGGACGTCT
GGGAGGCCAACAACATGGCCGCCGCCTTCACTCCCCACCCTTGCACCGTG
ATCGGCCAGTCGCGCTGCGAGGGCGACTCGTGCGGCGGTACCTACAGCAC
CGACCGCTATGCCGGCATCTGCGACCCCGACGGATGCGACTTCAACTCGT
ACCGCCAGGGCAACAAGACCTTCTACGGCAAGGGCATGACGGTCGACACG
ACCAAGAAGATCACGGTCGTCACCCAGTTCCTCAAGAACTCGGCCGGCGA
GCTCTCCGAGATCAAGCGGTTCTACGTCCAGAACGGCAAGGTCATCCCCA
ACTCCGAGTCCACCATCCCGGGCGTCGAGGGCAACTCCATCACCCAGGAC
TGGTGCGACCGCCAGAAGGCCGCCTTCGGCGACGTGACCGACTTCCAGGA
CAAGGGCGGCATGGTCCAGATGGGCAAGGCCCTCGCGGGGCCCATGGTCC
TCGTCATGTCCATCTGGGACGACCACGCCGTCAACATGCTCTGGCTCGAC
TCCACCTGGCCCATCGACGGCGCCGGCAAGCCGGGCGCCGAGCGCGGTGC
CTGCCCCACCACCTCGGGCGTCCCCGCTGAGGTCGAGGCCGAGGCCCCCA
ACTCCAACGTCATCTTCTCCAACATCCGCTTCGGCCCCATCGGCTCCACC
GTCTCCGGCCTGCCCGACGGCGGCAGCGGCAACCCCAACCCGCCCGTCAG
CTCGTCCACCCCGGTCCCCTCCTCGTCCACCACATCCTCCGGTTCCTCCG
GCCCGACTGGCGGCACGGGTGTCGCTAAGCACTATGAGCAATGCGGAGGA
ATCGGGTTCACTGGCCCTACCCAGTGCGAGAGCCCCTACACTTGCACCAA
GCTGAATGACTGGTACTCGCAGTGCCTGTAA
(SEQ ID NO: 131)
MYAKFATLAALVAGAAAQNACTLTAENHPSLTWSKCTSGGSCTSVQGSIT
IDANWRWTHRTDSATNCYEGNKWDTSWCSDGPSCASKCCIDGADYSSTYG
ITTSGNSLNLKFVTKGQYSTNIGSRTYLMESDTKYQMFQLLGNEFTFDVD
VSNLGCGLNGALYFVSMDADGGMSKYSGNKAGAKYGTGYCDSQCPRDLKF
INGEANVENWQSSTNDANAGTGKYGSCCSEMDVWEANNMAAAFTPHPCTV
IGQSRCEGDSCGGTYSTDRYAGICDPDGCDFNSYRQGNKTFYGKGMTVDT
TKKITVVTQFLKNSAGELSEIKRFYVQNGKVIPNSESTIPGVEGNSITQD
WCDRQKAAFGDVTDFQDKGGMVQMGKALAGPMVLVMSIWDDHAVNMLWLD
STWPIDGAGKPGAERGACPTTSGVPAEVEAEAPNSNVIFSNIRFGPIGST
YSGLPDGGSGNPNPPVSSSTPVPSSSTTSSGSSGPTGGTGVAKHYEQCGG
IGFTGPTQCESPYTCTKLNDWYSQCL
(SEQ ID NO: 132)
QNACTLTAENHPSLTWSKCTSGGSCTSVQGSITIDANWRWTHRTDSATNC
YEGNKWDTSWCSDGPSCASKCCIDGADYSSTYGITTSGNSLNLKFVTKGQ
YSTNIGSRTYLMESDTKYQMFQLLGNEFTFDVDVSNLGCGLNGALYFVSM
DADGGMSKYSGNKAGAKYGTGYCDSQCPRDLKFINGEANVENWQSSTNDA
NAGTGKYGSCCSEMDVWEANNMAAAFTPHPCTVIGQSRCEGDSCGGTYST
DRYAGICDPDGCDFNSYRQGNKTFYGKGMTVDTTKKITVVTQFLKNSAGE
LSEIKRFYVQNGKVIPNSESTEPGVEGNSITQDWCDRQKAAFGDVTDFQD
KGGMVQMGKALAGPMVLVMSIWDDHAVNMLWLDSTWPIDGAGKPGAERGA
CPTTSGVPAEVEAEAPNSNVIFSNIRFGPIGSTVSGLPDGGSGNPNPPVS
SSTPVPSSSTTSSGSSGPTGGTGVAKHYEQCGGIGFTGPTQCESPYTCTK
LNDWYSQCL

The polynucleotide (SEQ ID NO:133) and amino acid (SEQ ID NO:134) sequences of a M. thermophila CBH1a variant (“Variant 983”) are provided below. The signal sequence is shown underlined in SEQ ID NO:134. SEQ ID NO:135 provides the sequence of this CBH1a variant, without the signal sequence.

(SEQ ID NO: 133)
ATGTACGCCAAGTTCGCGACCCTCGCCGCCCTTGTGGCTGGCGCCGCTGC
TCAGAACGCCTGCACTCTGAACGCTGAGAACCACCCCTCGCTGACGTGGT
CCAAGTGCACGTCTGGCGGCAGCTGCACCAGCGTCCAGGGTTCCATCACC
ATCGACGCCAACTGGCGGTGGACTCACCGGACCGATAGCGCCACCAACTG
CTACGAGGGCAACAAGTGGGATACTTCGTACTGCAGCGATGGTCCTTCTT
GCGCCTCCAAGTGCTGCATCGACGGCGCTGACTACTCGAGCACCTATGGC
ATCACCACGAGCGGTAACTCCCTGAACCTCAAGTTCGTCACCAAGGGCCA
GTACTCGACCAACATCGGCTCGCGTACCTACCTGATGGAGAGCGACACCA
AGTACCAGATGTTCCAGCTCCTCGGCAACGAGTTCACCTTCGATGTCGAC
GTCTCCAACCTCGGCTGCGGCCTCAATGGCGCCCTCTACTTCGTGTCCAT
GGATGCCGATGGTGGCATGTCCAAGTACTCGGGCAACAAGGCAGGTGCCA
AGTACGGTACCGGCTACTGTGATTCTCAGTGCCCCCGCGACCTCAAGTTC
ATCAACGGCGAGGCCAACGTAGAGAACTGGCAGAGCTCGACCAACGATGC
CAACGCCGGCACGGGCAAGTACGGCAGCTGCTGCTCCGAGATGGACGTCT
GGGAGGCCAACAACATGGCCGCCGCCTTCACTCCCCACCCTTGCACCGTG
ATCGGCCAGTCGCGCTGCGAGGGCGACTCGTGCGGCGGTACCTACAGCAC
CGACCGCTATGCCGGCATCTGCGACCCCGACGGATGCGACTTCAACTCGT
ACCGCCAGGGCAACAAGACCTTCTACGGCAAGGGCATGACGGTCGACACG
ACCAAGAAGATCACGGTCGTCACCCAGTTCCTCAAGAACTCGGCCGGCGA
GCTCTCCGAGATCAAGCGGTTCTACGTCCAGAACGGCAAGGTCATCCCCA
ACTCCGAGTCCACCATCCCGGGCGTCGAGGGCAACTCCATCACCCAGGAG
TACTGCGACCGCCAGAAGGCCGCCTTCGGCGACGTGACCGACTTCCAGGA
CAAGGGCGGCATGGTCCAGATGGGCAAGGCCCTCGCGGGGCCCATGGTCC
TCGTCATGTCCATCTGGGACGACCACGCCGACAACATGCTCTGGCTCGAC
TCCACCTGGCCCATCGACGGCGCCGGCAAGCCGGGCGCCGAGCGCGGTGC
CTGCCCCACCACCTCGGGCGTCCCCGCTGAGGTCGAGGCCGAGGCCCCCA
ACTCCAACGTCATCTTCTCCAACATCCGCTTCGGCCCCATCGGCTCCACC
GTCTCCGGCCTGCCCGACGGCGGCAGCGGCAACCCCAACCCGCCCGTCAG
CTCGTCCACCCCGGTCCCCTCCTCGTCCACCACATCCTCCGGTTCCTCCG
GCCCGACTGGCGGCACGGGTGTCGCTAAGCACTATGAGCAATGCGGAGGA
ATCGGGTTCACTGGCCCTACCCAGTGCGAGAGCCCCTACACTTGCACCAA
GCTGAATGACTGGTACTCGCAGTGCCTGTAA
(SEQ ID NO: 134)
MYAKFATLAALVAGAAAQNACTLNAENHPSLTWSKCTSGGSCTSVQGSIT
IDANWRWTHRTDSATNCYEGNKWDTSYCSDGPSCASKCCIDGADYSSTYG
ITTSGNSLNLKFVTKGQYSTNIGSRTYLMESDTKYQMFQLLGNEFTFDVD
VSNLGCGLNGALYFVSMDADGGMSKYSGNKAGAKYGTGYCDSQCPRDLKF
INGEA1WENWQSSTNDANAGTGKYGSCCSEMDVWEANNMAAAFTPHPCTV
IGQSRCEGDSCGGTYSTDRYAGICDPDGCDFNSYRQGNKTFYGKGMTVDT
TKKITVVTQFLKNSAGELSEIKRFYVQNGKVIPNSESTIPGVEGNSITQE
YCDRQKAAFGDVTDFQDKGGMVQMGKALAGPMVLVMSIWDDHADNMLWLD
STWPIDGAGKPGAERGACPTTSGVPAEVEAEAPNSNVIFSNIRFGPIGST
VSGLPDGGSGNPNPPVSSSTPVPSSSTTSSGSSGPTGGTGVAKHYEQCGG
IGFTGPTQCESPYTCTKLNDWYSQCL
(SEQ ID NO: 135)
QNACTLNAENHPSLTWSKCTSGGSCTSVQGSITIDANWRWTHRTDSATNC
YEGNKWDTSYCSDGPSCASKCCIDGADYSSTYGITTSGNSLNLKFVTKGQ
YSTNIGSRTYLMESDTKYQMFQLLGNEFTFDVDVSNLGCGLNGALYFVSM
DADGGMSKYSGNKAGAKYGTGYCDSQCPRDLKFINGEANVENWQSSTNDA
NAGTGKYGSCCSEMDVWEANNMAAAFTPRPCTVIGQSRCEGDSCGGTYST
DRYAGICDPDGCDFNSYRQGNKTFYGKGMTVDTTKKITVVTQFLKNSAGE
LSEIKRFYVQNGKVIPNSESTIPGVEGNSITQEYCDRQKAAFGDVTDFQD
KGGMVQMGKALAGPMVLVMSIWDDHADNMLWLDSTWPIDGAGKPGAERGA
CPTTSGVPAEVEAEAPNSNVIFSNIRFGPIGSTVSGLPDGGSGNPNPPVS
SSTPVPSSSTTSSGSSGPTGGTGVAKHYEQCGGIGFTGPTQCESPYTCTK
LNDWYSQCL

The polynucleotide (SEQ ID NO:136) and amino acid (SEQ ID NO:137) sequences of wild-type M. thermophile CBH2b are provided below. The signal sequence is shown underlined in SEQ ID NO:137. SEQ ID NO:138 provides the sequence of this CBH2b, without the signal sequence.

(SEQ ID NO: 136)
ATGGCCAAGAAGCTTTTCATCACCGCCGCGCTTGCGGCTGCCGTGTTGGC
GGCCCCCGTCATTGAGGAGCGCCAGAACTGCGGCGCTGTGTGGACTCAAT
GCGGCGGTAACGGGTGGCAAGGTCCCACATGCTGCGCCTCGGGCTCGACC
TGCGTTGCGCAGAACGAGTGGTACTCTCAGTGCCTGCCCAACAGCCAGGT
GACGAGTTCCACCACTCCGTCGTCGACTTCCACCTCGCAGCGCAGCACCA
GCACCTCCAGCAGCACCACCAGGAGCGGCAGCTCCTCCTCCTCCTCCACC
ACGCCCCCGCCCGTCTCCAGCCCCGTGACCAGCATTCCCGGCGGTGCGAC
CTCCACGGCGAGCTACTCTGGCAACCCCTTCTCGGGCGTCCGGCTCTTCG
CCAACGACTACTACAGGTCCGAGGTCCACAATCTCGCCATTCCTAGCATG
ACTGGTACTCTGGCGGCCAAGGCTTCCGCCGTCGCCGAAGTCCCTAGCTT
CCAGTGGCTCGACCGGAACGTCACCATCGACACCCTGATGGTCCAGACTC
TGTCCCAGGTCCGGGCTCTCAATAAGGCCGGTGCCAATCCTCCCTATGCT
GCCCAACTCGTCGTCTACGACCTCCCCGACCGTGACTGTGCCGCCGCTGC
GTCCAACGGCGAGTTTTCGATTGCAAACGGCGGCGCCGCCAACTACAGGA
GCTACATCGACGCTATCCGCAAGCACATCATTGAGTACTCGGACATCCGG
ATCATCCTGGTTATCGAGCCCGACTCGATGGCCAACATGGTGACCAACAT
GAACGTGGCCAAGTGCAGCAACGCCGCGTCGACGTACCACGAGTTGACCG
TGTACGCGCTCAAGCAGCTGAACCTGCCCAACGTCGCCATGTATCTCGAC
GCCGGCCACGCCGGCTGGCTCGGCTGGCCCGCCAACATCCAGCCCGCCGC
CGAGCTGTTTGCCGGCATCTACAATGATGCCGGCAAGCCGGCTGCCGTCC
GCGGCCTGGCCACTAACGTCGCCAACTACAACGCCTGGAGCATCGCTTCG
GCCCCGTCGTACACGTCGCCTAACCCTAACTACGACGAGAAGCACTACAT
CGAGGCCTTCAGCCCGCTCTTGAACTCGGCCGGCTTCCCCGCACGCTTCA
TTGTCGACACTGGCCGCAACGGCAAACAACCTACCGGCCAACAACAGTGG
GGTGACTGGTGCAATGTCAAGGGCACCGGCTTTGGCGTGCGCCCGACGGC
CAACACGGGCCACGAGCTGGTCGATGCCTTTGTCTGGGTCAAGCCCGGCG
GCGAGTCCGACGGCACAAGCGACACCAGCGCCGCCCGCTACGACTACCAC
TGCGGCCTGTCCGATGCCCTGCAGCCTGCCCCCGAGGCTGGACAGTGGTT
CCAGGCCTACTTCGAGCAGCTGCTCACCAACGCCAACCCGCCCTTCTAA
(SEQ ID NO: 137)
MAKKLFITAALAAAVLAAPVIEERQNCGAVWTQCGGNGWQGPTCCASGST
CVAQNEWYSQCLPNSQVTSSTTPSSTSTSQRSTSTSSSTTRSGSSSSSST
TPPPVSSPVTSIPGGATSTASYSGNPFSGVRLFANDYYRSEVHNLAIPSM
TGTLAAKASAVAEVPSFQWLDRNVTIDTLMVQTLSQVRALNKAGANPPYA
AQLVVYDLPDRDCAAAASNGEFSIANGGAANYRSYIDAIRKHIIEYSDIR
IILVIEPDSMANMVTNMNVAKCSNAASTYHELTVYALKQLNLPNVAMYLD
AGHAGWLGWPANIQPAAELFAGIYNDAGKPAAVRGLATNVANYNAWSIAS
APSYTSPNPNYDEKHYIEAFSPLLNSAGFPARFIVDTGRNGKQPTGQQQW
GDWCNVKGTGFGVRPTANTGHELVDAFVWVKPGGESDGTSDTSAARYDYH
CGLSDALQPAPEAGQWFQAYFEQLLTNANPPF
(SEQ ID NO: 138)
APVIEERQNCGAVWTQCGGNGWQGPTCCASGSTCVAQNEWYSQCLPNSQV
TSSTIPSSTSTSQRSTSTSSSTTRSGSSSSSSTTPPPVSSPVTSIPGGAT
STASYSGNPFSGVRLFANDYYRSEVHNLAIPSMTGTLAAKASAVAEVPSF
QWLDRNVTIDTLMVQTLSQVRALNKAGANPPYAAQLVVYDLPDRDCAAAA
SNGEFSIANGGAANYRSYIDAIRKHIIEYSDIRIILVIEPDSMANMVTNM
NVAKCSNAASTYHELTVYALKQLNLPNVAMYLDAGHAGWLGWPANIQPAA
ELFAGIYNDAGKPAAVRGLATNVANYNAWSIASAPSYTSPNPNYDEKHYI
EAFSPLLNSAGFPARFIVDTGRNGKQPTGQQQWGDWCNVKGTGFGVRPTA
NTGHELVDAFVWVKPGGESDGTSDTSAARYDYHCGLSDALQPAPEAGQWF
QAYFEQLLTNANPPF

The polynucleotide (SEQ ID NO:139) and amino acid (SEQ ID NO:140) sequences of a M. thermophila CBH2b variant (“Variant 196”) are provided below. The signal sequence is shown underlined in SEQ ID NO:140. SEQ ID NO:141 provides the sequence of this CBH2b variant, without the signal sequence.

(SEQ ID NO: 139)
ATGGCCAAGAAGCTTTTCATCACCGCCGCGCTTGCGGCTGCCGTGTTGGC
GGCCCCCGTCATTGAGGAGCGCCAGAACTGCGGCGCTGTGTGGACTCAAT
GCGGCGGTAACGGGTGGCAAGGTCCCACATGCTGCGCCTCGGGCTCGACC
TGCGTTGCGCAGAACGAGTGGTACTCTCAGTGCCTGCCCAACAGCCAGGT
GACGAGTTCCACCACTCCGTCGTCGACTTCCACCTCGCAGCGCAGCACCA
GCACCTCCAGCAGCACCACCAGGAGCGGCAGCTCCTCCTCCTCCTCCACC
ACGCCCACCCCCGTCTCCAGCCCCGTGACCAGCATTCCCGGCGGTGCGAC
CTCCACGGCGAGCTACTCTGGCAACCCCTTCTCGGGCGTCCGGCTCTTCG
CCAACGACTACTACAGGTCCGAGGTCCACAATCTCGCCATTCCTAGCATG
ACTGGTACTCTGGCGGCCAAGGCTTCCGCCGTCGCCGAAGTCCCTAGCTT
CCAGTGGCTCGACCGGAACGTCACCATCGACACCCTGATGGTCCCGACTC
TGTCCCGCGTCCGGGCTCTCAATAAGGCCGGTGCCAATCCTCCCTATGCT
GCCCAACTCGTCGTCTACGACCTCCCCGACCGTGACTGTGCCGCCGCTGC
GTCCAACGGCGAGTTTTCGATTGCAAACGGCGGCGCCGCCAACTACAGGA
GCTACATCGACGCTATCCGCAAGCACATCATTGAGTACTCGGACATCCGG
ATCATCCTGGTTATCGAGCCCGACTCGATGGCCAACATGGTGACCAACAT
GAACGTGGCCAAGTGCAGCAACGCCGCGTCGACGTACCACGAGTTGACCG
TGTACGCGCTCAAGCAGCTGAACCTGCCCAACGTCGCCATGTATCTCGAC
GCCGGCCACGCCGGCTGGCTCGGCTGGCCCGCCAACATCCAGCCCGCCGC
CGAGCTGTTTGCCGGCATCTACAATGATGCCGGCAAGCCGGCTGCCGTCC
GCGGCCTGGCCACTAACGTCGCCAACTACAACGCCTGGAGCATCGCTTCG
GCCCCGTCGTACACGTCGCCTAACCCTAACTACGACGAGAAGCACTACAT
CGAGGCCTTCAGCCCGCTCTTGAACTCGGCCGGCTTCCCCGCACGCTTCA
TTGTCGACACTGGCCGCAACGGCAAACAACCTACCGGCCAACAACAGTGG
GGTGACTGGTGCAATGTCAAGGGCACCGGCTTTGGCGTGCGCCCGACGGC
CAACACGGGCCACGAGCTGGTCGATGCCTTTGTCTGGGTCAAGCCCGGCG
GCGAGTCCGACGGCACAAGCGACACCAGCGCCGCCCGCTACGACTACCAC
TGCGGCCTGTCCGATGCCCTGCAGCCTGCCCCCGAGGCTGGACAGTGGTT
CCAGGCCTACTTCGAGCAGCTGCTCACCAACGCCAACCCGCCCTTCTAA
(SEQ ID NO: 140)
MAKKLFITAALAAAVLAAPVIEERQNCGAVWTQCGGNGWQGPTCCASGST
CVAQNEWYSQCLPNSQVTSSTTPSSTSTSQRSTSTSSSTTRSGSSSSSST
TPTPVSSPVTSIPGGATSTASYSGNPFSGVRLFANDYYRSEVHNLAIPSM
TGTLAAKASAVAEVPSFQWLDRNVTIDTLMVPTLSRVRALNKAGANPPYA
AQLVVYDLPDRDCAAAASNGEFSIANGGAANYRSYIDAIRKHIIEYSDIR
IILVIEPDSMANMVTNMNVAKCSNAASTYHELTVYALKQLNLPNVAMYLD
AGHAGWLGWPANIQPAAELFAGIYNDAGKPAAVRGLATNVANYNAWSIAS
APSYTSPNPNYDEKHYIEAFSPLLNSAGFPARFIVDTGRNGKQPTGQQQW
GDWCNVKGTGFGVRPTANTGHELVDAFVWVKPGGESDGTSDTSAARYDYH
CGLSDALQPAPEAGQWFQAYFEQLLTNANPPF
(SEQ ID NO: 141)
APVIEERQNCGAVWTQCGGNGWQGPTCCASGSTCVAQNEWYSQCLPNSQV
TSSTTPSSTSTSQRSTSTSSSTTRSGSSSSSSTTPTPVSSPVTSIPGGAT
STASYSGNPFSGVRLFANDYYRSEVHNLAIPSMTGTLAAKASAVAEVPSF
QWLDRNVIIDTLMVPTLSRVRALNKAGANPPYAAQLVVYDLPDRDCAAAA
SNGEFSIANGGAANYRSYIDAIRKHIIEYSDIRIILVIEPDSMANMVTNM
NVAKCSNAASTYHELTVYALKQLNLPNVAMYLDAGHAGWLGWPANIQPAA
ELFAGIYNDAGKPAAVRGLATNVANYNAWSIASAPSYTSPNPNYDEKHYI
EAFSPLLNSAGFPARFIVDTGRNGKQPTGQQQWGDWCNVKGTGFGVRPTA
NTGHELVDAFVWVIUGGESDGTSDTSAARYDYHCGLSDALQPAPEAGQWF
QAYFEQLLTNANPPF

The polynucleotide (SEQ ID NO:142) and amino acid (SEQ ID NO:143) sequences of a M. thermophila CBH2b variant (“Variant 287”) are provided below. The signal sequence is shown underlined in SEQ ID NO:143. SEQ ID NO:144 provides the sequence of this CBH2b variant, without the signal sequence.

(SEQ ID NO: 142)
ATGGCCAAGAAGCTTTTCATCACCGCCGCGCTTGCGGCTGCCGTGTTGGC
GGCCCCCGTCATTGAGGAGCGCCAGAACTGCGGCGCTGTGTGGACTCAAT
GCGGCGGTAACGGGTGGCAAGGTCCCACATGCTGCGCCTCGGGCTCGACC
TGCGTTGCGCAGAACGAGTGGTACTCTCAGTGCCTGCCCAACAGCCAGGT
GACGAGTTCCACCACTCCGTCGTCGACTTCCACCTCGCAGCGCAGCACCA
GCACCTCCAGCAGCACCACCAGGAGCGGCAGCTCCTCCTCCTCCTCCACC
ACGCCCCCGCCCGTCTCCAGCCCCGTGACCAGCATTCCCGGCGGTGCGAC
CTCCACGGCGAGCTACTCTGGCAACCCCTTCTCGGGCGTCCGGCTCTTCG
CCAACGACTACTACAGGTCCGAGGTCCACAATCTCGCCATTCCTAGCATG
ACTGGTACTCTGGCGGCCAAGGCTTCCGCCGTCGCCGAAGTCCCTAGCTT
CCAGTGGCTCGACCGGAACGTCACCATCGACACCCTGATGGTCCCGACTC
TGTCCCGCGTCCGGGCTCTCAATAAGGCCGGTGCCAATCCTCCCTATGCT
GCCCAACTCGTCGTCTACGACCTCCCCGACCGTGACTGTGCCGCCGCTGC
GTCCAACGGCGAGTTTTCGATTGCAAACGGCGGCGCCGCCAACTACAGGA
GCTACATCGACGCTATCCGCAAGCACATCAAGGAGTACTCGGACATCCGG
ATCATCCTGGTTATCGAGCCCGACTCGATGGCCAACATGGTGACCAACAT
GAACGTGGCCAAGTGCAGCAACGCCGCGTCGACGTACCACGAGTTGACCG
TGTACGCGCTCAAGCAGCTGAACCTGCCCAACGTCGCCATGTATCTCGAC
GCCGGCCACGCCGGCTGGCTCGGCTGGCCCGCCAACATCCAGCCCGCCGC
CGAGCTGTTTGCCGGCATCTACAATGATGCCGGCAAGCCGGCTGCCGTCC
GCGGCCTGGCCACTAACGTCGCCAACTACAACGCCTGGAGCATCGCTTCG
GCCCCGTCGTACACGTCGCCTAACCCTAACTACGACGAGAAGCACTACAT
CGAGGCCTTCAGCCCGCTCTTGAACGACGCCGGCTTCCCCGCACGCTTCA
TTGTCGACACTGGCCGCAACGGCAAACAACCTACCGGCCAACAACAGTGG
GGTGACTGGTGCAATGTCAAGGGCACCGGCTTTGGCGTGCGCCCGACGGC
CAACACGGGCCACGAGCTGGTCGATGCCTTTGTCTGGGTCAAGCCCGGCG
GCGAGTCCGACGGCACAAGCGACACCAGCGCCGCCCGCTACGACTACCAC
TGCGGCCTGTCCGATGCCCTGCAGCCTGCCCCCGAGGCTGGACAGTGGTT
CCAGGCCTACTTCGAGCAGCTGCTCACCAACGCCAACCCGCCCTTCTAA
(SEQ ID NO: 143)
MAKKLFITAALAAAVLAAPVIEERQNCGAVWTQCGGNGWQGPTCCASGST
CVAQNEWYSQCLPNSQVTSSTTPSSTSTSQRSTSTSSSTTRSGSSSSSST
TPPPVSSPVTSIPGGATSTASYSGNPFSGVRLFANDYYRSEVHNLAIPSM
TGTLAAKASAVAEVPSFQWLDRNVTIDTLMVPTLSRVRALNKAGANPPYA
AQLVVYDLPDRDCAAAASNGEFSIANGGAANYRSYIDAIRKHIKEYSDIR
IILVIEPDSMANMVTNMNVAKCSNAASTYHELTVYALKQLNLPNVAMYLD
AGHAGWLGWPANIQPAAELFAGIYNDAGKPAAVRGLATNVANYNAWSIAS
APSYTSPNPNYDEKHYIEAFSPLLNDAGFPARFIVDTGRNGKQPTGQQQW
GDWCNVKGTGEGVRPTANTGHELVDAFVWVKPGGESDGTSDTSAARYDYH
CGLSDALQPAPEAGQWFQAYFEQLLTNANPPF
(SEQ ID NO: 144)
APVIEERQNCGAVWTQCGGNGWQGPTCCASGSTCVAQNEWYSQCLPNSQV
TSSTTPSSTSTSQRSTSTSSSTTRSGSSSSSSTTPPPVSSPVTSIPGGAT
STASYSGNPFSGVRLFANDYYRSEVHNLAIPSMTGTLAAKASAVAEVPSF
QWLDRNVTIDTLMVPTLSRVRALNKAGANPPYAAQLVVYDLPDRDCAAAA
SNGEFSIANGGAANYRSYIDAIRKHIKEYSDIRIILVIEPDSMANMVTNM
NVAKCSNAASTYHELTVYALKQLNLPNVAMYLDAGHAGWLGWPANIQPAA
ELFAGIYNDAGKPAAVRGLATNVANYNAWSIASAPSYTSPNPNYDEKHYI
EAFSPLLNDAGFPARFIVDTGRNGKQPTGQQQWGDWCNVKGTGFGVRPTA
NTGHELVDAFVWVKPGGESDGTSDTSAARYDYHCGLSDALQPAPEAGQWF
QAYFEQLLTNANPPF

The polynucleotide (SEQ ID NO:145) and amino acid (SEQ ID NO:146) sequences of a M. thermophila CBH2b variant (“Variant 962”) are provided below. The signal sequence is shown underlined in SEQ ID NO:146. SEQ ID NO:147 provides the sequence of this CBH2b variant, without the signal sequence.

(SEQ ID NO: 145)
ATGGCCAAGAAGCTTTTCATCACCGCCGCGCTTGCGGCTGCCGTGTTGGC
GGCCCCCGTCATTGAGGAGCGCCAGAACTGCGGCGCTGTGTGGACTCAAT
GCGGCGGTAACGGGTGGCAAGGTCCCACATGCTGCGCCTCGGGCTCGACC
TGCGTTGCGCAGAACGAGTGGTACTCTCAGTGCCTGCCCAACAGCCAGGT
GACGAGTTCCACCACTCCGTCGTCGACTTCCACCTCGCAGCGCAGCACCA
GCACCTCCAGCAGCACCACCAGGAGCGGCAGCTCCTCCTCCTCCTCCACC
ACGCCCACCCCCGTCTCCAGCCCCGTGACCAGCATTCCCGGCGGTGCGAC
CTCCACGGCGAGCTACTCTGGCAACCCCTTCTCGGGCGTCCGGCTCTTCG
CCAACGACTACTACAGGTCCGAGGTCATGAATCTCGCCATTCCTAGCATG
ACTGGTACTCTGGCGGCCAAGGCTTCCGCCGTCGCCGAAGTCCCTAGCTT
CCAGTGGCTCGACCGGAACGTCACCATCGACACCCTGATGGTCACCACTC
TGTCCCAGGTCCGGGCTCTCAATAAGGCCGGTGCCAATCCTCCCTATGCT
GCCCAACTCGTCGTCTACGACCTCCCCGACCGTGACTGTGCCGCCGCTGC
GTCCAACGGCGAGTTTTCGATTGCAAACGGCGGCAGCGCCAACTACAGGA
GCTACATCGACGCTATCCGCAAGCACATCATTGAGTACTCGGACATCCGG
ATCATCCTGGTTATCGAGCCCGACTCGATGGCCAACATGGTGACCAACAT
GAACGTGGCCAAGTGCAGCAACGCCGCGTCGACGTACCACGAGTTGACCG
TGTACGCGCTCAAGCAGCTGAACCTGCCCAACGTCGCCATGTATCTCGAC
GCCGGCCACGCCGGCTGGCTCGGCTGGCCCGCCAACATCCAGCCCGCCGC
CGAGCTGTTTGCCGGCATCTACAATGATGCCGGCAAGCCGGCTGCCGTCC
GCGGCCTGGCCACTAACGTCGCCAACTACAACGCCTGGAGCATCGCTTCG
GCCCCGTCGTACACGCAGCCTAACCCTAACTACGACGAGAAGCACTACAT
CGAGGCCTTCAGCCCGCTCTTGAACTCGGCCGGCTTCCCCGCACGCTTCA
TTGTCGACACTGGCCGCAACGGCAAACAACCTACCGGCCAACAACAGTGG
GGTGACTGGTGCAATGTCAAGGGCACCGGCTTTGGCGTGCGCCCGACGGC
CAACACGGGCCACGAGCTGGTCGATGCCTTTGTCTGGGTCAAGCCCGGCG
GCGAGTCCGACGGCACAAGCGACACCAGCGCCGCCCGCTACGACTACCAC
TGCGGCCTGTCCGATGCCCTGCAGCCTGCCCCCGAGGCTGGACAGTGGTT
CCAGGCCTACTTCGAGCAGCTGCTCACCAACGCCAACCCGCCCTTCTAA
(SEQ ID NO: 146)
MAKKLFITAALAAAVLAAPVIEERQNCGAVWTQCGGNGWQGPTCCASGST
CVAQNEWYSQCLPNSQVTSSTTPSSTSTSQRSTSTSSSTTRSGSSSSSST
TPTPVSSPVTSIPGGATSTASYSGNPFSGVRLFANDYYRSEVMNLAIPSM
TGTLAAKASAVAEVPSFQWLDRNVTIDTLMVTTLSQVRALNKAGANPPYA
AQLVVYDLPDRDCAAAASNGEFSIANGGSANYRSYIDAIRKHIIEYSDIR
IILVIEPDSMANMVTNMNVAKCSNAASTYHELTVYALKQLNLPNVAMYLD
AGHAGWLGWPANIQPAAELFAGIYNDAGKPAAVRGLATNVANYNAWSIAS
APSYTQPNPNYDEKHYEEAFSPLLNSAGFPARFIVDTGRNGKQPTGQQQW
GDWCNVKGTGFGVRPTANTGHELVDAFVWVKPGGESDGTSDTSAARYDYH
CGLSDALQPAPEAGQWFQAYFEQLLTNANPPF
(SEQ ID NO: 147)
APVIEERQNCGAVWTQCGGNGWQGPTCCASGSTCVAQNEWYSQCLPNSQV
TSSTTPSSTSTSQRSTSTSSSTTRSGSSSSSSTTPTPVSSPVTSIPGGAT
STASYSGNPFSGVRLFANDYYRSEVMNLAIPSMTGTLAAKASAVAEVPSF
QWLDRNVTIDTLMVTTLSQVRALNKAGANPPYAAQLVVYDLPDRDCAAAA
SNGEFSIANGGSANYRSYIDAIRKHIIEYSDIRIILVIEPDSMANMVTNM
NVAKCSNAASTYHELTVYALKQLNLPNVAMYLDAGHAGWLGWPANIQPAA
ELFAGIYNDGKPAAVRGLATNVANYNAWSIASAPSYTQPNPNYDEKHYIE
AFSPLLNSAGFPARFIVDTGRNGKQPTGQQQWGDWCNVKGTGFGVRPTAN
TGHELVDAFVWVKPGGESDGTSDTSAARYDYHCGLSDALQPAPEAGQWFQ
AYFEQLLTNANPPF

The polynucleotide (SEQ ID NO:148) and amino acid (SEQ ID NO:149) sequences of a wild-type M. thermophila xylanase (“Xyl3”) are provided below. The signal sequence is shown underlined in SEQ ID NO:149. SEQ ID NO:150 provides the sequence of this xylanase without the signal sequence.

(SEQ ID NO: 148)
ATGCACTCCAAAGCTTTCTTGGCAGCGCTTCTTGCGCCTGCCGTCTCAGG
GCAACTGAACGACCTCGCCGTCAGGGCTGGACTCAAGTACTTTGGTACTG
CTCTTAGCGAGAGCGTCATCAACAGTGATACTCGGTATGCTGCCATCCTC
AGCGACAAGAGCATGTTCGGCCAGCTCGTCCCCGAGAATGGCATGAAGTG
GGATGCTACTGAGCCGTCCCGTGGCCAGTTCAACTACGCCTCGGGCGACA
TCACGGCCAACACGGCCAAGAAGAATGGCCAGGGCATGCGTTGCCACACC
ATGGTCTGGTACAGCCAGCTCCCGAGCTGGGTCTCCTCGGGCTCGTGGAC
CAGGGACTCGCTCACCTCGGTCATCGAGACGCACATGAACAACGTCATGG
GCCACTACAAGGGCCAATGCTACGCCTGGGATGTCATCAACGAGGCCATC
AATGACGACGGCAACTCCTGGCGCGACAACGTCTTTCTCCGGACCTTTGG
GACCGACTACTTCGCCCTGTCCTTCAACCTAGCCAAGAAGGCCGATCCCG
ATACCAAGCTGTACTACAACGACTACAACCTCGAGTACAACCAGGCCAAG
ACGGACCGCGCTGTTGAGCTCGTCAAGATGGTCCAGGCCGCCGGCGCGCC
CATCGACGGTGTCGGCTTCCAGGGCCACCTCATTGTCGGCTCGACCCCGA
CGCGCTCGCAGCTGGCCACCGCCCTCCAGCGCTTCACCGCGCTCGGCCTC
GAGGTCGCCTACACCGAGCTCGACATCCGCCACTCGAGCCTGCCGGCCTC
TTCGTCGGCGCTCGCGACCCAGGGCAACGACTTCGCCAACGTGGTCGGCT
CTTGCCTCGACACCGCCGGCTGCGTCGGCGTCACCGTCTGGGGCTTCACC
GATGCGCACTCGTGGATCCCGAACACGTTCCCCGGCCAGGGCGACGCCCT
GATCTACGACAGCAACTACAACAAGAAGCCCGCGTGGACCTCGATCTCGT
CCGTCCTGGCCGCCAAGGCCACCGGCGCCCCGCCCGCCTCGTCCTCCACC
ACCCTCGTCACCATCACCACCCCTCCGCCGGCATCCACCACCGCCTCCTC
CTCCTCCAGTGCCACGCCCACGAGCGTCCCGACGCAGACGAGGTGGGGAC
AGTGCGGCGGCATCGGATGGACGGGGCCGACCCAGTGCGAGAGCCCATGG
ACCTGCCAGAAGCTGAACGACTGGTACTGGCAGTGCCTG
(SEQ ID NO: 149)
MHSKAFLAALLAPAVSGQLNDLAVRAGLKYFGTALSESVINSDTRYAAIL
SDKSMFGQLVPENGMKWDATEPSRGQFNYASGDITANTAKKNGQGMRCHT
MVWYSQLPSWVSSGSWTRDSLTSVIETHMNNVMGHYKGQCYAWDVINEAI
NDDGNSWRDNVFLRTFGTDYFALSFNLALKADPDTKLYYNDYNLEYNQAK
TDRAVELVKMVQAAGAPIDGVGFQGHLIVGSTPTRSQLATALQRFTALGL
EVAYTELDIRHSSLPASSSALATQGNDFANVVGSCLDTAGCVGVTVWGFT
DAHSWIPNTFPGQGDALIYDSNYNKKPAWTSISSVLAAKATGAPPASSST
TLVTITTPPPASTTASSSSSATPTSVPTQTRWGQCGGIGWTGPTQCESPW
TCQKLNDWYWQCL
(SEQ ID NO: 150)
QLNDLAVRAGLKYFGTALSESVINSDTRYAAILSDKSMFGQLVPENGMKW
DATEPSRGQFNYASGDITANTAKKNGQGMRCHTMVWYSQLPSWVSSGSWT
RDSLTSVIETHMNNVMGHYKGQCYAWDVINEAINDDGNSWRDNVFLRTFG
TDYFALSFNLAKKADPDTKLYYNDYNLEYNQAKTDRAVELVKMVQAAGAP
IDGVGFQGHLIVGSTPTRSQLATALQRFTALGLEVAYTELDIRHSSLPAS
SSALATQGNDFANVVGSCLDTAGCVGVTVWGFTDAHSWIPNTFPGQGDAL
IYDSNYNKKPAWTSISSVLAAKATGAPPASSSTTLVTITTPPPASTTASS
SSSATPTSVPTQTRWGQCGGIGWTGPTQCESPWTCQKLNDWYWQCL

The polynucleotide (SEQ ID NO:151) and amino acid (SEQ ID NO:152) sequences of a wild-type M. thermophila xylanase (“Xyl 2”) are provided below. The signal sequence is shown underlined in SEQ ID NO:152. SEQ ID NO:153 provides the sequence of this xylanase without the signal sequence.

(SEQ ID NO: 151)
ATGGTCTCGTTCACTCTCCTCCTCACGGTCATCGCCGCTGCGGTGACGAC
GGCCAGCCCTCTCGAGGTGGTCAAGCGCGGCATCCAGCCGGGCACGGGCA
CCCACGAGGGGTACTTCTACTCGTTCTGGACCGACGGCCGTGGCTCGGTC
GACTTCAACCCCGGGCCCCGCGGCTCGTACAGCGTCACCTGGAACAACGT
CAACAACTGGGTTGGCGGCAAGGGCTGGAACCCGGGCCCGCCGCGCAAGA
TTGCGTACAACGGCACCTGGAACAACTACAACGTGAACAGCTACCTCGCC
CTGTACGGCTGGACTCGCAACCCGCTGGTCGAGTATTACATCGTGGAGGC
ATACGGCACGTACAACCCCTCGTCGGGCACGGCGCGGCTGGGCACCATCG
AGGACGACGGCGGCGTGTACGACATCTACAAGACGACGCGGTACAACCAG
CCGTCCATCGAGGGGACCTCCACCTTCGACCAGTACTGGTCCGTCCGCCG
CCAGAAGCGCGTCGGCGGCACTATCGACACGGGCAAGCACTTTGACGAGT
GGAAGCGCCAGGGCAACCTCCAGCTCGGCACCTGGAACTACATGATCATG
GCCACCGAGGGCTACCAGAGCTCTGGTTCGGCCACTATCGAGGTCCGGGA
GGCC
(SEQ ID NO: 152)
MVSFTLLLTVIAAAVTTASPLEVVKRGIQPGTGTHEGYFYSFWTDGRGSV
DFNPGPRGSYSVTWNNVNNWVGGKGWNPGPPRKIAYNGTWNNYNVNSYLA
LYGWTRNPLVEYYIVEAYGTYNPSSGTARLGTIEDDGGVYDIYKTTRYNQ
PSIEGTSTFDQYWSVRRQKRVGGTIDTGKHFDEWKRQGNLQLGTWNYMIM
ATEGYQSSGSATIEVREA
(SEQ ID NO: 153)
MVSFTLLLTVIAAAVTTASPLEVVKRGIQPGTGTHEGYFYSFWTDGRGSV
DFNPGPRGSYSVTWNNVNNWNGGKGWNPGPPRKIAYNGTWNNYNVNSYLA
LYGWTRNPLVEYYIVEAYGTYNPSSGTARLGTIEDDGGVYDIYKTTRYNQ
PSIEGTSTFDQYWSVRRQKRVGGTIDTGKHFDEWKRQGNLQLGTWNYMIM
ATEGYQSSGSATIEVREA

The polynucleotide (SEQ ID NO:154) and amino acid (SEQ ID NO:155) sequences of another wild-type M. thermophila xylanase (“Xyl1”) are provided below. The signal sequence is shown underlined in SEQ ID NO:155. SEQ ID NO:156 provides the sequence of this xylanase without the signal sequence.

(SEQ ID NO: 154)
ATGCGTACTCTTACGTTCGTGCTGGCAGCCGCCCCGGTGGCTGTGCTTGC
CCAATCTCCTCTGTGGGGCCAGTGCGGCGGTCAAGGCTGGACAGGTCCCA
CGACCTGCGTTTCTGGCGCAGTATGCCAATTCGTCAATGACTGGTACTCC
CAATGCGTGCCCGGATCGAGCAACCCTCCTACGGGCACCACCAGCAGCAC
CACTGGAAGCACCCCGGCTCCTACTGGCGGCGGCGGCAGCGGAACCGGCC
TCCACGACAAATTCAAGGCCAAGGGCAAGCTCTACTTCGGAACCGAGATC
GATCACTACCATCTCAACAACAATGCCTTGACCAACATTGTCAAGAAAGA
CTTTGGTCAAGTCACTCACGAGAACAGCTTGAAGTGGGATGCTACTGAGC
CGAGCCGCAATCAATTCAACTTTGCCAACGCCGACGCGGTTGTCAACTTT
GCCCAGGCCAACGGCAAGCTCATCCGCGGCCACACCCTCCTCTGGCACTC
TCAGCTGCCGCAGTGGGTGCAGAACATCAACGACCGCAACACCTTGACCC
AGGTCATCGAGAACCACGTCACCACCCTTGTCACTCGCTACAAGGGCAAG
ATCCTCCACTGGGACGTCGTTAACGAGATCTTTGCCGAGGACGGCTCGCT
CCGCGACAGCGTCTTCAGCCGCGTCCTCGGCGAGGACTTTGTCGGCATCG
CCTTCCGCGCCGCCCGCGCCGCCGATCCCAACGCCAAGCTCTACATCAAC
GACTACAACCTCGACATTGCCAACTACGCCAAGGTGACCCGGGGCATGGT
CGAGAAGGTCAACAAGTGGATCGCCCAGGGCATCCCGATCGACGGCATCG
GCACCCAGTGCCACCTGGCCGGGCCCGGCGGGTGGAACACGGCCGCCGGC
GTCCCCGACGCCCTCAAGGCCCTCGCCGCGGCCAACGTCAAGGAGATCGC
CATCACCGAGCTCGACATCGCCGGCGCCTCCGCCAACGACTACCTCACCG
TCATGAACGCCTGCCTCCAGGTCTCCAAGTGCGTCGGCATCACCGTCTGG
GGCGTCTCTGACAAGGACAGCTGGAGGTCGAGCAGCAACCCGCTCCTCTT
CGACAGCAACTACCAGCCAAAGGCGGCATACAATGCTCTGATTAATGCCT
TGTAA
(SEQ ID NO: 155)
MRTLTFVLAAAPVAVLAQSPLWGQCGGQGWTGPTTCVSGAVCQFVNDWYS
QCVPGSSNPPTGTTSSTTGSTPAPTGGGGSGTGLHDKFKAKGKLYFGTEI
DHYHLNNNALTNIVKKDFGQVTHENSLKWDATEPSRNQFNFANADAVVNF
AQANGKLIRGHTLLWHSQLPQWVQNINDRNTLTQVIENHVTTLVTRYKGK
ILHWDVVNEIFAEDGSLRDSVFSRVLGEDFVGIAFRAARAADPNAKLYIN
DYNLDIANYAKVTRGMVEKVNKWIAQGIPIDGIGTQCHLAGPGGWNTAAG
VPDALKALAAANVKETAITELDIAGASANDYLTVMNACLQVSKCVGITVW
GVSDKDSWRSSSNPLLFDSNYQPKAAYNALINAL
(SEQ ID NO: 156)
QSPLWGQCGGQGWTGPTTCVSGAVCQFVNDWYSQCVPGSSNPPTGTTSSI
TGSTPAPTGGGGSGTGLHDKFKAKGKLYFGTEIDHYHLNNNALTNIVKKD
FGQVTHENSLKWDATEPSRNQFNFANADAVVNFAQANGKLIRGHTLLWHS
QLPQWVQNINDRNTLTQVIENHVTTLVTRYKGKILHWDVVNEIFAEDGSL
RDSVFSRVLGEDFVGIAFRAARAADPNAKLYINDYNLDIANYAKVTRGMV
EKVNKWIAQGIPIDGIGTQCHLAGPGGWNTAAGVPDALKALAAANVKEIA
ITELDIAGASANDYLTVMNACLQVSKCVGITVWGVSDKDSWRSSSNPLLF
DSNYQPKAAYNALINAL

The polynucleotide (SEQ ID NO:157) and amino acid (SEQ ID NO:158) sequences of another wild-type M. thermophila xylanase (“Xyl6”) are provided below. The signal sequence is shown underlined in SEQ ID NO:158. SEQ ID NO:159 provides the sequence of this xylanase without the signal sequence.

(SEQ 1D NO: 157)
ATGGTCTCGCTCAAGTCCCTCCTCCTCGCCGCGGCGGCGACGTTGACGGC
GGTGACGGCGCGCCCGTTCGACTTTGACGACGGCAACTCGACCGAGGCGC
TGGCCAAGCGCCAGGTCACGCCCAACGCGCAGGGCTACCACTCGGGCTAC
TTCTACTCGTGGTGGTCCGACGGCGGCGGCCAGGCCACCTTCACCCTGCT
CGAGGGCAGCCACTACCAGGTCAACTGGAGGAACACGGGCAACTTTGTCG
GTGGCAAGGGCTGGAACCCGGGTACCGGCCGGACCATCAACTACGGCGGC
TCGTTCAACCCGAGCGGCAACGGCTACCTGGCCGTCTACGGCTGGACGCA
CAACCCGCTGATCGAGTACTACGTGGTCGAGTCGTACGGGACCTACAACC
CGGGCAGCCAGGCCCAGTACAAGGGCAGCTTCCAGAGCGACGGCGGCACC
TACAACATCTACGTCTCGACCCGCTACAACGCGCCCTCGATCGAGGGCAC
CCGCACCTTCCAGCAGTACTGGTCCATCCGCACCTCCAAGCGCGTCGGCG
GCTCCGTCACCATGCAGAACCACTTCAACGCCTGGGCCCAGCACGGCATG
CCCCTCGGCTCCCACGACTACCAGATCGTCGCCACCGAGGGCTACCAGAG
CAGCGGCTCCTCCGACATCTACGTCCAGACTCACTAG
(SEQ ID NO: 158)
MVSLKSLLLAAAATLTAVTARPFDFDDGNSTEALAKRQVTPNAQGYHSGY
FYSWWSDGGGQATFTLLEGSHYQVNWRNTGNFVGGKGWNPGTGRTINYGG
SFNPSGNGYLAVYGWTHNPLIEYYVVESYGTYNPGSQAQYKGSFQSDGGT
YNIYVSTRYNAPSIEGTRTFQQYWSIRTSKRVGGSVTMQNHFNAWAQHGM
PLGSHDYQIVATEGYQSSGSSDIYVQTH
(SEQ ID NO: 159)
RPFDFDDGNSTEALAKRQVTPNAQGYHSGYFYSWWSDGGGQATFTLLEGS
HYQVNWRNTGNFVGGKGWNPGTGRTINYGGSFNPSGNGYLAVYGWTHNPL
IEYYVVESYGTYNPGSQAQYKGSFQSDGGTYNIYVSTRYNAPSIEGTRTF
QQYWSIRTSKRVGGSVTMQNHFNAWAQHGMPLGSHDYQIVATEGYQSSGS
SDIYVQTH

The polynucleotide (SEQ ID NO:160) and amino acid (SEQ ID NO:161) sequences of another wild-type M. thermophila xylanase (“Xyl5”) are provided below. The signal sequence is shown underlined in SEQ ID NO:161. SEQ ID NO:162 provides the sequence of this xylanase, without the signal sequence.

(SEQ ID NO: 160)
ATGGTTACCCTCACTCGCCTGGCGGTCGCCGCGGCGGCCATGATCTCCAG
CACTGGCCTGGCTGCCCCGACGCCCGAAGCTGGCCCCGACCTTCCCGACT
TTGAGCTCGGGGTCAACAACCTCGCCCGCCGCGCGCTGGACTACAACCAG
AACTACAGGACCAGCGGCAACGTCAACTACTCGCCCACCGACAACGGCTA
CTCGGTCAGCTTCTCCAACGCGGGAGATTTTGTCGTCGGGAAGGGCTGGA
GGACGGGAGCCACCAGAAACATCACCTTCTCGGGATCGACACAGCATACC
TCGGGCACCGTGCTCGTCTCCGTCTACGGCTGGACCCGGAACCCGCTGAT
CGAGTACTACGTGCAGGAGTACACGTCCAACGGGGCCGGCTCCGCTCAGG
GCGAGAAGCTGGGCACGGTCGAGAGCGACGGGGGCACGTACGAGATCTGG
CGGCACCAGCAGGTCAACCAGCCGTCGATCGAGGGCACCTCGACCTTCTG
GCAGTACATCTCGAACCGCGTGTCCGGCCAGCGGCCCAACGGCGGCACCG
TCACCCTCGCCAACCACTTCGCCGCCTGGCAGAAGCTCGGCCTGAACCTG
GGCCAGCACGACTACCAGGTCCTGGCCACCGAGGGCTGGGGCAACGCCGG
CGGCAGCTCCCAGTACACCGTCAGCGGCTGA
(SEQ ID NO: 161)
MVTLTRLAVAAAAMISSTGLAAPTPEAGPDLPDFELGVNNLARRALDYNQ
NYRTSGNVNYSPTDNGYSVSFSNAGDFVVGKGWRTGATRNITFSGSTQHT
SGTVLVSVYGWTRNPLIEYYVQEYTSNGAGSAQGEKLGTVESDGGTYEIW
RHQQVNQPSIEGTSTFWQYISNRVSGQRPNGGTVTLANHFAAWQKLGLNL
GQHDYQVLATEGWGNAGGSSQYTVSG
(SEQ ID NO: 162)
APTPEAGPDLPDFELGVNNLARRALDYNQNYRTSGNVNYSPTDNGYSVSF
SNAGDFVVGKGWRTGATRNITFSGSTQHTSGTVLVSVYGWTRNPLIEYYV
QEYTSNGAGSAQGEKLGTVESDGGTYEIWRHQQVNQPSIEGTSTFWQYIS
NRVSGQRPNGGTVTLANHFAAWQKLGLNLGQHDYQVLATEGWGNAGGSSQ
YTVSG

The polynucleotide (SEQ ID NO:163) and amino acid (SEQ ID NO:164) sequences of a wild-type M. thermophila beta-xylosidase are provided below. The signal sequence is shown underlined in SEQ ID NO:164. SEQ ID NO:165 provides the sequence of this xylanase without the signal sequence.

(SEQ ID NO: 163)
ATGTTCTTCGCTTCTCTGCTGCTCGGTCTCCTGGCGGGCGTGTCCGCTTC
ACCGGGACACGGGCGGAATTCCACCTTCTACAACCCCATCTTCCCCGGCT
TCTACCCCGATCCGAGCTGCATCTACGTGCCCGAGCGTGACCACACCTTC
TTCTGTGCCTCGTCGAGCTTCAACGCCTTCCCGGGCATCCCGATTCATGC
CAGCAAGGACCTGCAGAACTGGAAGTTGATCGGCCATGTGCTGAATCGCA
AGGAACAGCTTCCCCGGCTCGCTGAGACCAACCGGTCGACCAGCGGCATC
TGGGCACCCACCCTCCGGTTCCATGACGACACCTTCTGGTTGGTCACCAC
ACTAGTGGACGACGACCGGCCGCAGGAGGACGCTTCCAGATGGGACAATA
TTATCTTCAAGGCAAAGAATCCGTATGATCCGAGGTCCTGGTCCAAGGCC
GTCCACTTCAACTTCACTGGCTACGACACGGAGCCTTTCTGGGACGAAGA
TGGAAAGGTGTACATCACCGGCGCCCATGCTTGGCATGTTGGCCCATACA
TCCAGCAGGCCGAAGTCGATCTCGACACGGGGGCCGTCGGCGAGTGGCGC
ATCATCTGGAACGGAACGGGCGGCATGGCTCCTGAAGGGCCGCACATCTA
CCGCAAAGATGGGTGGTACTACTTGCTGGCTGCTGAAGGGGGGACCGGCA
TCGACCATATGGTGACCATGGCCCGGTCGAGAAAAATCTCCAGTCCTTAC
GAGTCCAACCCAAACAACCCCGTGTTGACCAACGCCAACACGACCAGTTA
CTTTCAAACCGTCGGGCATTCAGACCTGTTCCATGACAGACATGGGAACT
GGTGGGCAGTCGCCCTCTCCACCCGCTCCGGTCCAGAATATCTTCACTAC
CCCATGGGCCGCGAGACCGTCATGACAGCCGTGAGCTGGCCGAAGGACGA
GTGGCCAACCTTCACCCCCATATCTGGCAAGATGAGCGGCTGGCCGATGC
CTCCTTCGCAGAAGGACATTCGCGGAGTCGGCCCCTACGTCAACTCCCCC
GACCCGGAACACCTGACCTTCCCCCGCTCGGCGCCCCTGCCGGCCCACCT
CACCTACTGGCGATACCCGAACCCGTCCTCCTACACGCCGTCCCCGCCCG
GGCACCCCAACACCCTCCGCCTGACCCCGTCCCGCCTGAACCTGACCGCC
CTCAACGGCAACTACGCGGGGGCCGACCAGACCTTCGTCTCGCGCCGGCA
GCAGCACACCCTCTTCACCTACAGCGTCACGCTCGACTACGCGCCGCGGA
CCGCCGGGGAGGAGGCCGGCGTGACCGCCTTCCTGACGCAGAACCACCAC
CTCGACCTGGGCGTCGTCCTGCTCCCTCGCGGCTCCGCCACCGCGCCCTC
GCTGCCGGGCCTGAGTAGTAGTACAACTACTACTAGTAGTAGTAGTAGTC
GTCCGGACGAGGAGGAGGAGCGCGAGGCGGGCGAAGAGGAAGAAGAGGGC
GGACAAGACTTGATGATCCCGCATGTGCGGTTCAGGGGCGAGTCGTACGT
GCCCGTCCCGGCGCCCGTCGTGTACCCGATACCCCGGGCCTGGAGAGGCG
GGAAGCTTGTGTTAGAGATCCGGGCTTGTAATTCGACTCACTTCTCGTTC
CGTGTCGGGCCGGACGGGAGACGGTCTGAGCGGACGGTGGTCATGGAGGC
TTCGAACGAGGCCGTTAGCTGGGGCTTTACTGGAACGCTGCTGGGCATCT
ATGCGACCAGTAATGGTGGCAACGGAACCACGCCGGCGTATTTTTCGGAT
TGGAGGTACACACCATTGGAGCAGTTTAGGGAT
(SEQ ID NO: 164)
MFFASLLLGLLAGVSASPGHGRNSTFYNPIFPGFYPDPSCIYVPERDHTF
FCASSSFNAFPGIPIHASKDLQNWKLIGHVLNRKEQLPRLAETNRSTSGI
WAPTLRFHDDTFWLVTTLVDDDRPQEDASRWDNIEFKAKNPYDPRSWSKA
VHFNFTGYDTEPFWDEDGKVYITGAHAWHVGPYIQQAEVDLDTGAVGEWR
IIWNGTGGMAPEGPHIYRKDGWYYLLAAEGGTGIDHMVTMARSRKISSPY
ESNPNNPVLTNANTTSYFQTVGHSDLFHDRHGNWWAVALSTRSGPEYLHY
PMGRETVMTAVSWPKDEWPTFTPISGKMSGWPMPPSQKDIRGVGPYVNSP
DPEHLTFPRSAPLPAHLTYWRYPNPSSYTPSPPGHPNTLRLTPSRLNLTA
LNGNYAGADQTFVSRRQQHTLFTYSVTLDYAPRTAGEEAGVTAFLTQNHR
LDLGVVLLPRGSATAPSLPGLSSSTTTTSSSSSRPDEEEEREAGEEEEEG
GQDLMIPHVRFRGESYVPVPAPVVYPIPRAWRGGKINLEIRACNSTHFSF
RVGPDGRRSERTVVMEASNEAVSWGFTGTLLGIYATSNGGNGTTPAYFSD
WRYTPLEQFRD
(SEQ ID NO: 165)
SPGHGRNSTFYNPIFPGFYPDPSCIYVPERDHTFFCASSSFNAFPGIPIH
ASKDLQNWKLIGHVLNRKEQLPRLAETNRSTSGIWAPTLRFHDDTFWLVI
TLVDDDRPQEDASRWDNIIFKAKNPYDPRSWSKAVHFNFTGYDTEPFWDE
DGKVYITGAHAWHVGPYIQQAEVDLDTGAVGEWRIIWNGTGGMAPEGPII
TYRKDGWYYLLAAEGGTGEDHMVTMARSRKISSPYESNPNNPVLTNANTT
SYFQTVGHSDLFHDRHGNWWAVALSTRSGPEYLHYPMGRETVMTAVSWPK
DEWPTFTPISGKMSGWPMPPSQKDIRGVGPYVNSPDPEHLTFPRSAPLPA
HLTYWRYPNPSSYTPSPPGHPNTLRLTPSRLNLTALNGNYAGADQTFVSR
RQQHTLFTYSVTLDYAPRTAGEEAGVTAFLTQNHHLDLGVVLLPRGSATA
PSLPGLSSSTTTTSSSSSRPDEEEEREAGEEEEEGGQDLMIPHVRFRGES
YVPVPAPVVYPIPRAWRGGKLVLEIRACNSITIFSFRVGPDGRRSERTVV
MEASNEAVSWGFTGTLLGIYATSNGGNGTTPAYFSDWRYTPLEQFRD

The polynucleotide (SEQ ID NO:166) and amino acid (SEQ ID NO:167) sequences of a wild-type M. thermophila acetylxylan esterase (“Axe3”) are provided below. The signal sequence is shown underlined in SEQ ID NO:167. SEQ ID NO:168 provides the sequence of this acetylxylan esterase without the signal sequence.

(SEQ ID NO: 166)
ATGAAGCTCCTGGGCAAACTCTCGGCGGCACTCGCCCTCGCGGGCAGCAG
GCTGGCTGCCGCGCACCCGGTCTTCGACGAGCTGATGCGGCCGACGGCGC
CGCTGGTGCGCCCGCGGGCGGCCCTGCAGCAGGTGACCAACTTTGGCAGC
AACCCGTCCAACACGAAGATGTTCATCTACGTGCCCGACAAGCTGGCCCC
CAACCCGCCCATCATAGTGGCCATCCACTACTGCACCGGCACCGCCCAGG
CCTACTACTCGGGCTCCCCTTACGCCCGCCTCGCCGACCAGAAGGGCTTC
ATCGTCATCTACCCGGAGTCCCCCTACAGCGGCACCTGTTGGGACGTCTC
GTCGCGCGCCGCCCTGACCCACAACGGCGGCGGCGACAGCAACTCGATCG
CCAACATGGTCACCTACACCCTCGAAAAGTACAATGGCGACGCCAGCAAG
GTCTTTGTCACCGGCTCCTCGTCCGGCGCCATGATGACGAACGTGATGGC
CGCCGCGTACCCGGAACTGTTCGCGGCAGGAATCGCCTACTCGGGCGTGC
CCGCCGGCTGCTTCTACAGCCAGTCCGGAGGCACCAACGCGTGGAACAGC
TCGTGCGCCAACGGGCAGATCAACTCGACGCCCCAGGTGTGGGCCAAGAT
GGTCTTCGACATGTACCCGGAATACGACGGCCCGCGCCCCAAGATGCAGA
TCTACCACGGCTCGGCCGACGGCACGCTCAGACCCAGCAACTACAACGAG
ACCATCAAGCAGTGGTGCGGCGTCTTCGGCTTCGACTACACCCGCCCCGA
CACCACCCAGGCCAACTCCCCGCAGGCCGGCTACACCACCTACACCTGGG
GCGAGCAGCAGCTCGTCGGCATCTACGCCCAGGGCGTCGGACACACGGTC
CCCATCCGCGGCAGCGACGACATGGCCTTCTTTGGCCTGTGA
(SEQ ID NO: 167)
MKLLGKLSAALALAGSRLAAAHPVFDELMRPTAPLVRPRAALQQVTNFGS
NPSNTKMFIYVPDKLAPNPPIIVAIHYCTGTAQAYYSGSPYARLADQKGF
IVIYPESPYSGTCWDVSSRAALTHNGGGDSNSIANMVTYTLEKYNGDASK
VFVTGSSSGAMMTNVMAAAYPELFAAGIAYSGVPAGCFYSQSGGTNAWNS
SCANGQINSTPQVWAKMVFDMYPEYDGPRPKMQIYHGSADGTLRPSNYNE
TIKQWCGVFGFDYTRPDTTQANSPQAGYTTYTWGEQQLVGIYAQGVGHTV
PIRGSDDMAFFGL
(SEQ ID NO: 168)
HPVFDELMRPTAPLVRPRAALQQVTNFGSNPSNTKMFIYVPDKLAPNPPI
RTAIHYCTGTAQAYYSGSPYARLADQKGFFVIYPESPYSGTCWDVSSRAA
LTHNGGGDSNSIANMVTYTLEKYNGDASKVFVTGSSSGAMMTNVMAAAYP
ELFAAGIAYSGVPAGCFYSQSGGTNAWNSSCANGQINSTPQVWAKMVFDM
YPEYDGPRPKMQIYHGSADGTLRPSNYNETIKQWCGVFGFDYTRPDTTQA
NSPQAGYTTYTWGEQQLVGIYAQGVGHTVPIRGSDDMAFFGL

The polynucleotide (SEQ ID NO:169) and amino acid (SEQ ID NO:170) sequences of a wild-type M. thermophila ferulic acid esterase (“FAE”) are provided below. The signal sequence is shown underlined in SEQ ID NO:170. SEQ ID NO:171 provides the sequence of this xylanase without the signal sequence

(SEQ ID NO: 169)
ATGATCTCGGTTCCTGCTCTCGCTCTGGCCCTTCTGGCCGCCGTCCAGGT
CGTCGAGTCTGCCTCGGCTGGCTGTGGCAAGGCGCCCCCTTCCTCGGGCA
CCAAGTCGATGACGGTCAACGGCAAGCAGCGCCAGTACATTCTCCAGCTG
CCCAACAACTACGACGCCAACAAGGCCCACAGGGTGGTGATCGGGTACCA
CTGGCGCGACGGATCCATGAACGACGTGGCCAACGGCGGCTTCTACGATC
TGCGGTCCCGGGCGGGCGACAGCACCATCTTCGTTGCCCCCAACGGCCTC
AATGCCGGATGGGCCAACGTGGGCGGCGAGGACATCACCTTTACGGACCA
GATCGTAGACATGCTCAAGAACGACCTCTGCGTGGACGAGACCCAGTTCT
TTGCTACGGGCTGGAGCTATGGCGGTGCCATGAGCCATAGCGTGGCTTGT
TCTCGGCCAGACGTCTTCAAGGCCGTCGCGGTCATCGCCGGGGCCCAGCT
GTCCGGCTGCGCCGGCGGCACGACGCCCGTGGCGTACCTAGGCATCCACG
GAGCCGCCGACAACGTCCTGCCCATCGACCTCGGCCGCCAGCTGCGCGAC
AAGTGGCTGCAGACCAACGGCTGCAACTACCAGGGCGCCCAGGACCCCGC
GCCGGGCCAGCAGGCCCACATCAAGACCACCTACAGCTGCTCCCGCGCGC
CCGTCACCTGGATCGGCCACGGGGGCGGCCACGTCCCCGACCCCACGGGC
AACAACGGCGTCAAGTTTGCGCCCCAGGAGACCTGGGACTTCTTTGATGC
CGCCGTCGGAGCGGCCGGCGCGCAGAGCCCGATGACATAA
(SEQ ID NO: 170)
MISVPALALALLAAVQVVESASAGCGKAPPSSGTKSMTVNGKQRQYILQL
PNNYDANKAHRVVIGYHWRDGSMNDVANGGFYDLRSRAGDSTIFVAPNGL
NAGWANVGGEDITFTDQIVDMLKNDLCVDETQFFATGWSYGGAMSHSVAC
SRPDVFKAVAVIAGAQLSGCAGGTTPVAYLGIHGAADNVLPIDLGRQLRD
KWLQTNGCNYQGAQDPAPGQQAHIKTTYSCSRAPVTWIGHGGGHVPDPTG
NNGVKFAPQETWDFFDAAVGAAGAQSPMT
(SEQ ID NO: 171)
ASAGCGKAPPSSGTKSMTVNGKQRQYILQLPNNYDANKAHRVVIGYHWRD
GSMNDVANGGFYDLRSRAGDSTIFVAPNGLNAGWANVGGEDITFTDQIVD
MLKNDLCVDETQFFATGWSYGGAMSHSVACSRPDVFKAVAVIAGAQLSGC
AGGTTPVAYLGIHGAADNVLPIDLGRQLRDKWLQTNGCNYQGAQDPAPGQ
QAHIKTTYSCSRAPVTWIGHGGGHVPDPTGNNGVKFAPQETWDFFDAAVG
AAGAQSPMT

### Example 1

### Wild-Type M. thermophila EG1b Gene Acquisition and Expression Vector Construction

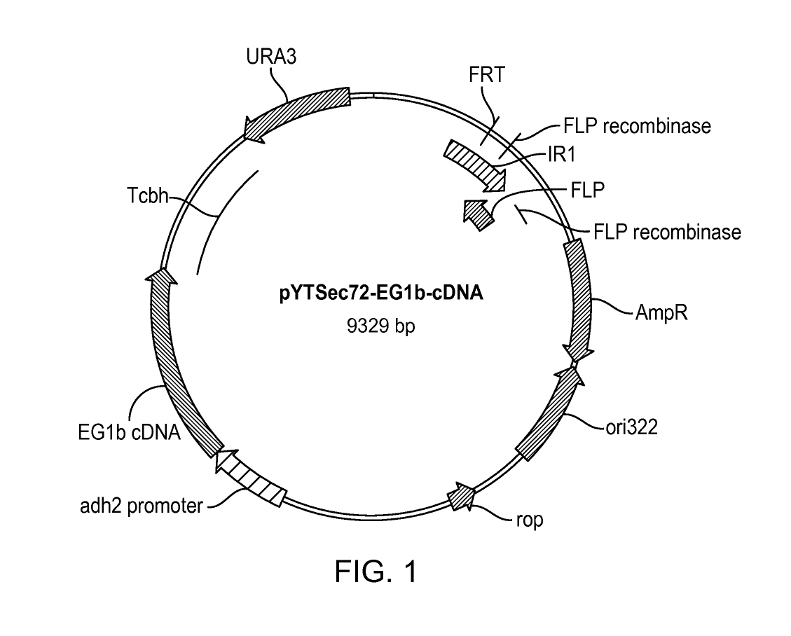

In this Example, production of an expression vector encoding the M. thermophila EG1b protein is described. cDNA coding the M. thermophila EG1b protein (“EG1b WT”; SEQ ID NO:1) was amplified from a cDNA library prepared using methods known in the art. Expression constructs were prepared in which the EG1b WT sequence was linked to its native signal peptide for secretion in M. thermophila. An EG1b cDNA construct was cloned into a pYTsec72 vector to create the vector pYTSec72-EG1b-cDNA, using standard methods known in the art. The vector includes EG1b and the native signal peptide of EG1b (See, FIG. 1).

Using standard methods known in the art, S. cerevisiae cells were transformed with the expression vector. Clones with correct EG1b sequences were identified and activity was confirmed using pNPL assay (4-Nitrophenyl beta-D-lactopyranoside; See, Example 3, infra).

### Example 2

### Production of M. thermophila EG1b

In this Example, production of the EB1b polypeptide is described. A single colony of S. cerevisiae containing a plasmid with the EG1b gene was inoculated into 3 ml of synthetic defined media (pH6.0) containing 60 g/L glucose, 6.7 g/L yeast nitrogen base without amino acids (Sigma Y0626), 3.06 g/L sodium phosphate (monobasic), 0.80 g/L sodium phosphate (dibasic), and 2 g/L amino acid drop-out mix minus uracil (USBio D9535). Cells were grown overnight (at least 16 hours) in an incubator at 30° C. with shaking at 250 rpm. Then, 0.5 ml of this culture was diluted into 50 ml of synthetic defined expression media (pH6.0) containing 20 g/L glucose, 6.7 g/L yeast nitrogen base without amino acids (Sigma Y0626), 3.06 g/L sodium phosphate (monobasic), 0.80 g/L sodium phosphate (dibasic), and 2 g/L amino acid drop-out mix minus uracil (USBio D9535). This was incubated for 72 hours and allowed to grow at 37° C. while shaking at 250 rpm. Cells were harvested by centrifugation (4000 rpm, 4° C., 15 minutes). The supernatant was decanted into a new tube and the activity of the WT EG1b was confirmed using the 4-Nitrophenyl beta-D-lactopyranoside (pNPL) assay described in Example 3.

### Example 3

### Assays

In this Example, assays used to determine EG1b activity are described. While certain pH and temperature conditions are exemplified, additional pH and temperature conditions find use in other assays (e.g., pH 5 and/or 55° C.).

1. 4-Nitrophenyl beta-D-Lactopyranoside (pNPL)

In a total volume of 300 μl, 30 n1 of 16 mM 4-Nitrophenyl beta-D-lactopyranoside (pNPL) in 100 mM sodium acetate (pH 4.5), and 40 μM of S. cerevisiae supernatant containing secreted EG1b protein was added to 230 μl of 100 mM sodium acetate, pH 4.5. The reaction was incubated for 20 hrs at 65° C., centrifuged briefly and 25 μl was transferred to 175 μl of 1 M Na₂CO₃ in a flat-bottom clear plate to terminate the reaction. The plate was mixed gently, then centrifuged for 1 min, and absorbance was measured at λ(lambda)=405 nm, with a Spectramax M2 (Molecular Devices). When a wild type EG1b produced as described in Example 2 was reacted with pNPL, the resulting mixture produced an absorbance of 0.40, while the negative control consisting of supernatant of S. cerevisiae containing empty vector produced an absorbance of 0.05 under the same reaction conditions.

2. AVICEL® Cellulose Assay

Activity on AVICEL® cellulose substrate (Sigma-Aldrich) was measured using a reaction mixture of 300 μl volume containing 30 mg of AVICEL® cellulose, 20 μl of supernatant produced as described in Examples 1 and 2, a glass bead, and 230 μl of 196 mM sodium acetate, pH 4.5. Beta-glucosidase, which converts cellobiose to glucose was subsequently added and conversion of Avicel to glucose was measured using a GOPOD assay. The reactions were incubated at 65° C. for 24 hours while shaking at 900 rpm, and then centrifuged. 160 μl of the supernatant was filtered using the Millipore filter plate (Millipore MSRL N4050). Then, 10 μl of the filtrate was added to 190 μl of the GOPOD mixture (Megazyme, containing glucose oxidase, peroxidase and 4-aminoantipyrine) and incubated at room temperature for 30 minutes. The amount of glucose was measured spectrophotometrically at 510 nm with a Spectramax M2 (Molecular Devices). The amount of glucose generated was calculated based on the measured absorbance at 510 nm and using the standard curve when the standards were measured on the same plate. When wild type EG1b produced as described in Examples 1 and 2 was tested in this assay, approximately 0.5 g/1 of glucose was produced. In some alternative embodiments, HPLC is used to detect cellobiose and glucose (without Bgl) or glucose (if coupled with Bgl).

3. Biomass Assay

Activity on pretreated wheat straw biomass substrate was measured using a reaction mixture containing 20 g/L of biomass, a total of 0.073% (with respect to glucan) protein mixture containing M. thermophila 25% of Cbh1a, 25% Cbh2b, 30% GH61, 10% EG2 and 10% EG1b protein (produced as described in Examples 1 and 2), and 81g/L xylose, in sodium acetate buffer, at pH 5. The reactions were incubated at 50° C. for 72 hours while shaking at 950 rpm, centrifuged and 50 μl of the reaction was added to 25 μl of a 25g/1 solution of A. niger β-glucosidase in 250 mM sodium acetate, pH 5. This reaction was incubated for 1.5 hours at 50° C. while shaking at 950 rpm to hydrolyze cellobiose to glucose. From this reaction, 30 μl was transferred to 170 μl of the GOPOD mixture (Megazyme, containing glucose oxidase, peroxidase and 4-aminoantipyrine) and incubated at room temperature for 20 minutes. The amount of glucose generated was measured spectrophotometrically at 510 nm with a Spectramax M2 (Molecular Devices). The amount of glucose generated was calculated based on the measured absorbance at 510 nm and using the standard curve when the standards were measured on the same plate. When wild type EG1b produced as described in Examples 1 and 2 was used in the described mixture and reaction, approximately 25 g/l of glucose was produced.

### Example 4

### Viscosity Reduction By EG1b

In this Example, experiments conducted to demonstrate the viscosity reduction properties of EG1b are described. Purified EG1b produced as described in Examples 1 and 2 was evaluated for reduction in cellulose chain length, thereby enabling a reduction in viscosity.

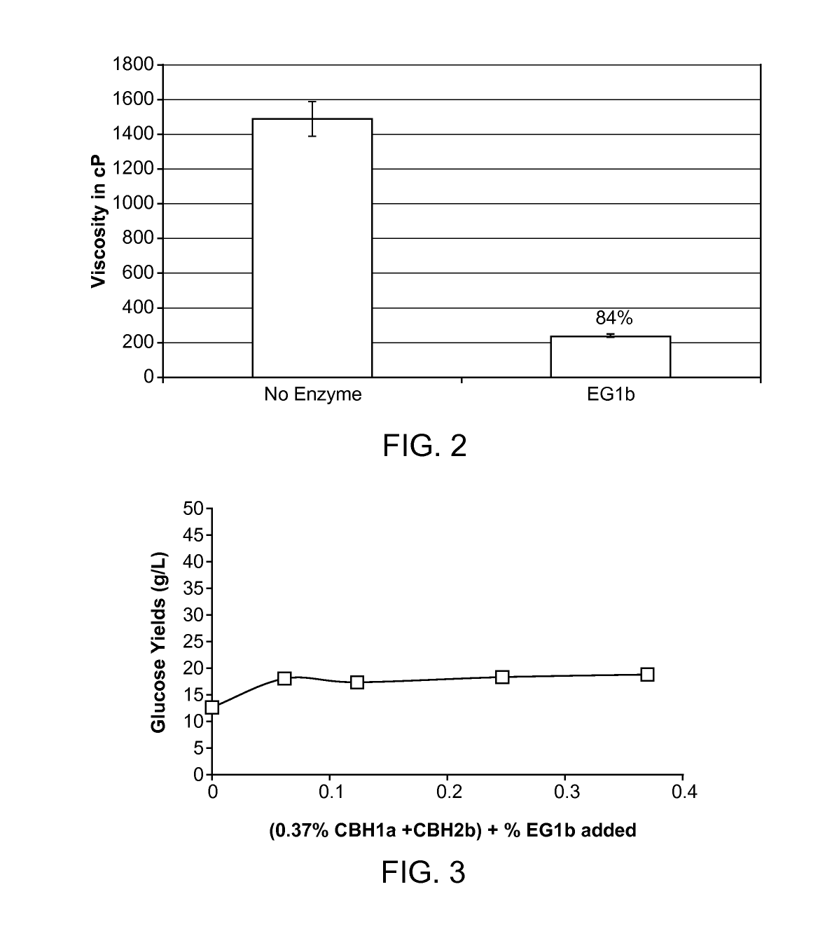

EG1b was tested for viscosity reduction by its action on unwashed pretreated wheat straw at glucan load of 75 g/L glucan and at pH 5.0, 55° C. The reactions were carried out in shake flasks for 72 hrs at a total weight of 50g. At 72 hrs, 16 g samples were transferred to the RVA-super4 viscometer (Newport). The viscosity was measured at end of 30 minutes at 30° C. FIG. 2 provides a graph showing the results. As indicated, addition of 0.09% EG1b in relation to glucan exhibited approximately 84% viscosity reduction at pH 5, 55° C.

### Example 5

### Use of EG1b Proteins to Promote Saccharification

The M. thermophila enzymes, CBH1a and CBH2b (1:1) at a protein load of 0.37% (w.r.t glucan) were combined with various concentrations of the EG1b protein to test the ability of the enzymes to convert glucan to glucose. The saccharification reactions were carried out at 93 g/L glucan load of pretreated wheat straw at pH 5.0 at a temperature of 55° C. for 24 hrs at 950 rpm in high throughput (HTP) 96 deep well plates, Excess (in relation to glucan) beta-glucosidase was also supplemented to relieve product inhibition from cellobiose. The individual enzymes were characterized by standard BCA assays for total protein quantification, as known in the art. Reactions were quenched by addition of 10 mM sulfuric acid. For glucose analysis, the samples were analyzed by HPLC using methods known in the art. The results indicated that addition of 0.062% EG1b with regard to glucan resulted in a 42% improvement in glucose yields, over enzyme mixtures of CBH1a and CBH2b without added EG1b.

### Example 6

### Addition of EG1b in a Minimal Enzyme Set

The M. thermophila enzymes, CBH1, CBH2, EG2, GH61a, EG1b, Bgl 1 were combined in two different proportions and tested for their ability to convert glucan to glucose. Culture supernatant from the strain CF-404 (a M. thermophila strain that comprises both cellulases and GH61 proteins) was also assayed for comparison. The saccharification reactions were carried out at 93 g/kg glucan load of unwashed pretreated wheat straw at pH 5.0 at a temperature of 55° C. at 250 rpm in a total weight of 30 g. The whole cellulase (broth from CF-404 cells), as well as the individual enzymes were characterized by standard BCA assays for total protein quantification, as known in the art. The total protein load was fixed to 0.81% (wt added protein/wt glucan). The proportions used were as follows for a total of 100%. As indicated in Table 6-1., only differences between the mixtures was the inclusion of EG1b in Mix 2 and its absence in Mix 1. As indicated in FIG. 4, the addition of EG1b improved saccharification yields by 28.7% over the control and 18.4% over Mix 1. Hence EG1b is an important component of the saccharification enzyme mix.

TABLE 6.1
Test Enzyme Mixtures

| Enzyme | Mix 1 | Mix 2 |
|---|---|---|
| CBH1a | 22.5 | 22.5 |
| CBH2b | 19.2 | 19.2 |
| EG2 | 3.3 | 3.3 |
| GH61a | 32.7 | 32.7 |
| EG1b | 0 | 12.3 |
| Bgl1 | 10 | 10 |

While particular embodiments of the present invention have been illustrated and described, it will be apparent to those skilled in the art that various other changes and modifications can be made without departing from the spirit and scope of the present invention. Therefore, it is intended that the present invention encompass all such changes and modifications with the scope of the present invention.

The present invention has been described broadly and generically herein. Each of the narrower species and subgeneric groupings falling within the generic disclosure also form part(s) of the invention. The invention described herein suitably may be practiced in the absence of any element or elements, limitation or limitations which is/are not specifically disclosed herein. The terms and expressions which have been employed are used as terms of description and not of limitation. There is no intention that in the use of such terms and expressions, of excluding any equivalents of the features described and/or shown or portions thereof, but it is recognized that various modifications are possible within the scope of the claimed invention. Thus, it should be understood that although the present invention has been specifically disclosed by some embodiments and optional features, modification and variation of the concepts herein disclosed may be utilized by those skilled in the art, and that such modifications and variations are considered to be within the scope of the present invention.

1. A cell comprising a recombinant nucleic acid sequence encoding (i) an endoglucanase 1b (EG1b) protein comprising SEQ ID NO:2 and (ii) an operably-linked heterologous promoter, wherein said cell further produces at least one recombinant cellulase protein selected from beta-glucosidases (BGLs), Type 1 cellobiohydrolases (CBH1s), Type 2 cellobiohydrolases (CBH2s), glycoside hydrolase 61s (GH61s), and/or endoglucanases (EGs).

2. The cell of claim 1 wherein the recombinant nucleic acid sequence comprises the nucleotide sequence set forth as SEQ ID NO:1.

3. The cell of claim 1, wherein said cell produces at least one recombinant cellulase protein selected from Myceliophthora thermophila endoglucanases (EGs), beta-glucosidases (BGLs), Type 1 cellobiohydrolases (CBH1s), Type 2 cellobiohydrolases (CBH2s), and/or glycoside hydrolase 61s (GH61s), and/or variants of said cellulase proteins.

4. (canceled)

5. The cell of claim 1, wherein said cell produces at least two, at least three, at least four or at least five recombinant cellulases.

6. The cell of claim 1, wherein said cell is a prokaryotic cell.

7-8. (canceled)

9. A composition comprising an EG1b protein comprising SEQ ID NO:2, and one or more cellulases selected from endoglucanases (EGs), beta-glucosidases (BGLs), Type 1 cellobiohydrolases (CBH1s), Type 2 cellobiohydrolases (CBH2s), and/or glycoside hydrolase 61s (GH61s), and/or variants of said cellulase proteins.

10-13. (canceled)

14. The composition of claim 9, wherein the GH61, CBH1, CBH2, EG, and/or BGL, are contained in a cell culture broth.

15. A recombinant nucleic acid sequence encoding a protein comprising SEQ ID NO:2.

16. (canceled)

17. The recombinant nucleic acid sequence of claim 15, wherein the protein-encoding sequence is operably linked to a heterologous signal sequence and/or a heterologous promoter.

18. The recombinant nucleic acid sequence of claim 15, comprising SEQ ID NO:1.

19. A vector comprising the recombinant nucleic acid of claim 15.

20. The vector of claim 19, further comprising at least one polynucleotide sequence encoding at least one EG, BGL, CBH1, CHB2, and/or GH61 protein.

21. A host cell comprising the vector of claim 19, and wherein said host cell produces at least one recombinant cellulase protein selected from EGs, BGLs, CBH1s, CBH2s, and GH61s.

22-23. (canceled)

24. The host cell of claim 21, wherein said cell is a prokaryotic cell.

25-27. (canceled)

28. A method for saccharification comprising (a) culturing a cell according claim 1, under conditions in which the EG1b protein is secreted into a culture broth, and (b) combining the broth and a biomass under conditions in which saccharification occurs, where (a) may take place before or simultaneously with (b).

29-30. (canceled)

31. A method for reducing viscosity during saccharification reactions comprising providing EG1b in a saccharification reaction mixture under conditions such that the viscosity of the saccharification reaction mixture is less viscous than a saccharification reaction mixture without said EG1b.

32. The method of claim 31, wherein said sachharification reaction mixture comprises at least one additional enzyme selected from CBH1, CBH2, BGL, EG2, and GH61.

33. The method of claim 31, wherein said saccharification reaction mixture does not comprise EG2.
